(19) United States
(12) Patent Application Publication
(10) Pub. No.: US 2004/0087653 A1
(43) Pub. Date: May 6, 2004

(54) Methods for the treatment of respiratory diseases and conditions with a selective iNOS inhibitor and a PDE inhibitor and compositions therefor

(76) Inventor: Pamela T. Manning, Chesterfield, MO (US)

Correspondence Address: Pharmacia Corporation, Corporate Patent Department, P.O. Box 1027, Chesterfield, MO, 63006, US

(21) Appl. No.: 10/439,679

(22) Filed: May 16, 2003

Related U.S. Application Data
(60) Provisional application No. 60/381,056, filed on May 16, 2002.

(51) Int. Cl. A61K 31/198

(52) U.S. Cl. 514/562

(57) ABSTRACT

Therapeutic methods for the prevention and treatment of respiratory diseases or conditions are described, the methods including administering to a subject in need thereof a respiratory disease or condition effective amount of a selective inhibitor of inducible nitric oxide synthase.

The present application claims priority under Title 35, United States Code, §119 of the U.S. Provisional application Serial No. 60/381,056, filed May 16, 2003.

### BACKGROUND OF THE INVENTION

The present invention relates in general to methods of medical treatment using selective inhibitors of the inducible form of nitric oxide synthase (iNOS) and inhibitors of phosphodiesterase (PDE), and more particularly to novel methods useful in the medical prevention and treatment of respiratory diseases and conditions including asthmatic conditions as well as the lung diseases referred to collectively as chronic obstructive pulmonary disease (COPD), and compositions therefor. Asthma affects about 150 million people world-wide and is the most prevalent chronic disease in childhood. High prevalence of childhood asthma observed during the last decades predicts the growing prevalence of asthma in the near future unless appropriate preventive measures are undertaken. Asthma affects about 10 million Americans, about a third of whom are under 18 years of age. In the United States alone billions of dollars are spent annually on asthma-related health care. The episodic breathing difficulty that characterizes asthma is brought about by a combination of three primary factors including 1) bronchospasm, that is to say, variable and reversible airway obstruction due to airway muscle contraction, 2) inflammation of the airway lining, and 3) bronchial hyper-responsiveness that results in excessive mucus in the airways. Triggers of asthma attacks vary among individuals, but include allergens such as dust mites and mold, environmental pollutants, viral agents, and physical exertion or exercise.

World estimates of COPD have been traditionally based primarily on mortality statistics. These provide misleading figures because COPD is under-diagnosed and often not listed either as a primary or contributory cause of death. Estimates of prevalence require measurement of airflow obstruction. Consequently, few countries have good population-based data on COPD prevalence. Nevertheless, estimates show death and disability due to COPD are increasing across most regions for males and females. The Mayo Clinic reports that chronic obstructive pulmonary disease (COPD), mostly emphysema or chronic bronchitis, kills 85,000 people a year in the United States. Chronic obstructive pulmonary disease actually refers collectively to several chronic or progressive lung diseases including asthmatic bronchitis, chronic bronchitis (with normal airflow), chronic obstructive bronchitis, bullous disease, and emphysema, all involving inflammation. For example, chronic bronchitis is an inflammation and eventual scarring of the lining of the bronchial tubes producing symptoms including chronic cough, increase of mucus, frequent clearing of the throat and shortness of breath. Emphysema results from the normal but chronic inflammatory response of the airway lining to chronic exposure to environmental pollutants such as cigarette smoke.

Drug treatment for asthma and COPD includes intravenous, oral, subcutaneous or inhaled administration of bronchodilators including beta-adrenergics, methyl xanthines, and anti-cholinergics, and also administration of corticosteroids, the mast cell mediator-release inhibitors known as Cromolyn and Tilade, or, more recently, anti-leukotrienes, for anti-inflammatory effects. However, the cellular and molecular mechanisms of inflammatory and immune processes that play a role in the pathogenesis and progression of asthma and COPD are not yet well understood.

Nitric oxide (NO) is a bioactive free radical gas produced by any one of several isoforms of the enzyme nitric oxide synthase (NOS). The physiological activity of what was later identified as NO was initially discovered in the early 1980's when it was found that vascular relaxation caused by acetylcholine is dependent on the presence of the vascular endothelium. The factor derived from the endothelium, then called endothelium-derived relaxing factor (EDRF), that mediates such vascular relaxation is now known to be NO that is generated in the vascular endothelium by one isoform of NOS. The activity of NO as a vasodilator has been known for well over 100 years. In addition, NO is the active species derived from known nitrovasodilators including amylnitrite, and glyceryltrinitrate. Nitric oxide is also an endogenous stimulator of soluble guanylate cyclase and thus stimulates cGMP production. When NOS is inhibited by N-monomethylarginine (L-NMMA), cGMP formation is completely prevented. In addition to endothelium-dependent relaxation, NO is known to be involved in a number of biological actions including cytotoxicity of phagocytic cells and cell-to-cell communication in the central nervous system.

The identification of EDRF as NO coincided with the discovery of a biochemical pathway by which NO is synthesized from the amino acid L-arginine by the enzyme NO synthase. There are at least three types of NO synthase as follows:

(i) a constitutive, Ca++/calmodulin dependent enzyme, located in the endothelium, that releases NO in response to receptor or physical stimulation.

(ii) a constitutive, Ca++/calmodulin dependent enzyme, located in the brain, that releases NO in response to receptor or physical stimulation.

(iii) a Ca++independent enzyme, a 130 kD protein, which is induced after activation of vascular smooth muscle, macrophages, endothelial cells, and a number of other cells by endotoxin and cytokines. Once expressed this inducible nitric oxide synthase (hereinafter “iNOS”) generates NO continuously for long periods.

Clinical studies have shown that NO production and iNOS expression are increased in a variety of chronic inflammatory diseases, such as rheumatoid and osteoarthritis, and iNOS is implicated as a major pathological factor in these chronic inflammatory diseases.

Thus, inhibition of excessive NO production by iNOS is likely to be anti-inflammatory. However, the production of NO from eNOS and nNOS is involved in normal physiology, and therefore any NOS inhibitor that is used for treating inflammation should be selective for iNOS so that normal physiological modulation of blood pressure by eNOS-generated NO, and non-adrenergic, non-cholinergic neuronal transmission by nNOS-generated NO remains unaffected.

Asthmatics and others with inflammatory airway disease exhibit an increased concentration of NO in exhaled air relative to normals, and exhaled NO has been proposed as a marker of airway inflammation. Increased expression of iNOS is observed in the epithelium of asthmatics and in lung macrophages in bronchiectasis. See, e.g., Barnes, P. J. and Liew, F. Y., Immunol. Today 16(3):128-30 (1995).

Overproduction of NO by iNOS has been implicated in the pathogenesis of the airway inflammation of asthma. See, e.g., Eissa, N. T. et al., Am. J. Resp. Cell and Mol. Biol. 24(5): 616-20 (2001). In a murine model of allergic asthma, administration of one of the NOS inhibitors L-NAME, S-ethylisothiourea, or 2-amino 5,6-dihydro 6-methyl 4H-1,3-thiazine suppressed airway inflammation by down-regulation of chemokine expression. See Trifilieff, A., et al., J. Immunol. 165(3) 1526-33 (2000). It has been suggested that therapeutic strategies for asthma as well as rhinitis might include selective inhibition of iNOS with aminoguanidine. See Schapowal, A. G., and Brunnenkant, W., Allergologie 19(1):49 (1996). Sustained production of high levels of iNOS-generated NO is thought to underlie disruption of airway endothelium, diminished ciliary function, shift in balance from a TH-1-dominated response to a TH-2-dominated response, and further thought to provide a chemoattractant for eosinophils, suggesting that selective inhibition of iNOS in asthma will result in decreased pulmonary inflammation and improved airway function. See Manning, P. T. et al., Prog. in Resp. Res. 31:156-59 (2001).

PCT Patent Application WO 01/05748 discloses new oligomeric amino acid derivatives as being useful selective iNOS inhibitors for the treatment of autoimmune or inflammatory conditions, including asthma.

Inhibition of nuclear factor-kappaB (NF-kB) activity has been also described for treating asthma, diabetic vascular disease, heart failure, and sepsis, in which heparin is administered to the patient to block translocation of NF-kB from the cellular cytoplasm to the nucleus, thereby inhibiting NF-kB expression. See PCT Publication 01/019376. Proteins believed to be subject to NF-kB-dependent gene expression include the cytokines THF, IL-1, IL-2, IL-6, IL-8, interferon-beta, interferon-gamma, tissue factor-1, complement, and iNOS. Id.

However, the cellular and molecular mechanisms underlying asthma and COPD are not yet well understood. In contrast to suggestions that such respiratory ailments be treated by inhibiting NOS activity, it has also been suggested that the increased concentration of NO observed in the exhaled air of asthmatics and others with pulmonary disease is an indicator of a compensatory mechanism involving NOS activity. Accordingly, in contrast to proposed treatments of asthma or COPD involving inhibition of NOS activity, it has also been suggested that treatment methods involving the administration of compounds that instead donate, transfer or release nitric oxide, or that stimulate endogenous production of NO are indicated. See, e.g., U.S. Pat. Nos. RE37,116, and 6,331,543.

Other work teaches away from treating asthma or COPD with iNOS inhibitors. The conversion of GTP to cGMP by guanyl cyclase is thought to be stimulated by iNOS, so that selective inhibition of iNOS should result in decreased guanylate cyclase activity and decreased levels of cGMP. However, U.S. Pat. No. 6,333,354 teaches treatment of acute or chronic obstruction of bronchi or acute or chronic inflammation, including asthma, using combinations of PDE inhibitors with guanyl cyclase agonists. Guanyl cyclase agonists would be expected to have the opposite effect of iNOS inhibitors, resulting in increased guanyl cyclase activity and increased production of cGMP, instead of decreased levels of cGMP.

Phosphodiesterase (PDE) is involved in numerous functional pathways in tissues throughout the body. Agents such as theophylline and caffeine have been recognized as non-specific PDE inhibitors for several decades. See GOODMAN & GILMAN's THE PHARMACOLOGICAL BASIS OF THERAPEUTICS, 832-4, (Joel G. Hardman et al. eds., 9^{th} ed. 1996). More recently, classes of PDE inhibitors exhibiting more or less specificity for one or more of the mutiple isoforms of PDE have been described, and produce function-specific effects. For example, PDE-III specific inhibitors produce vascular and airway dilation, inhibition of platelet aggregation, stimulation of lipolysis, and inhbition of cytokine production. Id. PDE-IV specific inhibitors produce airway smooth muscle relaxation, inhbition of inflammatory mediator release, CNS modulation, and gastric acid secretion. Id.

However, known methods that involve PDE inhibitors in the treatment of asthma and COPD have involved compounds that would have effects opposed to those produced by iNOS inhibition. U.S. Pat. No. 6,333,354, described supra, teaches treatment of acute or chronic obstruction of bronchi or acute or chronic inflammation, including asthma, using combinations of PDE inhibitors with guanyl cyclase agonists that would enhance cGMP levels rather than reduce cGMP. Others have described methods and compositions involving nitrosated and nitrosylated PDE inhibitors that enhance rather than reduce endogenous levels of NO for the treatment of, inter alia, asthma, bronchitis and COPD. See, for example, U.S. Pat. No. 6,331,543.

Against this background, increasing interest has developed in finding novel agents and methods for the treatment and prevention of various pulmonary and respiratory diseases and conditions involving inflammation and airway obstruction that may be related to an excess of iNOS activity and PDE activity, and further for improved overall treatment efficacy with minimal toxicity and adverse side effects. It would therefore be advantageous to find and describe new compositions and therapeutic methods for treating and preventing inflammation-related lung diseases and conditions.

### SUMMARY OF THE INVENTION

A method for the treatment, prevention or inhibition of a respiratory disease or condition in a subject in need of such treatment, prevention or inhibition, comprising administering an iNOS inhibitor or pharmaceutically acceptable salt or prodrug thereof and a phosphodiesterase (PDE) inhibitor or pharmaceutically acceptable salt or prodrug thereof, and compositions therefor, are described.

In an exemplary embodiment, the iNOS inhibitor is any inhibitor selective for the iNOS isoform of NOS.

The PDE inhibitor or pharmaceutically acceptable salt or prodrug thereof is any PDE inhibitor including isozyme-selective inhibitors of PDE-I, PDE-II, PDE-III, PDE-IV, PDE-V, PDE-VI and PDE-VII, and also PDE-III/IV dual inhibitors. In an exemplary embodiment, the PDE inhbitor is a PDE-III or a PDE-IV inhibitor.

The respiratory disease or condition is selected from the group consisting of asthmatic conditions and COPD including allergen-induced asthma, exercise-induced asthma, pollution-induced asthma, cold-induced asthma, viral-induced-asthma, chronic bronchitis with normal airflow, chronic obstructive bronchitis, emphysema, asthmatic bronchitis, bullous disease, cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, post-cardiac surgery, acute pulmonary hypertension, persistent pulmonary hypertension of the newborn, perinatal aspiration syndrome, hyaline membrane disease, acute pulmonary thromboembolism, heparin—

The iNOS selective inhibitor or pharmaceutically acceptable salt or prodrug thereof and the PDE inhibitor or pharmaceutically acceptable salt or prodrug thereof are administered to the subject orally, by inhalation, enterally or parenterally in at least one dose per day, either substantially simultaneously, or sequentially.

In another embodiment, the invention is directed toward a method for the treatment, prevention or inhibition of a respiratory disease or condition having an inflammatory component in a subject in need of such treatment, prevention or inhibition, the method comprising administering to the subject a dose of an iNOS selective inhibitor or pharmaceutically acceptable salt or prodrug thereof and a dose of a PDE inhibitor or pharmaceutically acceptable salt or prodrug thereof, wherein together the dose of the iNOS selective inhibitor or pharmaceutically acceptable salt or prodrug thereof and the dose of the PDE inhibitor or pharmaceutically acceptable salt or prodrug thereof constitute a therapeutically effective dose for the treatment, prevention or inhibition of the respiratory disease or condition.

The invention is also directed toward a composition for the treatment, prevention or inhibition of a respiratory disease or condition in a subject in need of such treatment, prevention or inhibition comprising an amount of an iNOS selective inhibitor or pharmaceutically acceptable salt or prodrug thereof and an amount of a PDE inhibitor or pharmaceutically acceptable salt or prodrug thereof.

The invention is also directed toward a kit for treating, preventing or inhibiting a respiratory disease or condition in a subject in need of such treatment, prevention or inhibition, the kit including a first dosage form including an iNOS selective inhibitor or pharmaceutically acceptable salt or prodrug thereof, and a second dosage form including a PDE inhibitor or pharmaceutically acceptable salt or prodrug thereof, wherein together the dosages comprise a therapeutically effective amount of the iNOS selective inhibitor or pharmaceutically acceptable salt or prodrug thereof and the PDE inhibitor or pharmaceutically acceptable salt or prodrug thereof for the treatment, prevention or inhibition of the respiratory disease or condition.

### BRIEF DESCRIPTION OF THE DRAWINGS

FIG. 1 is a graph of media nitrate content after human primary airway epithelial cells were cultured for 24 h in the presence of 50 ng/ml IL-1 β, TNF-α and IFN-γ (cyt) in the presence or absence of L-NIL;

FIG. 2 shows results of resolution of cellular proteins 3-8% tris-acetate polyacrylamide gels and immunoblot for iNOS protein;

FIG. 3 shows change in exhaled breath nitric oxide (NO) levels following oral administration of (A) 20 mg of an iNOS selective inhibitor (compund NN) and (B) 200 mg of compound NN in patients with mild-to-moderate asthma (closed triangles) compared with placebo (open triangles) and in healthy subjects (closed circles) compared with placebo (open circles); and

FIG. 4 shows the effects of oral administration compound NN on FEV₁, blood pressure and heart rate.

### DETAILED DESCRIPTION OF INVENTION

The following detailed description is provided to aid those skilled in the art to practice the present invention. However, this detailed description should not be construed to unduly limit the present invention, inasmuch as modifications and variations in the exemplary embodiments discussed herein can be made by those of ordinary skill in the art without departing from the scope of the appended claims.

The contents of each of the primary references cited herein, including the contents of the references cited within the primary references, are herein incorporated by reference in their entirety.

The present invention encompasses therapeutic methods using a selective iNOS inhibitor and a phosphodiesterase (PDE) inhibitor to treat, prevent or inhibit a respiratory disease or condition, and compositions therefor. The compositions and methods are for use in medicine for preventing, treating or inhibiting a respiratory disease or condition including: asthmatic conditions including allergen-induced asthma, exercise-induced asthma, pollution-induced asthma, cold-induced asthma, and viral-induced-asthma, chronic obstructive pulmonary diseases including chronic bronchitis with normal airflow, chronic bronchitis with airway obstruction (chronic obstructive bronchitis), emphysema, asthmatic bronchitis, and bullous disease, and other pulmonary diseases involving inflammation including cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, post-cardiac surgery, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus and hypoxia.

### a. Definitions

The terms “nitric oxide synthase” and “NOS” as used interchangeably herein refer to any of the isoforms of isoforms of the enzyme nitric oxide synthase, including eNOS, nNOS and iNOS.

The terms “inducible nitric oxide synthase,” “NOS-2” and “iNOS” as used interchangeably herein refer to the Ca⁺²-independent, inducible isoform of the enzyme nitric oxide synthase.

The terms “nitric oxide synthase inhibitor” and “NOS inhibitor” as used interchangeably herein denote a compound that reduces the physiological effect of a nitric oxide synthase enzyme. Such an inhibitor may be selective for a particular isoform of nitric oxide synthase, or may be substantially non-selective, that is, effective to a large extent on two or more isoforms of nitric oxide synthase.

The terms “selective nitric oxide synthase inhibitor” and “selective NOS inhibitor denote a compound capable of reducing the physiological effect of a particular isoform of nitric oxide synthase preferentially over other isoforms of nitric oxide synthase.

The terms “selective inducible nitric oxide synthase inhibitor,” “selective NOS-2 inhibitor,” and “selective iNOS inhibitor” denote a compound capable of reducing the physiological effect of the calcium ion independent isoform of nitric oxide synthase preferentially over other isoforms of nitric oxide synthase. In one embodiment, a selective iNOS inhibitor produces the selective inhibition of iNOS compared to either endothelial NOS or neuronal NOS such that in vivo administration results in efficacy (ED₅₀) of less than 100 mg/kg. In another embodiment, a selective iNOS inhibitor produces the selective inhibition of iNOS compared to either endothelial NOS or neuronal NOS such that in vivo administration results in efficacy (ED₅₀) of less than 10 mg/kg in a rodent endotoxin model). In a further embodiment, an iNOS inhibitor exhibits selectivity of about 20-fold with respect to eNOS as measured by elevation in mean arterial blood pressure. In yet another embodiment, an iNOS inhibitor exhibits 100-fold or greater selectivity fold with respect to eNOS as measured by elevation in mean arterial blood pressure. In still another embodiment, an iNOS inhibitor exhibits selectivity of at about 20-fold with respect to nNOS as measured by reductions in gastrointestinal transit or penile erection. In another embodiment, an iNOS inhibitor exhibits about 100-fold or greater selectivity with respect to nNOS as measured by reductions in gastrointestinal transit or penile erection.

The terms “phosphodiestrease inhibitor” and “PDE inhibitor” as used interchangeably herein denote a compound that reduces the physiological effect of a phosphodisterase enzyme, thus slowing the degradation of cyclic AMP (cAMP) and cyclic (cGMP). Such an inhibitor may be specific (that is, selective) for a particular isozyme of phosphodiesterase, or may be substantially non-specific (non-selective), that is, effective to a large extent on two or more isoforms of phosphodiesterase.

The term “PDE-I inhibitor” denotes a compound that is capable of reducing the physiological effect of the PDE-I isoform of phosphodiesterase preferentially over other isoforms of phosphodiesterase.

The term “PDE-II inhibitor” denotes a compound that is capable of reducing the physiological effect of the PDE-II isoform of phosphodiesterase preferentially over other isoforms of phosphodiesterase.

The term “PDE-III inhibitor” denotes a compound that is capable of reducing the physiological effect of the PDE-III isoform of phosphodiesterase preferentially over other isoforms of phosphodiesterase.

The term “PDE-IV inhibitor” denotes a compound that is capable of reducing the physiological effect of the PDE-IV isoform of phosphodiesterase preferentially over other isoforms of phosphodiesterase.

A PDE IV inhibitor may show different in vitro IC₅₀ values with respect to different isoforms of PDE. The in vitro IC₅₀ value exhibited by a compound for the inhibition of another isoform of PDE (herein, “PDE Z) divided by the IC₅₀ value for the inhibition of PDE IVis referred to herein as “inter-isoform selectivity” with respect to that other PDE isoform.

The term “inter-isoform selective PDE IV inhibitor” refers to a PDE IV inhibitor for which its inter-isoform selectivity with respect to another PDE isoform is greater than one.

It is believed that there are at least two binding forms on human monocyte recombinant PDE IV (human PDE IV) at which inhibitors bind. One explanation for these observations is that human PDE IV exists in two distinct forms. One binds rolipram with high affinity while the other binds rolipram with low affinity. Herein we distinguish these forms by referring to them as the high affinity rolipram binding form (HPDE IV) and the low affinity binding form (LPDE IV). It has been reported that certain compounds which potently compete for HPDE IV have more side effects or more intense side effects than those which more potently compete with LPDE IV (see, for example, U.S. Pat. No. 5,998,428, herein incorporated by reference). Further data indicate that compounds can be targeted to the low affinity binding form of PDE IV and that this form is distinct from the binding form for which rolipram is a high affinity binder. Compounds that interact with LPDE IV are reported to have anti-inflammatory activity, whereas those that interact with the HPDE IV produce side effects or exhibit more intensely those side effects. Rolipram binds to one catalytic site of one form with a high affinity (HPDE IV), defined herein as having a Kᵢ less than 10 nanomolar, and to the other form with a low affinity (LPDE IV), defined here as having a Kᵢ of greater than 100 nanomolar. U.S. Pat. No. 5,998,428 describes a method of measuring the in vitro IC₅₀ ratios for a compound with respect to HPDE IV and LPDE IV.

As used herein, the term “intra-isoform selectivity” with respect to a particular compound refers to its in vitro IC₅₀ with respect to HPDE IV divided by its in vitro IC₅₀ with respect to LPDE IV.

The term “intra-isoform selective PDE IV inhibitor” means a PDE IV inhibitor for which the intra-isoform selectivity is about 0.1 or greater.

The term “PDE-V inhibitor” denotes a compound that is capable of reducing the physiological effect of the PDE-V isoform of phosphodiesterase preferentially over other isoforms of phosphodiesterase.

The term “PDE-VI inhibitor” denotes a compound that is capable of reducing the physiological effect of the PDE-VI isoform of phosphodiesterase preferentially over other isoforms of phosphodiesterase.

The term “PDE-VII inhibitor” denotes a compound that is capable of reducing the physiological effect of the PDE-VII isoform of phosphodiesterase preferentially over other isoforms of phosphodiesterase.

The term “PDE-III/IV dual inhibitor” denotes a compound that is capable of reducing the physiological effect of the PDE-III and PDE-IV isoforms of phosphodiesterase preferentially over other isoforms of phosphodiesterase.

The term “alkyl”, alone or in combination, means an acyclic alkyl radical, linear or branched, preferably containing from 1 to about 10 carbon atoms and more preferably containing from 1 to about 6 carbon atoms. “Alkyl” also encompasses cyclic alkyl radicals containing from 3 to about 7 carbon atoms, preferably from 3 to 5 carbon atoms. Said alkyl radicals can be optionally substituted with groups as defined below. Examples of such radicals include methyl, ethyl, chloroethyl, hydroxyethyl, n-propyl, isopropyl, n-butyl, cyanobutyl, isobutyl, sec-butyl, tert-butyl, pentyl, aminopentyl, isoamyl, hexyl, octyl and the like.

The term “alkenyl” refers to an unsaturated, acyclic hydrocarbon radical, linear or branched, in so much as it contains at least one double bond. Such radicals containing from 2 to about 6 carbon atoms, preferably from 2 to about 4 carbon atoms, more preferably from 2 to about 3 carbon atoms. Said alkenyl radicals may be optionally substituted with groups as defined below. Examples of suitable alkenyl radicals include propenyl, 2-chloropropylenyl, buten-1-yl, isobutenyl, penten-1-yl, 2-methylbuten-1-yl, 3-methylbuten-1-yl, hexen-1-yl, 3-hydroxyhexen-1-yl, hepten-1-yl, and octen-1-yl, and the like.

The term “alkynyl” refers to an unsaturated, acyclic hydrocarbon radical, linear or branched, in so much as it contains one or more triple bonds, such radicals containing 2 to about 6 carbon atoms, preferably from 2 to about 4 carbon atoms, more preferably from 2 to about 3 carbon atoms. Said alkynyl radicals may be optionally substituted with groups as defined below. Examples of suitable alkynyl radicals include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl radicals and the like.

The term “alkoxy” embraces linear or branched oxy-containing radicals each having alkyl portions of 1 to about 6 carbon atoms, preferably 1 to about 3 carbon atoms, such as a methoxy radical. The term “alkoxyalkyl” also embraces alkyl radicals having one or more alkoxy radicals attached to the alkyl radical, that is, to form monoalkoxyalkyl and dialkoxyalkyl radicals. Examples of such radicals include methoxy, ethoxy, propoxy, butoxy and tert-butoxy alkyls. The “alkoxy” radicals may be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylthio” embraces radicals containing a linear or branched alkyl radical, of 1 to about 6 carbon atoms, attached to a divalent sulfur atom. An example of “lower alkylthio” is methylthio (CH₃—S—).

The term “alkylthioalkyl” embraces alkylthio radicals, attached to an alkyl group. Examples of such radicals include methylthiomethyl.

The term “halo” means halogens such as fluorine, chlorine, bromine or iodine atoms.

The term “heterocyclyl” means a saturated or unsaturated mono- or multi-ring carbocycle wherein one or more carbon atoms is replaced by N, S, P, or O. This includes, for example, the following structures:

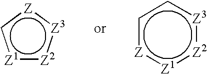

wherein Z, Z¹, Z² or Z³ is C, S, P, O, or N, with the proviso that one of Z, Z¹, Z² or Z³ is other than carbon, but is not 0 or S when attached to another Z atom by a double bond or when attached to another O or S atom. Furthermore, the optional substituents are understood to be attached to Z, Z¹, Z² or Z³ only when each is C. The term “heterocyclyl” also includes fully saturated ring structures such as piperazinyl, dioxanyl, tetrahydrofuranyl, oxiranyl, aziridinyl, morpholinyl, pyrrolidinyl, piperidinyl, thiazolidinyl, and others. The term “heterocyclyl” also includes partially unsaturated ring structures such as dihydrofuranyl, pyrazolinyl, imidazolinyl, pyrrolinyl, chromanyl, dihydrothiophenyl, and others.

The term “heteroaryl” means a fully unsaturated heterocycle.

In either “heterocycle” or “heteroaryl,” the point of attachment to the molecule of interest can be at the heteroatom or elsewhere within the ring.

The term “cycloalkyl” means a mono- or multi-ringed carbocycle wherein each ring contains three to about seven carbon atoms, preferably three to about five carbon atoms. Examples include radicals such as cyclopropyl, cyclobutyl, cyclopentyl, cyclohexyl, cycloalkenyl, and cycloheptyl. The term “cycloalkyl” additionally encompasses spiro systems wherein the cycloalkyl ring has a carbon ring atom in common with the seven-membered heterocyclic ring of the benzothiepine.

The term “oxo” means a doubly bonded oxygen.

The term “alkoxy” means a radical comprising an alkyl radical that is bonded to an oxygen atom, such as a methoxy radical. More preferred alkoxy radicals are “lower alkoxy” radicals having one to about ten carbon atoms. Still more preferred alkoxy radicals have one to about six carbon atoms. Examples of such radicals include methoxy, ethoxy, propoxy, isopropoxy, butoxy and tert-butoxy.

The term “aryl” means a fully unsaturated mono- or multi-ring carbocycle, including, but not limited to, substituted or unsubstituted phenyl, naphthyl, or anthracenyl.

The phrase “optionally substituted” means that the indicated radical may, but need not be substituted for hydrogen. Thus, the phrase “optionally substituted by one or more” means that if a substitution is made at the indicated moiety, more than one substitution is contemplated as well. In this regard, if more than one optional substituent exists, either substituent may be selected, or a combination of substituents may be selected, or more than one of the same substituent may be selected. By way of example, and not limitation, the phrase “C₁-C₅ alkyl optionally substituted by one or more halo or alkoxy” should be taken to mean, for example, that methyl, ethyl, propyl, butyl, or pentyl may have at all substitutable positions: hydrogen, fluorine, chlorine or other halogen, methoxy, ethoxy, propoxy, iso butoxy, tert-butoxy, pentoxy or other alkoxy radicals, and combinations thereof. Non-limiting examples include: propyl, iso-propyl, methoxypropyl, fluoromethyl, fluoropropyl, 1-fluoro-methoxymethyl and the like.

When a compound is described by both a structure and a name, the name is intended to correspond to the indicated structure, and similarly the structure is intended to correspond with the indicated name.

The term “subject” as used herein refers to an animal, in one embodiment a mammal, and in an exemplary embodiment particularly a human being, who is the object of treatment, observation or experiment.

The terms “dosing” and “treatment” as used herein refer to any process, action, application, therapy or the like, wherein a subject, particularly a human being, is rendered medical aid with the object of improving the subject's condition, either directly or indirectly.

The term “therapeutic compound” as used herein refers to a compound useful in the prophylaxis or treatment of a respiratory disease or condition.

The term “therapeutically effective” as used herein refers to a characteristic of an amount of a therapeutic compound, or a characteristic of amounts of combined therapeutic compounds in combination therapy. The amount or combined amounts achieve the goal of preventing, avoiding, reducing or eliminating the respiratory disease or condition.

The term “prodrug” refers to a compound that is a drug precursor which, following administration to a subject and subsequent absorption, is converted to an active species in vivo via some process, such as a metabolic process. Other products from the conversion process are easily disposed of by the body. The more preferred prodrugs are those involving a conversion process that produces products that are generally accepted as safe.

The term “combination therapy” means the administration of two or more therapeutic agents to treat a condition. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the condition.

The term “asthma” refers to a respiratory disorder characterized by episodic difficulty in breathing brought on by any one or a combination of three primary factors including 1) bronchospasm, i.e. variable and reversible airway obstruction due to airway muscle contraction, 2) inflammation of the airway lining, and 3) bronchial hyper-responsiveness resulting in excessive mucus in the airways, which may be triggered by exposure to an allergen or combination of allergens such as dust mites and mold, viral or bacterial infection especially infection with a “common cold” virus, environmental pollutants such as chemical fumes or smoke, physical over exertion such as during exercise, stress, or inhalation of cold air.

The term “asthmatic condition” refers to the characteristic of an individual to suffer from an attack of asthma upon exposure to any one or a number of asthma triggers for that individual. An individual may be characterized as suffering from, for example, allergen-induced asthma, exercise-induced asthma, pollution-induced asthma, viral-induced asthma or cold-induced asthma.

The terms “chronic obstructive pulmonary disease” and “COPD” as used interchangeably herein refers to a chronic disorder or combination of disorders characterised by reduced maximal expiratory flow and slow forced emptying of the lungs that does not change markedly over several months and is not, or is only minimally, reversible with traditional bronchodilators. Most commonly, COPD is a combination of chronic bronchitis, i.e. the presence of cough and sputum for more than three months for about two consecutive years, and emphysema, i.e. alveolar damage. However, COPD can involve chronic bronchitis with normal airflow, chronic bronchitis with airway obstruction (chronic obstructive bronchitis), emphysema, asthmatic bronchitis, and bullous disease, and combinations thereof.

The term “respiratory” refers to the process by which oxygen is taken into the body and carbon dioxide is discharged, through the bodily system including the nose, throat, larynx, trachea, bronchi and lungs.

The term “respiratory disease or condition” refers to any one of several ailments that involve inflammation and affect a component of the respiratory system including especially the trachea, bronchi and lungs. Such ailments include asthmatic conditions including allergen-induced asthma, exercise-induced asthma, pollution-induced asthma, cold-induced asthma, stress-induced asthma and viral-induced-asthma, chronic obstructive pulmonary diseases including chronic bronchitis with normal airflow, chronic bronchitis with airway obstruction (chronic obstructive bronchitis), emphysema, asthmatic bronchitis, and bullous disease, and other pulmonary diseases involving inflammation including cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, post-cardiac surgery, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus and hypoxia.

The term “respiratory condition effective” as used herein refers to a characteristic of an amount of a therapeutic compound, or a characteristic of amounts of combined therapeutic compounds in combination therapy. The amount or combined amounts achieve the goal of preventing, avoiding, reducing or eliminating a respiratory disease or condition.

The invention contemplates use of any iNOS selective inhibitor without specific regard for the mechanism by which the compound exerts its inhibitory effect. Inducible NOS selective inhibitors mentioned by way of example include S-(2-Aminoethyl)isothiourea, Aminoguanidine, 2-Amino-4-methylpyridine, AMT, L-Canavanine, 2-Iminopiperidine, S-Isopropylisothiourea, S-Methyl isothiourea, L-NIL, and 1400W, or pharmaceutically acceptable salts, prodrugs or solvates thereof.

The invention contemplates use of any inhibitor of the iNOS isoform of the NOS enzyme, whether the inhibitor is selective or non-selective for iNOS.

In an exemplary embodiment, the iNOS inhibitor is selective for iNOS. One illustrative example of a selective iNOS inhibitor, treatment is facilitated through compounds having Formula I:

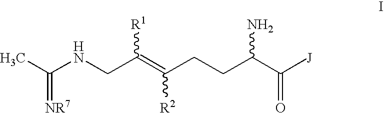

or a pharmaceutically acceptable salt or prodrug thereof, wherein:

R¹ is selected from the group consisting of H, halo and alkyl which may be optionally substituted by one or more halo;

R² is selected from the group consisting of H, halo and alkyl which may be optionally substituted by one or more halo; with the proviso that at least one of R¹ or R² contains a halo;

R⁷ is selected from the group consisting of H and hydroxy; and

J is selected from the group consisting of hydroxy, alkoxy, and NR³R⁴ wherein;

R³ is selected from the group consisting of H, lower alkyl, lower alkylenyl and lower alkynyl; and R⁴ is selected from the group consisting of H, and a heterocyclic ring in which at least one member of the ring is carbon and in which 1 to about 4 heteroatoms are independently selected from oxygen, nitrogen and sulfur and said heterocyclic ring may be optionally substituted with heteroarylamino, N-aryl-N-alkylamino, N-heteroarylamino-N-alkylamino, haloalkylthio, alkanoyloxy, alkoxy, heteroaralkoxy, cycloalkoxy, cycloalkenyloxy, hydroxy, amino, thio, nitro, lower alkylamino, alkylthio, alkylthioalkyl, arylamino, aralkylamino, arylthio, alkylsulfinyl, alkylsulfonyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, alkanoyl, alkenoyl, aroyl, heteroaroyl, aralkanoyl, heteroaralkanoyl, haloalkanoyl, alkyl, alkenyl, alkynyl, alkylenedioxy, haloalkylenedioxy, cycloalkyl, cycloalkenyl, lower cycloalkylalkyl, lower cycloalkenylalkyl, halo, haloalkyl, haloalkoxy, hydroxyhaloalkyl, hydroxyaralkyl, hydroxyalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, cyanoalkyl, dicyanoalkyl, carboxamidoalkyl, dicarboxamidoalkyl, cyanocarboalkoxyalkyl, carboalkoxyalkyl, dicarboalkoxyalkyl, cyanocycloalkyl, dicyanocycloalkyl, carboxamidocycloalkyl, dicarboxamidocycloalkyl, carboalkoxycyanocycloalkyl, carboalkoxycycloalkyl, dicarboalkoxycycloalkyl, formylalkyl, acylalkyl, dialkoxyphosphonoalkyl, diaralkoxyphosphonoalkyl, phosphonoalkyl, dialkoxyphosphonoalkoxy, diaralkoxyphosphonoalkoxy, phosphonoalkoxy, dialkoxyphosphonoalkylamino, diaralkoxyphosphonoalkylamino, phosphonoalkylamino, dialkoxyphosphonoalkyl, diaralkoxyphosphonoalkyl, guanidino, amidino, and acylamino.

In another embodiment, the present invention provides treatment utilizing a compound or a salt thereof, the compound having a structure corresponding to Formula II:

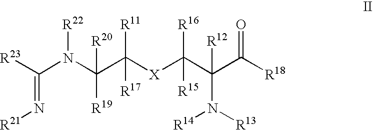

or a pharmaceutically acceptable salt or prodrug thereof.

In the structure of Formula II, X is selected from the group consisting of —S—, —S(O)—, and —S(O)₂—. Preferably, X is —S—. R¹² is selected from the group consisting of C₁-C₆ alkyl, C₂-C₆ alkenyl, C₂-C₆ alkynyl, C₁-C₅ alkoxy-C₁ alkyl, and C₁-C₅ alkylthio-C₁ alkyl wherein each of these groups is optionally substituted by one or more substituent selected from the group consisting of —OH, alkoxy, and halogen. Preferably, R¹² is C₁-C₆ alkyl optionally substituted with a substituent selected from the group consisting of —OH, alkoxy, and halogen. With respect to R¹³ and R¹⁸, R¹⁸ is selected from the group consisting of —OR²⁴ and —N(R²⁵)(R²⁶), and R¹³ is selected from the group consisting of —H, —OH, —C(O)—R²⁷, —C(O)—O—R²⁸, and —C(O)—S—R²⁹; or R¹⁸ is —N(R³⁰)—, and R¹³ is —C(O)—, wherein R¹⁸ and R¹³ together with the atoms to which they are attached form a ring; or R¹⁸ is —O—, and R¹³ is —C(R³¹)(R³²)—, wherein R¹⁸ and R¹³ together with the atoms to which they are attached form a ring. If R¹³ is —C(R3²¹)(R³²)—, then R¹⁴ is —C(O)—O—R³³; otherwise R¹⁴ is —H. R¹¹, R¹⁵, R¹⁶, and R¹⁷ independently are selected from the group consisting of —H, halogen, C₁-C₆ alkyl, C₂-C₆ alkenyl, C₂-C₆ alkynyl, and C₁-C₅ alkoxy-C₁ alkyl. R¹⁹ and R²⁰ independently are selected from the group consisting of —H, C₁-C₆ alkyl, C₂-C₆ alkenyl, C₂-C₆ alkynyl, and C₁-C₅ alkoxy-C₁ alkyl. With respect to R²¹ and R²², R²¹ is selected from the group consisting of —H, —OH, —C(O)—O—R³⁴, and —C(O)—S—R³⁵, and R²² is selected from the group consisting of —H, —OH, —C(O)—O—R³⁶, and —C(O)—S—R³⁷; or R²¹ is —O—, and R²² is —C(O)—, wherein R²¹ and R²² together with the atoms to which they are attached form a ring; or R²¹ is —C(O)—, and R²² is —O—, wherein R²¹ and R²² together with the atoms to which they are attached form a ring. R²³ is C₁ alkyl. R²⁴ is selected from the group consisting of —H and C₁-C₆ alkyl, wherein when R²⁴ is C₁-C₆ alkyl, R²⁴ is optionally substituted by one or more moieties selected from the group consisting of cycloalkyl, heterocyclyl, aryl, and heteroaryl. With respect to R²⁵ and R²⁶, R²⁵ is selected from the group consisting of —H, alkyl, and alkoxy, and R²⁶ is selected from the group consisting of —H, —OH, alkyl, alkoxy, —C(O)—R³⁸, —C(O)—O—R³⁹, and —C(O)—S—R⁴⁰; wherein when R²⁵ and R²⁶ independently are alkyl or alkoxy, R²⁵ and R²⁶ independently are optionally substituted with one or more moieties selected from the group consisting of cycloalkyl, heterocyclyl, aryl, and heteroaryl; or R²⁵ is —H; and R²⁶ is selected from the group consisting of cycloalkyl, heterocyclyl, aryl, and heteroaryl. R²⁷, R²⁸, R²⁹, R³⁰, R³¹, R³², R³³, R³⁴, R³⁵, R³⁶, R³⁷, R³⁸, R³⁹, and R⁴⁰ independently are selected from the group consisting of —H and alkyl, wherein alkyl is optionally substituted by one or more moieties selected from the group consisting of-cycloalkyl, heterocyclyl, aryl, and heteroaryl. When any of R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, and R⁴⁰ independently is a moiety selected from the group consisting of alkyl, alkenyl, alkynyl, alkoxy, alkylthio, cycloalkyl, heterocyclyl, aryl, and heteroaryl, then the moiety is optionally substituted by one or more substituent selected from the group consisting of —OH, alkoxy, and halogen.

In a preferred compound, R¹⁸ is —OH. When R¹⁸ is —OH, preferably X is S. In a further compound, R¹¹, R¹⁵, R¹⁶, R¹⁷, R¹⁹, and R²⁰ independently are selected from the group consisting of —H and C₁-C₃ alkyl. Preferably R¹⁵, R¹⁶, R¹⁷, R¹⁹, R²⁰ each are —H. R²³ can be a variety of groups, for example fluoromethyl or methyl. R¹¹ can be C₁-C₆ alkyl optionally substituted with a substituent selected from the group consisting of —OH and halogen; preferably R¹¹ is C₁ alkyl optionally substituted with halogen; more preferably R¹¹ is selected from the group consisting of fluoromethyl, hydroxymethyl, and methyl. In one important compound, R¹¹ can be methyl. Alternatively, R¹¹ can be fluoromethyl. In another alternative R¹¹ can be hydroxymethyl. In another compound, R¹² is C₁-C₆ alkyl optionally substituted with a substituent selected from the group consisting of —OH, alkoxy, and halogen. In one preferred compound R¹² is C₁ alkyl optionally substituted with halogen. For example, R¹² can be methyl. Alternatively, R¹² can be fluoromethyl. In yet another example, R¹² can be hydroxymethyl. In still another example, R¹² can be methoxymethyl.

In this exemplary compound, it is preferred that R¹³, R¹⁴, R²¹ and R²² each is —H. In this compound, it is further preferred that R¹¹, R¹⁵, R¹⁶, R¹⁷, R¹⁹, and R²⁰ independently are selected from the group consisting of —H and C₁-C₃ alkyl. Preferably R¹⁵, R¹⁶, R¹⁷, R¹⁹, R²⁰ each is —H. In this further compound, R²³ can be, for example, fluoromethyl, or in another example R²³ can be methyl. In preferred compounds of these examples, R¹² is C₁-C₆ alkyl optionally substituted with a substituent selected from the group consisting of -OH, alkoxy, and halogen. Preferably R¹² is C₁ alkyl optionally substituted with halogen. In one such example R¹² is fluoromethyl. In another example R¹² is methyl. Alternatively R¹² can be hydroxymethyl. In another alternative, R¹² can be methoxymethyl.

When R²³ is methyl, R¹¹ can be, for example, —H or C₁-C₆ alkyl optionally substituted with a substituent selected from the group consisting of —OH and halogen. In a preferred compound R¹¹ is —H. Alternatively, R¹¹ can be C₁-C₆ alkyl optionally substituted with a substituent selected from the group consisting of —OH and halogen. For example R¹¹ can be methyl, ethyl, n-propyl, i-propyl, n-butyl, sec-butyl, isobutyl, t-butyl, a pentyl isomer, or a hexyl isomer. For example, R¹¹ can be ethyl. Alternatively, R¹¹ can be C₁ alkyl optionally substituted with a substituent selected from the group consisting of —OH and halogen; for example R¹¹ can be methyl. Alternatively, R¹¹ can be fluoromethyl. In another alternative, R¹¹ can be hydroxymethyl.

In another compound R¹⁸ can be —OR²⁴. R²⁴ can be as defined above. Preferably R²⁴ is C₁-C₆ alkyl optionally substituted by one or more moieties selected from the group consisting of cycloalkyl, heterocyclyl, aryl, and heteroaryl; more preferably R²⁴ is C₁-C₃ alkyl; and more preferably still R²⁴ is methyl. In yet another example of compound II, R¹⁸ can be —N(R²⁵)(R²⁶), wherein R²⁵ and R²⁶ are as defined above. In still another compound, R¹⁸ can be —N(R³⁰)—, and R¹³ can be —C(O)—, wherein R¹⁸ and R¹³ together with the atoms to which they are attached form a ring. In another example still, R¹⁸ can be —O—, and R¹³ can be —C(R³¹)(R³²)—, wherein R¹⁸ and R¹³ together with the atoms to which they are attached form a ring.

In a compound of Formula II, R²¹ can be selected from the group consisting of —OH, —C(O)—O—R³⁴, and —C(O)—S—R³⁵. Preferably R²¹ is —OH. In a further example, R²² is —H when R²¹ is —OH.

However, the present example also provides useful compounds of Formula II in which R²¹ is —O—, and R²² is —C(O)—, wherein R²¹ and R²² together with the atoms to which they are attached form a ring. In another useful compound, R²¹ is —C(O)—, and R²² is —O—, wherein R²¹ and R²² together with the atoms to which they are attached form a ring. Alternatively, R²² can be selected from the group consisting of —OH, —C(O)—O—R³⁶, and —C(O)—S—R³⁷. In this alternative, R²¹ is preferably —H.

In another selective iNOS inhibitor useful in the practice of the present invention, a compound is represented by Formula III:

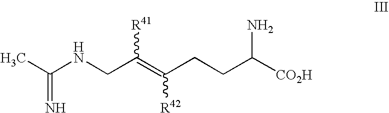

or a pharmaceutically acceptable salt or prodrug thereof, wherein:

R⁴¹ is H or methyl; and

R⁴² is H or methyl.

Another selective iNOS inhibitor useful in the practice of the present invention is represented by a compound of formula IV

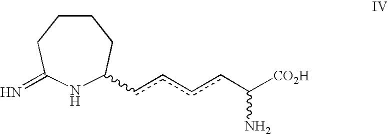

or a pharmaceutically acceptable salt or prodrug thereof.

Another exemplary selective iNOS inhibitor useful in the present invention is represented by Formula V:

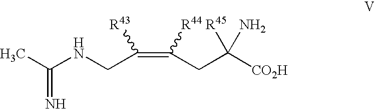

or a pharmaceutically acceptable salt or prodrug thereof, wherein:

R⁴³ is selected from the group consisting of hydrogen, halo, C₁-C₅ alkyl and

C₁-C₅ alkyl substituted by alkoxy or one or more halo;

R⁴⁴ is selected from the group consisting of hydrogen, halo, C₁-C₅ alkyl and

C₁-C₅ alkyl substituted by alkoxy or one or more halo;

R⁴⁵ is C₁-C₅ alkyl or C₁-C₅ alkyl be substituted by alkoxy or one or more halo.

A further illustrative selective iNOS inhibitor is represented by Formula VI:

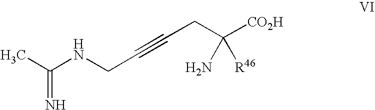

or a pharmaceutically acceptable salt or prodrug thereof, wherein:

R⁴⁶ is C₁-C₅ alkyl, said C₁-C₅ alkyl optionally substituted by halo or alkoxy, said alkoxy optionally substituted by one or more halo.

Another exemplary selective iNOS inhibitor useful in the present invention is represented by Formula VII

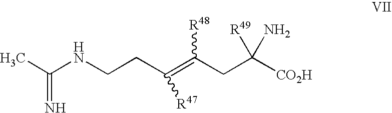

or a pharmaceutically acceptable salt or prodrug thereof, wherein:

R⁴⁷ is selected from the group consisting of hydrogen, halo, C₁-C₅ alkyl and

C₁-C₅ alkyl substituted by alkoxy or one or more halo;

R⁴⁸ is selected from the group consisting of hydrogen, halo, C₁-C₅ alkyl and

C₁-C₅ alkyl substituted by alkoxy or one or more halo;

R⁴⁹ is C₁-C₅ alkyl or C₁-C₅ alkyl be substituted by alkoxy or one or more halo.

Another exemplary selective iNOS inhibitor useful in the present invention is represented by Formula VIII

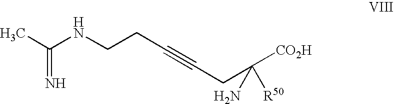

or a pharmaceutically acceptable salt or prodrug thereof, wherein:

R⁵⁰ is C₁-C₅ alkyl, said C₁-C₅ alkyl optionally substituted by halo or alkoxy, said alkoxy optionally substituted by one or more halo.

Another selective iNOS inhibitor useful in the practice of the present invention is represented by a compound of formula IX

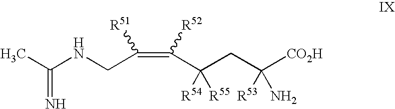

or a pharmaceutically acceptable salt or prodrug thereof, wherein:

R⁵¹ is selected from the group consisting of hydrogen, halo, and C₁-C₅ alkyl, said C₁-C₅ alkyl optionally substituted by halo or alkoxy, said alkoxy optionally substituted by one or more halo;

R⁵² is selected from the group consisting of hydrogen, halo, and C₁-C₅ alkyl, said C₁-C₅ alkyl optionally substituted by halo or alkoxy, said alkoxy optionally substituted by one or more halo;

R⁵³ is C₁-C₅ alkyl, said C₁-C₅ alkyl optionally substituted by halo or alkoxy, said alkoxy optionally substituted by one or more halo;

R⁵⁴ is selected from the group consisting of hydrogen, halo, and C₁-C₅ alkyl, said C₁-C₅ alkyl optionally substituted by halo or alkoxy, said alkoxy optionally substituted by one or more halo; and

R⁵⁵ is selected from the group consisting of halo and C₁-C₅ alkyl, said C₁-C₅ alkyl optionally substituted by halo or alkoxy, said alkoxy optionally substituted by one or more halo.

Yet another selective iNOS inhibitor useful in the practice of the present invention is represented by a compound of formula X

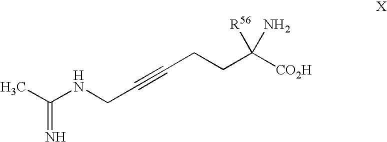

or a pharmaceutically acceptable salt or prodrug thereof, wherein:

R⁵⁶ is C₁-C₅ alkyl, said C₁-C₅ alkyl optionally substituted by halo or alkoxy, said alkoxy optionally substituted by one or more halo.

In another exemplary compound, the inducible nitric oxide synthase selective inhibitor is the compound having the formula XI, or a pharmaceutically acceptable thereof. Compound XI has previously been described in International Publication Number WO 00/26195, published May 11, 2000, which is herein incorporated by reference.

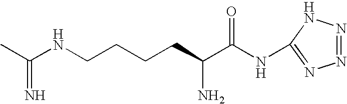

2S-amino-6-[(1-iminoethyl)amino]-N-(1H-tetrazol-5-yl) hexanamide, hydrate, dihydrochloride XI

The invention also contemplates use of other selective iNOS inhibitors. By way of example, iNOS selective inhibitors also useful in the present invention are described in U.S. Pat. No. 6,355,689, Beswick et al., filed Nov. 29, 2000 and issued Mar. 12, 2002, which describes and claims a selective iNOS inhibitor with the formula XI:

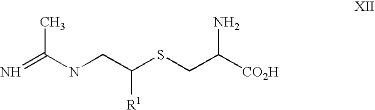

wherein R¹ is selected from C₁₋₄ alkyl, C₃₋₄ cycloalkyl, C₁₋₄ hydroxyalkyl, and C₁₋₄ haloalkyl. The description of U.S. Pat. No. 6,355,689 states that R¹ is preferably C₁₋₄ alkyl, and most preferably, methyl. Specific embodiments disclosed in U.S. Pat. No. 6,355,689 and suitable for use in the present methods and compositions include:

S—((R)-2-(1-iminoethylamino)propyl)-L-cysteine;

S—((S)-2-(1-iminoethylamino)propyl)-L-cysteine;

S—((R/S)-2-(1-iminoethylamino)propyl)-L-cysteine;

S—((R)-2-(1-iminoethylamino)propyl )-D-cysteine;

S—((S)-2-(1-iminoethylamino)propyl)-D-cysteine;

S—((R/S)-2-(1-iminoethylamino)propyl)-D-cysteine;

S—((R/S)-2-(1-iminoethylamino)butyl)-L-cysteine;

S—((R/S)-2-(1-iminoethylamino,2-cyclopropyl)ethyl)-L-cysteine; and

S—((R/S)-2-(1-iminoethylamino,3-hydroxy)propyl)-L-cysteine,

or a pharmaceutically acceptable salt, solvate, or physiologically functional derivative thereof.

INOS inhibitors that are believed to exert their inhibitory effect by inhibiting the dimerization of iNOS are also contemplated for use in the present ivention and include those compounds disclosed in international publication number WO 9837079, published Aug. 27, 1998, international patent application PCT/US98/03176 by Berlex Laboratories, Inc., 15049 San Pablo Avenue, P.O. Box 4099, Richmond, Calif. 94804-0099, and Pharmacopeia, Inc., Princeton Forrestal Center, 101 College Road East, Princeton, N.J. 08540. Briefly, that publication discloses compounds of formulae XIII, XIV and XV:

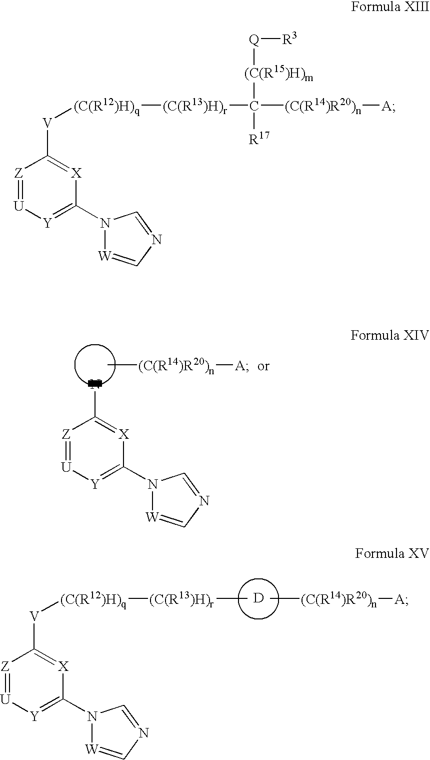

A is —R¹, —OR¹, C(O)N(R¹)R², P(O)[N(R¹)R²]₂, —N(R¹)C(O)R², —N(R¹⁶)C(O)OR², —N(R¹)R²¹, —N(R¹⁶)C(O)N(R¹)R¹⁶, —S(O)ₜR¹, —SO₂NHC(O)R¹, —NHSO₂R²², —SO₂NH(R¹)H, —C(O)NHSO₂R²², and —CH═NOR¹;

each X, Y and Z are independently N or C(R¹⁹);

each U is N or C(R5), provided that U is N only when X is N and Z and Y are CR¹⁹;

V is N(R⁴), S, O or C(R⁴)H;

Each W is N or CH;

Q is chosen from the group consisting of a direct bond, —C(O)—, —O—, —C(═N—R¹)—, S(O)ₜ, and —N(R⁶)—;

m is zero or an integer from 1 to 4;

n is zero or an integer from 1 to 3;

q is zero or one;

r is zero or one, provided that when Q and V are heteroatoms, m, q, and r cannot all be zero;

when A is —OR¹, N(R¹)C(O)R², —N(R¹⁶)C(O)OR², —N(R¹)R²¹, —N(R¹⁶)C(O)N(R¹)R¹⁶, —S(O)ₜR¹ (where t is zero), or —NHSO₂R²², n, q, nd r cannot all be zero; and when Q is a heteroatom and A is —OR¹, N(R¹)C(O)R², —N(R¹⁶)C(O)OR², —N(R¹)R²¹, N(R¹⁶)C(O)N(R¹)R¹⁶, —S(O)ₜR¹ (when t is zero), or —NHSO₂R²² , m and n cannot both be zero;

t is zero, one or two;

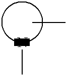

is an optionally substituted N-heterocyclyl;

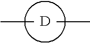

is an optionally substituted carbocyclyl or optionally substituted N-heterocyclyl;

each R¹ and R² are independently chosen from the group consisting of hydrogen, optionally substituted C₁-C₂₀ alkyl, optionally substituted cycloalkyl,

—[C₀-C₈ alkyl]-R⁹, —[C₂-C₈ alkenyl]-R⁹, —[C₂-C₈ alkynyl]-R⁹, —[C₂-C₈ alkyl]-R¹⁰ (optionally substituted by hydroxy), —[C₁-C₈]-R¹¹ (optionally substituted by hydroxy), optionally substituted heterocyclyl;

or R¹ and R² together with the nitrogen atom to which they are attached is an optionally substituted N-heterocyclyl;

R³ is chosen from the group consisting of hydrogen, alkyl, cycloalkyl, optionally substituted aryl, haloalkyl, —[C₁-C₈ alkyl]-C(O)N(R¹)R², —[C₁-C₈ alkyl]-N(R¹)R², —[C₁-C₈ alkyl]-R⁸, —[C₂-C₈ alk2yl]-R¹⁰, —[C₁-C₈ alkyl]-R¹¹, and heterocyclyl (optionally substituted by one or more substitutents selected from the group consisting of halo, alkyl, alkoxy and imidazolyl);

or when Q is —N(R⁶)— or a direct bond to R³, R³ may additionally be aminocarbonyl,

alkoxycarbonyl, alkylsulfonyl, monoalkylaminocarbonyl, dialkylaminocarbonyl and —C(═NR¹⁸)—NH₂;

or -Q-R³ taken together represents —C(O)OH, —C(O)N(R¹)R² or

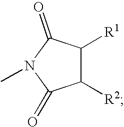

R⁴ is chosen from the group consisting of hydrogen, alkyl, aryl, aralkyl and cycloalkyl;

Provided that when A is —R¹ or —OR¹, R⁴ cannot be hydrogen, and when V is CH, R⁴ may additionally be hydroxy;

R⁵ is chosen from the group consisting of hydrogen, alkyl, aryl, aralkyl, haloalkyl,

optionally substituted aralkyl, optionally substituted aryl, —OR¹⁶, —S(O)ₜ—R¹⁶, N(R¹⁶)R²¹, N(R¹⁶)C(O)N(R¹)R¹⁶, N(R¹⁶)C(O)OR¹⁶, N(R¹⁶)C(O)R¹⁶, —[C₀-C₈ alkyl]-C(H)[C(O)R¹⁶]₂ and —[C₀-C₈ alkyl]-C(O)N(R¹)R¹⁶;

R⁶ is chosen from the group consisting of hydrogen, alkyl, cycloalkyl, —[C₁-C₈ alkyl]-R⁸, —[C₂-C₈]alkyl]-R¹⁰, —[C₁-1C₈ alkyl]-R¹¹, acyl, —C(O)R⁸, —C(O)——[C₁-C₈ alkyl]-R⁸, alkoxycarbonyl, optionally substituted aryloxycarbonyl, optionally substituted aralkoxycarbonyl, alkylsulfonyl, optionally substituted aryl, optionally substituted heterocyclyl, alkoxycarbonylalkyl, carboxyalkyl, optionally substituted arylsulfonyl, aminocarbonyl, monoalkylaminocarbonyl, dialkylaminocarbonyl, optionally substituted arylaminocarbonyl, aminosulfonyl, monoalkylaminosulfonyl dialkylaminosulfonyl, arylaminosulfonyl, arylsulfonylaminocarbonyl, optionally substituted N-heterocyclyl, —C(═NH)—N(CN)R¹, —C(O)R²³—N(R¹)R , —C(O)—N(R¹)R²³—C(O)OR¹;

each R⁸ and R⁹ are independently chosen from the group consisting of haloalkyl,

cycloalkyl, (optionally substituted with halo, cyano, alkyl or alkoxy), carbocyclyl (optionally substituted with one or more substituents selected from the group consisting of halo, alkyl and alkoxy) and heterocyclyl (optionally substituted with alkyl, aralkyl or alkoxy);

each R¹⁰ is independently chosen from the group consisting of halo, alkoxy, optionally

substituted aryloxy, optionally substituted aralkoxy, optionally substituted —S(O)ₜ—R²², acylamino, amino, monoalkylamino, dialkylamino, (triphenylmethyl)amino, hydroxy, mercapto, alkylsulfonamido;

each R¹¹ is independently chosen from the group consisting of cyano, di(alkoxy)alkyl,

carboxy, alkoxycarbonyl, aminocarbonyl,

monoalkylaminocarbonyl and dialkylaminocarbonyl;

each R¹², R¹³, R¹⁴, R¹⁵, R¹⁷, and R²⁰ are independently hydrogen or alkyl;

each R¹⁶ is independently hydrogen, alkyl, optionally substituted aryl, optionally

substituted aralkyl or cycloalkyl;

R¹⁸ is hydrogen, NO₂, or toluenesulfonyl;

each R¹⁹ is independently hydrogen, alkyl (optionally substituted with hydroxy),

cyclopropyl, halo or haloalkyl;

each R²¹ is independently hydrogen, alkyl, cycloalkyl, optionally substituted aryl,

optionally substituted aralkyl, —C(O)R²² or —SO₂R²²;

or R²¹ taken together with R¹ and the nitrogen to which they are attached is an optionally substituted N-heterocyclyl;

or R²¹ taken together with R¹⁶ and the nitrogen to which they are attached is an optionally

substituted N-heterocyclyl;

each R²² is independently alkyl, cycloalkyl, optionally substituted aryl or optionally

substituted aralkyl; and

R²³ is an amino acid residue;

as a single stereoisomer or mixture thereof, or a pharmaceutically acceptable salt thereof.

Another iNOS dimerization inhibitor, 3-(2,4-difluorophenyl)-6-{2-[4-(1H-imidazol-1-ylmethyl) phenoxy]ethoxy}-2-phenylpyridine (PPA250) has been described in Ohtsuka et al., J Phamacol Exp Ther Vol. 303, Issue 1, 52-57, October 2002. PPA250 has the structure:

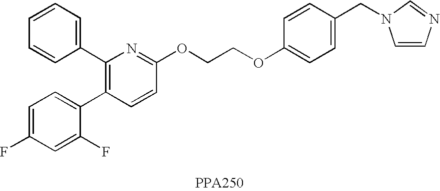

Therefore, in another embodiment of the present invention, the compound PPA250 may be employed as the selective iNOS inhibitor.

It has also been proposed that antisense oligonucleotides may effectively block mRNA levels in vertebrates, including humans, and thus decrease or inhibit the expression of iNOS. For example, international application PCT/US01/01381, by ISIS Pharmaceuticals, Inc. and published as WO 01/52902 on Jul. 26, 2001, describes anti-sense compounds for modulating the expression of iNOS, particularly antisense oligonucleotides targeted to nucleic acids encoding iNOS. The invention also comtemplates use of such antisense oilgonucleotides as the iNOS selective inhibitor in the methods and compositions of the present invention.

PDE inhibitors used in the methods and compositions of the present invention include specific (i.e. selective) and non-specific (i.e non-selective) PDE inhibitors. However, selective inhibitors of PDE isozymes known to be specifically involved in airway dilation or airway smooth muscle relaxation are especially suitable. For example, selective inhibitors of the PDE-III isozyme produce airway dilation. See GOODMAN & GILMAN'S THE PHARMACOLOGICAL BASIS OF THERAPEUTICS, 832-4, (Joel G. Hardman et al. eds., 9^{th} ed. 1996). Selective inhibitors of the PDE-IV isozyme produce airway smooth muscle relaxation. Id.

Therefore, in one exemplary embodiment, the PDE inhbitor is selected from the group of PDE-III inhibitors. In another exemplary embodiment, the PDE inhibitor is selected from the group of PDE-IV inhibitors. In an alternative embodiment, the PDE inhibitor is selected from the group of PDE-III/IV dual inhibitors. In still another embodiment, the PDE inhibitor is selected from the group of PDE-II inhibitors.

Non-specific PDE inhibitors mentioned by way of example include Theophylline, Dipyridamole, TRENTAL (pentoxifylline), Hoechst Marion Roussel, (Bad Soden, Germany); and Isobutyl methylxanthine (IBMX).

Specific PDE-I inhibitors mentioned by way of example include VINPOCETINE, KS-505a, W-7, and Phenothiazines.

A specific PDE-II inhibitor mentioned by way of example is EHNA. To determine the inter-isoform selectivity of a PDE IV inhibitor, the putative inhibitor compound is typically incubated together with each individual isoform of phosphodiesterase and simultaneously with substrate cyclic nucleotides. PDE inhibition is then determined by the presence or absence of substrate degradation products. See e.g. Hatzelmann, A., et al., J. Pharm. Exper. Therap., 297(1):267-279 (2001). The relative ability of an inhibitory compound to slow or prevent the degradation of tritiated cyclic nucleotides is one test that is indicative of how well the compound in question selects one or more of each isoform to inhibit. Representative PDE isoform enzymes and other reaction substrates can be obtained by isolation from appropriate tissues and their purchase has been reported.

In practice, the in vitro selectivity of a PDE IV inhibitor may vary depending upon the condition under which the test is performed and on the inhibitors being tested. However, for the purposes of this specification, the selectivity of a PDE IV inhibitor can be measured as a ratio of the in vitro IC₅₀ value for inhibition of any other isoform of the phosphodiesterase enzyme (Z) other than PDE IV, divided by the IC₅₀ value for inhibition of PDE IV (PDE Z IC₅₀/PDE IV IC₅₀), where Z identifies any PDE other than PDE IV. As used herein, the term “IC₅₀” refers to the concentration of a compound that is required to produce 50% inhibition of phosphodiesterase activity. A PDE IV selective inhibitor is any inhibitor for which the ratio of PDE Z IC₅₀ to PDE IV IC₅₀ is greater than 1. In a preferred embodiment, this ratio is greater than 2, more preferably greater than 10, yet more preferably greater than 100, and more preferably still greater than 1000.

By way of example, in Hatzelmann, A., et al., J. Pharm. Exper. Therap., 297(1):267-279 (2001), the IC₅₀ for roflumilast activity on PDE IV was reported to be 0.0008 μM, while the IC₅₀ for roflumilast activity on PDE I was reported to be >10 μM. Accordingly, the selectivity of roflumilast for PDE IV as compared with PDE I would be >10/0.0008 or at least about 12,500. Likewise, the selectivity of roflumilast for PDE IV as compared with PDE V would be 8/0.0008 or at least about 10,000.

Thus, preferred PDE IV selective inhibitors of the present invention have a PDE IV IC₅₀ of less than about 1 μM, more preferred of less than about 0.1 μM, even more preferred of less than about 0.01 μM, and more preferred still of less than about 0.001 μM. Preferred PDE IV selective inhibitors have a PDEZ IC₅₀ of greater than about 1 μM, and more preferably of greater than 10 μM. An example of a selective PDE IV inhibitor that is particularly preferred for use in the present invention has been recently described for use in treating pulmonary inflammation is the pyridyl benzamide derivative, roflumilast (3-cyclopropylmethoxy-4-difluoromethoxy-N-[3,5-dichloropyrid-4-yl]-benzamide), a novel, highly potent, and selective PDE4 inhibitor. See U.S. Pat. No. 5,712,298, which in herein incorporated by reference.

PDE IV inhibitors are classified into three main chemical classes 1) Catechol Ethers (in which are grouped a wide variety of flexible molecules of inhibitors structurally related to rolipram) 2) Quinazolinediones which are structurally related to Nitraquazone and 3) Xanthines, to which theophylline belongs. Inside this class, two subclasses can be distinguished quinazolindiones and xanthines.

Preferably the PDE IV inhibitor is selected from the group consisting of rolipram, roflumilast, cilomilast, and ZK-117137, bamifylline, dyphylline, ibudilast, and Theophylline. Further individual PDE IV inhibitors useful in the present invention are individually listed in Table I.

TABLE I

| | Structure | | Structure | |
|---|---|---|---|---|
| No. | I.D. | Structure | Name | Reference |
| 1. | cilomilast Ariflo SB-207499 CAS RN: 153259-65-5 | 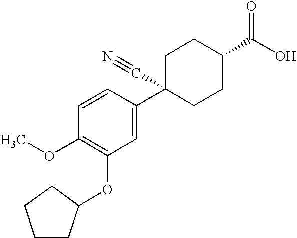 | 4-cyano-4-[3-cyclopentyloxy)-4- methoxy phenyl]cyclohexane carboxylic acid | Dal Piaz, V., et. al, Eur. J. Med. Chem. 35 (2000) 463-480 |
| 2. | roflumilast BY-217 CAS RN: 162401-32-3 | 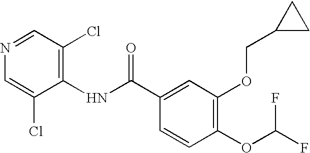 | 3-(cyclopropylmethoxy)-N-(3,5- dichloropyridin-4-yl)-4- (difluoromethoxy) benzamide | Souness, J., et al., Immunopharmacology 47 (2000) 127-162 |
| 3. | Pumafentrin BYK-33043 BY-343 CAS RN: 207993-12-2 | 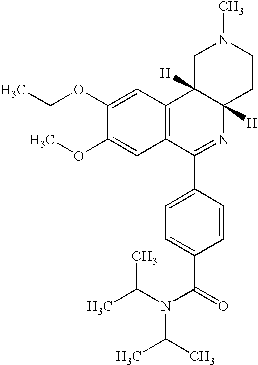 | 4-(9-Ethoxy-8-methoxy-2-methyl- 1,2,3,4,4a,10b-hexahydro-benzo [c][1,6] napthyridin-6-yl)-N,N- diisopropyl-benzamide | Norman P., Expert Opin. Ther. Patents (2002) 12(1): 93-111 |
| 4. | L-869298 CT-2450 Analogue: CT-2820 CT-3883 L-826141 Analogue: L-791943 CT-5210 CAS RN: 225919-29-9 | 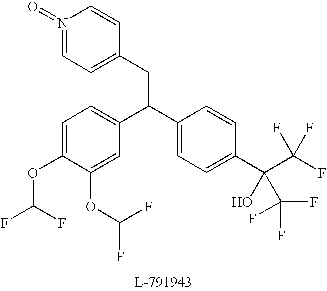 | 2-{4-[1-[3,4-bis(difluoromethoxy) phenyl]-2-(1-oxidopyridin-4-yl) ethyl]phenyl}-1,1,1,3,3,3- hexafluoropropan-2-ol | Norman P., Expert Opin. Ther. Patents (2002) 12(1): 93-111 |
| 5. | ZK-117137 SH-636 Trade Name: Mesopram CAS RN: 189940-24-7 | 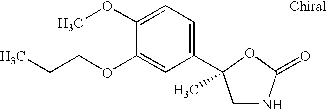 | 5-(4-methoxy-3-propoxyphenyl)-5- methyl-1,3-oxazolidin-2-one | US 2002/0103 106 A1 |
| 6. | rolipram ME-3167 ZK-62711 CAS RN: 61413-54-5 | 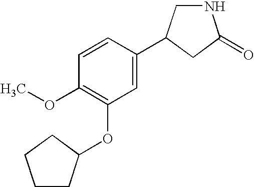 | 4-(3-cyclopentyloxy-4-methoxy- phenyl)-pyrrolidan-2-one | Dal Piaz, V. et. al., Eur. J. Med. Chem. 35 (2002) 463-480 |
| 7. | YM-976 CAS RN: 191219-80-4 | 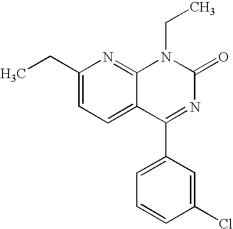 | 4-(3-Chloro-phenyl)-1,7-diethyl- 1H-pyrido[2,3-d]pyrimidin-2-one | US 2002/0103 106 A1 |
| 8. | Sch-351591 D-4396 | 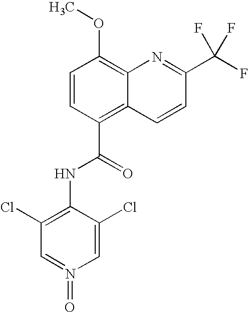 | N-(3,5-dichloro-1-oxidopyridin-4- yl)-8-methoxy-2-(trifluoromethyl) quinoline-5-carboxamide | US 2002/0103 106 A1 |
| 9. | 1C-485 | 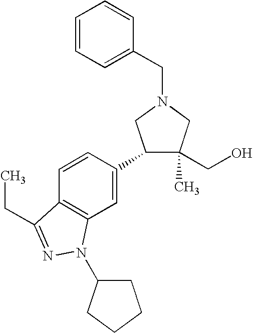 | [1-benzyl-4-(1-cyclopentyl-3- ethyl-1H-indazol-6-yl)-3- methylpyrrolidin-3-yl]methanol | US 2002/0103 106 A1 |
| 10. | D-4418 Sch-365351 CAS RN: 257892-34-5 | 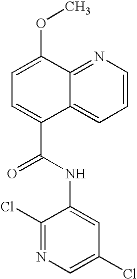 | 8-methoxy-quinoline-5-carboxylic acid (2,5-dichloropyridin- 3-yl) amide | US 2002/0103 106 A1 |
| 11. | PD-189659 CI-1044 Analogue: PD-168787 CI-1018 Analogue: PD-190749 Analogue: PD-190036 CAS RN: 197894-84-1 (Pfizer) | N-[9-amino-4-oxo-7-phenyl-1,2,4,5- tetrahydroazepino[3,2,1-hi]indol- 5-yl]nicotinamide | Dal Piaz, V., et. al., Eur. J. Med. Chem. 35 (2000) 463-480 | |
| 12. | CP-77059 CAS RN: 114918-24-0 | 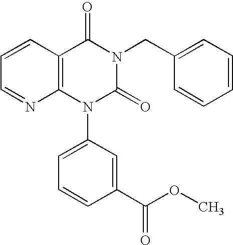 | 3-(3-benzyl-2,4-dioxo-3,4-dihydro- 2H-pyrido[2,3-d]pyrimidin-1- yl) benzoic acid methyl ester | Dal Piaz, V., et. al., Eur. J. Med. Chem. 35 (2000) 463-480 |
| 13. | RS-14203 CAS RN: 150347-75-4 | 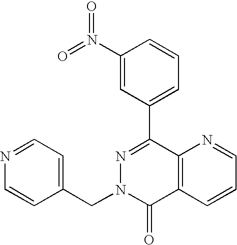 | 8-(3-nitrophenyl)-6-(pyridin-4- ylmethyl)pyrido[2,3-d]pyridazin-5(6H)-one | Dal Piaz, V., et. al., Eur. J. Med. Chem. 35 (2000) 463-480 |
| 14. | AWD-12-281 Analogue: AWD-12-343 CAS RN: 257892-33-4 | 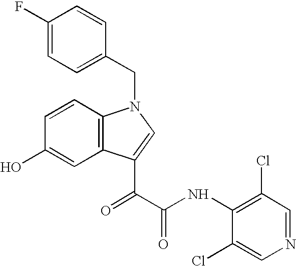 | N-(3,5-dichloropyridin-4-yl)-2- [1-(4-fluorobenzyl)-5-hydroxy-1H- indol-3-yl]-2-oxoacetamide | US 2002/0103 106 A1 |
| 15. | D-22888 Analogue: AWD-12-232 CAS RN: 182282-60-6 | 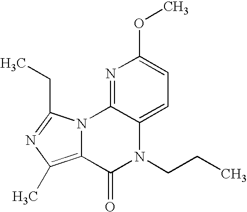 | 9-ethyl-2-methoxy-7-methyl-5- propylimidazo[1,5-a]pyrido[3,2- e]pyrazin-6(5H)-one | Dal Piaz, V., et. al., Eur. J. Med. Chem. 35 (2000) 463-480 |
| 16. | YM-58977 | 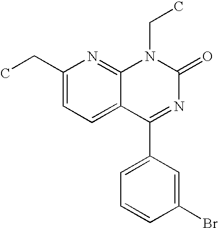 | 4-(3-bromophenyl)-1,7- diethylpyrido[2,3-d]pyrimidin- 2(1H)-one | Dal Piaz, V., et. al., Eur. J. Med. Chem. 35 (2000) 463-480 |
| 17. | Theophylline CAS RN: 58-55-9 | 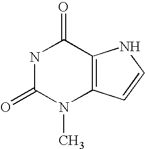 | 3,7-Dihydro-1,3-dimethyl-1H-purine- 2,6-dione | Dal Piaz, V., et. al., Eur. J. Med. Chem. 35 (2000) 463-480 |
| 18. | Cipamfylline HEP-688 BRL-61063 CAS RN: 132210-43-6 | 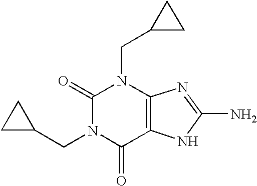 | 8-amino-1,3-bis-cyclopropylmethyl- 3,7-dihydro-purine-2,6- dione | Dal Piaz, V., et. al., Eur. J. Med. Chem. 35 (2000) 463-480 |
| 19. | Arofylline LAS-31025 CAS RN: 136145-07-8 | 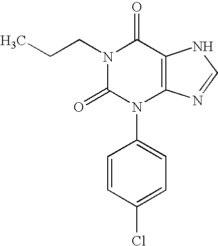 | 3-(4-chlorophenyl)-1-propyl-3,7- dihydro-1H-purine-2,6-dione | Dal Piaz, V., et. al., Eur. J. Med. Chem. 35 (2000) 463-480 |
| 20. | V-11294A CAS RN: 162278-09-3 | 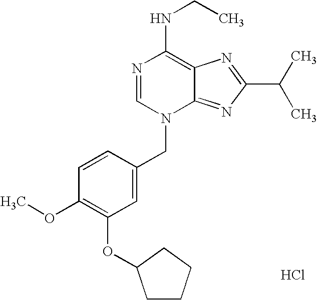 | [3-(3-cyclopentyloxy-4 methoxybenzyl]-8-isopropyl-3H- purin-6-yl]-ethyl amine hydrochloride | Dal Piaz, V., et. al., Eur. J. Med. Chem. 35 (2000) 463-480 |
| 21. | RPR-132294 Analogue: RPR-132703 | 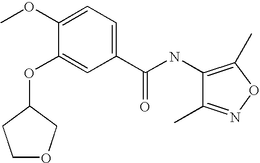 | N-(3,5-dimethylisoxazol-4-yl)-4- methoxy-3-(tetrahydrofuran-3- yloxy)benzamide | Dal Piaz, V., et. al., Eur. J. Med. Chem. 35 (2000) 463-480 |
| 22. | IBMX CAS RN: 28822-58-4 | 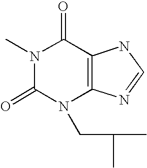 | 3-isobutyl-1-methyl-3,7-dihydro-1H- purine-2,6-dione | Dal Piaz, V., et. al., Eur. J. Med. Chem. 35 (2000) 463-480 |
| 23. | Isbufylline CAS RN: 90162-60-0 | 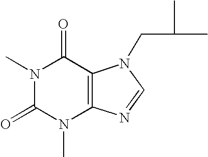 | 7-isobutyl-1,3-dimethyl-3,7- dihydro-1H-purine-2,6-dione | Dal Piaz, V., et. al., Eur. J. Med. Chem. 35 (2000) 463-480 |
| 24. | Doxofylline Trade Names: Ansimar Maxivent CAS RN: 69975-86-6 | 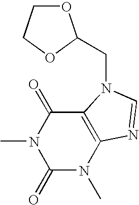 | 7-(1,3-dioxolan-2-ylmethyl)-1,3- dimethyl-3,7-dihydro-1H-purine- 2,6-dione | Dal Piaz, V., et. al., Eur. J. Med. Chem. 35 (2000) 463-480 |
| 25. | Dyphylline CAS RN: 479-18-5 | 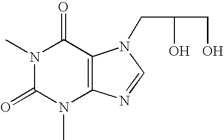 | 7-(2,3-dihydroxypropyl)-1,3- dimethyl-3,7-dihydro-1H- purine-2,6-dione | Dal Piaz, V., et. al., Eur. J. Med. Chem. 35 (2000) 463-480 |
| 26. | Verolylline CAS RN: 65029-11-0 | 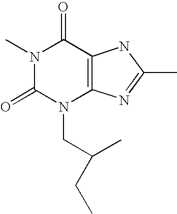 | 1,8-dimethyl-3-(2-methylbutyl)-3,7- dihydro-1H-purine- 2,6-dione | Dal Piaz, V., et. al., Eur. J. Med. Chem. 35 (2000) 463-480 |
| 27. | Bamifylline CAS RN: 2016-63-9 | 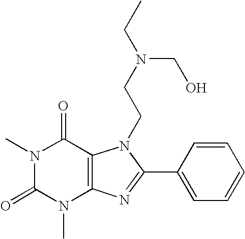 | 7-{2-[ethyl(hydroxymethyl)amino]ethyl}-1,3-dimethyl-8-phenyl-3,7- dihydro-1H-purine-2,6-dione | Dal Piaz, V., et. al., Eur. J. Med. Chem. 35 (2000) 463-480 |
| 28. | Pentoxifylline CAS RN: 6493-05-6 | 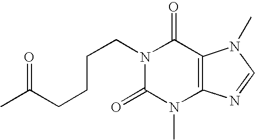 | 3,7-dimethyl-1-(5-oxohexyl)-3,7- dihydro-1H-purine-2,6-dione | Dal Piaz, V., et. al., Eur. J. Med. Chem. 35 (2000) 463-480 |
| 29. | Enprofylline CAS RN: 41078-02-8 | 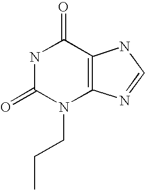 | 3-propyl-3,7-dihydro-1H-purine- 2,6-dione | Dal Piaz, V., et. al., Eur. J. Med. Chem. 35 (2000) 463-480 |
| 30. | Denbufylline CAS RN: 57076-71-8 | 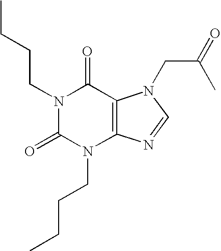 | 1,3-dibutyl-7-(2-oxopropyl)-3,7- dihydro-1H-purine-2,6-dione | Dal Piaz, V., et. al., Eur. J. Med. Chem. 35 (2000) 463-480 |
| 31. | Chiroscience 245412 | 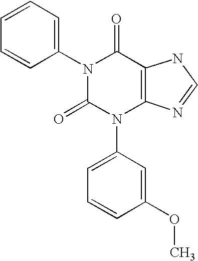 | 3-(3-methoxyphenyl)-1-phenyl-3,7- dihydro-1H-purine-2,6-dione | Dal Piaz, V., et. al., Eur. J. Med. Chem. 35 (2000) 463-480 |
| 32. | ICI 63197 CAS RN: 27277-00-5 | 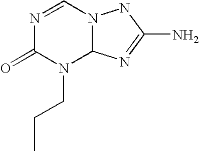 | 2-amino-4-propyl-3a,4-dihydro [1,2,4]triazalo [1,5- a][1,3,5]triazin-5(1H)-one | Dal Piaz, V., et. al., Eur. J. Med. Chem. 35 (2000) 463-480 |
| 33. | SCA 40 | 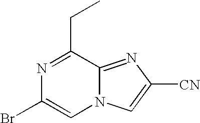 | 6-bromo-8-ethylimidazo[1,2- a]pyrazin-2-amine | Dal Piaz, V., et. al., Eur. J. Med. Chem. 35 (2000) 463-480 |
| 34. | Ibudilast CAS RN: 50847-11-5 | 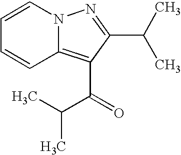 | 1-(2-isopropylpyrazolo[1,5-a]pyridin-3-yl)-2-methylpropan-1- one | Dal Piaz, V., et. al., Eur. J. Med. Chem. 35 (2000) 463-480 |
| 35. | N-cyclopentyl-8- cyclopropyl-3- propyl-3H-purin-6- amine CAS RN: 162278-16-2 162278-06-0 | 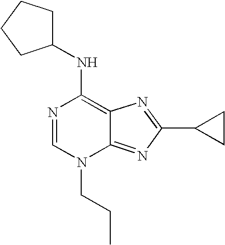 | N-cyclopentyl-8-cyclopropyl-3- propyl-3H-purin-6-amine | Dal Piaz, V., et. al., Eur. J. Med. Chem. 35 (2000) 463-480 |
| 36. | 8-cyclopropyl-N,3- diethyl-3H-purin-6- amine CAS RN: 126149-38-0 126252-48-0 126371-20-0 | 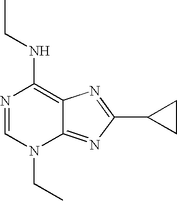 | 8-cyclopropyl-N,3-diethyl-3H- purin-6-amine | Dal Piaz, V., et. al., Eur. J. Med. Chem. 35 (2000) 463-480 |
| 37. | INN: lirimilast BAY-19-8004 CAS RN: 329306-27-6 | 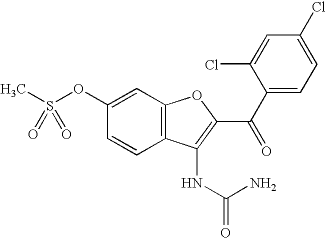 | Methane sulfonic acid 2-(2,4- dichloro-benzoyl-3-ureido- benzofuran-6-yl ester | Dal Piaz, V., et. al., Eur. J. Med. Chem. 35 (2000) 463-480 |
| 38. | (4-chlorophenyl)[3- (3,3-dihydroxybutyl)-6- hydroxy-1- benzofuran-2- yl]methanone | 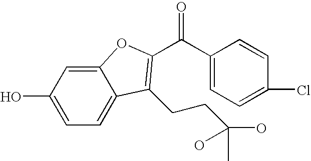 | (4-chlorophenyl)[3-(3,3- dihydroxybutyl)-6-hydroxy-1- benzofuran-2-yl]methanone | Dal Piaz, V., et. al., Eur. J. Med. Chem. 35 (2000) 463-480 |
| 39. | 1-{3- dimethylamino)-4- [(dimethylamino)methyl]- 7-hydroxy-5,6- dimethyl-1-benzofuran- 2-yl}ethanone | 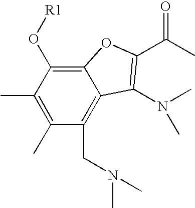 | 1-{3-(dimethylamino)-4- [(dimethylamino)-methyl]- 7-hydroxy-5,6-dimethyl-1- benzofuran-2-yl}ethanone | Dal Piaz, V., et. al., Eur. J. Med. Chem. 35 (2000) 463-480 |
| 40. | N-(3,5- dichloropyridin-4- yl)-8-methoxy-2,2- dimethylchromane- 5-carboxamide | 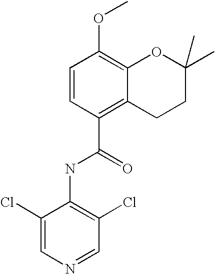 | N-(3,5-dichloropyrin-4-yl)-8- methoxy-2,2- dimethylchromane-5- carboxamide | Dal Piaz, V., et. al., Eur. J. Med. Chem. 35 (2000) 463-480 |
| 41. | 2-acetyl-N-benzyl-7- methoxy-1- benzofuran-4- sulfonamide | 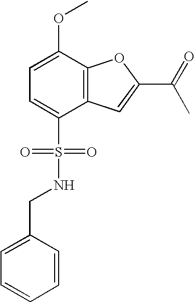 | 2-acetyl-N-benzyl-7-methoxy-1- benzofuran-4-sulfonamide | Dal Piaz, V., et. al., Eur. J. Med. Chem. 35 (2000) 463-480 |
| 42. | 1-cyclopentyl-N-(3,5- dichloropyridin- 4-yl)-3-ethyl-1H- indazole-6- carboxamide | 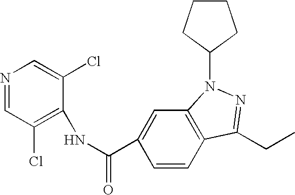 | 1-cyclopentyl-N-(3,5-dichloropyridin- 4-yl)-3-ethyl-1H- indazole-6-carboxamide | Dal Piaz, V., et. al., Eur. J. Med. Chem. 35 (2000) 463-480 |
| 43. | 1-cyclopentyl-3- ethyl-6-(2- methylphenyl)- 1,3a,4,5,6,7a-hexahydro-7H- pyrazolo[3,4-c]pyridin- 7-one | Dal Piaz, V., et. al., Eur. J. Med. Chem. 35 (2000) 463-480 | | |
| 44. | N-(4-oxo-1- phenyl-3,4,6,7- tetrahydro[1, 4]diazepino [6,7,1-hi]indol-3-yl)- 1H-indole- 2-carboxamide | 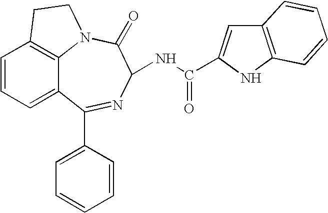 | N-(4-oxo-1-phenyl-3,4,6,7-tetrahydro [1,4]diazepino[6,7,1-hi]indol-3- yl)-1H-indole-2-carboxamide | Dal Piaz, V., et. al., Eur. J. Med. Chem. 35 (2000) 463-480 |
| 45. | CI-1118 | 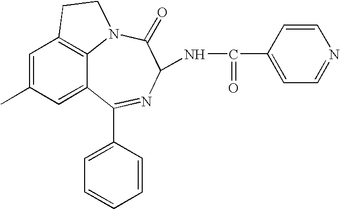 | N-(9-methyl-4-oxo-1-phenyl-3,4,6,7- tetrahydro[1,4]diazepino[6,7,1- hi]indol-3-yl)isonicotinamide | Dal Piaz, V., et. al., Eur. J. Med. Chem. 35 (2000) 463-480 |
| 46. | [4-cyclopropyl-6- (cyclopropylamino)- 1,3,5-triazin- 2-yl]-1lambda˜4˜,4- thiazine-1,1- diol | 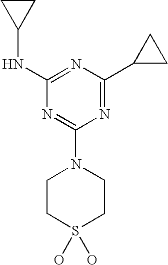 | 4-[4-cyclopropyl-6- (cyclopropylamino)- 1,3,5-triazin-2-yl]- 1lambda˜4˜,4- thiazinane-1,1-diol | Dal Piaz, V., et. al., Eur. J. Med. Chem. 35 (2000) 463-480 |
| 47. | N-cyclopropyl-4- (2-methylcyclopropyl)- 6-(2- methylmorpholin- 4-yl)-1,3,5- triazin-2- amine | 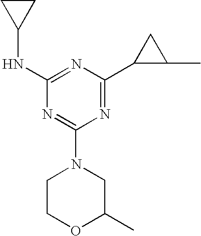 | N-cyclopropyl-4-(2- methylcyclopropyl)-6-(2- methylmorpholin-4-yl)-1,3,5- triazin-2-amine | Dal Piaz, V., et. al., Eur. J. Med. Chem. 35 (2000) 463-480 |
| 48. | Atizoram CP 80633 CAS RN: 135637-46-6 | 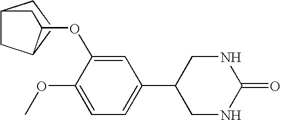 | 2(1H)-Pyrimidinone, 5-[3- [(1S,2S,4R)- bicyclo[2.2.1 ]hept- 2-yloxy]-4- methoxyphenyl]tetrahydro- | Souness, J., et al., Immunopharmacology 47 (2000) 127-162 |
| 49. | Filaminast WAY-PDA-641 CAS RN: 141184-34-1 | 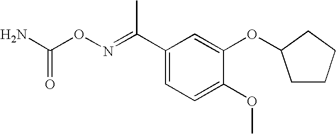 | Ethanone, 1-(3-(cyclopentyloxy)-4- methoxyphenyl-,O-(aminocarbonyl) oxime, (E) | Souness, J., et al., Immunopharmacology 47 (2000) 127-162 |
| 50. | Piclamilast RP 73401 RPR 73401 CAS RN: 144035-83-6 | 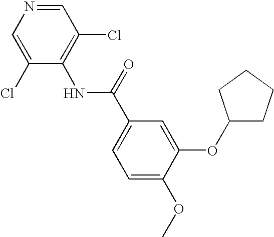 | Benzamide, 3-(cyclopentyloxy)-N- (3,5-dichloro-4-pyridinyl)-4-methoxy | Dal Piaz, V., et. al., Eur. J. Med. Chem. 35 (2000) 463-480 |
| 51. | Tibenelast Sodium LY 186655 CAS RN: 105102- 18-9 | 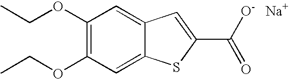 | Sodium 5,6-diethoxybenzo(b) thiophene-2-carboxylate | Souness, J., et al., Immunopharmacology 47 (2000) 127-162 |
| 52. | CDP 840 CAS RN: 162542-90-7 | 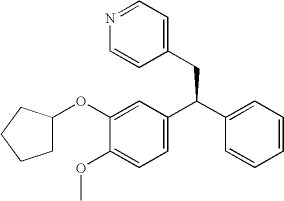 | Pyridine, 4-[(2R)-2-[3- (cyclopentyloxy)-4-methoxyphenyl]- 2-phenylethyl]- | Souness, J., et al., Immunopharmacology 47 (2000) 127-162 |
| 53. | GW 3600 GL 193600X CAS RN: 173258-94-1 | 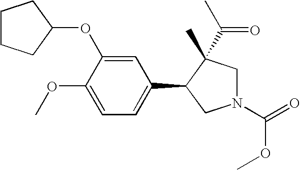 | 1-Pyrrolidinecarboxylic acid, 3- acetyl-4-[3-(cyclopentyloxy)-4- methoxyphenyl]-3-methyl-, methyl ester, (3R,4R) | US 2002/0103 106 A1 |
| 54. | NCS 613 CAS RN: 190377-71-0 | 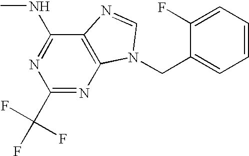 | 9H-Purin-6-amine, 9-[(2- fluorophenyl)methyl]-N- methyl-2-(trifluoromethyl)- | US 2002/0103 106 A1 |
| 55. | PDB 093 | No Structure | | US |
| | CAS RN: | | | 2002/0103 |
| | 444657-05-0 | | | 106 A1 |
| 56. | Ro 20-1724 CAS RN: 29925-17-5 | 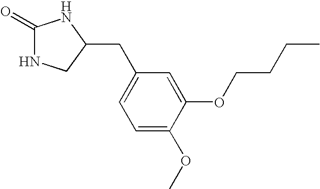 | 2-Imidazolidinone, 4-[(3-butoxy-4-methoxyphenyl) methyl] | US 2002/0103 106 A1 |
| 57. | RS 25344-000 CAS RN: 152814-89-6 | 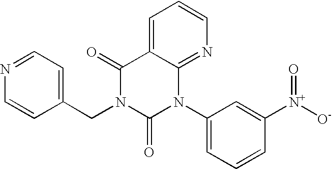 | Pyrido[2,3-d]pyrimidine-2,4(1H,3H)- dione, 1-(3-nitrophenyl)-3-(4- pyridinylmethyl) | Dal Piaz, V., et. al., Eur. J. Med. Chem. 35 (2000) 463-480 |
| 58. | SKF 107806 | No Structure | | US |
| | CAS RN: | | | 2002/0103 |
| | 444615-76-3 | | | 106 A1 |
| 59. | XT-44 CAS RN: 135462-05-4 | 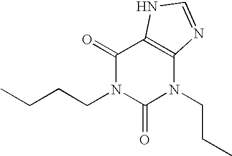 | 1-n-butyl-3-n-propylxanthine | Waki, Y., et al., Jpn J Pharmacol 79(4): 477-83 (1999) |
| 60. | tolafentrine | 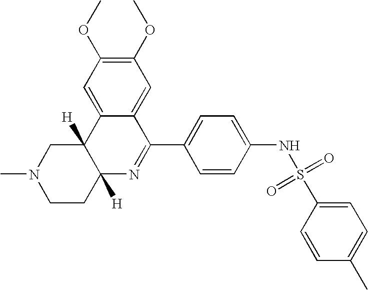 | Benzenesulfonamide, N-[4-[(4aR, 10bS)-1,2,3,4,4a,10b- hexahydro-8,9-dimethoxy-2- methylbenzo[c]8 6]naphthyridin-6-yl]phenyl]-4-methyl | US 2002/0103 106 A1 |
| 61. | zardaverine | 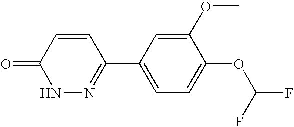 | 3(2H)-Pyridazinone,6-[4- (difluoromethoxy)-3- methoxyphenyl] | Souness, J., et al., Immunopharmacology 47 (2000) 127-162 |
| 62. | T-2585 | 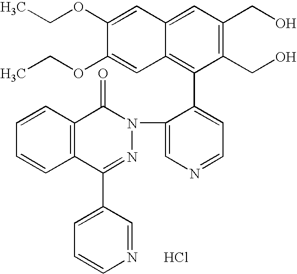 | 2-[4-(6,7-Diethoxy-2,3-bis- hydroxymethyl-napthalen-1-yl)- pyridin-3-yl]-4-pyridin-3-yl-2H- phthalazin-1-one; compound with generic inorganic neutral component | US 2002/0103 106 A1 |
| 63. | SDZ-ISQ-844 | 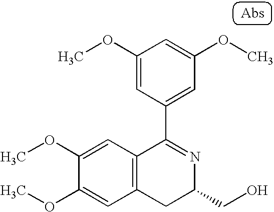 | [1-(3,5-Dimethoxy-phenyl)-6,7- dimethoxy-3,4-dihydro- isoquinolin-3-yl]-methanol | US 2002/0103 106 A1 |
| 64. | SB 207499 | 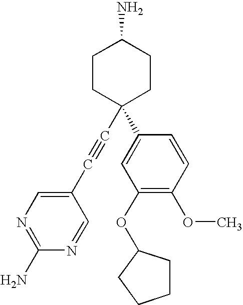 | 5-[4-Amino-1-(3-cyclopentyloxy- 4-methoxy-phenyl)- cyclohexylethynyl]- pyrimidin-2-ylamine | Souness, J., et al., Immunopharmacology 47 (2000) 127-162 |
| 65. | RPR- 117658A | 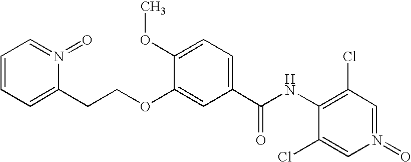 | N-(3,5-Dichloro-1-oxy-pyridin-4- yl)-4-methoxy-3-[2-(1-oxy-pyridin-2- yl)-ethoxy]-benzamide | US 2002/0103 106 A1 |
| 66. | L-787258 | No structure | | US |
| | | | | 2002/0103 |
| | | | | 106 A1 |
| 67. | E-4021 | 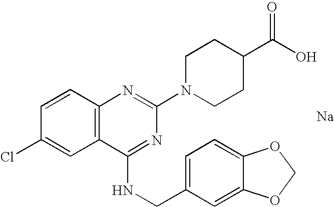 | 1-{4-[(Benzo[1,3]dioxol-5- ylmethyl)-amino]-6-chloro- quinazolin-2-yl}-piperidine-4- carboxylic acid; compound with generic inorganic neutral component | US 2002/0103 106 A1 |
| 68. | GF-248 | 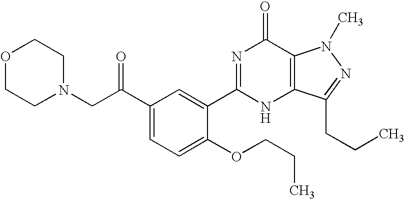 | 1-Methyl-5-[5-(2-morpholin-4-yl- axetyl)-2-propoxy-phenyl]-3- propyl-1,4-dihydro- pyrazolo[4,3-d]pyrimidin- 7-one | US 2002/0103 106 A1 |
| 69. | IPL-4088 | No structure | | US |
| | | | | 2002/0103 |
| | | | | 106 A1 |
| 70. | CP- 353164 | 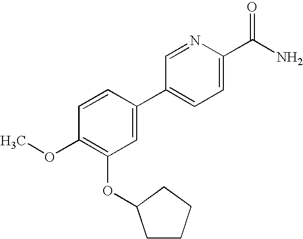 | 5-(3-Cyclopentyloxy-4-methoxy- phenyl)-pyridine-2- carboxylic acid amide | US 2002/0103 106 A1 |
| 71. | CP- 146523 | 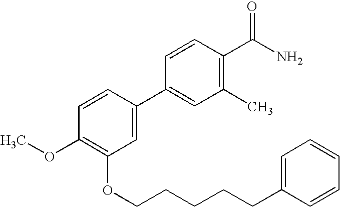 | 4′-Methoxy-3-methyl-3′-(5- phenyl-pentyloxy)-biphenyl-4- carboxylic acid | US 2002/0103 106 A1 |
| 72. | CP- | No structure | | US |
| | 293321 | | | 2002/0103 |
| | | | | 106 A1 |
| 73. | XT-611 | 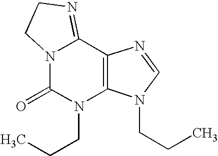 | 3,4-Dipropyl-3,4,6,7-tetrahydro- 1,3,4,5a,8-pentaaza-as-indacen- 5-one | US 2002/0103 106 A1 |
| 74. | WAY- | No structure | | US |
| | 126120 | | | 2002/0103 |
| | | | | 106 A1 |
| 75. | WAY- 122331 | 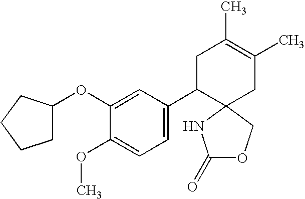 | 1-(3-Cyclopentoxy-4-methoxy-phenyl)- 7,8-methyl-3-oxa-1-aza- spiro[4.5]dec-7-en-2-one | US 2002/0103 106 A1 |
| 76. | WAY- 127093B | 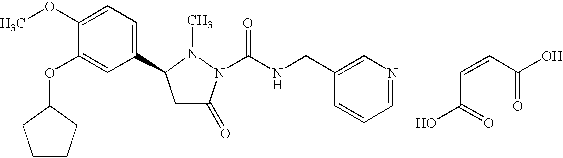 | 3-(3-Cyclopentyloxy-4-methoxy- phenyl)-2-methyl-5-oxo- pyrazolidine-1-carboxylic acid (pyridin-3-ylmethyl)-amide; compound with but- 2-enedioic acid | US 2002/0103 106 A1 |
| 77. | PDB-093 | No structure | | US |
| | | | | 2002/0103 |
| | | | | 106 A1 |
| 78. | CDC-801 | 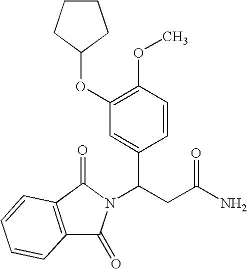 | 3-(3-Cyclopentyloxy-4-methoxy- phenyl)-3-(1.3-dioxo-1,3-dihydro- isoindol-2-yl)-propionamide | US 2002/0103 106 A1 |
| 79. | CC-7085 | No structure | | US |
| | | | | 2002/0103 |
| | | | | 106 A1 |
| 80. | CDC-998 | No structure | | US |
| | | | | 2002/0103 |
| | | | | 106 A1 |
| 81. | CH-3697 | No structure | | US |
| | | | | 2002/0103 |
| | | | | 106 A1 |
| 82. | CH-3442 | No structure | | US |
| | | | | 2002/0103 |
| | | | | 106 A1 |
| 83. | CH-2874 | No structure | | US |
| | | | | 2002/0103 |
| | | | | 106 A1 |
| 84. | CH-4139 | No structure | US | |
| | | | | 2002/0103 |
| | | | | 106 A1 |
| 85. | RPR- 114597 | 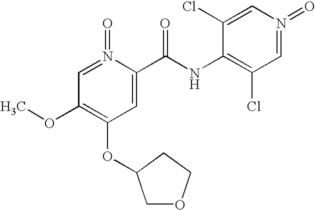 | 5-Methoxy-1-oxy-4-(tetrahydro- furan-3-yloxy)-pyridine-2- carboxylic acid (3,5-dicloro- 1-oxy-pyridin-4-yl) amide | US 2002/0103 106 A1 |
| 86. | RPR- 122818 | 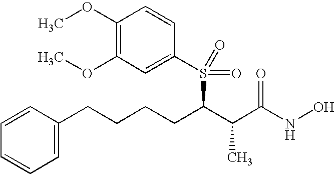 | 3-3(3,4-Dimethoxy- bemzenesulfonyl)-2-methyl-7-phenyl- heptanoic acid hydroxamide | US 2002/0103 106 A1 |
| 87. | KF-19514 | 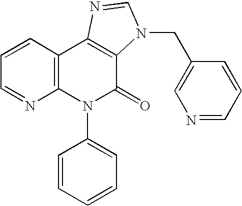 | 5-Phenyl-3-pyridin- 3-ylmethyl-3,5-dihydro-1,3,5,6- tetraaza-cyclopenta[a]naphthalene-4-one | US 2002/0103 106 A1 |
| 88. | CH-422 | No structure | | US |
| | | | | 2002/0103 |
| | | | | 106 A1 |
| 89. | CH-673 | No structure | | US |
| | | | | 2002/0103 |
| | | | | 106 A1 |
| 90. | CH-928 | No structure | | US |
| | | | | 2002/0103 |
| | | | | 106 A1 |
| 91. | KW-4490 | No structure | | US |
| | | | | 2002/0103 |
| | | | | 106 A1 |
| 92. | Org 20241 | 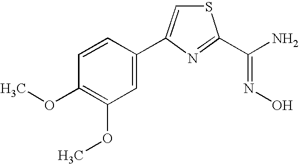 | 4-(3,4-Dimethoxy-phenyl)-N- hydroxy-thiazole-2- carboxamidine | US 2002/0103 106 A1 |
| 93. | Org 30029 | 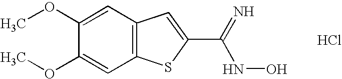 | N-Hydroxy-5,6-dimethoxy- benzo[b]thiopene- 2carboxamidine; compound with a generic inorganic neutral component | US 2002/0103 106 A1 |
| 94. | VMX 554 | No Structure | | New Drugs for |
| | VMX 565 | | | Respiratory |
| | | | | Diseases, 5^{th} |
| | | | | International |
| | | | | Conference, |
| | | | | San Diego, |
| | | | | CA, USA, |
| | | | | July 3-5, |
| | | | | 2002 |
| 95. | Benafentrine | 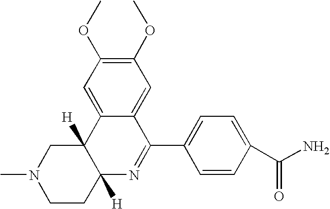 | Acetamide, N-[4-[(4aR,10bS)- 1,2,3,4,4a,10b-hexahydro-8,9- dimethoxy-2- methylbenzo[c][1,6]napthyridin-6-yl]phenyl] | U.S. Pat. No. 6,333,354 B1 |
| 96. | Trequinsin | 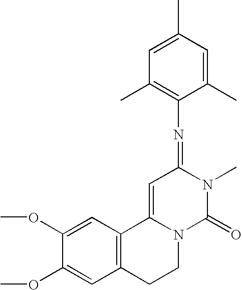 | 4H-Pyrimido[6,1-a]isoquinolin-4-one, 2,3,6,7-tetrahydro-9,10-dimethoxy-3- methyl-2-[(2,4,6- trimethylphenyl)imino] | U.S. Pat. No. 6,333,354 B1 |
| 97. | EMD 54622 | 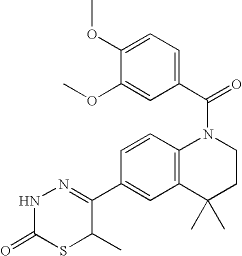 | Quinoline, 6-(3,6-dihydro-6-methyl-2- oxo-2H-1,3,4-thiadiazin-5-yl)-1- (3,4-dimethoxybenzoyl)- 1,2,3,4-tetrahydro-4,4-dimethyl | U.S. Pat. No. 6,333,354 |
| 98. | RS 17597 | 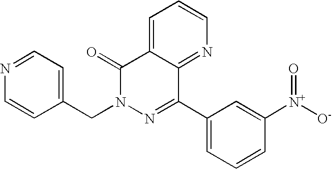 | Pyrido[2,3-d]pyridazin-5(6H)-one, 8-(3-nitrophenyl)-6-(4- pyridinylmethyl) | US 2002/0103 106 A1 |
| 99. | Nitraquazone | 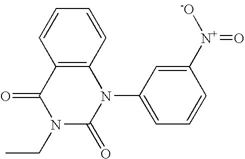 | 2,4(1H,3H)-Quinazolinedione, 3-ethyl-1-(3-nitrophenyl) | Dal Piaz, V., et. al., Eur. J. Med. Chem. 35 (2000) 463-480 |
| 100. | Oxagrelate | 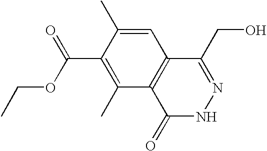 | 6-Phthalazinecarboxylic acid, 3,4-dihydro-1-(hydroxymethyl)- 5,7-dimethyl-4-oxo-, ethyl ester | U.S. Pat. No. 6,333,354 B1 |

In one embodiment the PDE IV inhibitor is a catechol ether selected from the group consisting of cilomilast, roflumilast, pumafentrin, L-869298, ZK-117137, and rolipram. In a preferred embodiment the PDE IV inhibitor is cilomilast. In another preferred embodiment the PDE IV inhibitor is roflumilast. In another preferred embodiment the PDE IV inhibitor is rolipram.

In another embodiment the PDE IV inhibitor is a quinazolidione or related compound selected from the group consisting of YM-976, Sch-351591, IC-485, Sch-365351, PD-189659, CP-77059, RS-14203 e, AWD-12-281, D-22888, and YM-58977.

In another embodiment the PDE IV inhibitor is a xanthine or related compound selected from the group consisting of Theophylline, cipamfylline, arofylline, V-11294A, RPR-132294, IBMX, isbufylline, doxofylline, dyphylline, verofylline, bamifylline, pentoxifylline, enprofylline, denbufylline, Chiroscience 245412, ICI-63197, SCA-40, ibudilast, N-cyclopentyl-8-cyclopropyl-3-propyl-3H-purin-6-amine, and 8-cyclopropyl-N,3-diethyl-3H-purin-6-amine. In a preferred embodiment the PDE IV inhibitor is theophylline. In another preferred embodiment the PDE IV inhibitor is arofylline. In another preferred embodiment the PDE IV inhibitor is doxofylline. In another preferred embodiment the PDE IV inhibitor is dyphylline. In another preferred embodiment the PDE IV inhibitor is bamifylline. In another preferred embodiment the PDE IV inhibitor is ibudilast.

In another embodiment the PDE IV inhibitor is a benzofuran, benzopyran or related compound selected from the group consisting of lirimilast, (4-chlorophenyl)[3-(3,3-dihydroxybutyl )-6-hydroxy-1-benzofuran-2-yl]methanone, 1-{3-(dimethylamino)-4-[(dimethylamino)methyl]-7-hydroxy-5,6-dimethyl-1-benzofuran-2-yl}ethanone, N-(3,5-dichloropyridin-4-yl)-8-methoxy-2,2-dimethylchromane-5-carboxamide, and 2-acetyl-N-benzyl-7-methoxy-1-benzofuran-4-sulfonamide. In another embodiment the PDE IV inhibitor is selected from the group consisting of 1-cyclopentyl-N-(3,5-dichloropyridin-4-yl )-3-ethyl-1H-indazole-6-carboxamide, 1-cyclopentyl-3-ethyl-6-(2-methylphenyl)-1,3a,4,5,6,7a-hexahydro-7H-pyrazolo[3,4-c]pyridin-7-one, N-(4-oxo-1-phenyl-3,4,6,7-tetrahydro[1,4]diazepino[6,7,1-hi]indol-3-yl)-1H-indole-2-carboxamide, Cl-1118, 4-[4-cyclopropyl-6-(cyclopropylamino)-1,3,5-triazin-2-yl]-1lambda˜4˜,4-thiazinane-1,1-diol, N-cyclopropyl-4-(2-methylcyclopropyl )-6-(2-methylmorpholin-4-yl )-1,3,5-triazin-2-amine, and atizoram, filaminast, piclamilast, tibenelast, CDP 840, GW 3600, NCS 613, PDB 093, Ro 20-1724, RS 25344-000, SKF 107806, XT-44, tolafentrine, zardaverine, T-2585, SDZ-ISQ-844, SB 207499, RPR-117658A, L-787258, E-4021, GF-248, IPL-4088, CP-353164, CP-146523, CP-293321, T-611,WAY-126120, WAY-122331,WAY-127093B, PDB-093, CDC-801, CC-7085, CDC-998, CH-3697, CH-3442, CH-2874, CH-4139, RPR-114597, RPR-122818, KF-19514, CH-422, CH-673, CH-928, KW-4490, Org 20241, Org 30029,VMX 554, VMX 565, benafentrine, trequinsin, EMD 54622, RS 17597, Nitraquazone, oxagrelate, T-440.

Specific PDE-IV inhibitors mentioned by way of example include RO-20-1724, DENBUFYLLINE, OXAGRELATE, NITRAQUAZONE, Y-590, DH-6471, SKF-94120, MOTAPIZONE, LIXAZINONE, INDOLIDAN, OLPRINONE, ATIZORAM, KS-506-G, DIPAMFYLLINE, BMY-43351, ATIZORAM, AROFYLLINE, FILAMINAST, PDB-093, UCB-29646, CDP-840 and the S-enantiomer thereof, CT1731, SKF-107806, PICLAMILAST, RS-17597, RS-25344-000, SB-207499, TIBENELAST, SB-210667, SB-211572, SB-211600, SB-212066, SB-212179 and GW-3600, in particular MOPIDAMOL, ANAGRELIDE, IBUDILAST, AMRINONE, PIMOBENDAN, CILOSTAZOL, LAS-31025-Almirall; Propentophylline (PPF also known as HWA-285); L-826,141; QUAZINONE and N-(3,5-dichloropyrid-4-yl)-3-cyclopropylmethoxy-4-difluoromethoxybenzamide; and

CILOMILAST (Ariflo®, SB 207499) c-4-cyano-4-(3-cyclopentyloxy-4-methoxyphenyl-r-1-cyclohexane carboxylic acid), SmithKline Beecham Pharmaceuticals plc, (Harlow, UK), having the structure:

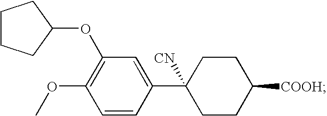

D4418; D4396; SCH351591; MESOPRAM, Chiroscience and Schering-Plough;

ROLIPRAM [4-(3-cyclopentenyloxy-4-methoxyphenyl)-2-pyrrolidone], CAS [61413-54-5], Schering A G (Berlin, Germany), having the structure:

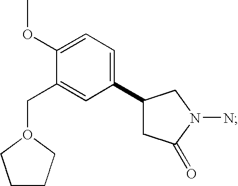

YM976 (4-(3-chlorophenyl)-1,7-diethylpyrido[2,3-d]pyrimidin-2(1H)-one-Yamanouchi Pharmaceutical Co. Ltd. (Tsukuba, Japan) having the structure:

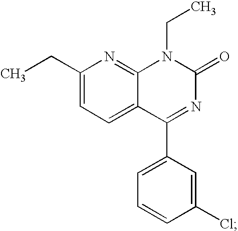

RP7340 1 (3-cyclopentyloxy-N-(3,5-dichloro-4-pyridyl )-4-methoxybenzamide);

CT-2450, ((R)-N-{4-[1-(3-cyclopentyloxy-4-methoxyphenyl )-2-(4-pyridyl)ethyl]phenyl}N′-ethylurea), Celltech Group plc (Berkshire, GB), having the structure:

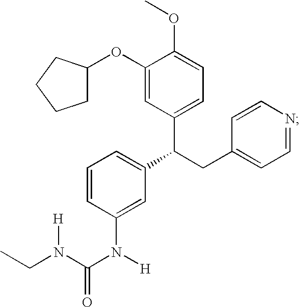

CT-3405, Celltech Group plc (Berkshire, GB), having the structure:

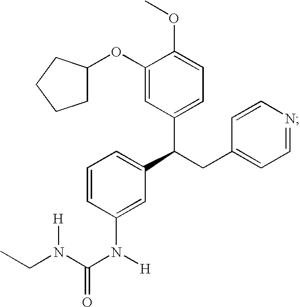

and compounds described in U.S. Pat. No. 5,712,298, Amschler, BYK Gulden Lomberg Chemische Fabrik GmbH (Konstanz, Germany), particularly the compound ROFLUMILAST (RP 73401), (benzamide 3-(cyclopropylmethoxy)-N-(3,5-dichloro-4-(difluoromethoxy)-(9Cl)), having the structure:

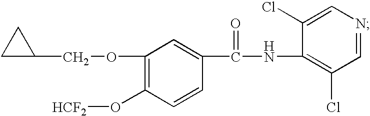

and BENAFENTRINE (6-(p-acetamidophenyl)-1,2,3,4,4a, 10b-hexahydro-8,9-dimethoxy-2-methyl-benzo[c][1,6]naphthyridine); BAY 19-8004, Bayer; Pumafentrine; INS-365; AWD 12-281, Asta Medica (now known as Elbion); compounds described in U.S. Patent No. 6,384,236, Pfizer; CDC-801 and CDC-998, Celgene; and 5CC (catechole hydrazine type derivatives), Cheil Je Dang Corp.

PDE-III/IV dual inhibitors mentioned by way of example include TREQUINSINE, ORG-30029, L-686398, SDZ-ISQ-844, ORG-20241, EMD-54622; ZARDAVERINE; TOLAFENTRINE, Byk Gulden Pharmaceuticals (Konstanz, Germany).

PDE-III inhibitors mentioned by way of example include AMRINONE, SULMAZOLE, AMPIZONE, CILOSTAMIDE, CARBAZERAN, PIROXIMONE, IMAZODAN, CI-930, SIGUAZODAN, ADIBENDAN, SATERINONE, SKF-95654, SDZ-MKS-492, 349-U-85, EMORADAN, EMD-53998, EMD-57033, NSP-306, NSP-307, REVIZINONE, NM-702, WIN-62582 and WIN-63291, in particular ENOXIMONE and MILRINONE; VESNARINONE; INDOLIDANE; QUAZINONE; MOTAPIZONE; SK&F 94836; MKS 492; CI-930 (4,5-dihydro-6-[4-(1H-imidazol-1-yl)-phenyl]-5-methyl-3(2H)-pyridazinone), Tanabe Seiyaku (Osaka, Japan); and

ATZ-1993 having the structure:

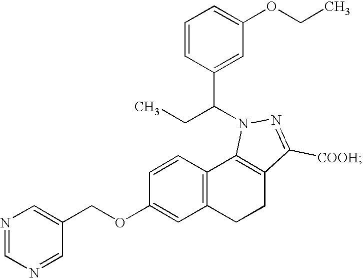

OLPRINONE (E-1020:1,2-Dihydro-6-methyl-2-oxo-5-[imidazo(1,2-a)pyridin-6-yl]-3-pyridine carbonitrile hydrochloride monohydrate); and CILOSTAZOL.

Specific PDE V Inhibitors mentioned by way of example include dipyridamole, MY-5445, RX-RA-69, SCH-51866, KT-734, VESNARINONE, ZAPRINAST, SKF-96231, ER-21355, BF/GP-385, NM-702 and SILDENAFIL.

Specific PDE VI Inhibitors mentioned by way of example include dipyridamole and zaprinast.

### Illustrative Examples of iNOS Selective Inhibitors

The following synthesis examples are shown for illustrative purposes and in no way intended to limit the scope of the invention. Where isomers are not defined, utilization of appropriate chromatography methods will afford single isomers.

### Example A

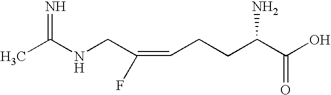

### (2S,5E)-2-amino-6-fluoro-7-[(1-iminoethyl)amino]-5-heptenoic acid, dihydrochloride, monohydrate

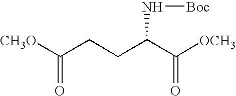

EX-A-1) Trimethylsilyl chloride (107.8 g, 1.00 mol) was added dropwise to a cooled solution of L-glutamic acid (30.00 g, 0.20 mol) in 300 mL of methanol at 0° C. The resulting clear, colorless solution was allowed to stir at room temperature. After 18 h, analysis by thin layer chromatography (30% ethyl acetate in hexane) showed that no starting material remained. The reaction was then cooled to 0° C., triethylamine (134 g, 1.33 mol) was added, and a white precipitate formed. Di-tert-butyldicarbonate (49 g, 0.23 mol) was added, and the mixture was allowed to warm to room temperature. After 3 h the solvent was removed, and 700 mL of diethyl ether was added. The solution was filtered, and the filter cake was rinsed with an additional 500 mL of diethyl ether. The filtrate was concentrated to 60.8 g (>95%) of a tan oil which was carried onto the next step without further purification. LCMS: m/z=298.1 [M+Na]⁺. HRMS calcd. for C₁₂H₂₁NO₆: 276.1447 [M+H]⁺, found: 276.1462. ¹H NMR (CDCl₃) δ1.45 (s, 9H), 1.95 (m, 1H), 2.50 (m, 1H), 2.40 (m, 2H), 3.69 (s, 3H), 3.75 (s, 3H), 4.32 (m,1H), 5.15 (m, 1H).

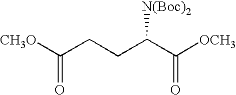

EX-A-2) To a solution of the crude product from EX-A-1 (60 g, 0.22 mol) in 300 mL of acetonitrile at room temperature was added 4-dimethylaminopyridine (5.3 g, 0.44 mol) and di-tert-butyldicarbonate (79.2 g, 0.36 mol). The resulting mixture was stirred for 2 days at room temperature, at which time analysis by thin layer chromatography (25% ethyl acetate in hexane) showed that most of the starting material was consumed. The solvent was removed in vacuo affording 85 g of a red oil. The crude material was purified by flash column chromatography on silica gel eluting with 1:10 ethyl acetate in hexane to give 66.4 g (81%) of the desired di-Boc product as a pale-yellow solid. LCMS: m/z=398.2 [M+Na]⁺. HRMS calcd. for C₁₇H₂₉NO₈: 398.1791 [M+Na]⁺, found: 398.1790. ¹H NMR (CDCl₃) δ1.48 (s, 18H), 2.19 (m, 1H), 2.41 (m, 2H), 2.46 (m, 1H), 3.66 (s, 3H), 3.70 (s, 3H), 4.91 (dd,1H).

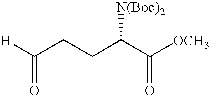

EX-A-3) A solution of DIBAL (64 mL of 1.0 M solution in hexanes, 63.9 mmol) was added dropwise to a cold solution of EX-A-2 (20 g, 53.3 mmol) in 400 mL of anhydrous diethyl ether at −78° C. over 30 min. After an additional 30 min at −78° C., the solution was quenched with water (12 mL, 666 mmol) and allowed to warm to room temperature. The cloudy mixture was diluted with 350 mL of ethyl acetate, dried over MgSO₄ and filtered through a pad of celite. The filtrate was concentrated to a yellow oil. The crude material, 18.9 g of yellow oil, was purified by flash column chromatography on silica gel eluting with 1:4 ethyl acetate in hexane to give 13.8 g (75%) of the desired aldehyde product as a clear oil. LCMS: m/z=368.2[M+Na]⁺. ¹H NMR (CDCl₃) δ1.48 (s, 18H), 2.19 (m, 1H), 2.41 (m, 2H), 2.46 (m,1H), 3.70 (s, 3H), 4.91 (dd,1 H), 9.8 (s,1H).

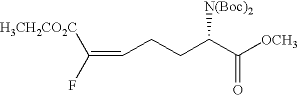

EX-A-4) To a cold (−78° C.) solution of triethyl 2-fluorophosphonoacetate (4.67 g, 19.3 mmol) in 20 mL of THF was added n-butyl lithium (10.9 mL of 1.6 M in hexane, 17.5 mmol). This mixture was stirred at −78° C. for 20 min producing a bright yellow solution. A solution of the product from EX-A-3 (6.0 g, 17.5 mmol) in 5 mL of THF was then added via syringe, and the resulting mixture was stirred for 2 h at −78° C., at which time analysis by thin layer chromatography (30% ethyl acetate in hexane) showed that no starting material remained. The reaction was quenched at −78° C. with sat. aqueous NH₄Cl (30 mL). The organic layer was collected, and the aqueous layer was extracted with diethyl ether (2×50 mL). The combined organics were washed with water (100 mL) and brine (100 mL), dried over MgSO₄, filtered and concentrated. The crude material, 8.6 g of a yellow oil, was purified by flash column chromatography on silica gel eluting with 1:4 ethyl acetate in hexane to give 6.05 g (79%) of the desired fluoro olefin product as a clear oil. ¹H NMR and ¹⁹F NMR indicated that the isolated product had an approximate E:Z ratio of 95:5. LCMS: m/z=456.2 [M+Na]⁺. HRMS calcd. for C₂₀H₃₂NO₈F: 456.2010 [M+Na]⁺, found: 456.2094. ¹H NMR (CDCl₃) δ1.48 (s, 18H), 2.0 (m, 1H), 2.25 (m, 1H), 2.6 (m, 2H), 3.7 (s, 3H), 4.25 (m, 2H), 4.9 (m, 1H), 5.9 (dt, vinyl, 1H, J=20 Hz), 6.2 (dt, vinyl, 1H, J=30 Hz). ¹⁹F NMR (CDCl₃) δ−129.12 (d, 0.09F, J=31 Hz, 9% Z-isomer), −121.6 (d, 0.91F, J=20 Hz, 91% E-isomer).

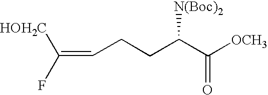

EX-A-5) To a solution of EX-A-4 (805 mg, 1.86 mmol) in 20 mL of methanol at room temperature was added solid NaBH₄ (844 mg, 22.3 mmol) in 200 mg portions. The reaction was stirred for 18 h at ambient temperature, at which time analysis by thin layer chromatography (30% ethyl acetate in hexane) showed that most of the starting material was consumed. The reaction was quenched with 20 mL of sat. aqueous NH₄Cl and extracted with ethyl acetate (2×35 mL). The organic layers were combined, dried over MgSO₄, filtered and concentrated. The crude material, 700 mg of clear oil, was purified by flash column chromatography on silica gel eluting with 1:4 ethyl acetate in hexane to give 353 mg (48%) of the desired allylic alcohol product as a clear oil, that contained primarily the desired E-isomer by ¹⁹F NMR. LCMS: m/z=414.2 [M+Na]⁺. ¹H NMR (CDCl₃) δ1.48 (s, 18H), 1.95 (m, 1H), 2.1 (m, 1H), 2.2 (m, 1H), 2.35 (t, 1H), 3.7 (s, 3H), 4.25 (m, 2H), 4.8 (m, 1H), 5.15 (dt, 1H, J=20 Hz). ¹⁹F NMR (CDCl₃) δ−119.1 (d, 0.02F, J=37 Hz, 2% Z-isomer), -111.8 (d, 0.98F, J=24 Hz, 98% E-isomer).

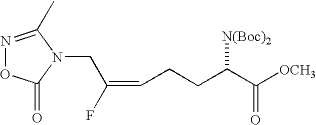

EX-A-6) To a mixture of EX-A-5 (1.37 g, 3.5 mmol), polymer-supported triphenylphosphine (3 mmol/g, 1.86 g, 5.6 mmol) and 3-methyl-1,2,4-oxadiazolin-5-one (450 mg, 4.55 mmol) in 50 mL of THF was added dropwise dimethylazodicarboxylate (820 mg, 5.6 mmol). The reaction was stirred for 1 h at room temperature, at which time analysis by thin layer chromatography (40% ethyl acetate in hexane) showed that no starting material remained. The mixture was filtered through celite, and the filtrate was concentrated. The resulting yellow oil was partitioned between 30 mL of methylene chloride and 30 mL of water. The organic layer was separated, washed with water (1×30 mL) and brine (1×30 mL), dried over MgSO₄, filtered and concentrated. The crude material, 1.8 g of a yellow oil, was purified by flash column chromatography on silica gel eluting with 1:4 ethyl acetate in hexane to give 670 mg (40%) of the desired protected E-allylic amidine product as a clear oil, that contained only the desired E-isomer by ¹⁹F NMR. LCMS: m/z=496.2 [M+Na]⁺. ¹H NMR (CDCl₃) δ1.48 (s, 18H), 1.85 (m, 1H), 2.2 (m, 3H), 2.25 (s, 3H), 3.64 (s, 3H), 4.25 (m, 2H), 4.8 (m, 1H), 5.3 (dt, 1H, J=20 Hz). ¹⁹F NMR (CDCl₃) δ−110.8 (q, 1F, J=20 Hz).

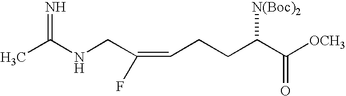

EX-A-7) The product from EX-A-6 (670 mg, 1.4 mmol) was dissolved in 25 mL of methanol and 25 mL of 25% acetic acid in water. Zinc dust (830 mg, 12.7 mmol) was added, and the mixture was agitated under sonication for 8 h, at which time HPLC analysis showed that only 20% of the starting material remained. The Zn dust was filtered from the reaction mixture, and the filtrate was stored at −20° C. for 12 h. The filtrate was warmed to room temperature, additional glacial acetic acid (7 mL) and zinc dust (400 mg, 6.1 mmol) were added, and the mixture was sonicated for 1 h at room temperature, at which time HPLC analysis showed 96% product. The mixture was filtered through celite, and the filtrate was concentrated. The crude material was purified by reverse-phase HPLC column chromatography on a YMC Combiprep column eluting over 8 min using a gradient of 20-95% A (A: 100% acetonitrile with 0.01% trifluoroacetic acid, B: 100% H₂O with 0.01% trifluoroacetic acid). Fractions containing product were combined and concentrated affording 344 mg (45%) of the desired acetamidine product as a trifluoroacetate salt, that contained only the desired E-isomer by ¹⁹F NMR. LCMS: m/z=432.3 [M+H]⁺. ¹H NMR (CD₃OD) δ1.52 (s, 18H), 2.9 (m, 1H), 2.2 (m, 3H), 2.27 (s, 3H), 4.2 (d, 1H), 5.4 (dt, vinyl, 1H, J=20 Hz). ¹⁹F NMR (CD₃OD) δ−110.83 (m, 1F, J=20 Hz).

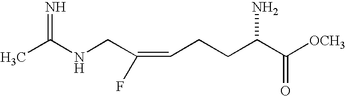

EX-A-8) A sample of the product of EX-A-7 is dissolved in glacial acetic acid. To this stirred solution is added 10 equivalents of 1N HCl in dioxane. After stirring this solution for ten minutes at room temperature, all solvent is removed in vacuo to generate the illustrated methyl ester dihydrochloride salt.

Example A) A solution of EX-A-7 (344 mg, 1.4 mmol) in 6 mL of 6.0 N HCl was refluxed for 1 h. The solvent was removed in vacuo. The resulting solid was dissolved in water and concentrated three additional times, followed by 5 subsequent times in 1.0 N HCl to remove any remaining TFA salts. Upon completion, 160 mg (37%) of the desired (2S,5E)-2-amino-6-fluoro-7-[(1-iminoethyl)amino]-5-heptenoic acid, dihydrochloride product was obtained as a white solid, m.p. 51.5-56.3° C., that contained only the desired E-isomer by ¹⁹F NMR. LCMS: m/z=218.1 [M+H]⁺. HRMS calcd. for C₉H₁₆FN₃O₂: 218.1305 ¹M+H]⁺, found: 218.1325. ¹H NMR (D₂O) δ1.8 (m, 2H), 2.05 (m, 2H), 2.1 (s, 3H), 3.7 (t, 1H), 4.00 (d, 2H), 5.3 (dt, vinyl, 1H, J=21 Hz). ¹⁹F NMR (D₂O) δ−109.9 (m, 1F, J=20 Hz).

### Example B

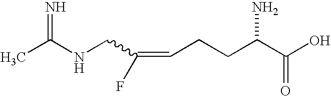

### (2S,5E/Z)-2-amino-6-fluoro-7-[(1-iminoethyl)amino]-5-heptenoic acid, dihydrochloride

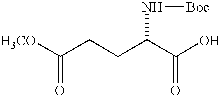

EX-B-1) To a cooled (0° C.) solution of L-glutamic acid 5-methyl ester (50.00 g, 0.31 mol) in 400 mL of 1:1 H₂O in dioxane was added triethylamine (38.35 g, 0.38 mol) followed by di-tert-butyldicarbonate (80.00 g, 0.37 mol). The resulting clear, colorless solution was allowed to stir at room temperature. After 18 h, analysis by thin layer chromatography (30% ethyl acetate in hexane) showed that no starting material remained. The reaction mixture was quenched with 200 mL of 1.0 N aqueous KHSO₄. The organic layer was removed, and the aqueous layer was extracted with ethyl acetate (3×100 mL). The organic layers were combined, dried over MgSO₄, filtered and concentrated to give 72.00 g (89%) of the desired product as a pale yellow oil. LCMS: m/z=284.1 [M+Na]⁺. ¹H NMR (CDCl₃) δ1.50 (s, 9H), 2.00 (m, 1H), 2.20 (m, 1H), 2.42 (m, 2H), 3.66 (s, 3H), 4.34 (d, 1H), 5.24 (d, 1H).

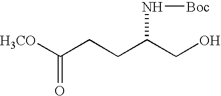

EX-B-2) To a solution of the product from EX-B-1 (72.60 g, 0.28 mol) in 300 mL of THF at −10° C. was quickly added 4-methylmorpholine (28.11 g, 0.28 mol) and isobutylchloroformate (37.95 g, 0.28 mol). The clear yellow solution immediately formed a white precipitate. After 4 min, the resulting cloudy yellow mixture was filtered, the filtrate was cooled to −10° C. and a solution of NaBH₄ (15.77 g, 0.42 mol) in 200 mL of H₂O was added dropwise while maintaining a subzero temperature. Once all of the NaBH₄ was added, the ice bath was removed, and the reaction was allowed to stir at room temperature for 1.5 h. The reaction mixture was quenched with 200 mL of H₂O. The organic layer was separated, and the aqueous layer was extracted with ethyl acetate (3×100 mL). The organic layers were combined, washed with brine, dried over MgSO₄, filtered and concentrated to give 58 g (85%) of the desired product as a yellow oil. LCMS: m/z=270.1 [M+Na]⁺. ¹H NMR (CDCl₃) δ1.42 (s, 9H), 1.65 (m, 1H), 1.85 (m, 2H), 2.42 (t, 2H), 3.66 (s, 3H), 4.8 (d, 1H).

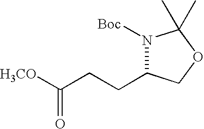

EX-B-3) To a solution of EX-B-2 (30.95 g, 0.13 mol) in 100 mL of benzene was added 2,2-dimethoxy propane (65.00 g, 0.63 mol) followed by p-toluenesulfonic acid (2.40 g, 12.5 mmol) and 5 g of 3 Å molecular sieves. The resulting mixture was refluxed for 2 h, at which time analysis by thin layer chromatography (30% ethyl acetate in hexane) showed complete reaction. The mixture was cooled to room temperature, diluted with diethyl ether (150 mL) and washed with sat. aqueous NaHCO₃ (100 mL) followed by brine (100 mL). The organic layer was dried over MgSO₄, filtered and concentrated. The crude material, 30.5 g of a yellow oil, was purified by flash column chromatography on silica gel eluting with 1:10 ethyl acetate in hexane to give 15.40 g (42%) of the desired product as a pale-yellow oil. LCMS: m/z=310.1 [M+Na]⁺. ¹H NMR (CDCl₃) δ1.42 (s, 12H), 1.56 (d, 3H), 1.85 (m, 2H), 2.38 (m, 2H), 3.66 (s, 3H), 3.7 (d, 1H), 3.95 (m, 2H).

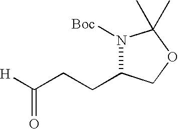

EX-B-4) DIBAL (6.0 mL of 1.0 M solution in toluene) was added dropwise to a cold (−78° C.) solution of the product from EX-B-3 (1.00 g, 3.00 mmol) in 10 mL of methylene chloride. After 30 min, the reaction was quenched with 5 mL sat. potassium sodium tartrate (Rochelle salt), then allowed to warm to room temperature. The mixture was then filtered through a pad of celite, dried over MgSO₄, re-filtered and concentrated to give a yellow oil. The crude material, 610 mg of a yellow oil, was purified by flash column chromatography on silica gel eluting with 1:4 ethyl acetate in hexane to give 550 mg (71%) of the desired product as a clear oil. ¹H NMR (CDCl₃) δ1.50 (s, 12H), 1.58 (d, 3H), 2.00 (m, 2H), 2.5 (m, 2H), 3.7 (d, 1H), 3.95 (m, 2H), 9.8 (s, 1H).

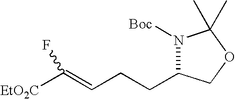

EX-B-5) To an ice cold (0° C.) solution of triethyl 2-fluoro-phosphonoacetate (6.70 g, 27.6 mmol) in 100 mL of methylene chloride was added 1,8-diazabicyclo[5.4.0]undec-7-ene (4.70 g, 31.0 mmol). The mixture was stirred at 0° C, for 1 h resulting in an orange solution. Then, a ice cold (0° C.) solution of the product from EX-B-4 (5.71 g, 22.2 mmol) in 15 mL of methylene chloride was added via syringe, and the resulting mixture was stirred for 18 h at ambient temperature, at which time analysis by thin layer chromatography (30% ethyl acetate in hexane) showed that no starting material remained. The solvent was removed in vacuo, and the resulting mixture was partitioned between 200 mL of ethyl acetate and 100 mL of water. The organic layer was collected, and the aqueous layer was extracted with ethyl acetate (2×50 mL). The combined organic layers were washed with 1.0 M aqueous KHSO₄ (100 mL), water (100 mL) and brine (100 mL), dried over MgSO₄, filtered and concentrated to give the desired fluoro olefin product as a yellow oil (8.0 g). ¹H NMR and ¹⁹F NMR indicated that the isolated product had an approximate Z:E ratio of 70:30. LCMS: m/z=368.2 [M+Na]⁺. ¹H NMR (CDCl₃) δ5.9-6.0 (dt, 1H, J=20 Hz), 6.05-6.20 (dt, 1H, J=33 Hz). ¹⁹F NMR (CDCl₃) δ−129.89 (d, 0.7F, J=38 Hz, 70% Z-isomer), −122.05 (d, 0.3F, J=20 Hz, 30% E-isomer). This mixture was carried on crude without further purification.

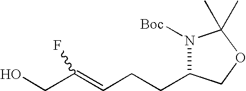

EX-B-6) To an ice cold (0° C.) solution of the product from EX-B-5 (8.0 g, 23.0 mmol) in 70 mL of THF was added LiBH₄ (12.7 mL of 2.0 M in THF, 25.0 mmol) via syringe. The reaction mixture was stirred for 18 h at ambient temperature at which time analysis by thin layer chromatography (30% ethyl acetate in hexane) showed that no starting material remained. The THF was removed, and the resulting mixture was dissolved in methylene chloride. After cooling to 0° C., 1.0 M aqueous KHSO₄ was slowly added to quench the reaction. The mixture was then extracted with ethyl acetate (3×50 mL). The organic layers were combined, dried over MgSO₄, filtered and concentrated. The crude material, 8.0 g of a clear oil, was purified by flash column chromatography on silica gel eluting with 1:4 ethyl acetate in hexane to give 900 mg (13%) of the desired product as a clear oil. LCMS: m/z=326.2 [M+Na]⁺. ¹H NMR (CDCl₃) δ4.79-4.94 (dm, 1H), 5.10-5.25 (dt, 1H). ¹⁹F NMR (CDCl₃) δ−119.82 (dt, 0.7F, J=38 Hz, 70% Z-isomer), −111.09 (dt, 0.3F, J=27 Hz, 30% E-isomer).

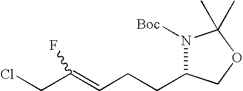

EX-B-7) To an ice cold (0° C.) solution of the product from EX-B-6 (950 mg, 3.1 mmol) in 5 mL of pyridine was added methanesulfonyl chloride (390 mg, 3.4 mmol). The reaction was stirred for 5 min at 0° C., then warmed to room temperature and stirred for 3 h, at which time analysis by thin layer chromatography (30% ethyl acetate in hexane) showed that no starting material remained. The reaction was diluted with diethyl ether (10 mL) and washed with sat. aqueous NaHCO₃ (20 mL) followed by 1.0 M citric acid (20 mL). The organic layer was dried over MgSO₄, filtered and concentrated to give 500 mg (51%) of the desired allylic chloride product as a white solid. This product was carried forward without further purification. LCMS: m/z=344.1 [M+Na]⁺.

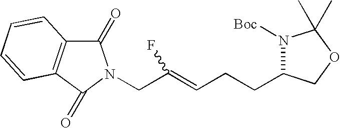

EX-B-8) To a stirring solution of the product from EX-B-7 (440 mg, 1.37 mmol) in 10 mL of DMF was added potassium phthalimide (290 mg, 1.57 mmol). The resulting mixture was heated under reflux for 18 h, at which time analysis by thin layer chromatography (30% ethyl acetate in hexane) showed that no starting material remained. The cooled mixture was diluted with 30 mL of water, extracted twice with ethyl acetate (30 mL), dried over MgSO₄, filtered and concentrated to give 540 mg (91%) of the desired product as a yellow oil. LCMS: m/z=455.2 [M+Na]⁺. HRMS calcd. for: 433.2139 [M+H]⁺, found: 433.2144. ¹H NMR (CDCl₃) δ1.4 (s, 18H), 1.6 (m, 6H), 2.05 (m, 2H), 3.6-4.42 (m, 4H), 4.9 (dt, vinyl, 1H), 5.2, (m, vinyl, 1H), 7.7 (m, 2H), 7.9 (m, 2H). ¹⁹F NMR (CDCl₃) δ−117.09 (m, 0.7F, J=38 Hz, 70% Z-isomer), −111.61 (m, 0.3F, J=22 Hz, 30% E-isomer).

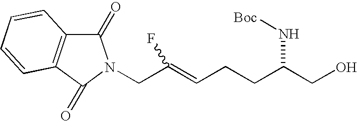

EX-B-9) The product from EX-B-8 (600 mg, 1.38 mmol) was dissolved in 8 mL of acetic acid and 2 mL of water. The mixture was stirred at room temperature overnight at which time analysis by thin layer chromatography (30% ethyl acetate in hexane) showed that no starting material remained. The solution was concentrated under a stream of nitrogen, and the crude product was purified by flash column chromatography on silica gel eluting with 1:2 ethyl acetate in hexane to give 248 mg (63%) of the desired product as a white solid. LCMS: m/z=415.1 [M+Na]⁺. ¹H NMR (CDCl₃) δ1.41 (s, 9H), 1.56 (m, 2H), 2.15 (m, 1H), 3.64 (m, 2H), 4.35 (d, 2H), 4.9 (dt, vinyl, 1H, J=37 Hz), 7.73 (m, 2H), 7.86 (m, 2H). ¹⁹F NMR (CDCl₃) δ−116.96 (dt, 0.8F, J=37 Hz, 80% Z-isomer), −111.09 (dt, 0.2F, J=22 Hz, 20% E-isomer).

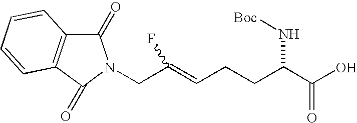

EX-B-10) To a stirring solution of the product from EX-B-9 (237 mg, 0.605 mmol) in 6 mL of DMF was added pyridinium dichromate (1.14 g, 3.03 mmol). The solution turned dark orange and was allowed to stir at room temperature for 18 H, at which time it was poured into 20 mL of H₂O. The mixture was extracted with ethyl acetate (4×25 mL). The combined organic layers were washed with 5% aqueous KHCO₃ (3×25 mL). The aqueous layer was acidified with 1.0 M KHSO₄ to pH=3 followed by extraction with ethyl acetate (3×50 mL). The combined organic layers were concentrated to yield 235 mg (95%) of the desired amino acid product. The resulting white solid was carried on crude without further purification. LCMS: m/z=429.1 [M+Na]⁺.

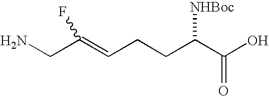

EX-B-11) To stirring solution of the product from EX-B-10 (230 mg, 0.56 mmol) in 7 mL of ethanol was added hydrazine hydrate (70 mg, 1.13 mmol), and the resulting solution was refluxed for 2 h forming a white precipitate. The solvent was removed in vacuo. The resulting white solid was dissolved in 8 mL of water and acidified to pH=4 with glacial acetic acid. It was then cooled in an ice bath and filtered. The filtrate was concentrated to give 136 mg (87%) of the desired allyl amine product as yellow crystals which were carried onto the next step without purification. LCMS: m/z=277.1 [M+H]⁺.

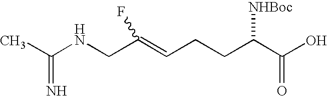

EX-B-12) To a stirring solution of the product from EX-B-11 (136 mg, 0.50 mmol) in 6 mL of DMF was added ethyl acetimidate (252 mg, 2.04 mmol) in 3 portions over 1.5 h intervals. After the addition was complete, the mixture was stirred overnight at room temperature. The pink solution was filtered, and the filter cake was washed with water. The solvent was removed in vacuo, and the resulting yellow oil was purified by reverse-phase HPLC using a YMC Combiprep ODS-A semi-prep column eluting with a 7 minute gradient of 1-50% A (A: 100 acetonitrile with 0.05% TFA, B: 100 water with 0.05% TFA). Fractions containing product were combined and concentrated to afford approximately 50 mg of the desired acetamidine product as a trifluoroacetate salt which was carried onto the next step. LCMS: m/z=318.2 [M+H]⁺.

Example B) The product from EX-B-12 was dissolved in 6 mL of 6.0 N HCl and stirred for 1 h at room temperature. The solvent was removed in vacuo. The resulting solid was dissolved in water and concentrated three additional times to remove TFA salts. When ¹⁹F NMR indicated that all of the TFA was removed, the product was dried in vacuo to give 30 mg (20%, combined yield over two steps) of a 20:80 E:Z mixture containing the desired (2S,5E)-2-amino-6-fluoro-7-[(1-iminoethyl)amino]-5-heptenoic acid, dihydrochloride and (2S,5Z)-2-amino-6-fluoro-7-[(1-iminoethyl)amino]-5-heptenoic acid, dihydrochloride as a foamy clear solid. HRMS calcd. for C₉H₁₆FN₃O₂: 218.1305 [M+H]⁺, found: 218.1309. ¹H NMR (D₂O) δ2.01 (m, 2H), 2.21 (s, 3H), 2.24 (m, 2H), 3.96 (t, 1H), 4.00 (d, 2H), 5.07 (dt, vinyl, 1H, J=37 Hz), 5.4 (dt, vinyl, 1 H, J=37 Hz). ¹⁹F NMR (D₂O) δ−116.8 (m, 0.8F, J=37 Hz, 80% Z-isomer), −109.6 (m, 0.2F, J=21 Hz, 20% E-isomer).

### Example C

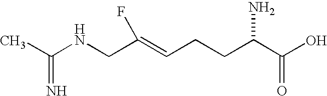

### (2S,5Z)-2-amino-6-fluoro-7-[(1-iminoethyl)amino]-5-heptenoic acid, dihydrochloride

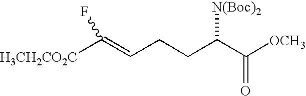

EX-C-1) Triethyl 2-fluoro-phosphonoacetate (3.54 g, 14.6 mmol) was dissolved in 20 mL of CH₂Cl₂ at 0° C., and 1,8-diazabicyclo[5.4.0]undec-7-ene (2.4 mL, 16.4 mmol) was added. The mixture was stirred at 0° C. for 20 min producing an orange solution. A solution of the aldehyde product from EX-A-3 (4.04 g, 11.7 mmol) was then added at 0° C., and the resulting brown mixture was stirred overnight at room temperature, at which time LCMS indicated that no starting material remained. The solvent was removed, and the residue was partitioned between water (60 mL) and ethyl acetate (120 mL). The organic layer was collected, and the aqueous layer was extracted with ethyl acetate (2×50 mL). The combined organic layers were washed with water (60 mL) and 10% aqueous KHSO₄ (60 mL), dried over MgSO₄, filtered and concentrated. The crude material, 5.7 g of an orange oil, was purified by flash column chromatography on silica gel eluting with 10% ethyl acetate in hexane to give 3.5 g (69%) of the desired fluoro olefin product as a clear oil. ¹H NMR and ¹⁹F NMR indicated that the isolated product had an Z/E ratio of 70:30. HRMS calcd. for C₂₀H₃₂O₈FN: 456.2010 [M+Na]⁺, found 456.2017. ¹H NMR (CDCl₃) δ1.48 (s, 18H), 2.0 (m, 1H), 2.25 (m, 1H), 2.6 (m, 2H), 3.7 (s, 3H), 4.25 (m, 2H), 4.9 (m, 1H), 5.9 (dt, vinyl, 1H, J=21.2 Hz), 6.1 (dt, vinyl, 1H, J=32.4 Hz). ¹⁹F NMR (CDCl₃) δ: −129.4 (d, 0.7F, J=34 Hz, 70% Z isomer), −121.6 (d, 0.3F, J=22 Hz, 30% E isomer).

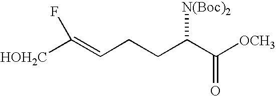

EX-C-2) The ester product from EX-C-1 (3.5 g, 8.1 mmol) was dissolved in 80 mL of methanol at room temperature, solid NaBH₄ (3 g, 80 mmol) was then added in portions. The mixture was stirred at room temperature for 18 h, at which time HPLC analysis indicated that the reaction was >90% complete. The reaction was quenched with sat NH₄Cl. The product was extracted with ethyl acetate and dried over Na₂SO₄. The organic layer was evaporated to give 3.2 g of crude product as a colorless oil, which was purified by Biotage flash column chromatography eluting with 20%-30% ethyl acetate in hexane to give 2.11 g (67%) of a Z/E mixture of the fluoro olefin product as a clear oil along with 0.41 g (13%) of the desired pure (Z:E=97:3 by ¹⁹F NMR) Z-isomer product as a clear oil. HRMS calcd. for C₁₈H₃₀NO₇F: 414.1904 [M+Na]⁺, found 414.1911. ¹H NMR (CDCl₃) δ1.48 (s, 18H), 2.0 (m, 1H), 2.2 (m, 3H), 3.7 (s, 3H), 4.1 (dd, 2H, J=17Hz), 4.8 (dt, 1H, J=39 Hz), 4.9 (m, 1H). ¹⁹F NMR (CDCl₃) δ−119.1 (dt, 1F, J=39 Hz, J=17 Hz).

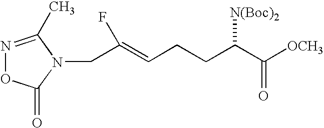

EX-C-3) The Z-alcohol product from EX-C-2 (390 mg, 1 mmol) and 3-methyl-1,2,4-oxadiazolin-5-one (130 mg, 1.3 mmol) were dissolved in 20 mL of THF. Then polymer supported-PPh₃ was added into the solution, and the mixture was gently stirred for 10 min. Then diethyl azodicarboxylate was added dropwise, and the mixture was stirred for 1 h at room temperature, at which time LCMS analysis indicated product formation and that no starting material was present. The polymer was filtered off through a celite pad, and the pad was washed with THF. The filtrate was evaporated to give 1.0 g of crude product which was purified by Biotage flash column chromatography eluting with 20% to 30% ethyl acetate in hexane to give 500 mg of product, contaminated with some hydrazide by-product. This material was further purified by Biotage flash column chromatography eluting with 98:2:0.01 of methylene chloride:methanol:ammon-ium hydroxide to give 180 mg (38%) of the desired protected amidine product as a clear oil, that contained only the desired Z-isomer by ¹⁹F NMR. HRMS calcd. for C₂₁H₃₂N₃O₈F: 491.2517 [M+NH₄]⁺, found 491.2523. ¹H NMR (CDCl₃) δ1.5 (s, 18H), 1.9 (m, 1H), 2.1 (m, 3H), 2.3 (s, 3H), 3.7 (s, 3H), 4.2 (d, 2H), 4.8 (m, 1H), 5.0 (dt, 1H, J=36 Hz). ¹⁹F NMR (CDCl₃) δ−116.5 (dt, 1 F, J=38 Hz).

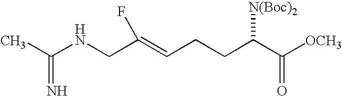

EX-C-4) The product from EX-C-3 (88 mg, 0.19 mmol) was dissolved in 4 mL of 25% acetic acid in water containing a few drops of methanol, and then Zn dust (109 mg, 1.67 mmol) was added. The mixture was agitated under sonication for 3 h. The Zn was filtered off through a celite pad, and the pad was washed with water. The filtrate was evaporated to dryness to give crude product which was purified by reverse-phase HPLC column chromatography on a YMC Combiprep column eluting over 8 min with a gradient of 20-80% A (A: 100% ACN with 0.01% TFA, B: 100% H₂O with 0.01% TFA). The desired product was collected in two fractions, and the combined fractions were concentrated. The product was obtained as a colorless oil as a mixture of trifluoroacetate salts that contained only the desired Z-isomer by ¹⁹F NMR: 30% was mono Boc-protected product: HRMS calcd. for C₁₅H₂₆N₃O₄F: 332.1986 [M+H]⁺, found 332.2001, and 70% was di-Boc-protected product: HRMS calcd. for C₂₀H₃₄N₃O₆F: 432.2510 [M+H]⁺, found 432.2503. ¹H NMR of the di-Boc product (D₂O) δ1.3 (s, 18H), 1.8 (m, 1H), 2.1 (m, 3H), 2.1 (s, 3H), 3.6 (s, 3H), 3.9 (d, 2H), 4.9 (dt, vinyl, 1H, J=37 Hz). ¹⁹F NMR (D₂O) δ−117.3 (dt, 1F, J=37 Hz).

Example C) The combined mono- and di-BOC products from EX-C-4 were dissolved in 30 mL of 6N HCl, and the solution was refluxed for 4 h, at which time LCMS analysis indicated complete reaction. The excess HCl and water was removed in vacuo. Upon completion, 9 mg (40% combined yield for two steps) of the desired (2S,5Z)-2-amino-6-fluoro-7-[(1-iminoethyl)amino]-5-heptenoic acid, dihydrochloride product was obtained as a light yellow, very hygroscopic foam, that contained only the desired Z-isomer by ¹⁹F NMR. HRMS calcd. for C₉H₁₆N₃O₂F: 218.1305 [M+H]⁺, found 218.1320. ¹H NMR (D₂O) δ1.3 (s, 18H), 1.9 (m, 2H), 2.1 (m, 2H), 2.1 (s, 3H), 3.8 (t, 1H), 3.9 (d, 2H), 4.9 (dt, vinyl, 1H, J=37 Hz). ¹⁹F NMR (D₂O) δ−117.3 (dt, 1F, J=37 Hz).

### Example D

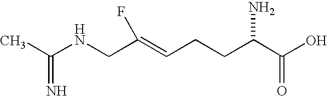

### (2S,5Z)-2-amino-6-fluoro-7-[(1-iminoethyl)amino]-5-heptenoic acid, trihydrochloride, dihydrate

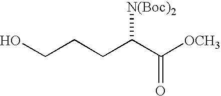

EX-D-1) The product from EX-D-2 (3.75 g, 10 mmol) was dissolved in 60 mL of methanol, and solid NaBH₄ (4 g, 106 mmol) was added in portions at room temperature over 10 h, at which time HPLC analysis indicated approximately 84% reduction. The reaction mixture was quenched with sat. NH₄Cl, and was then extracted with ethyl acetate three times. The combined organic layers were dried over MgSO₄, filtered, and evaporated to give 3.2 g of crude product as a yellow oil. HRMS calcd. for C₁₆H₂₉NO₇: 348.2022 [M+H]⁺, found: 348.2034. ¹H NMR (CD₃OD) δ4.9 (q, 1H), 3.7 (s, 3H), 3.5 (t, 2H), 3.2 (m, 1H), 2.1 (m, 1H), 1.9 (m, 2H), 1.5 (s, 18H).

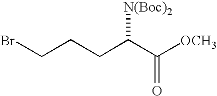

EX-D-2) The alcohol product from EX-D-1 (3.2 g, 9.0 mmol) was dissolved in 100 mL of THF and cooled in an ice bath. Carbon tetrabromide (4.27 g, 12.9 mmol) was added, and the resulting solution was stirred at 0° C. for 30 min under nitrogen. Polymer-supported PPh₃ was added, and the mixture was gently stirred at 0° C. for 1 h and then overnight at room temperature. The polymer was removed by filtration through celite, and the celite pad was washed with THF. The filtrate was evaporated to give crude product, which was purified by Biotage flash column chromatography eluting with 1:3 ethyl acetate in hexane to give 2.0 g (54%, combined yield over 2 steps) of the desired bromo product as a colorless oil. HRMS calcd. for C₁₆H₂₈NO₆Br: 410.1178 [M+H]⁺, found: 410.1137. ¹H NMR (CDCl₃) δ4.9 (q, 1H), 3.7 (s, 3H), 3.4 (m, 2H), 2.2 (m, 2H), 1.9 (m, 2H), 1.5 (s, 18H).

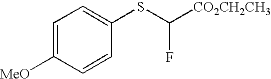

EX-D-3) A solution of NaOEt (21% in EtOH, 41.1 mL, 0.11 mol) in 60 mL of ethanol was treated with p-methoxy benzenethiol (14.0 g, 0.1 mol), followed by ethyl chlorofluoroacetate (18.3 g, 0.13 mol). The mixture was stirred at room temperature for 2 h and diluted with 250 mL of 1:1 hexane in ethyl acetate. The organic layer was washed with water three times, and dried over Na₂SO₄. The dried organic layer was evaporated to give 25 g of crude product which was carried forward without further purification. LCMS for C₁₁H₁₃O₃SF: m/z=267.10 [M+Na]⁺.¹ H NMR (CDCl₃) δ7.5 (d, 2H), 6.9 (d, 2H), 6.0 (d, 1H, J=51.9 Hz), 4.2 (q, 2H), 3.8 (s, 3H), 1.2 (t, 3H). ¹⁹F NMR (CDCl₃) δ−146.2 (d, 1F, J=53.6 Hz).

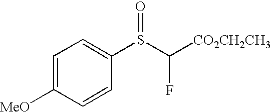

EX-D-4) A solution of the crude product from EX-D-3 (24 g, 0.1 mol) in 200 mL of methylene chloride was cooled to −78° C. and treated with 3-chloroperbenzoic acid (27 g, 0.12 mol) in 200 mL of methylene chloride. The reaction mixture was slowly warmed to room temperature and stirred overnight, at which time LCMS analysis indicated product formation and that no starting material remained. The solid was filtered off, and the filtrate was washed with sat. NaHCO₃ and NH₄Cl. The organic layer was dried over MgSO₄ and evaporated to give 30 g of an orange oil, which was purified by Biotage flash column chromatography eluting with 2:1 hexane in ethyl acetate to give 17.5 g (70%) of the desired sulfoxide product as an off-white oil. HRMS calcd. for C₁₁H₁₃O₄FS: 261.0597 [M+H]⁺, found: 261.0598. ¹H NMR (CDCl₃) δ7.6 (m, 2H), 7.0 (m, 2H), 5.6 (d, 1H, J=50 Hz major diastereomer), 5.4 (d, 1H, J=49 Hz minor diastereomer), 4.2 (q, 2H), 3.8 (s, 3H), 1.2 (t, 3H). ¹⁹F NMR (CDCl₃) δ−194.3 (d, 1F, J=53.6 Hz major diastereomer), −191.7 (d, 1F, J=50.4 Hz minor diastereomer).

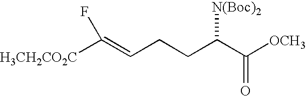

EX-D-5) A suspension of NaH (60% in mineral oil, 212 mg, 5.3 mmol) in 6 mL of dried DMF was cooled to 0° C. under nitrogen and treated with a solution of the sulfoxide product from EX-D-4 (1.25 g, 4.8 mmol) in 2 mL of DMF. After stirring at room temperature for 20 min, the mixture was cooled to 5° C., and the bromo product from EX-D-2 (2.17 g, 5.3 mmol) was added in one portion. The reaction was stirred at room temperature for 3 h, then heated at reflux at 95° C. for 1 h, at which time LCMS analysis indicated product formation. The mixture was poured into an ice/aqueous NH₄Cl mixture. The product was extracted with 1:1 hexane in ethyl acetate. The organic layer was dried over Na₂SO₄ and evaporated to give 3.17 g of a crude yellow oil, which was purified by Biotage flash column chromatography eluting with 10% ethyl acetate in hexane to give 1.05 g (50%) of the desired fluoro olefin ester product as a colorless oil. ¹⁹F NMR indicated that the isolated product contained 95:5 the desired Z-isomer. HRMS calcd. for C₂₀H₃₂O₈FN: 456.2010 [M+Na]⁺, found: 456.2017. ¹H NMR (CDCl₃) δ1.5 (s, 18H), 2.0 (m, 1H), 2.3 (m, 4H), 3.7 (s, 3H), 4.3 (m, 2H), 4.9 (m, 1H), 6.1 (dt, vinyl, 1H, J=32.4 Hz, Z isomer). ¹⁹F NMR (CDCl₃) δ−129.4 (d, 0.95F, J=34.8 Hz, 95% Z isomer), −121.6 (d, 0.05F, J=21.6 Hz, 5% E isomer).

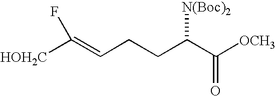

EX-D-6) The ester product from EX-D-5 (1.05 g, 2.4 mmol) was dissolved in methanol at room temperature, and solid NaBH₄ was added in portions. The mixture was stirred at room temperature for 18 h, then 2 mL of water was added, and the mixture was stirred for an additional 3 h, at which time HPLC analysis indicated the reaction was >95% complete. The reaction was quenched with sat NH₄Cl. The product was extracted with ethyl acetate, and the organic layer was dried over Na₂SO₄ and evaporated to give 0.95 g of crude product as colorless oil. ¹⁹F NMR indicated that the isolated crude product contained only the desired Z-isomer. HRMS calcd. for C₁₈H₃₀NO₇F: 414.1904 [M+Na]⁺, found: 414.1949. ¹H NMR (CDCl₃) δ1.48 (s, 18H), 2.0 (m, 1H), 2.2 (m, 3H), 3.7 (s, 3H), 4.1 (dd, 2H, J=17 Hz), 4.8 (dt, 1H, J=36 Hz), 4.9 (m, 1H). ¹⁹F NMR (CDCl₃) δ−119.1 (dt, 1F, J=38 Hz, J=17 Hz).

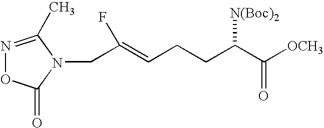

EX-D-7) The alcohol product from EX-D-6 (0.95 g, 2.4 mmol) and 3-methyl-1,2,4-oxadiazolin-5-one (290 mg, 2.9 mmol) were dissolved in 60 mL of THF. Polymer-bound triphenyl phosphine was added, and the mixture was gently stirred for 10 min. Then dimethyl azodicarboxylate was added dropwise, and the mixture was stirred for 1 h at room temperature, at which time LCMS analysis indicated product formation and that no starting material remained. The polymer was filtered off through a celite pad, and the pad was washed with THF. The filtrate was evaporated to give a residue which was partitioned between methylene chloride and water. The organic layer was washed with water twice, dried over MgSO₄, and evaporated to give 1.3 g of crude product which was purified by Biotage flash column chromatography eluting with 20% to 30% ethyl acetate in hexane to give 390 mg (34%, combined yield over 2 steps) of the desired protected amidine product as a colorless oil. ¹⁹F NMR indicated that the isolated product contained only the desired Z-isomer. HRMS calcd. for C₂₁H₃₂N₃O₈F: 491.2517 [M+NH₄]⁺, found: 491.2523. ¹H NMR (CDCl₃) δ1.5 (s, 18H), 1.9 (m, 1H), 2.1 (m, 3H), 2.3 (s, 3H), 3.7 (s, 3H), 4.2 (d, 2H), 4.8 (m, 1H), 5.0 (dt, 1H, J=36 Hz). ¹⁹F NMR (CDCl₃) δ−116.5 (dt, 1F, J=38 Hz).

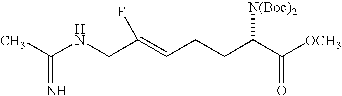

EX-D-8) The product from EX-D-7 (390 mg, 0.82 mmol) was dissolved in 20 mL of 25% HOAc in water containing 4 mL of methanol, and Zn dust (482 mg, 7.42 mmol) was added in two portions. The mixture was agitated under sonication for 3 h. The Zn was filtered off through a celite pad, and the pad was washed with water. The filtrate was evaporated to dryness to give crude product which was purified by reverse-phase-HPLC. Fractions containing the desired products were collected, combined and concentrated. The products were obtained as colorless oils as a mixture of trifluoroacetate salts, that contained only the desired Z-isomer by ¹⁹F NMR: 30% was mono-Boc protected product: HRMS calcd. for C₁₅H₂₆N₃O₄F: 332.1986 [M+H]⁺, found 332.2001; 70% was diBoc protected product: HRMS calcd. for C₂₀H₃₄N₃O₆F: 432.2510 [M+H]⁺, 432.2503. ¹H NMR of diBoc product (D₂O) δ1.3 (s, 18H), 1.8 (m, 1H), 2.1 (m, 3H), 2.1 (s, 3H), 3.6 (s, 3H), 3.9 (d, 2H), 4.9 (dt, vinyl, 1H, J=37 Hz). ¹⁹F NMR (D₂O) δ−117.3 (dt, 1F, J=37 Hz).

Example D) The mono and diBOC products from EX-D-8 were dissolved in 80 mL of 6N HCl and the solution was heated at reflux for 1 hour, at which time LCMS analysis indicated complete reaction. The excess HCl and water was removed in vacuo to give 150 mg (50% combined yield over 2 steps) of the desired (2S,5Z)-2-amino-6-fluoro-7-[(1-iminoethyl)amino]-5-heptenoic acid, trihydrochloride, dihydrate product as a light yellow very hygroscopic foam. HRMS calcd. for C₉H₁₆N₃O₂F: 218.1305 [M+H]⁺, found 218.1290. ¹H NMR (D₂O) δ1.3 (s, 18H), 1.9 (m, 2H), 2.1 (m, 2H), 2.1 (s, 3H), 3.8 (t, 1H), 3.9 (d, 2H), 4.9 (dt, vinyl, 1H, J=37 Hz). ¹⁹F NMR (D₂O) δ−117.3 (dt, 1F, J=37 Hz). Anal. Calcd. for C₉H₁₆N₃O₂F.3HCl.2H₂O: C, 29.81; H, 6.39; N, 11.59; found C, 29.80; H, 6.11; N, 11.20.

### Example E

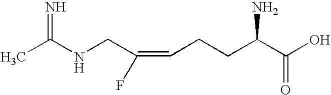

### (2R,5E)-2-amino-6-fluoro-7-[(1-iminoethyl)amino]-5-heptenoic acid, dihydrochloride, monohydrate

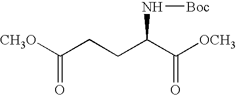

EX-E-1) Trimethylsilyl chloride is added dropwise to a cooled solution of D-glutamic acid in methanol at 0° C. The resulting clear, colorless solution is allowed to stir at room temperature until analysis by thin layer chromatography shows that no starting material remains. The reaction is then cooled to 0° C., triethylamine is added, and a white precipitate forms. Di-tert-butyldicarbonate is added, and the mixture is allowed to warm to room temperature. After 3 h the solvent is removed, and diethyl ether is added. The solution is filtered, and the filter cake is rinsed with additional diethyl ether. The filtrate is concentrated to give the desired mono-Boc diester product which is carried onto the next step without further purification.

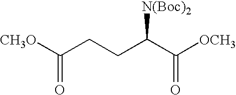

EX-E-2) To a solution of the crude product from EX-E-1 in acetonitrile at room temperature is added 4-dimethylaminopyridine and di-tert-butyldicarbonate. The resulting mixture is stirred at room temperature, until analysis by thin layer chromatography shows that most of the starting material is consumed. The solvent is removed in vacuo, and the resulting residue is purified by flash column chromatography on silica gel to give the desired di-Boc protected diester product.

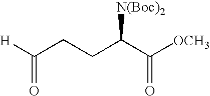

EX-E-3) A solution of DIBAL is added dropwise to a cold solution of EX-E-2 in anhydrous diethyl ether at −78° C. After 30 min at −78° C., the solution is quenched with water and allowed to warm to room temperature. The resulting cloudy mixture is diluted with ethyl acetate, dried over MgSO₄ and filtered through a pad of celite. The filtrate is concentrated, and the resulting residue is purified by flash column chromatography on silica gel to give the desired aldehyde product.

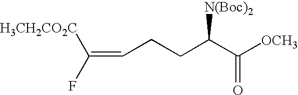

EX-E-4) To a cold (−78° C.) solution of triethyl 2-fluorophosphonoacetate in THF is added n-butyl lithium. This mixture is stirred at −78° C. producing a bright yellow solution. A solution of the product from EX-E-3 in THF is then added via syringe, and the resulting mixture is stirred at −78° C., until analysis by thin layer chromatography shows that no starting material remains. The reaction is quenched at −78° C. with sat. aqueous NH₄Cl. The organic layer is collected, and the aqueous layer is extracted with diethyl ether. The combined organics are washed with water and brine, dried over MgSO₄, filtered and concentrated. The crude material is then purified by flash column chromatography on silica gel to give the desired fluoro olefin product.

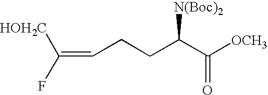

EX-E-5) To a solution of EX-E-4 in methanol at room temperature is added solid NaBH₄ in portions. The reaction is stirred at ambient temperature until analysis by thin layer chromatography shows that most of the starting material is consumed. The reaction is quenched with sat. aqueous NH₄Cl and extracted with ethyl acetate. The organic layers are combined, dried over MgSO₄, filtered and concentrated. The crude material is purified by flash column chromatography on silica gel to give the desired allylic alcohol product.

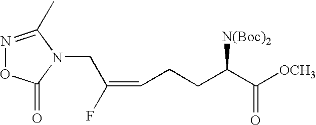

EX-E-6) To a mixture of EX-E-5, polymer-supported triphenylphosphine and 3-methyl-1,2,4-oxadiazolin-5-one in THF is added dropwise dimethylazodicarboxylate. The reaction mixture is stirred at room temperature until analysis by thin layer chromatography shows that no starting material remains. The mixture is filtered through celite, and the filtrate is concentrated. The resulting yellow oil is partitioned between methylene chloride and water. The organic layer is separated, washed with water and brine, dried over MgSO₄, filtered and concentrated. The crude material is purified by flash column chromatography on silica gel to give the desired protected E-allylic amidine product.

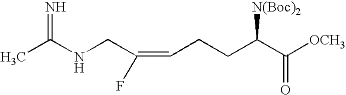

EX-E-7) The product from EX-E-6 is dissolved in methanol and acetic acid in water. Zinc dust is added, and the mixture is agitated under sonication until HPLC analysis shows that little of the starting material remains. The Zn dust is filtered through celite from the reaction mixture, and the filtrate is concentrated. The crude material is purified by reverse-phase HPLC column chromatography. Fractions containing product are combined and concentrated affording the desired acetamidine product as a trifluoroacetate salt.

Example E) A solution of EX-E-7 in 6.0 N HCl is refluxed for 1 h. The solvent is removed in vacuo. The resulting solid is dissolved in water and concentrated repeatedly from 1.0 N HCl to remove any remaining TFA salts to give the desired (2R,5E)-2-amino-6-fluoro-7-[(1-iminoethyl)amino]-5-heptenoic acid, dihydrochloride product.

### Example F

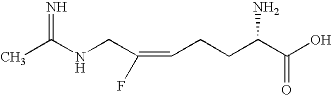

### (2S,5E)-2-amino-6-fluoro-7-[(1-iminoethyl)amino]-5-heptenoic acid, dihydrochloride, monohydrate

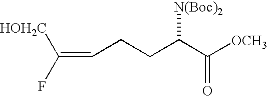

EX-F-1) To a THF (45 ml) solution of the product of EX-A-3 (5.0 g, 11.5 mmol) under nitrogen was added dropwise a solution of Red-Al (5.22 ml, 17.4 mmol) in 5.6 mL THF over 30 minutes. The internal temperature was kept below −10° C. After 5 minutes, the reaction was quenched with 33.7 ml of 1.3M Na.K tartrate. Toluene (11 mL) was added to the mixture to improve separation. The organic layer was washed with 33.7 ml of 1.3M Na.K tartrate followed by brine (40 mL). The organic layers were combined, dried over MgSO4, filtered and concentrated. The crude material, 3.8 g (84%) of light yellow oil, was carried on directly into the next step. LCMS: m/z=414.2 [M+Na]⁺. ¹H NMR (CDCl₃) δ1.48 (s, 18H), 1.95 (m, 1H), 2.1 (m, 1H), 2.2 (m, 1H), 2.35 (t, 1H), 3.7 (s, 3H), 4.25 (m, 2H), 4.8 (m, 1H), 5.15 (dt, 1H, J=20 Hz). ¹⁹F NMR (CDCl₃) δ−119.1 (d, 0.02F, J=37 Hz, 2% Z-isomer), −111.8 (d, 0.98F, J=24 Hz, 98% E-isomer).

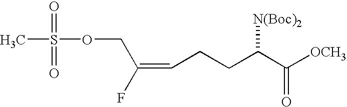

EX-F-2) To a solution of the product of EX-F-1 (50.0 g, 0.128 mol) in 500 mL of methylene chloride at −10° C. was added triethylamine (18.0 g, 0.179 mol). A solution of methanesulfonyl chloride (17.5 g, 0.153 mol) in 50 mL methylene chloride was added slowly to maintain temperature at −10° C. The reaction was stirred for 45 min at −10° C., at which time analysis by thin layer chromatography (50% ethyl acetate in hexane) and LCMS showed that most of the starting material was consumed. The reaction was quenched with 600 mL of 1.0 M citric acid and extracted with ethyl acetate (2×400 mL). The organic layers were combined, dried over MgSO₄, filtered and concentrated. The crude material, 70 g of yellow oil, was carried directly into the next step. LCMS: m/z=492.2 [M+Na].

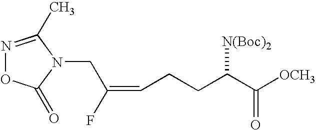

EX-F-3) To a solution of the product of EX-F-2 (70.0 g, 0.128 mol) in 400 mL of dimethyl formamide at room temperature was added potassium 3-methyl-1,2,4-oxadiazolin-5-onate (28.7 g, 0.192 mol). The reaction was stirred for 2.5 h at room temperature, at which time analysis by thin layer chromatography (30% ethyl acetate in hexane) and LCMS showed that the starting material was consumed. The reaction was diluted with 400 mL of water and extracted with ethyl acetate (5×400 mL). The organic layers were combined, washed with 400 mL water, 400 mL brine, dried over MgSO₄, filtered and concentrated. The crude material, 70 g of yellow oil, was purified by flash column chromatography on silica gel eluting with 1:4 ethyl acetate in hexane to give 38 g (63%) of a slightly yellow oil.

EX-F-4) A combination of product of several duplicate preparations of EX-F-3 was purified by HPLC column chromatography on Merk silica gel MODCOL column at a flow of 500 mL/min isocratic at 60:40 MtBE:heptane. A second purification on the 63 g recovered was a chiral HPLC column chromatography on a Chiral Pak-AD column running at a flow of 550 mL/min isocratic at 10:90 A:B (A: 100% ethanol, B: 100% heptane). Fractions containing product were combined and concentrated affording 41 g (68%) of the desired protected L,E-allylic amidine product as a clear oil, that contained only the desired L and E-isomer by ¹⁹F NMR and chiral chromatography. LCMS: m/z=496.2 [M+Na]⁺. [M+NH₄]⁺. HRMS calcd. for C₂₁H₃₂FN₃O₈: 491.2507 [M+NH₄]⁺, found: 491.2517. ¹H NMR (CDCl₃) δ1.48 (s, 18H), 1.85 (m, 1H), 2.2 (m, 3H), 2.25 (s, 3H), 3.64 (s, 3H), 4.25 (m, 2H), 4.8 (m, 1H), 5.3 (dt, 1H, J=20 Hz). ¹⁹F NMR (CDCl₃) δ−110.8 (q, 1F, J=20 Hz).

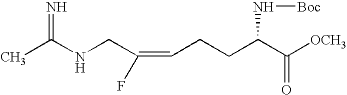

EX-F-5) The product from EX-F-4 (22.5 g, 0.047 mol) was dissolved in 112 mL of methanol. Vigorous stirring was begun and 225 mL of 40% acetic acid in water followed by zinc dust (11.5 g, 0.177 mmol) was added. The stirring reaction was placed under reflux (approx. 60° C.) for 2.5 h, at which time HPLC analysis showed that most of the starting material had been consumed. The reaction was cooled and the Zn was filtered from the reaction mixture through celite, washing the celite well with additional methanol. The filtrate and methanol washings were combined and concentrated. The resulting oily-white solid was washed with methylene chloride (2×500 mL) and filtered through a celite pad, an additional 500 mL methylene chloride wash was performed. The filtrates were combined and concentrated to provide a light yellow oil. The crude material, 39 g of a light-yellow oil, was purified by plug filtration on 200 mL silica gel eluting with 80:19:1 methanol: methylene chloride: acetic acid to give 13 g (83%) of the desired product. LCMS: m/z=432.3 [M+H]⁺. 1 [M+H]⁺. HRMS calcd. for C₁₅H₂₆FN₃O₄: 332.1986 [M+H]⁺, found: 332.1982. ¹H NMR (CD₃OD) δ1.42 (s, 9H), 1.7 (m, 1H), 1.9 (m, 1H), 2.17 (m, 2H), 2.22 (s, 3H), 3.3 (m, 1H), 3.7 (s, 3H), 4.2 (d, 2H), 5.1 (dt, vinyl, 1H, J=21 Hz). ¹⁹F NMR (CD₃OD) δ−110.83 (m, 1F, J=21 Hz).

Example F) A solution of the product of EX-F-5 (22 g, 0.066 mol) in 750 mL of 6.0 N HCl was refluxed for 45 min. The solvent was removed in vacuo. The resulting solid was dissolved in water and concentrated three additional times. The crude material was purified by reverse-phase HPLC column chromatography on a YMC ODS-AQ column eluting over 60 min pumping 100% isocratic B for 30 min followed by a gradient of 0-100% A for 10 min and a 100% A wash for 20 min (A: 100% acetonitrile, B: 100% H₂O with 0.0025% acetic acid). Fractions containing product were combined and concentrated affording 3.5 g (68%) of the desired acetamidine product as a dihydorchloride salt, that contained only the desired (2S,5E)-2-amino-6-fluoro-7-[(1-iminoethyl)amino]-5-heptenoic acid, dihydrochloride product was obtained as a white solid, m.p. 51.5-56.3° C., that contained only the desired E-isomer by ¹⁹F NMR. LCMS: m/z=218.1 [M+H]⁺. HRMS calcd. for C₉H₁₆FN₃O₂: 218.1305 [M+H]⁺, found: 218.1325. ¹H NMR (D₂O) δ1.8 (m, 2H), 2.05 (m, 2H), 2.1 (s, 3H), 3.7 (t, 1H), 4.00 (d, 2H), 5.3 (dt, vinyl, 1H, J=21 Hz). ¹⁹F NMR (D₂O) δ−109.9 (m, 1F, J=20 Hz). [δ]₅₈₉=+15.3 (C, 0.334, (H₂O);) [δ]₃₆₅=+52.8 (C, 0.334, (H₂0)

### Example G

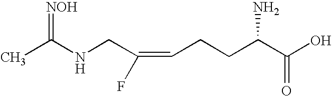

### (2S,5E)-2-amino-6-fluoro-7-[(1-hydroximinoethyl)amino]-5-heptenoic acid

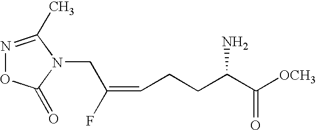

EX-G-1) Gaseous HCl was bubbled for 5 min through a stirring cold (0° C.) solution of the product of EX-F-3 (14 g, 30.0 mmol) in 100 mL of methanol. The resulting dark yellow solution was stirred an additional 30 min, at which time HPLC indicated complete consumption of starting material. The resulting mixture was neutralized with saturated NaHCO₃ to pH=8, and the product was extracted out with EtOAc. The organic layer was dried over MgSO₄ and concentrated to give the desired amino ester product as a dark yellow oil that was carried on crude to the next step. LCMS: m/z=274 [M+Na]⁺. ¹H NMR (CDCl₃) δ1.8 (m, 4H), 2.25 (s, 3H), 3.42 (bm, 1H), 3.80 (s, 3H), 4.4 (dd, 2H), 5.40 (dt, vinyl, 1H, J=21 Hz). ¹⁹F NMR (CDCl₃) δ−110.38 (m, 1F, J=21 Hz).

Example G) A solution of the product of EX-G-1 (8 g, 30 mmol) in 70 mL of 2.5N NaOH was stirred for 10 min, at which time HPLC analysis indicated the complete consumption of starting material. The resulting solution was neutralized with 12N HCl (approximately 50 mL) to pH=7-8 and concentrated. The resulting slurry was washed with methanol, filtered to remove salts and concentrated to a brownish oil. The crude material was purified by reverse-phase HPLC column chromatography on a YMC ODS-AQ column eluting over 60 min pumping 100% isocratic B for 30 min followed by a gradient of 0-100% A for 10 min and a 100% A wash for 20 min (A: 100% acetonitrile, B: 100%). Fractions containing product were combined and concentrated affording 1.0 g (14%) of the desired product as a white solid. The product was recrystallized from hot water and isopropyl alcohol and collected by filtration to afford pure (2S,5E)-2-amino-6-fluoro-7-[(1-hydroximinoethyl)amino]-5-heptenoic acid as a white crystalline solid. Melting point: 198.00-200.00° C. LCMS: m/z=234.1 [M+H]⁺. ¹H NMR (D₂O) δ1.8 (m, 4H), 2.05 (m, 2H), 3.6 (t, 1H), 3.9 (d, 2H), 5.2 (dt, vinyl, 1H, J=21 Hz). ¹⁹F NMR (D₂O) δ−112.1 (m, 1F, J=20 Hz).). Anal. calcd. for C₉H₁₆FN₃O₃: C, 46.35; H, 6.91; N, 18.02; O, 20.58. Found: C, 46.44; H, 6.95; N, 17.94; O, 20.78. Chiral analysis >97.7%: CrownPak CR(+) at 0.8 mL/min isocratic with 100% A (A: aqueous HClO₄, pH=1.5).

### Example H

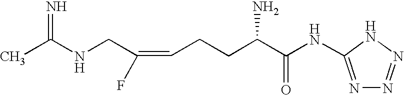

### (2S,5E)-2-amino-6-fluoro-7-[(1-iminoethyl)amino]-N-(1H-tetrazol-5-yl) 5-heptenamide, dihydrochloride

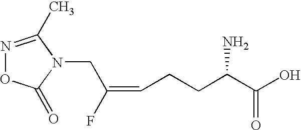

EX-H-1) The product from EX-F-3 (6.1 g, 0.013 mol) was dissolved in 4 mL of methanol. Vigorous stirring was begun and 10 mL of 6N HCl was added. The stirring reaction was placed under reflux (approx. 60° C.) for 18 h, at which time HPLC analysis showed that most of the starting material had been consumed. The reaction was cooled and concentrated to 3.3 g (100%) of orange oil. LCMS: m/z=282 [M+Na]⁺.

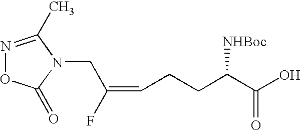

EX-H-2) The product from EX-H-1 (3.3 g, 0.013 mol) was dissolved in 12 mL of 1:1 H₂O:dioxane. Stirring was begun and triethylamine (1.95 g, 0.019 mol) was added. The reaction was cooled to 0° C. and di-tert-butyldicarbonate (3.4 g, 0.016 mol) was added. The reaction was allowed to warm to room temperature at which time acetonitrile (4 mL) was added to dissolve solids. The reaction was stirred at room temperature for 18 h at which time HPLC analysis showed that most of the starting material had been consumed. The reaction was quenched with 1.0N KHSO₄ (25 mL), extracted with ethyl acetate (3×50 mL) and the organic layers dried over MgSO₄ and concentrated. The crude material, 3.5 g of a dark oil, was purified by flash chromatography eluting with 4:95:1 methanol: methylene chloride: acetic acid to give 2.4 g (52%) of desired product as a light-yellow oil. LCMS: m/z=382 [M+Na]⁺.

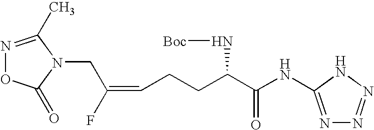

EX-H-3) The product from EX-H-2 (2.4 g, 0.007 mol) was dissolved in 13 mL THF. Stirring was begun and 5-aminotetrazole monohydrate (0.83 g, 0.008 mol) was added followed by 1,3-diisopropylcarbodiimide (1.0 g, 0.008 mol). The resulting mixture was allowed to stir at room temperature for 3 h at which time HPLC showed that most of the starting material had been consumed. To the reaction was added 12 mL water and the THF was removed by vaccum distillation. Ethanol (30 mL) was added and the reaction was heated to reflux. After 15 min at reflux, the reaction was cooled to −10° C. at which time the desired product precipitated from solution. The product was collected by filtration to afford 1.25 g (50%) of a white solid. LCMS: m/z=449 [M+Na]⁺.

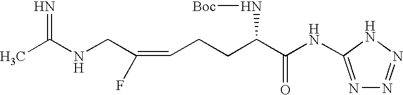

EX-H-4) The product from EX-H-3 (1.0 g, 0.0023 mol) was dissolved in 5 mL of methanol. Vigorous stirring was begun and 10 mL of 40% acetic acid in water followed by zinc dust (0.5 g, 0.008 mol) was added. The stirring reaction was placed under reflux (approx. 60° C.) for 1.5 h, at which time HPLC analysis showed that most of the starting material had been consumed. The reaction was cooled and the Zn was filtered from the reaction mixture through celite, washing the celite well with additional methanol. The filtrate and methanol washings were combined and concentrated. The resulting oily-white solid was purified by reverse-phase HPLC column chromatography on a YMC ODS-AQ column eluting over 60 min pumping 100% isocratic B for 30 min followed by a gradient of 0-100% A for 10 min and a 100% A wash for 20 min (A: 100% acetonitrile, B: 100% H₂O with 0.0025% acetic acid). Fractions containing product were combined and concentrated affording 0.390 g (44%) of the desired acetamidine product as a white solid. LCMS: m/z=407.3 [M+Na].

Example H) The product from EX-H-4 (0.30 g, 0.780 mmol) was dissolved in 5 mL of conc HOAc. To this was added 1 mL of 4N HCl in dioxane. The reaction was stirred 5 min. at room temperature. The solvent was removed in vacuo. The resulting solid was dissolved in water and concentrated three additional times. HPLC indicated amounts of starting material. The solid was dissolved in 1N HCl and stirred 3h at which time HPLC indicated that most of the starting material had been consumed. The solution was concentrated affording 290 mg (98%) of the desired acetamidine product as a dihydorchloride salt. LCMS: m/z=285.1 [M+H].

### Example I

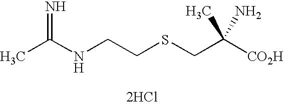

### S-[2-[(1-Iminoethyl)amino]ethyl]-2-methyl-L-cysteine, dihydrochloride

### Example-I-1)

### (2R,4R)-Methyl-2-tert-butyl-1,3-thiazoline-3-formyl-4-carboxylate

See Jeanguenat and Seebach, J. Chem. Soc. Perkin Trans. 1, 2291 (1991) and Pattenden et al. Tetrahedron, 49, 2131 (1993): (R)-cysteine methyl ester hydrochloride (8.58 g, 50 mmol), pivalaldehyde (8.61 g, 100 mmol), and triethylamine (5.57 g, 55 mmol) were refluxed in pentane (800 ml) with continuous removal of water using a Dean-Stark trap. The mixture was filtered and evaporated. The resultant thiazolidine (9.15 g, 45 mmol) and sodium formate (3.37 g, 49.5 mmol) were stirred in formic acid (68 ml) and treated with acetic anhydride (13 mL, 138 mmol), dropwise over 1 hour at 0-5° C. The solution was allowed to warm to RT and stir overnight. The solvents were evaporated and the residue was neutralized with aqueous 5% NaHCO₃ and extracted with ether (3×). The combined organic layers were dried (anhy. MgSO₄), filtered, and evaporated to give the title compound which was crystallized from hexane-ether as white crystals (8.65 g) (80% overall, 8:1 mixture of conformers). ¹H NMR (CDCl₃) δ major conformer: 1.04 (s, 9H), 3.29 (d, 1H), 3.31 (d, 1H), 3.78 (s, 3H), 4.75 (s, 1H), 4.90 (t, 1H), 8.36 (s, 1H). MS m/z (electrospray) 232 (M+H)⁺ (100%), 204 (10) 164 (24).

### Example-I-2)

### (2R,4R)-Methyl-2-tert-butyl-1,3-thiazoline-3-formyl-4-methyl-4-carboxylate

To a solution of the product of Example-I-1, (2R,4R)-Methyl-2-tert-butyl-1,3-thiazoline-3-formyl-4-carboxylate (8.65 g, 37.4 mmol), in anhydrous tetrahydrofuran (130 mL) under N₂ at −78° C. was added DMPU (25 mL) and the mixture stirred for 5 min. Lithium bis(trimethylsilyl)amide, 1 M in tetrahydrofuran, (37.5 mL), was added, and the mixture stirred for 30 min. After methyl iodide (5.84 g, 41.1 mmol) was added, the mixture was held at −78° C. for 4 hr and then warmed to room temperature with continuous stirring. The solvents were evaporated in vacuo and brine and ethyl acetate was added. The aqueous phase was extracted 3× EtOAc, and the combined organic layers were washed with 10% KHSO₄, water, and brine. They were then dried (anhy. MgSO₄), filtered, and stripped of all solvent under reduced pressure. Chromatography of the residual oil on silica with 1-10% EtOAc/hexane yielded the title compound (5.78 g, 63%, 2.4:1 mixture of conformers). ¹H NMR (CDCl₃) δ major conformer, 1.08 (s, 9H), 1.77 (s, 3H), 2.72 (d, 1H), 3.31 (d, 1H), 3.77 (s, 3H), 4.63 (s, 1H), 8.27 (s, 1H); minor conformer, 0.97 (s, 9H), 1.79 (s, 3H), 2.84 (d, 1H), 3.63 (d, 1H), 3.81 (s, 3H), 5.29 (s, 1H), 8.40 (s, 1H); MS m/z (electrospray) 246 (M+H)⁺ (100%), 188 (55) 160 (95). Retention time of 16.5 min on a Daicel Chemical Industries Chiracel OAS column, 10-40% IPA/hexane 0-25 min, >95% ee.

### Example-I-3)

### (2R) 2-Methyl-L-cysteine hydrochloride

The product of Example-1-2, (2R,4R)-Methyl-2-tert-butyl-1,3-thiazoline-3-formyl-4-methyl-4-carboxylate, (5.7 g, 23.2 mmol) was stirred with 6N HCl (100 mL) under N₂ and held at vigorous reflux for 2 days. The solution was cooled, washed with EtOAc and evaporated to yield the product (2R) 2-methyl-cysteine hydrochloride (3.79 g, 95%) as a light yellow powder. ¹H NMR (DMSO-d₆) δ1.48 (s, 3H,) 2.82 (t, 1H), 2.96 (bs, 2H), 8.48 (s, 3H). MS m/z (electrospray) 136 [M+H⁺].

### Example-I-4)

### S-[2-[[(1,1-dimethylethoxy)carbonyl]amino]ethyl]-2-methyl-L-cysteine trifluoroacetate

Sodium hydride (2.6 g, 60% in mineral oil, 65 mmol) was added to an oven-dried, vacuum-cooled RB flask, containing oxygen-free 1-methyl-2-pyrrolidinone (5 mL). The mixture was cooled to −10° C. and stirred under N₂. The product of Example-I-3, 2-Methyl-L-cysteine hydrochloride, (3.6 g, 21.0 mmol) dissolved in oxygen-free 1-methyl-2-pyrrolidinone (25 ml), was added in portions. After all H₂ evolution ceased, 2-[(1,1-dimethylethoxycarbonyl)-amino]ethyl bromide (4.94 g, 21 mmol) in oxygen-free 1-methyl-2-pyrrolidinone (15 mL) was added at −10° C. The reaction was then stirred for 4 hr allowing warming to room temperature. The solution was neutralized with 1 N HCl and the 1-methyl-2-pyrrolidinone was removed by evaporation in vacuo. Reverse-phase chromatography with 1-20% acetonitrile in 0.05% aqueous trifluoro acetic acid solution yielded the title compound (5.9 g), recovered by freeze-drying appropriate fractions. ¹H NMR (DMSO-d₆/D₂O) δ1.31 (s, 9H), 1.39 (s, 3H), 2.55 (m, 2H), 2.78 (d, 1H), 3.04 (d, 1H), 3.06 (t, 2H). HRMS calc. for C₁₁H₂₂N₂O₄S: 279.1375 (M+H⁺), found 279.1379.

### Example-I-5)

### S-(2-aminoethyl)-2-methyl-L-cysteine hydrochloride

The product of Example-I-4, S-[2-[[(1,1-dimethylethoxy)carbonyl]amino]ethyl]-2-methyl-L-cysteine trifluoroacetate, (5.5 g, 14.0 mmol) was dissolved in 1 N HCl (100 mL) and stirred at room temperature under nitrogen overnight. The solution was removed by freeze-drying to give the title S-(2-aminoethyl)-2-methyl-L-cysteine hydrochloride, ¹H NMR (DMSO-d₆/D₂O) δ1.43 (s, 3H), 2.72 (m, 2H), 2.85 (d, 1H), 2.95 (t, 2H), 3.07 (d, 1H). m/z [M+H⁺] 179.

Example I) The product of Example-I-5, was dissolved in H₂O, the pH adjusted to 10 with 1 N NaOH, and ethyl acetimidate hydrochloride (1.73 g, 14.0 mmol) was added. The reaction was stirred 15-30 min, the pH was raised to 10, and this process repeated 3 times. The pH was adjusted to 3 with HCl and the solution loaded onto a washed DOWEX 50WX4-200 column. The column was washed with H₂O and 0.25 M NH₄OH, followed by 0.5 M NH₄OH. Fractions from the 0.5 M NH₄OH wash were immediately frozen, combined and freeze-dried to give an oil that was dissolved in 1N HCl and evaporated to give the title compound as a white solid (2.7 g). ¹H NMR (DMSO-d₆/D₂O) δ1.17 (s, 3H), 2.08 (s, 3H), 2.52 (d, 1H), 2.68 (m, 2H), 2.94 (d, 1H), 3.23 (t, 2H). HRMS calc. for C₈H₁₈N₃O₂S: 220.1120 [M+H⁺], found 220.1133.

### Example J

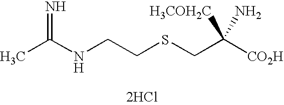

### 2-[[[2-[(1-Iminoethyl)amino]ethyl]thio]methyl]-O-methyl-D-serine, dihydrochloride

The procedures and methods utilized in this example were identical to those of Example I except that in step Example-I-2 methoxymethyl iodide was used instead of methyl iodide. These procedures yielded the title product as a white solid (2.7 g). ¹H NMR (D₂O) δ2.06 (s, 3H), 2.70 (m, 3H), 3.05 (d, 1H), 3.23 (s, 3H), 3.32 (t, 2H), 3.46 (d, 1H), 3.62 (d, 1H). HRMS calc. for C₉H₂₀N₃O₃S: 250.1225 [M+H⁺], found 250.1228.

### Example K

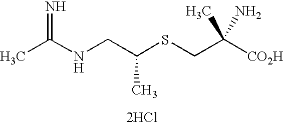

### S-[(1R)-2-[(1-Iminoethyl)amino]-1-methylethyl]-2-methyl-L-cysteine, dihydrochloride

### Example-K-1)

### (S)-1-[(benzyloxycarbonyl)amino]-2-propanol

To a solution of (S)-1-amino-2-propanol (9.76 g, 130 mmol) in anhydrous benzene (60 mL) at 0° C. was added benzyl chloroformate (10.23 g, 60 mmol) in anhydrous benzene (120 mL) slowly, in portions, over a period of 20 min while vigorously stirring under an atmosphere of nitrogen. The mixture was stirred for 1 hour at 0° C., then allowed to warm to room temperature and stirred for a further 2 hours. The mixture was washed with water (2×) and brine (2×) before the organic layer was dried over anhydrous MgSO₄. Evaporation of all solvent gave the title product as an oil. ¹H NMR (CDCl₃) δ1.22 (d, 3H,) 2.40 (bs, 1H), 3.07 (m, 1H), 3.37 (m, 1H)), 3.94 (m, 1H), 5.16 (s, 2H), 5.27 (m, 1H), 7.38 (m, 5H). MS m/z (electrospray) 232 [M+23]⁺ (100%), 166 (96).

### Example-K-2)

### (S)-1-[(benzyloxycarbonyl)amino]-2-propanol tosylate

To a solution of the product of Example-K-1, (S)-1-[(benzyloxycarbonyl)amino]-2-propanol, (9.74 g, 46.7 mmol) and triethylamine 7.27 g, 72 mmol) in methylene chloride (60 mL) at 0° C. was added toluene sulfonyl chloride (9.15 g, 48 mmol) in methylene chloride (18 mL) slowly, in portions, over a period of 20 min while vigorously stirring under nitrogen. The mixture allowed to warm to room temperature and stirred for a further 36 hours under nitrogen. The organic layer was washed with 1N HCl, water, 5% NaHCO₃ solution, water and brine before it was dried over anhydrous MgSO₄. Evaporation of all solvent gave a white solid which was passed though a silica plug with ethyl acetate/hexane (1:4) to remove excess toluene sulfonyl chloride and then with ethyl acetate/hexane (1:3) to give the title product as white crystals. This material was recrystallized from ethyl acetate/hexane to give white needles (10.8 g). ¹H NMR (CDCl₃) δ1.22 (d, 3H,) 2.39 (s, 3H), 3.20 (m, 1H), 3.43 (dd, 1H)), 4.66 (m, 1H), 5.02 (m, 1H), 5.04 (ABq, 2H), 7.34 (m, 7H), 7.77 (d, 2H). MS m/z (electrospray) 386 [M+23]⁺ (100%), 320 (66). The product was examined on a Regis Technologies Inc. Perkle Covalent (R,R)-GEM1 HPLC column using mobile phase of isopropanol/hexane and a gradient of 10% isopropanol for 5 min, then 10 to 40% isopropanol over a period of 25 min, and using both UV and Laser Polarimetry detectors. Retention time major peak: 22.2 min, >98% ee.

### Example-K-3)

### S-[(1R)-2-(Benzyloxycarbonylamino)-1-methylethyl]-2-methyl-L-cysteine trifluoroacetate

The product of Example-1-3, 2-methyl-L-cysteine hydrochloride, (1 g, 6.5 mmol) was added to an oven dried, N₂ flushed RB flask, dissolved in oxygen-free 1-methyl-2-pyrrolidinone (5 mL), and the system was cooled to 0° C. Sodium hydride (0.86 g, 60% in mineral oil, 20.1 mmol) was added and the mixture was stirred at 0° C. for 15 min. A solution of the product of Example-K-2, (2S)-1-[(N-benzyloxycarbonyl)amino]-2-propanol tosylate (2.5 g, 7 mmol) dissolved in oxygen-free 1-methyl-2-pyrrolidinone (10 mL) was added over 10 min. After 15 min at 0° C., the reaction mixture was stirred at room temperature for 4.5 hours. The solution was then acidified to pH 4 with 1N HCl and 1-methyl-2-pyrrolidinone was removed by evaporation in vacuo. Reverse phase chromatography with 20-40% acetonitrile in 0.05% aqueous trifluoro acetic acid solution yielded the title compound in (0.57 g), recovered by freeze-drying. ¹H NMR (H₂O, 400 MHz) δ1.0 (d, 3H), 1.4 (s, 3H), 2.6 (m, 2H), 2.8 (m, 1H), 3.1 (m, 2H), 3.6 (s, 1H), 5.0 (ABq, 2H), 7.3 (m, 5H). MS m/z (electrospray): 327 [M+H⁺] (100%), 238 (20), 224 (10), and 100 (25).

### Example-K-4)

### S-[(1R)-2-Amino-1-methylethyl]-2-methyl-L-cysteine hydrochloride

The product of Example-K-3, S-[(1R)-2-(Benzyloxycarbonylamino)-1-methylethyl]-2-methyl-L-cysteine trifluoroacetate, (0.5 g, 1.14 mmol) was dissolved in 6N HCl and refluxed for 1.5 hour. The mixture was then cooled to room temperature and extracted with EtOAc. The aqueous layer was concentrated in vacuo to give the title product, (2R, 5R)-S-(1-amino-2-propyl)-2-methyl-cysteine hydrochloride (0.29 g), which was used without further purification. ¹H NMR (H₂O, 400 MHz) δ1.2 (m, 3H), 1.4 (m, 3H), 2.7 (m, 1H), 2.8-3.2 (m, 2H), 3.4 (m, 1H). (some doubling of peaks due to rotameric forms). MS m/z (electrospray): 193 [M+H⁺] (61%), 176 (53), 142 (34), 134 (100), and 102 (10).

Example K) The product of Example-K-4, S-[(1R)-2-Amino-1-methylethyl]-2-methyl-L-cysteine hydrochloride, (0.2 g, 0.76 mmol) was dissolved in 2 mL of H₂O, the pH was adjusted to 10.0 with 1N NaOH, and ethyl acetimidate hydrochloride (0.38 g, 3 mmol) was added in four portions over 10 minutes, adjusting the pH to 10.0 with 1N NaOH as necessary. After 1 h, the pH was adjusted to 3 with 1N HCl. The solution was loaded onto a water-washed DOWEX 50WX4-200 column. The column was washed with H₂O and 0.5N NH₄OH. The basic fractions were pooled and concentrated to dryness in vacuo. The residue was acidified with 1N HCl and concentrated to the Example K title product, (49 mg). ¹H NMR (H₂O, 400 MHz) δ1.3−1.0 (m, 3H), 1.5 (m, 3H), 2.1−1.8 (m, 3H), 3.4−2.6 (m, 5H), 3.6 (m, 1H) (rotamers observed). MS m/z (electrospray): 234 [M+H⁺] (100%), 176 (10), and 134 (10).

### Example L

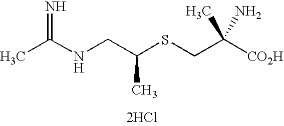

### S-[(1S)-2-[(1-Iminoethyl)amino]-1-methylethyl]-2-methyl-L-cysteine, dihydrochloride

The procedures and methods employed here were identical to those of Example K, except that in step Example-K-1 (R)-1-amino-2-propanol was used instead of (S)-1-amino-2-propanol to give the title material, S-[(1S)-2-[(1-Iminoethyl)amino]-1-methylethyl]-2-methyl-L-cysteine hydrochloride. ¹H NMR (H₂O, 400 MHz) δ3.6 (m, 1H), 3.4−2.6 (m, 5H), 2.1−1.8 (m, 3H), 1.5 (m, 3H), and 1.3−1.0 (m, 3H). HRMS calc for C₉H₁₉N₃O₂S [M+H⁺]: 234.1276. Found: 234.1286.

### Example M

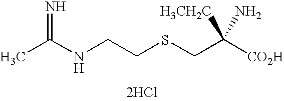

### S-[2-[(1-Iminoethyl)amino]ethyl]-2-ethyl-L-cysteine, dihydrochloride

The procedures and methods used in this synthesis were the same as those used in Example I except that ethyl triflate was used in Example-I-2 instead of methyl iodide. Reverse phase chromatography, using a gradient of 10-40% acetonitrile in water, was used to purify the title product (20% yield). ¹H NMR (D₂O) δ0.83 (t, 3H), 1.80 (m, 2H), 2.08 (s, 3H), 2.68 (m, 1H), 2.78 (m, 1H), 2.83 (m, 1H), 3.11 (m, 1H), 3.36 (t, 2H). HRMS calc. for C₉H₂₀N₃O₂S: 234.1276 [M+H⁺], found 234.1284.

### Example N

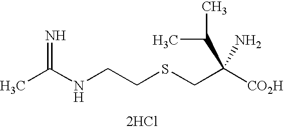

### 2-[[[[2-(1-Iminoethyl)amino]ethyl]thio]methyl]-D-valine, dihydrochloride

### Example-N-1)

### Isopropyl triflate

Silver triflate (25.25 g, 98.3 mmol) stirred in diethyl ether (300 mL) under nitrogen was treated with isopropyl iodide (16.54 g, 98.5 mmol) in ether (200 mL) over 15 minutes. The mixture was stirred for 10 minutes and then filtered. The filtrate was distilled at reduced pressure. The distillate was redistilled at atmospheric pressure to remove the majority of the diethyl ether, leaving a mixture of the title isopropyl triflate-diethyl ether (84:16 by weight) (15.64 g, 70% corrected) as a colorless liquid. ¹H NMR (CDCl₃, 400 MHz) δ1.52 (d, 6H), 5.21 (septet, 1H).

The procedures and methods utilized here were the same as those used in Example I except that isopropyl triflate replaced methyl iodide in Example-I-2. The crude title product was purified by reversed phase chromatography using a gradient elution of 10-40% acetonitrile in water. ¹H NMR (H₂O, 400 MHz) δ0.94 (dd, 6H), 2.04 (septet, 1H), 2.10 (s, 3H), 2.65, 2.80 (d m, 2H), 2.85, 3.10 (dd, 2H), 3.37 (t, 2H). HRMS calc. for C₁₀H₂₂N₃O₂S: 248.1433 [M+H⁺], found 248.1450.

### Example O

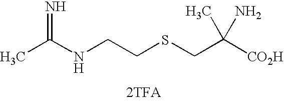

### S-[2-(1-Iminoethylamino)ethyl]-2-methyl-(D/L)-cysteine, bistrifluoroacetate

### Example-O-1)

### S-(2-aminoethyl)-L-cysteine, methyl ester

A 10 g (50 mmol) sample of S-(2-aminoethyl)-L-cysteine was dissolved in 400 mL of methanol. Into this cooled solution was bubbled in anhydrous HCl for 30 minutes. After stirring at room temperature overnight, the solution was concentrated to afford 12.7 g of the title compound.

### Example-O-2)

### N-{4-chlorophenyl)methylene]-S-[2-[[(4-chlorophenyl)methylene]amino]ethyl]-L-cysteine, methyl ester

A 12.7 g (50 mmol) sample of the product of Example-O-1, S-(2-aminoethyl)-L-cysteine methyl ester, was dissolved in acetonitrile. To this solution was added 12.2 g (100 mmol) of anhydrous MgSO₄, 14 g (100 mmol) of 4-chlorobenzaldehyde and 100 mmol of triethylamine. This mixture was stirred for 12 hours, concentrated to a small volume and diluted with 500 mL of ethyl acetate. The organic solution was washed successively with (0.1%) NaHCO₃, (2N) NaOH, and brine solution. The organic was dried (anhy. MgSO₄), filtered and concentrated to afford 7.5 g of the title compound. [M+H⁺] =179.

### Example-O-3)

### N-[4-chlorophenyl)methylene]-S-[2-[[(4-chlorophenyl)methylene]amino]ethyl]-2-methyl-D/L-cysteine methyl ester

A sample of the product of Example-O-2, N-{4-chlorophenyl)methylene]-S-[2-[[(4-chlorophenyl)methylene]amino]ethyl]-L-cysteine methyl ester (7.5 g, 17 mmol), in anhydrous THF was treated with 17 mmol of sodium bis(trimethylsilyl)amide at −78° C. under nitrogen, followed by 2.4 g (17 mmol) of methyl iodide. The solution was held at −78° C. for 4 hr and then warmed to room temperature with continuous stirring. The solvents were evaporated in vacuo and brine and ethyl acetate was added. The aqueous phase was extracted 3× EtOAc, and the combined organic layers were washed with 10% KHSO₄, water, and brine before it was dried (anhy. MgSO₄), filtered, and evaporated to afford the title compound.

### Example-O-4)

### S-(2-aminoethyl)-2-methyl-D/L-cysteine, hydrochloride

A sample of the product of Example-O-3, N-[4-chlorophenyl)methylene]-S-[2-[[(4-chlorophenyl)methylene]amino]ethyl]-2-methyl-D/L-cysteine methyl ester (4.37 g, 10 mmol), was stirred and heated (60° C.) with 2N HCl overnight and the solution washed (3×) with ethyl acetate. The aqueous solution was freeze-dried to give the title compound.

Example O) A sample of the product of Example-O-4, S-(2-aminoethyl)-2-methyl-D/L-cysteine dihydrochloride (2.5 g (10 mmol), was dissolved in H₂O and the pH was adjusted to 10 with 1 N NaOH. Ethyl acetimidate hydrochloride (1.24 g, 10.0 mmol) was then added to the reaction mixture. The reaction was stirred 15-30 min, the pH was raised to 10, and this process repeated 3 times. The pH was reduced to 4 with HCl solution and the solution evaporated. The residue was purified on reverse phase HPLC with H₂O containing 0.05% trifluoroacetic acid as the mobile phase to afford the Example O title product. M+H=220.

### Example P

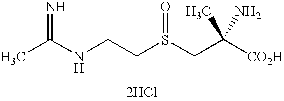

### (2R)-2-Amino-3[[2-[(1-iminoethyl)amino]ethyl]sulfinyl]-2-methylpropanoic acid, dihydrochloride

A solution of S-[2-[(1-iminoethyl)amino]ethyl]-2-methyl-L-cysteine, dihydrochloride (Example I, 0.2 g, 0.73 mmol) in 3 mL of water was stirred and cooled to 0° C. and a solution of 3% H₂O₂ (0.8 mL, 0.73 mmol) in formic acid (0.4 mL, 0.73 mmol) was added in 0.3 mL portions. The cold bath was removed and the reaction mixture was stirred at room temperature for 48 hours. The solution was concentrated in vacuo, diluted with of water (10 mL) and concentrated again to give the crude sulfone. This residue was chromatographed (C-18 reverse phase, with mobile phase H₂O containing 0.05% trifluoroacetic acid) to give the pure sulfone. The sulfone was treated with 1M HCl (10 mL) and concentrated in vacuo to give 140 mg of a mixture of 2 diastereomers of the title compound as a colorless oil of the HCl salts. ¹H NMR (300 MHz, D₂O) δ1.5 (s, 2H), 1.6 (s, 1H), 2.0 (s, 3H), 3.1 (m, 2H), 3.3 (m, 2H) 3.6 (m, 2H). HRMS calc. for C₈H₁₈N₃O₃S: 236.1069 [M+H⁺], found: 236.1024.

### Example Q

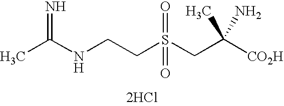

### (2R)-2-Amino-3[[2-[(1-iminoethyl)amino]ethyl]sulfonyl]-2-methylpropanoic acid dihydrochloride

A solution of S-[2-[(1-Iminoethyl)amino]ethyl]-2-methyl-L-cysteine dihydrochloride, the product of Example I, (0.15 g, 0.54 mmol) in 2 mL of water was cooled to 0° C. and a solution of 3% H₂O₂ (1.6 mL, 1.46 mmol) in formic acid (0.8 mL, 14.6 mmol) was added. The cold bath was removed and the reaction mixture was stirred at room temperature for 18 hours. The solution was concentrated in vacuo, diluted with 10 mL of water and concentrated again to give the crude sulfoxide. The residue was diluted with 4 mL of water and was adjusted to pH 9 with 2.5 N NaOH. Acetone (5 mL) was added, followed by Boc₂O (0.2 g), and the reaction was stirred for 48 h at room temperature. The reaction mixture was adjusted to pH 6 with 1 M HCl and was concentrated in vacuo. This residue was chromatographed (C-18 reverse phase; 40 to 50% ACN: H₂O, 0.05% TFA) to give the pure Boc protected material. The fractions were concentrated in vacuo and the residue was treated with 1 N HCl (3 mL) for 1 h. The solution was concentrated to give 30 mg of the title compound as colorless oil. ¹H NMR (400 MHz, D₂O) δ4.0 (d, 1H), 3.7 (d, 1H), 3.6 (t, 2H), 3.5 (t, 2H), 2.1 (s, 3H), and 1.5 (s, 3H) ppm. HRMS calc. for C₈H₁₈N₃O₄S: 252.1018 [M+H⁺], found: 252.0992.

### Example R

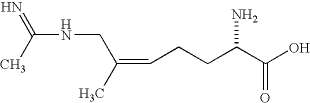

### (2S,5Z)-2-amino-6-methyl-7-[(1-iminoethyl)amino]-5-heptenoic acid, dihydrochloride

### Example R-1)

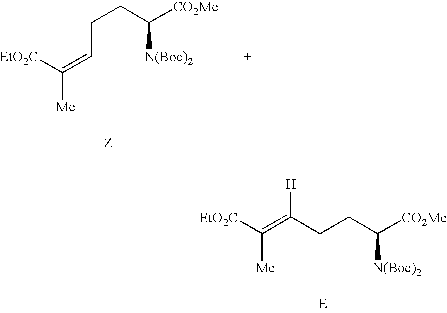

A solution of triethyl-2-phosphonopropionate (6.5 mg, 27.1 mmol) in toluene (60 ML) was treated with 0.5 M potassium bis(trimethylsilyl) amide (50.0 ML, in toluene) and the resulting anion was condensed with the aldehyde product of Example U-3 by the method of Example U-4 (see

Example U infra). This produced, after chromatography, 8 g of a 3:7 mixture respectively of the desired Z and E diesters.

(¹H)NMR (300 MHz, CDCl3) 6.7-6.8 ppm (m, 1H), 5.9 ppm (m, 1H), 4.9 ppm (m, 1H), 4.2 ppm (q, 2H), 3.7 ppm (s, 3H), 2.5 ppm (m, 1H), 2.2-2.3 ppm (m, 2H), 2.0 ppm (m, 1H), 1.9 ppm (s, 3H), 1.8 ppm (s, 3H), 1.5 ppm (s, 18H), 1.3 ppm (t, 3H).

### Example R-2)

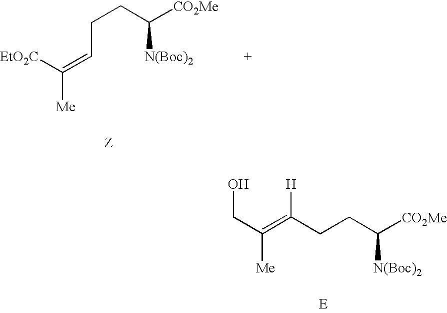

The product mixture of Example R-1 (850 mg, 2.0 mmol) in Et₂O (30 mL) was reduced over a period of twenty minutes with diisobutyl aluminum/hydride (DIBAL) by the method of Example U-5 to produce the crude illustrated desired mixture of E-alcohol and unreduced Z-ester. This mixture was chromatographed on silica gel eluting with n-hexane:EtOAc (9:1) to n-hexane:EtOAc (1:1) providing samples of the Z-ester (530 mg) and the E-alcohol desired materials.

Z-ester: (¹H)NMR (300 MHz, CDCl3) 5.9 ppm (m, 1H), 4.9 ppm (m, 1H), 4.2 ppm (q, 2H), 3.7 ppm (s, 3H), 2.5 ppm (m, 1H), 2.2-2.3 ppm (m, 2H), 1.9 ppm (s, 3H), 1.5 ppm (s, 18H), 1.3 ppm (t, 3H).

E-alcohol: (¹H)NMR (300 MHz, CDCl3) 5.35 ppm (m, 1H), 4.9 ppm (m, 1H), 3.95 ppm (s, 1H), 3.7 ppm (s, 3H), 1.8-2.2 ppm (m, 6H), 1.6 ppm (s, 3H), 1.5 ppm (s, 18H).

### Example R-3)

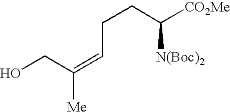

The product Z-ester of Example R-2 (510 mg, 1.2 mmol) in Et₂O (30 ML) was reduced over a period of two hours with diisobutyl aluminum/hydride (DIBAL) by the method of Example U-5 to produce the crude illustrated desired Z-alcohol. This material was chromatographed on silica gel eluting with n-hexane:EtOAc (9:1) to n-hexane:EtOAc (8:2) to yield 340 mg of the desired Z-alcohol product.

(¹H)NMR (300 MHz, CDCl₃) δ5.3 ppm (m, 1H), 4.9 ppm (m, 1H), 4.2 ppm (d, 1H), 4.0 ppm (d, 1H), 2.2 ppm (m, 3H), 1.95 ppm (m, 1H), 1.8 ppm (s, 3H), 1.5 ppm (s, 18H).

### Example R-4)

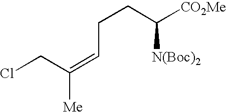

A CH₂Cl₂ solution (5 ML) of the product alcohol of Example R-3 (340 mg, 0.9 mmol) was treated with triethylamine (151 mg, 1.5 mmol). To this solution cooled in an ice bath was added a CH₂Cl₂ solution (1.5 ML) of methanesulfonyl chloride. After fifteen minutes the ice bath was removed and the reaction was stirred at ambient temperature for 20 h. The reaction mixture was then washed with 10% KHSO₄, dried over Na₂SO₄, and stripped of all solvent under reduced pressure to produce 350 mg of the desired Z-allylic chloride.

(¹H)NMR (300 MHz, CDCl₃) δ5.4 ppm (m, 1H), 4.9 ppm (m, 1H), 4.1 ppm (d, 1H), 4.0 ppm (d, 1H), 2.1 ppm (m, 3H), 1.95 ppm (m, 1H), 1.8 ppm (s, 3H), 1.5 ppm (s, 18H).

### Example R-5)

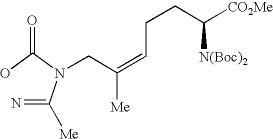

A suspension of potassium 3-methyl-1,2,4-oxa-diazoline-5-one in DMF is reacted with a DMF solution of the product of Example R-4 by the method of Example S-2 infra to produce the material.

### Example R-6)

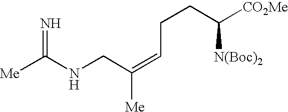

The product of Example R-5 is reacted with zinc in HOAc by the method of Example U-7 to yield the amidine.

### Example R-7)

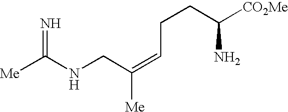

The product of Example R-6 was reacted with 4NHCl in dioxane in glacial HOAc to yield the amidine.

### Example R)

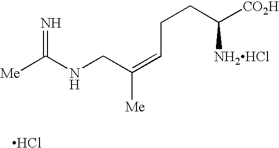

The product of Example R-7 is deprotected to yield the amino acid, dihydrochloride.

### Example S

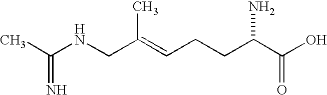

### (2S,5E)-2-amino-6-methyl-7-[(1-iminoethyl)amino]-5-heptenoic acid, dihydrochloride

### Example S-1)

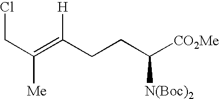

The E-alcohol product of Example R-2 (1.3 g, 3.3 mmol) was reacted with triethylamine (525 mg, 5.2 mmol) and methanesulfonyl chloride (560 mg, 5.2 mmol) by the method of Example R-4 to yield 1.4 g of the desired E-allylic chloride.

(¹H)NMR (400 MHz, CDCl3) δ5.5 ppm (m, 1H), 4.9 ppm (m, 1H), 4.0 ppm (s, 2H), 3.7 ppm (s, 3H), 2.1-2.3 ppm (m, 3H), 1.9 ppm (m, 1H), 1.7 ppm (s, 3H), 1.5 ppm (s, 18H).

### Example S-2)

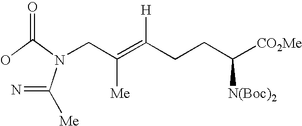

A suspension of potassium 3-methyl-1,2,4-oxa-diazoline-5-one (460 mg, 3.35 mmol) in 5 mL of DMF was treated with a DMF (15 mL) solution of the product of Example S-1. This reaction mixture was stirred at 50° C. for 17 h before an additional 50 mg (0.04 mmol) of the diazoline-5-one salt was added. Heating of the stirred reaction was continued for an additional 3 h before it was cooled to room temperature and diluted with 180 mL of water. This mixture was extracted with EtOAc and the extracts were diluted with 120 mL of n-hexane, washed with water, dried over Na₂SO₄ and stripped of all solvent under reduced pressure to yield 1.3 g of the material.

(¹H)NMR (400 MHz, CDCl₃) 5.5 ppm (m, 1H), 4.9 ppm (m, 1H), 4.2 ppm (s, 3H), 3.7 ppm (s, 3H), 2.2 ppm (m, 3H), 1.95 ppm (m, 1H), 1.8 ppm (s, 3H), 1.5 ppm (s, 18H).

### Example S-3)

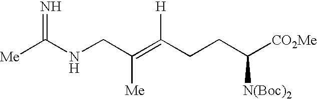

The product of Example S-2 (460 mg, 1.0 mmol) was reacted with zinc in HOAc by the method of Example U-7 (see Example U infra) to yield 312 mg of the desired amidine after HPLC purification.

### Example S)

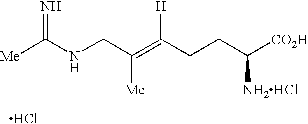

The product of Example S-3 (77 mg, 0.2 mmol) was deprotected with 2N HCl by the method of Example U to yield 63 mg the E-amino acid, dihydrochloride.

### Example T

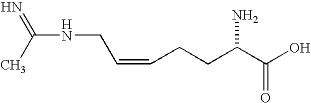

### (2S,5Z)-2-amino-7-[(1-iminoethyl)amino]-5-heptenoic acid, dihydrochloride

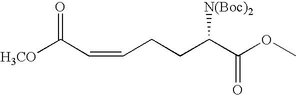

Example T-1) Methyl bis(trifluoroethyl)phosphonoacetate (4.77 g, 15 mmol) and 23.7 g (90 mmol) of 18-crown-6 were dissolved in 80 mL of anhydrous THF and cooled to −78° C. To this soution was added 30 mL (15 mmol) of potassium bis(trimethylsilyl) amide, followed by 5.1 g (14.7 mmol) of N,N-diBoc glutamic aldehyde methyl ester from Example U-3 (see Example U infra). After stirring for 30 minutes at −78° C., the reacion was quenched with aqueous KHSO₄ . Extraction of the reaction mixture with EtOAc and concentration afforded 2.95 g (49%) of the desired compound. Mass spectra M+H=402.

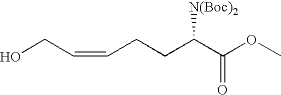

Example T-2) The product from Example T-1 was reduced by the method of Example U-5 to afford the desired compound.

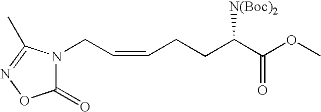

Example T-3) The product from Example T-2 was allowed to react with 3-methyl-1,2,4-oxadiazolin-5-one by the method of Example U-6 to afford the desired compound.

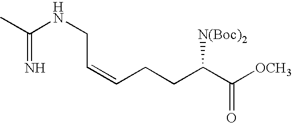

Example T-4) The product from Example T-3 was deprotected by the method of Example U-7 to afford the desired compound.

Example T) The product from Example T-4 was dissolved in 2 N HCl and heated at reflux. The reaction mixture was cooled and concentrated to afford 0.12 g of the desired product. H¹-NMR 1.8-2.0 (m, 2H); 2.05 (s, 3H); 2.15 (q, 2H); 3.75 (d, 2H); 3.9 (t, 1H); 5.45 (m, 1H); 5.6 (m, 1H).

### Example U

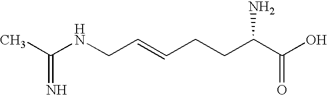

### (2S,5E)-2-amino-7-[(1-iminoethyl)amino]-5-heptenoic acid, dihydrochloride

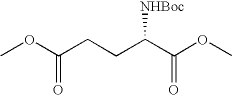

Example U-1) L-glutamic acid (6.0g, 40.78 mmol) was dissolved in methanol (100 mL). To the reaction mixture trimethylsilyl chloride (22.9 mL, 180 mmol) was added at 0° C. under nitrogen and allowed to stir overnight. To the reaction mixture at 0° C. under nitrogen triethylamine (37 mL, 256 mmol) and di-tert-butyldicarbonate (9.8 g, 44.9 mmol) was added and stirred two hours. The solvent was removed and the residue was triturated with ether (200 mL). The triturated mixture was filtered. The filtrate was evaporated to an oil and chromatographed on silica, eluting with ethyl acetate and hexane, to give the mono boc L-glutamic diester (10.99 g, 98%).

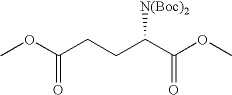

Example U-2) Mono boc L-glutamic acid (10.95 g, 39.8 mmol) was dissolved in acetonitrile (130 mL). To the reaction mixture 4-dimethylaminopyridine (450 mg, 3.68 mmol) and di-tert-butyldicarbonate (14.45 g, 66.2 mmol) was added and stirred for 20 hours. The solvent was evaporated and the residue chromatographed on silica and eluting with ethyl acetate and hexane to give the di-boc-L-glutamic diester (14.63 g, 98%).

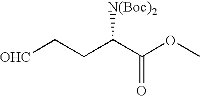

Example U-3) The product from Example U-2 (10.79 g, 28.7 mmol) was dissolved in diethyl ether (200 mL) and cooled in a dry ice bath to −80 C. To the reaction mixture Diisobutylaluminum hydride (32.0 mL, 32.0 mmol) was added and stirred 25 minutes. The reaction mixture was removed from the dry ice bath and water (7.0 mL) was added. Ethyl acetate (200 mL) was added to the reaction mixture and stirred 20 minutes. Magnesium sulfate (10 g) was added to the reaction mixture and stirred 10 minutes. The reaction mixture was filtered through celite and concentrated to give a clear yellow oil (11.19 g). The yellow oil was chromatographed on silica and eluting with ethyl acetate and hexane. The product (8.61, 87%) was a clear light yellow oil.

Mass Spectrometry: M+H 346, M+Na 378

(¹H)NMR (400 MHz, CDCl₃) 9.74 ppm (s, 1H), 4.85 ppm (m, 1H), 3.69 ppm (s, 3H), 2.49 ppm (m, 3H), 2.08 ppm (m, 1H), 1.48 ppm (s, 18H).

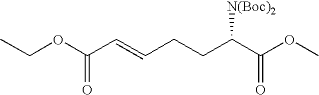

Example U-4) Triethyl phosphonoacetate (6.2 mL, 31.2 mmol) was dissolved in toluene (30 mL) and placed in an ice bath under nitrogen and cooled to 0° C. To the reaction mixture, potassium bis(trimethylsilyl) amide (70 mL, 34.9 mmol) was added and stirred 90 minutes. To the reaction mixture the product from Example U-3 (8.51 g, 24.6 mmol) dissolved in toluene (20 mL) was added and stirred 1 hour. The reaction mixture was warmed to room temperature. To the reaction mixture Potassium hydrogen sulfate ( 25 mL, 25 mmol) was added and stirred 20 minutes. The mixture was extracted with ethyl acetate (3×100 mL), dried over Magnesium sulfate and concentrated to give a cloudy brownish yellow oil (12.11 g). The oil was chromatographed on silica, eluted with ethyl acetate and toluene to give a light yellow oil (7.21 g, 70 %).

Mass Spectrometry: M+H 416, M+NH₄ 433, −boc 316, −2 boc, 216.

(¹H)NMR (400 MHz, CDCl₃) 6.88 ppm (m, 1H), 5.82 ppm (d, 1H), 4.81 ppm (m, 1H), 5.76 ppm (s, 3H), 2.50 ppm (m, 3H), 2.21 ppm (m, 1H), 1.45 ppm (s, 18H).

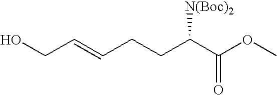

Example U-5) The product from Example U-4 (5.0 g, 12.03 mmol) was dissolved in diethyl ether (100 mL) and placed in a dry ice bath and cooled to −80° C. To the reaction mixture was added diisobutylaluminum hydride (21.0 mL, 21.0 mmol). And stirred 30 minutes. To the reaction mixture water (10 mL) was added, removed from dry ice bath, and stirred 60 minutes. To the reaction mixture magnesium sulfate (10 g) was added and stirred 10 minutes. The reaction mixture was filtered over celite and concentrated to give a yellow oil (5.0 g). The oil was chromatographed on silica, eluted with ethyl acetate and hexane, to give a light yellow oil (2.14 g, 47%).

Mass Spectrometry: M+H 374, M+NH₄ 391

(¹H)NMR (400 MHz, CDCl₃) 5.63 ppm (m, 2H), 4.88 ppm (m, 1H), 4.02 ppm (s, 2H), 3.68 ppm (s, 3H), 2.12 ppm (m, 4H), 1.47 ppm (s, 18H).

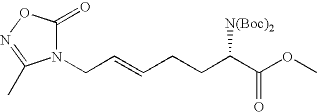

Example U-6) The product from Example U-5 was dissolved in tetrahydrofuran (50 mL). To the reaction mixture triphenyl phosphine on polymer (3.00 g, 8.84 mmol), oxadiazolinone (720 mg, 7.23 mmol), and azodicarboxylic acid dimethyl ester (1.17 g, 3.21 mmol) were added and stirred six hours at room temperature. The reaction mixture was filtered over celite and concentrated to give a cloudy yellow oil (2.81 g). The oil was chromatographed on silica, eluting with ethyl acetate in hexane, to give a clear colorless oil (1.66 g, 68%).

Mass Spectrometry: M+H 456, M+NH₄ 473, −boc 356, −2 boc 256

(¹H)NMR (400 MHz, CDCl₃) 5.65 ppm (m, 1H), 5.45 ppm (m, 1H), 4.79 ppm (m, 1H), 4.11 ppm (d, 2H), 3.68 ppm (s, 3H), 2.17 ppm (m, 4H), 1.47 ppm (s, 18 H).

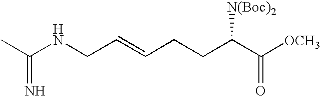

Example U-7) Product from Example U-6 (300 mg, 0.66 mmol) was dissolved in a solution of acetic acid and water (10 mL, 25/75) containing zinc metal and sonicated for 3 hours. The reaction mixture was filtered over celite and chromatographed on reverse phase HPLC to give a clear colorless residue (13 mg, 4%).

(¹H)NMR (400 MHz, CDCl₃) 8.89 ppm (m, 1H), 5.68 ppm (m, 1H), 5.47 ppm (m, 1H), 3.80 ppm (d, 2H), 3.71 ppm (s, 3H), 2.18 ppm (m, 4H), 1.41 ppm (s, 18 H).

Example U) The product from Example U-7 (13.0 mg, 0.031 mmol) was dissolved in 2 N HCl (1.22 mL, 2.44 mmol) and refluxed 1 hour. The reaction mixture was cooled, concentrated, to give a clear colorless oil (6.6 mg, 95%)

Mass Spectrometry: M+H 200,

(¹H)NMR (400 MHz, D₂0) 5.65 ppm (m, 1H), 5.47 ppm (m,1H), 3.80 ppm (t, 1H), 3.72 ppm (d, 2H), 2.0 ppm (m, 5H), 1.87 ppm (m, 2H).

### Example V:

### (α,2S)-.α-aminohexahydro-7-imino-1H-azepine-2-hexanoic acid, trihydrate hydrochloride

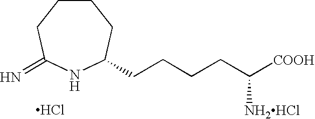

### Example V-1)

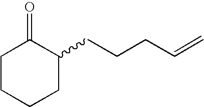

A three neck 3L flask was purged with nitrogen before it was charged with cyclohexanone (1.27 mol, 132 mL) and 500 mL of toluene. This stirred mixture was cooled to 0° C. and 157.2 g (1.1 eq) of potassium t-butoxide was added. After stirring this mix for 1 hr, a color and texture change was noted before a solution of 5-pentenyl bromide (1.27 mol, 136 mL) in 100 mL toluene was added dropwise over 1 h to the mechanically stirred reaction mixture. The reaction mixture was allowed to warm to 25° C. and stir overnight. It was then diluted with 800 mL of 1 N KHSO₄ and the organic phase was dried (MgSO₄), filtered and evaporated to dryness to yield 208.5 g of crude product. This material was then purified by vacuum distillation (under water aspirator pressure) to give the title product in 47% yield.

¹H NMR (CDCl₃, δ ppm): 1.0-2.4 (m, 13H), 4.9-5.1 (m, 2H), 5.7-5.9 (m, 1H).

### Example V-2)

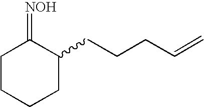

The product of Example V-1 (93.67 g, 0.563 mole) along with EtOH (600 mL), water (300 mL), NaOAc (101.67 g, 1.24 mole), and NH₂OH.HCl (78.31 g, 1.13 mole) were combined in a three neck 3 L flask. This stirred reaction mixture was refluxed for 16 h and then stirred at 25° C. for another 24 h. All solvent was removed under reduced pressure and the residue was partitioned between diethylether (Et₂O, 500 mL) and water (200 mL). The aqueous layer was extracted 3×200 mL ether. The combined organic layers were dried over MgSO₄, filtered, and stripped in vacuo to give the title oxime (121.3 g, 100% crude yield).

¹H NMR (CDCl₃, δ ppm): 1.2- 2.6 (m, 13H), 4.9-5.1 (m, 2H), 5.7-5.9 (m, 1H).

### Example V-3)

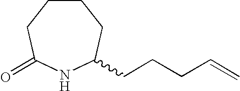

A three neck 3 L flask was purged with nitrogen and then charged with hexamethydisiloxane (471.7 mL, 2.2 moles), toluene (500 mL), and phosphorous pentoxide (203.88 g, 1.4 moles). This heterogeneous mixture was refluxed until a clear solution was obtained (about 1.5 h). After cooling this mixture to room temperature, the oxime product of Example V-1 (102.1 g, 0.563 moles) in 200 mL of toluene was added to the above reaction mixture over a 1 h period at 25° C. The reaction mixture was stirred for another 4-6 h (checked by TLC: 50% EA in Hex, I₂) before it was poured into ice water with thorough mixing. To this ice slurry mixture was added 250 g of NaCl and the resulting mixture was adjusted to pH 5 by adding solid potassium carbonate. This slurry was extracted with 3×500 mL of diethylether (Et₂O) and the combined organic fractions were dried over MgSO₄, filtered and stripped in vacuo to give the crude mixture of regioisomeric lactams (84.6 g).

### Example V-4)

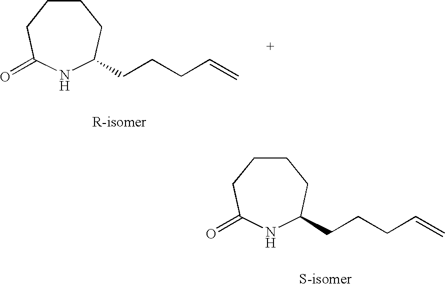

The product of Example V-3 was then subjected to chromatography (silica: acetonitrile) for purification and regioisomeric separation. From the crude sample, the 7-pentenyl regioisomer was isolated in 50% yield and after chiral chromatography, the desired single enantiomers were isolated in 43% yield each.

R-isomer:

Elemental analyses Calcd for C₁₁H₁₉NO: C, 71.99; H, 10.57; N, 7.63.

Found: C, 71.97; H, 10.58; N, 7.52

¹H NMR (CDCl₃, δ ppm): 1.3-1.6 (m, 7H), 1.75-1.9 (m, 2H), 1.95-2.15 (m, 3H), 2.4-2.5 (m, 2H), 3.25-3.35 (m, 1H), 4.95-5.05 (m, 2H), 5.7-5.85 (m, 1H).

¹³C NMR (CDCl₃, δ ppm): 23.166, 25.169, 29.601, 33.209, 35.475, 35.624, 36.783, 53.600, 114.976, 137.923, 177.703

[α]²⁵=+26.90 (CHCl₃) at 365 nm.

S-isomer:

Elemental analyses Calcd for C₁₁H₁₉NO: C, 71.99; H, 10.57; N, 7.63.

Found: C, 72.02; H, 10.61; N, 7.57

¹H NMR (CDCl₃, δ ppm): 1.3-1.6 (m, 7H), 1.75-1.9 (m, 2H), 1.95-2.15 (m, 3H), 2.4-2.5 (m, 2H), 3.25-3.35 (m, 1H), 4.95-5.05 (m, 2H), 5.7-5.85 (m, 1H).

¹³C NMR (CDCl₃, δ ppm): 23.187, 25.178, 29.630, 33.230, 35.526, 35.653, 36.778, 53.621, 115.032, 137.914, 177.703

[α]²⁵=−25.7° (CHCl₃) at 365 nm.

### Example V-5)

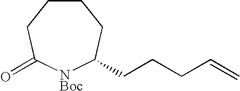

The R-isomer product of Example V-4 (102.1 g, 0.56 mol), dry THF (800 mL), DMAP (68.9 g, 0.56 mol), Di-t-butyl dicarbonate (Boc₂O, 99 g, 0.45 mol) were combined in a three neck 3L flask purged with argon. The reaction mixture was warmed to 70° C. within 30 min before an additional 52.8 g of Boc₂O and 200 mL of dry THF were added. After 30 min. another 32 g of Boc₂O was added and the mixture was stirred for 1 h at 70° C. Another 36 g of Boc₂O was added and the mixture was stirred for 1 h. The reaction mixture was cooled to room temperature and stripped of THF at 18° C. to 20° C. under reduced pressure. A precipitate was filtered and washed with 100 mL of ethylacetate (EA) and discarded (˜45 g). The EA filtrate was diluted with 500 mL of additional EA before it was washed with 500 mL of 1N KHSO₄, 500 mL of saturated aq. NaHCO₃, and 500 mL of brine and then dried over anhydrous Na₂SO₄ for 12 h. This EA extract was then treated with 20 g of DARCO, filtered through celite topped with MgSO₄, and concentrated in vacuo to give 150 g of title product as a dark brown oil.

¹H NMR (CDCl₃, δ ppm): 1.3-1.6 (m, 4H), 1.5 (s, 9H), 1.6-1.9 (m, 6H), 1.95-2.05 (m, 2H), 2.5-2.7 (m, 2H), 4.2-4.25 (m, 1H), 4.95-5.05 (m, 2H), 5.7-5.85 (m, 1H).

### Example V-6)

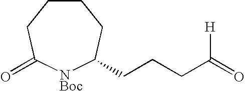

A three neck 3L flask containing the product of Example V-5 (150 g, 0.533) dissolved in 3 L of CH₂Cl₂ was cool to −78° C. A stream of O₃ was passed through the solution for 2.5 h until the color of the reaction mixture turned blue. Argon was then bubbled through the solution maintained at −60° C. to −70° C. until the solution became clear and colorless (˜30 min.). Dimethylsulfide (DMS, 500 mL) was then added before the reaction was brought to reflux and this reflux was continued for 24 h. Another 100 mL of DMS was added and reflux was continued for 12 h. Another 100 mL of DMS was added and reflux continued for an additional 12 h. The solvent and excess DMS were then stripped on a rotary evaporator at 20° C. The residual yellow oil obtained was diluted with 500 mL of DI water and extracted with 3×300 mL of EA. The EA layer was dried over anhydrous MgSO₄, treated with 20 g of DARCO, filtered through a thin layer of celite topped with anhydrous MgSO₄, and stripped of all solvent under reduced pressure to yield 156 g of the crude title product as orange yellow oil.

¹H NMR (CDCl₃, δ ppm): 1.3-1.6 (m, 4H), 1.5 (s, 9H), 1.6-1.9 (m, 6H), 2.45-2.75 (m, 4H), 4.2-4.25 (m, 1H), 9.75 (s, 1H).

### Example V-7)

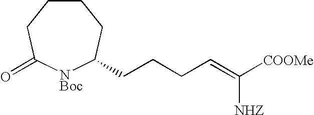

To a sample of N-(Benzyloxycarbonyl)-alpha-phosphonoglycine trimethyl ester (160 g, 0.48 mol) dissolved in 1L of dichloromethane (CH₂Cl₂) and cooled to 0° C. was added a solution of DBU (110.29 g, 0.72.mol) in 100 mL of CH₂Cl₂. This clear colorless reaction mixture was stirred for 1 h at 0° C. to 6° C. before the Boc-aldehyde product of Example V-6 (150 g, 0.53 mol) in 600 mL of CH₂Cl₂ was added drop wise at −5° C. to −1° C. The reaction mixture was stirred for 30 min. at this temperature before it was slowly warmed to 10° C. in approximately 1 h. The reaction mixture was washed with 1 N KHSO₄ (500 mL), saturated aq. NaHCO₃ (200 mL) and 50 aq. NaCl (200 mL). The organic layer was then dried over anhydrous MgSO₄, treated with 40 g of DARCO, filtered through a thin layer of celite topped with anhydrous MgSO₄, and concentrated to give 258 g of the crude title product as an yellow oil. Chromatographic purification of this material gave 130 g (55%) of the pure title product. Elemental analyses Calcd for C₂₆H₃₆N₂O₇: C, 63.96; H, 7.42; N, 5.77. Found: C, 63.42; H, 8.16; N, 5.31.

¹H NMR (CDCl₃, δ ppm): 1.25 (m, 2H), 1.5 (s, 9H), 1.51-1.9 (bm, 8H), 2.25 (m, 2H), 2.5 (m, 1H), 2.65 (m, 1H), 3.75 (s, 3H), 4.12 (m, 1H), 5.15 (s, 2H), 6.3 (bs, 1H), 6.55 (t, 1H), 7.45 (m,5H).

¹³C NMR (CDCl₃, δ ppm): 14.04, 22.62, 23.46, 24.08, 25.27, 27.89, 27.92, 28.34, 28.95, 31.81, 31.86, 32.05, 39.18, 52.31, 54.65, 67.27, 82.62, 128.07, 128.18, 128.46, 135.98, 136.82, 154.50, 164.92, 176.68.

[α]²⁵=+10.9° (CHCl₃) at 365 nm.

### Example V-8)

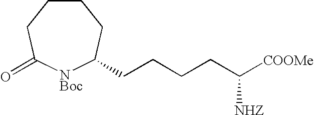

To a MeOH (1 L) solution of the product of Example V-7 (91.3 g, 0.19 mol) was added 2.5 g of S,S-Rh-DIPAMP catalyst followed by hydrogen. The hydrogenation was carried out at 25° C. in 1.5 h in a Parr apparatus. The reaction mixture was filtered through celite before concentrating to provide the crude title product (90 g, 98%) as a brown oil.

¹H NMR (CDCl₃, δ ppm): 1.35 (m, 4H), 1.5 (s, 9H), 1.55-1.95 (m, 10H), 2.4-2.7 (m, 2H), 3.75 (s, 3H), 4.2 (m, 1H), 4.4 (m, 1H), 5.1 (m, 2H), 5.35 (d, 1H), 7.35 (m, 5H).

### Example V-9)

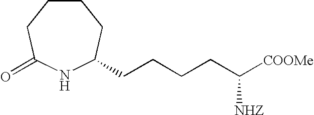

To a solution of the product of Example V-8 (90 g,) in 200 mL of glacial acetic acid was added 200 mL of 4N HCl in dioxane. The reaction mixture was stirred at 25° C. for 20 min. before it was stripped of all solvent under reduced pressure at 40° C. to give a red brown oil. This oily product was treated with 500 mL of water and extracted 2×300 mL of dichloromethane. The combined organic layer was washed with satd. sodium bicarbonate solution (100 mL), dried over magnesium sulfate, filtered and stripped of all solvent to give the crude title product. This material was chromatographed to provide 45 g (62%) of the pure title product.

Elemental analyses Calcd for C₂₁H₃₀N₂O₅: C, 64.02; H, 7.68; N, 7.17.

Found: C, 63.10; H, 7.88; N, 6.60.

¹H NMR (CDCl₃, δ ppm): 1.2-2.0 (m, 14H), 2.45 (t, 2H), 3.25 (m,1H), 3.75 (s, 3H), 4.38 (m, 1H), 5.1 (s, 2H), 5.3 (d, 1H), 5.45 (bs, 1H), 7.35 (m, 5H).

¹³C NMR (CDCl₃, δ ppm): 14.09, 23.11, 24.89, 25.41, 29.53, 32.33, 35.52, 35.79, 36.68, 52.26, 53.51, 53.55, 53.60, 60.26, 66.86, 127.97, 128.05, 128.40, 136.18, 155.85, 172.85, 177.80.

[α]²⁵=−9.9° (CHCl₃) at 365 nm.

### Example V-10)

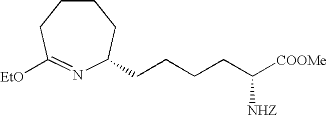

To a 45.0 g (0.115 mol) sample of the product of Example V-9 in 300 mL of dichloromethane purged with argon was added 23.0 g (0.121 mol) of triethyloxonium tetrafluoroborate. This mixture was stirred for 1 h at 25° C. before 150 mL of satd. aq. sodium bicarbonate solution was added. The dichloromethane layer was separated, washed with 150 mL of 50% aq. NaCl solution, dried over sodium sulfate, filtered through celite and concentrated at 25° C. to give a clear yellow oil, 47.0 g (97%) of the title product

Elemental analyses Calcd for C₂₃H₃₄N₂O₅: C, 60.01; H, 8.19; N, 6.69.

Found: C, 65.13; H, 8.45; N, 6.64.

¹H NMR (CDCl₃, δ ppm): 1.2 (t, 3H), 1.25-1.74 (m, 12H), 1.75-1.95 (m, 2H), 2.2-2.3 (m, 1H), 2.4-2.5 (m, 1H), 3.1 (m, 1H), 3.7 (s, 3H), 3.9-4.0 (m, 2H), 4.35 (m, 1H), 5.1 (s, 2H), 5.25 (d, 1H), 7.35 (m, 5H).

¹³C NMR (CDCl₃, δ ppm): 14.23, 23.38, 25.01, 25.21, 26.10, 30.24, 32.16, 32.77, 33.92, 39.15, 52.22, 53.91, 58.05, 60.19, 66.92, 128.11, 128.33, 128.48, 136.27, 155.83,166.29, 173.11, 177.64.

### Example V-11)

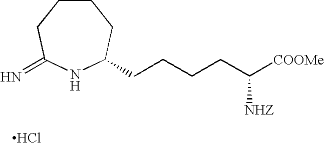

To 7.0 g (0.130 mol) of ammonium chloride in 500 mL methanol was added 31.2 g of the title material of Example V-10 (45.0 g, 0.107 mol). The reaction was refluxed at 65° C. for 5 h before all solvent was removed under reduced pressure to yield 40 g (87%) of the crude product as a foamy viscous mass. This material was purified by column chromatography to provide 37 g (81%) of the title product.

Elemental analyses Calcd for C₂₁H₃₁N₃O₄: C, 59.22; H, 7.57; N, 9.86; Cl, 8.32. Found for C₂₁H₃₁N₃O₄+1.2 HCl+0.5 H₂O: C, 57.20; H, 7.99; N 9.66; Cl, 9.62.

IR (Neat, λ max cm⁻¹): 2935, 1716, 1669.

¹H NMR (CDCl₃, δ ppm): 1.2-2.0 (m, 13H), 2.5 (t, 1H), 2.95 (m, 1H), 3.4 (bs, 1H), 3.7 (s, 3H), 4.3 (m, 1H), 5.1 (s, 2H), 5.55 (d, 1H), 7.3 (m, 5H), 8.75 (bs, 1H), 8.9 (bs, 1H), 9.5 (s, 1H).

¹³C NMR (CDCl₃, δ ppm): 23.20, 24.95, 25.22, 28.94, 31.80, 32.05, 33.75, 34.89, 52.33, 53.76, 56.07, 66.83, 127.93, 128.04, 128.43, 136.26, 156.00, 172.24, 172.87.

Mass (ESI): M/Z, 390.

[α]²⁵=+31.5° at 365 nm.

### Example V)

The title product of Example V-11 (36.0 g, 0.084 mol) in 1 L of 2.3 N HCl was refluxed for 3 h. After cooling to room temperature, the solution was washed with 2×150 mL of CH₂Cl₂ and then stripped of all solvent in vacuo to give 25.6 g (96%) of the title amino acid product as a pale yellow foam.

Elemental analyses Calcd for C₁₂H₂₃N₃O₂.2HCl: C, 46.02; H, 8.01; N, 13.39; Cl 22.45. Found for C₁₂H₂₃N₃O₂+2.2 HCl+0.1 H₂O: C, 42.76; H, 8.02; N, 12.41; Cl, 22.79.

IR (Neat, λ max, cm⁻¹): 2930, 2861, 1738, 1665.

¹H NMR (CD₃OD, δ ppm): 1.3-2.5 (m, 16H), 2.6 (dd, 1H), 2.8 (t, 1H), 3.65 (m, 1H), 4.0 (t, 1H), 7.85 (s, 1H), 8.85 (s, 1H), 8.95 (s, 1H).

¹³C NMR (CD₃OD, δ ppm): 24.49, 25.67, 26.33, 29.71, 31.26, 32.45, 35.04, 35.87, 53.73, 57.21, 171.77, 173.96.

UV, 282 nm, abs 0.015.

Mass (M⁺¹)=242.

[α]²⁵=−47.4° (MeOH) at 365 nm.

ee=91% as determined by CE at λ=214 nm.

### Example W:

### (αS,2R)-α-aminohexahydro-7-imino-1H-azepine-2-hexanoic acid, trihydrate hydrochloride

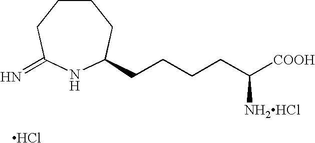

### Example W-1)

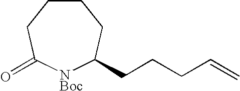

The S-isomer product of Example V-4 (5.45 g, 0.030 mol) was converted to its Boc derivative by the method of Example V-5. After chromatography, this reaction yielded 6.3 g (75%) of the desired title product.

¹H NMR (CDCl₃, δ ppm): 1.3-1.6 (m, 4H), 1.5 (s, 9H), 1.6-1.9 (m, 6H), 1.95-2.05 (m, 2H), 2.5-2.7 (m, 2H), 4.2-4.25 (m, 1H), 4.95-5.05 (m, 2H), 5.7-5.85 (m, 1H).

### Example W-2)

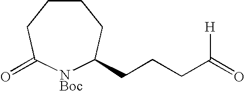

The product of Example W-1 (6.3 g, 0.025 mol) was ozonized by the method of Example V-6 to produce 8.03 g of the crude title aldehyde that was used without further purification.

¹H NMR (CDCl₃, δ ppm): 1.3-1.6 (m, 4H), 1.5 (s, 9H), 1.6-1.9 (m, 6H), 2.45-2.75 (m, 4H), 4.2-4.25 (m, 1H), 9.75 (s, 1H).

### Example W-3)

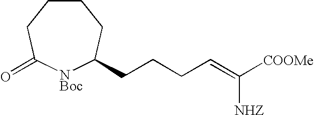

The product of Example W-2 (8.03 g, 0.024 mol) was condensed with N-(Benzyloxycarbonyl)-alpha-phosphonoglycine trimethyl ester (7.9 g, 0.024 mol) utilizing the procedure of Example V-7 to produce 4.9 g (44%) of the desired title product after chromatography.

¹H NMR (CDCl₃, δ ppm): 1.25 (m, 2H), 1.5 (s, 9H), 1.51-1.9 (bm, 8H), 2.25 (m, 2H), 2.5 (m, 1H), 2.65 (m, 1H), 3.75 (s, 3H), 4.15-4.25 (m, 1H), 5.15 (s, 2H), 6.3-6.4 (bs, 1H), 6.45-6.55 (t, 1H), 7.3-7.4 (m, 5H).

### Example W-4)

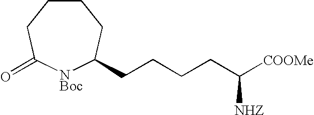

The product of Example W-3 (4.8 g, 0.010 mol) was reduced in the presence of

R,R-Rh-DIPAMP catalyst by the method of Example V-8 to produce 2.9 g (60%) of the desired title product after chromatography.

### Example W-5)

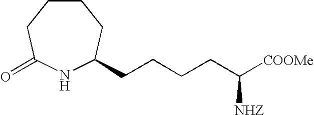

The product of Example W-4 (2.9 g, 0.006 mol) was deprotected by treatment with HCl using the method of Example V-9 to produce 2.3 g (100%) of the desired title product.

¹H NMR (CDCl₃, δ ppm): 1.3-2.0 (m, 14H), 2.45 (t, 2H), 3.25 (m, 1H), 3.75 (s, 3H), 4.38 (m, 1H), 5.1 (s, 2H), 5.3 (d, 1H), 5.45 (bs, 1H), 7.35 (m, 5H).

### Example W-6)

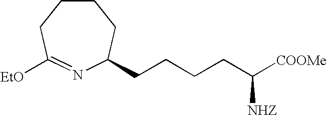

The product of Example W-5 (0.56 g, 0.0015 mol) was alkylated with triethyloxonium tetrafluoroborate using the method of Example V-10 to produce 0.62 g (98%) of the desired title product.

### Example W-7)

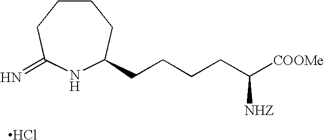

The product of Example W-6 (0.62 g, 0.0015 mol) was treated with ammonium chloride in methanol using the method of Example V-11 to produce 0.50 g (88%) of the desired title product after chromatographic purification.

### Example W-8)

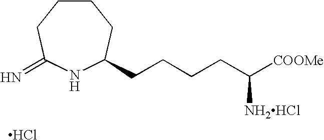

The product of Example W-7 (0.37 g, 0.0009 mol) dissolved in MeOH was added to a Parr hydrogenation apparatus. To this vessel was added a catalytic amount of 5% Pd/C. Hydrogen was introduced and the reaction was carried out at room temperature at pressure of 5 psi over a 7 hr period. The catalyst was removed by filtration and all solvent was removed under reduced pressure from the filtrate to produce 0.26 g (quantitative) of the desired title product.

### Example W)

A solution of the product of Example W-8 dissolved in 2N HCl (30 mL) was maintained at reflux for 2 h before it was cooled to room temperature. All solvent was removed under reduced pressure and the residue was dissolved in 50 mL of water. This solution was again stripped of all solvent under reduced pressure before it was again dissolved in 12 mL of water and then lyophilized to generated 0.245 g (71%) of the title compound.

Elemental analyses Calcd for C₁₂H₂₃N₃O₂.2.3 HCl.1.9 H₂O: C, 40.10; H, 8.16; N, 11.69; Cl 22.69. Found for C₁₂H₂₃N₃O₂+2.1 HCl+0.7 H₂O: C, 40.27; H, 8.28; N, 11.62; Cl, 22.70.

¹H NMR (CD₃OD, δ ppm): 1.4-2.1 (m, 16H), 2.6 (dd, 1H), 2.8 (t, 1H), 3.65 (m, 1H), 4.0 (t, 1H), 7.85 (s, 1H), 8.45 (s, 1H), 8.9 (s, 1H).

¹³C NMR (CD₃OD, δ ppm): 24.46, 25.64, 26.31, 29.69, 31.24, 32.54, 35.00, 35.83, 53.75, 57.20, 171.85, 173.93.

[α]²⁵=+25.7° (MeOH) at 365 nm.

### Example X:

### (αS,2S)-α-aminohexahydro-7-imino-1H-azepine-2-hexanoic acid, trihydrate hydrochloride

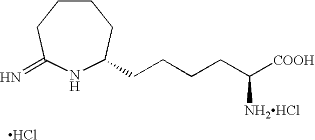

### Example X-1)

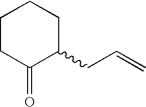

To a 22L round bottom flask equipped with overhead stirrer, half moon shape paddle, heating mantle, thermocouple, and a silver vacuum jacketed distillation column (5 plates) was charged cyclohexanone (4500.0 g, 45.85 mol), acetone dimethyl acetal (5252.6 g, 50.43 mol), allyl alcohol (6390.87 g, 110.04 mol) and p-toluene sulfonic acid (PTSA) (0.256 g, 0.001 mol). After the stirring was started (137 rpm) the pot was heated slowly with the initial set point being 70° C. Heating was increased step wise to a final pot temperature of 150° C. The decision to increase the reactor set point was made based on distillation rate. If the rate of distillate slowed or stopped, additional heat was applied. The additional heating to 150° C. allowed the Claisen rearrangement to occur. After the pot temperature was raised to 150° C. and no distillate was observed, the heating mantle was lowered and the reaction mixture allowed to cool to 130° C. The PTSA was then neutralized with 3 drops of 2.5 N NaOH. The vacuum stripping was then started with the heating mantle lowered away from the flask. Evaporative cooling was used to lower the pot temperature, and the pressure was gradually lowered to 40 mm Hg. When the pot temperature had decreased to ˜100° C., the heating mantle was raised back into the proper position for heating. Unreacted cyclohexanone and low boiling impurities were distilled off. The pot temperature was slowly raised (the maximum temperature deferential between the pot and vapor was ˜12° C.). The product was isolated at 109-112° C. @ 40 mm Hg. Typical yields were 40-45%. Fractions which were <95% by area (GC) were combined and redistilled to afford the title product in a total yield of 55%.

¹H NMR (CDCl₃, δ ppm): 5.8−5.6 (m, 1H), 4.8-5.0 (m, 2H), 2.5−2.4 (m, 1H), 2.3−2.1 (m, 3H), 2.1−1.2 (m, 7H).

¹³C NMR (CDCl₃, δ ppm): 212.53, 136.62, 116.32, 50.39, 42.18, 33.91, 33.52, 28.09, 25.10.

GC/MS m/z=138.

### Example X-2)

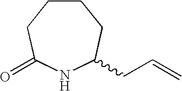

Hydroxyl amine-O-sulfonic acid (91.8 g) dissolved in acetic acid (470 g) was added to a 1 L Bayer flask equipped with a mechanical stirrer, thermocouple, condenser chilled to 0° C., and an addition funnel and heated to 70° C. The allyl cyclohexone (100 g) was added dropwise in approximately 40 min to the above solution while maintaining the temperature between 70 and 78° C. During the addition, the reaction appearance changed from a white slurry to a clear orange solution. After the addition, the reaction was heated and stirred for an additional 5 h at 75° C. An IPC sample was taken each hour. After the reaction was complete, the acetic acid was stripped at 50° C. under reduced pressure on a rotary evaporator. Water (200 mL) was then added to the residue and the solution extracted with toluene (2×300 mL). The organic layers were combined, treated with water (150 ml) and stirred for 10 min. A sodium hydroxide solution (79.4 g of 50 solution) was added until the aqueous layer turned basic (pH 12). The neutralization was carried out in the reactor by controlling the temperature below 40° C. The layers were then separated and the toluene layer was passed through a filter to remove any solids or tarry material. The organic solution was then stripped at 50° C. under reduced pressure on a rotary evaporator. The residue was taken up in a mixture of toluene (510 mL) and heptanes (2040 mL) and heated to 60° C. in a 3 L reactor. A clear yellow-orange solution was obtained. The title product began to crystallize at 53° C. as the solution was slowly cooled to 5° C. while being stirred. The solid was filtered, washed with heptanes (50 mL) and dried over night at 40° C. under house vacuum to produce 66.3 g (60%) of title product as off-white crystals obtained. A portion of this material was recrystallized from toluene and heptane to generate the title product as a white crystalline solid.

¹H NMR (CDCl₃, δ ppm): 5.8−5.6 (m, 1H), 5.5 (bs, 1H), 4.8-5.0 (m, 2H), 3.4−3.3 (m, 1H), 2.5−2.3 (m, 2H), 2.3−2.1 (m, 2H) 2.0−1.2 (m, 6H)

¹³C NMR (CDCl₃, δ ppm): 117.73, 133.83, 119.31, 52.88, 40.95, 37.20, 35.75, 29.96, 23.33.

GC/MS (EI mode)=153.

m.p.=97-99° C.

### Example X-3)

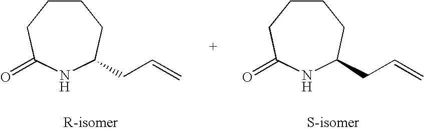

The racemic product mixture of Example X-2 was subjected to chiral chromatographic separation on a Chiralpac AS 20 um column eluting with 100% acetonitrile. A 220 nM wavelength was employed in the detector. A sample loading of 0.08 g/mL of acetonitrile was used to obtain 90% recovery of separated isomers each with >95% ee. A portion of the R-isomer material was recrystallized from toluene and heptane to generate the R-isomer title product as a white crystalline solid.

R-isomer: m.p.=81-82° C.

### Example X-4)

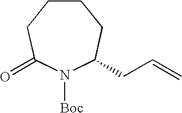

A five necked flat bottom flask equipped with dropping funnel, thermometer and mechanical overhead stirrer was evacuated and purged with nitrogen three times. The R-isomer product lactam of Example X-3 (100.0 g, 0.653 mol), DMAP (7.98 g, 65 mmol) and N-diisopropylethyl amine (Hünigs base, 113.3 g, 0.876 mol) were dissolved in toluene (350 mL) and Di-tert-butyl dicarbonate (170.2 g, 0.78 mol) dissolved in toluene (100 mL) was added. (Note: the reaction works better, when 2.0 eq of Hünigs base were used). The mixture was heated to 65° C. (Note: Steady offgasing during the reaction was observed). After 1.5 h another 86.25 g of Di-tert-butyl-dicarbonate (0.395 mol) dissolved in toluene (50 mL) were added. Heating was continued for 17 h and IPC by HPLC showed 75 conversion. Another 42.78 g of Di-tert-butyl dicarbonate (0.196 mol) in toluene (30 mL) were added and the brown mixture was heated 5.5 h. After cooling to ambient temperature, the mixture was treated with 4M HCl (215 mL), and the aqueous layer was extracted with toluene (2×80 mL). The combined organic layers were washed with NaHCO₃ (170 mL) and 250 ml of water (Note: the internal temperature during the quench was controlled by external cooling with ice/water). Gas evolution was observed. The organic layer was evaporated to give 257.4 g brown liquid. This crude material was purified by plug filtration over SiO₂ (950 g) using toluene/EtOAc 9/1 (6 L) and toluene/AcOEt 1/1 (0.5 L) as eluent giving 139.5 g (51%) of the yellow liquid title product.

### Example X-5)

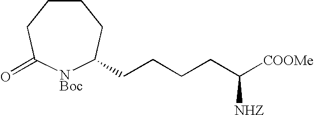

### Example X-6)

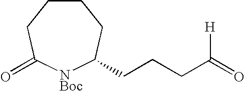

### Example 1f

Into a 2-L stainless steel autoclave equipped with baffles and a six-bladed gas dispersing axial impeller was charged Rh(CO)₂(acac) (0.248 g, 0.959 mmol), BIPHEPHOS (structure shown below and prepared as described in Example 13 of U.S. Pat. No. 4,769,498, 2.265 g, 2.879 mmol), the product of Example X-4 (N-(tert-butoxycarbonyl)-S-7-allylcaprolactam

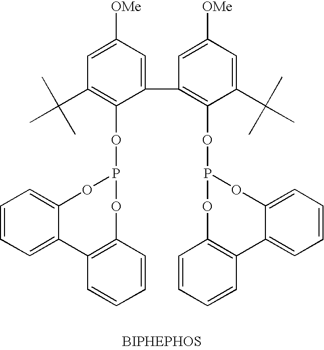

(242.9 g, 0.959 mol), and toluene (965 g). The reactor was sealed and purged 100% carbon monoxide (8×515 kPa). The reactor was pressurized to 308 kPa (30 psig) with 100% carbon monoxide and then a 1:1 CO/H₂ gas mixture was added to achieve a total pressure of 515 kPa (60 psig). With vigorous mechanical agitation, the mixture was heated to 50° C. with a 1:1 CO/H₂ gas mixture added so as to maintain a total pressure of about 515 kPa (60 psig). After 22 h, the mixture was cooled to about 25° C. and the pressure was carefully released. Vacuum filtration of the product mixture and evaporation of the filtrate under reduced pressure afforded a 267.7 g of a light yellow oil. Analysis by ¹H NMR was consistent with essentially quantitative conversion of the starting material with about 96% selectivity to the corresponding aldehyde product of Example V-6. This oil was used without further purification in the following example.

¹H NMR (CDCl₃) δ1.47 (s, 9H), 1.6-1.80 (m, 9H), 1.84-1.92 (m, 1H), 2.41-2.58 (m, 3H), 2.61-2.71 (m, 1H), 4.2 (d, J=5.2 Hz, 1H), 9.74 (s, 1H).

### Example X-8)

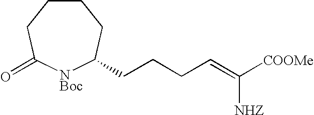

### Example 1g

To a sample of N-(Benzyloxycarbonyl)-alpha-phosphonoglycine trimethyl ester (901.8 g, 2.7 mol) dissolved in CH₂Cl₂ and cooled to 0° C. was added a solution of DBU (597.7 g, 3.9 mol) in CH₂Cl₂. This clear colorless reaction mixture was stirred for 1 h at 0° C. to 6° C. before a sample of the Boc-aldehyde product Example V-6 (812.0 g, 2.9 mol) in CH₂Cl₂ was added drop wise at −5° C. to −1° C. The reaction, work up, and purification was completed as described in Example V-7 to give 1550 g of the title product of Example V-7 containing a small amount of CH₂Cl₂.

### Example X-9)

To a MeOH (1 L) solution of the product of Example V-7 (100 g, 0.20 mol) was added 3 g of RR-Rh-DIPAMP catalyst. The hydrogenation was carried out at 25° C. in 1.5 h in a Parr apparatus. The reaction mixture was filtered through celite before concentrating to provide the crude Example X-9 title product as a brown oil (100 g).

¹H NMR (CDCl₃, δ ppm): 1.35 (m, 4H), 1.5 (s, 9H), 1.6-1.9 (m, 10H), 2.5-2.8 (m, 2H), 3.75 (s, 3H), 4.25 (m, 1H), 4.45 (m, 1H), 5.1 (m, 2H), 5.65 (d, 1H), 7.35 (m, 5H).

### Example X-10)

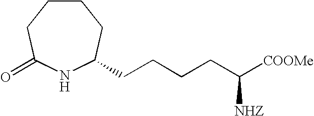

To a solution of the product of Example V-8 (100 g) in 200 mL glacial acetic acid was added 25 mL 4N HCl in dioxane. The reaction mixture was stirred at 25° C. for 20 min. before it was stripped of all solvent under reduced pressure at 40° C. to give 105 g of red brown oil. This oily product was treated with 500 mL of water and extracted 2×300 mL of dichloromethane. The combined organic layer was washed with satd. sodium bicarbonate solution (100 mL), dried over magnesium sulfate, filtered and stripped of all solvent to give 99.9 g of the title product as a red brown oil.

¹H NMR (CDCl₃, δ ppm): 1.25-2.0 (m, 14H), 2.45 (t, 2H), 3.25 (m,1H), 3.7 (s, 3H), 4.35 (m, 1H), 5.1 (s, 2H), 5.5 (d, 1H), 6.45 (bs, 1H), 7.35 (m, 5H). ee=95% as determined by chiral HPLC.

### Example X-11)

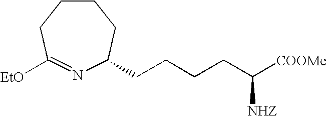

To a 30.0 g (0.077 mol) sample of the product of Example X-10 in 600 mL dichloromethane purged with argon was added 15.7 g (0.082 mol) of triethyloxonium tetrafluoroborate. This mixture was stirred for 1 h at 25° C. before 300 mL of satd. aq. sodium bicarbonate solution was added. The dichloromethane layer was separated, washed with 300 mL 50% aq. NaCl solution, dried over sodium sulfate, filtered through celite and concentrate at 25° C. to give a clear yellow oil, 31.2 g (˜97%) of the title product.

Elemental analyses Calcd for C₂₃H₃₄N₂O₅: C, 60.01; H, 8.19; N, 6.69.

Found for C₂₃H₃₄N₂O₅+0.5 H₂O: C, 64.66; H, 8.24; N, 6.59.

¹H NMR (CDCl₃, δ ppm): 1.2 5 (t, 3H), 1.28-1.75 (m, 12H), 1.8-1.98 (m, 2H), 2.2-2.3 (m, 1H), 2.4-2.5 (m, 1H), 3.1 (m, 1H), 3.78 (s, 3H), 3.9-4.0 (m, 2H), 4.35 (m, 1H), 5.1 (s, 2H), 5.25 (d, 1H), 7.35 (m, 5H).

¹³C NMR (CDCl₃, δ ppm): 14.27, 23.36, 25.21, 25.53, 26.09, 30.22, 32.15, 32.73, 33.90, 39.14, 52.21, 53.89, 58.04, 60.33, 66.89, 128.11, 128.35, 128.48, 136.29, 155.86, 166.30, 173.14, 177.69.

IR (Neat, λ max, cm⁻¹): 3295, 2920, 1739, 1680.

UV, 257 nm, abs 0.015.

[α]²⁵=+39.8° (CHCl₃) at 365 nm.

### Example X-12)

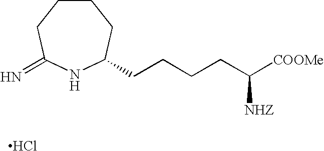

To 4.2 g (0.078 mol) of ammonium chloride in 500 mL methanol was added 31.2 g of the title material of Example X-11. The reaction was refluxed at 65° C. for 5 h before all solvent was removed under reduced pressure to yield 29 g (92%) of the crude product as a foamy viscous mass. This material was purified by column chromatography to provide 23 g (70%) of the title product.

Elemental analyses Calcd for C₂₁H₃₁N₃O₄.1HCl) C, 59.28; H, 7.57; N, 9.89; Cl, 8.39. Found (For C₂₁H₃₁N₃O₄+1HCl+1 H₂O): C, 56.73; H, 7.74; N, 9.40; Cl, 8.06.

IR (Neat, A max cm⁻¹): 3136, 30348, 2935, 1716, 1669.

¹H NMR (CDCl₃, δ ppm): 1.3-2.05 (m, 13H), 2.5 (t, 1H), 2.98 (m, 1H), 3.4 (bs, 1H), 3.75 (s, 3H), 4.35 (m, 1H), 5.1 (s, 2H), 5.5 (d, 1H), 7.35 (m, 5H), 8.75 (s, 1H), 9.0 (s, 1H), 9.5 (s, 1H).

¹³C NMR (CDCl₃, δ ppm): 23.25, 25.01, 25.34, 29.01, 31.88, 32.26, 33.89, 35.06, 52.33, 53.73, 56.20, 66.89, 127.95, 128.06, 128.45, 136.27, 155.93, 172.27, 172.80.

UV, 257 nm, abs 0.009.

Mass (ESI): M/Z, 390.

[α]²⁵=−42.80 (MeOH) at 365 nm.

ee=96% as determined by chiral HPLC.

### Example X)

The title product of Example X-12 (23 g) in 500 mL 2N HCl was refluxed for 5 h. All solvent was then removed in vacuo and the residue redissolved in water was washed with 2×300 mL of CH₂Cl₂. The aqueous was then concentrated in vacuo to give 17 g (100%) of the light brown hygroscopic solid title product.

Elemental analyses Calcd for C₁₂H₂₃N₃O₂.2HCl: C, 45.86; H, 8.02; N, 13.37; Cl 22.56. Found for C₁₂H₂₃N₃O₂+2.1 HCl+0.7 H₂O: C, 43.94; H, 8.65; N, 12.52; Cl, 22.23.

IR (Neat, λ max, cm⁻¹): 2936, 1742, 1669.

¹H NMR (CD₃OD, δ ppm): 1.3-2.1 (m, 16H), 2.6 (dd, 1H), 2.8 (t, 1H), 3.65 (m, 1H), 4.0 (t, 1H), 7.85 (s, 1H), 8.4 (s, 1H), 8.95 (s, 1H).

¹³C NMR (CD₃OD, δ ppm): 24.49, 25.67, 26.33, 29.71, 31.26, 32.45, 35.04, 35.87, 53.73, 57.21, 171.77, 173.96.

UV, 209 nm, abs 0.343.

Mass (M⁺¹)=242.

[α]²⁵=+60.0° (MeOH) at 365 nm.

ee=92% as determined by CE at λ=210 nm.

### Example Y

### (αR,2S)-α-aminohexahydro-7-imino-1H-azepine-2-hexanoic acid, trihydrate hydrochloride

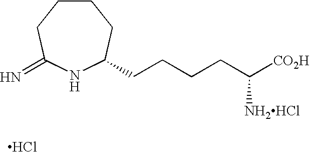

### Example Y-1)

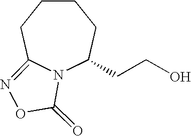

A solution of Example X-3 (3.0 g, 0.015 mol) in methylene chloride and methanol (75/45 mL) was cooled to −78° C. in a dry ice bath. The reaction stirred as ozone was bubble through the solution at a 3 ml/min flow rate. When the solution stayed a consistent deep blue, the ozone was remove and the reaction was purged with nitrogen. To the cold solution was added sodium borohydride (2.14 g, 0.061 mol) very slowly to minimize the evolution of gas at one time. To the reaction was added glacial acetic acid slowly to bring the pH to 3. The reaction was then neutralized with saturated sodium bicarbonate. The oraganics were then washed 3×50 mL with brine, dried over magnesium sulfate anhydrous, removed under reduced pressure. The pale oil was run through a plug of silica (15 g) to afford the alcohol 5.15 g, 0.026 mol (64%). C₉H₁₄N₂O₃.

¹H NMR (CDCl₃, δ ppm) 1.18-2.15 (m, 8H), 3.59 (m, 2H), 4.39 (m, 1H).

¹³C NMR (CDCl₃, δ ppm) 24.45, 25.71, 26.47, 32.56, 34.67, 51.16, 58.85, 160.66, 160.89.

### Example Y-2)

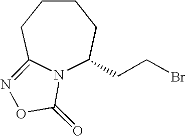

To a solution of Example Y-1 (5.15 g, 0.026 mol) in methylene chloride (100 mL) at 0° C. in an ice bath was added carbon tetrabromide (10.78 g, 0.033 mol). The solution was cooled to 0° C. in an ice bath. Then triphenylphosphine (10.23 g, 0.39 mol) was added portion wise as not to allow the temperature raise above 3° C. The reaction was stirred for 2 hours and the solvent was removed in vacuo. The crude was purified by flash chromatography to yield the bromide (5.9 g, 0.023 mol) in 87% yield.

Elemental analysis calculated for C₁₀H₁₆N₂O₃: C, 41.40; H, 5.02; N, 10.73; Br, 30.60. Found: C, 41.59; H, 5.07; N, 10.60, Br, 30.86.

¹H NMR (CDCl₃, δ ppm) 1.50-2.60 (m, 9H), 2.99 (dd, 1H), 3.35 (m, 2H), 4.41 (m, 1H).

¹³C NMR (CDCl₃, δppm) 23.89, 25.33, 26.04, 28.06, 31.59, 35.05, 52.79, 159.3, 160.2.

### Example Y-3)

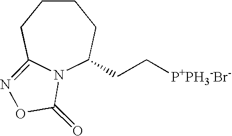

To a solution of Example Y-2 (5.71 g, 0.026 mol) in toluene (25 mL) was added triphenyl phosphine (7.17 g, 0.027 mol). The reaction refluxed in an oil bath for 16 hours. After cooling, the toluene was decanted from the glassy solid. The solid was triturated with diethyl ether overnight to afford the phosphonium bromide (10.21 g, 0.020 mol) in 90% yield.

¹H NMR (CDCl₃, δ ppm): 1.50-2.9 (m, 11H), 3.58 (m, 1H), 4.16 (m, 1H), 4.41 (m, 1H), 7.6-8.0 (m, 15H).

³C NMR (CDCl₃, δ ppm): 24.43, 24.97, 25.50, 55.08, 55.27, 116.9, 118.1, 130.4, 130.6, 133.5, 135.1, 135.2, 159.4,160.

³¹P NMR (CDCl₃, δ ppm) 26.0.

### Example Y-4)

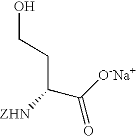

To a 1L Round Bottom Flask was added N-benzyloxycarbonyl-D-homoserine lactone (97 g, 0.442 mol) in ethanol (500 mL). To the reaction was added solution of sodium hydroxide (1M, 50 mL). The reaction was monitored by thin layer chromatography for 12 hours until the starting material had been consumed. Toluene (60 mL) was added and then solvent was removed in vacuo. The residue was carried on with no further purification.

### Example Y-5)

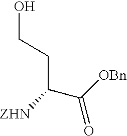

The residue from Example Y-4 was suspended in DMF in a 1L Round Bottom Flask. To the suspension was added benzyl bromide (76.9 g, 0.45 mol, 53.5 mL) and the mixture was stirred for 1 hour. A sample was quenched and analyzed by mass spec to indicate the consumption of the starting material and that there was no lactone reformation. To the reaction was added 1L of ethyl acetate and 500 mL of brine. The aqueous layer was washed 2 additional times with 500 mL of ethyl acetate. The organics were combined, dried over MgSO₄ and concentrated. Silica gel chromatography provided N-benzyloxycarbonyl-S-homoserine benzyl ester as a white solid (80 g).

### Example Y-6)

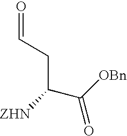

To a 2L Round Bottom Flask was added pyridinium chlorochromate (187 g, 0.867 mol) and silica gel (197 g) suspended in CH₂Cl₂ (600 mL). To the slurry was added a solution of the product of Example Y-5 (80 g, 0.233 mol) in CH₂Cl₂ (600 mL). The mixture was stirred for 4 hours. Thin layer chromatography indicated that the starting material was consumed. To the reaction was added 1 L of diethyl ether. The solution was then filtered through a pad of ceilite followed by a pad of silica gel. The solvent was removed in vacuo and the resulting oil was purified by silica gel chromatography to afford the aldehyde (58.8 g) in 38% overall yield.

MH⁺342.5, MH+NH₄⁺359.5.

¹H NMR (CDCl₃, δ ppm) 3.15 (q, 2H), 4.12 (m, 1H), 5.15 (s, 2H), 5.20 (s, 2H), 7.31 (m, 10H), 9.72 (s, 1H).

### Example Y-7)

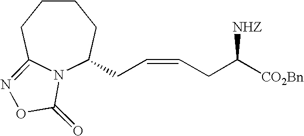

To a 3L 3-neck flask was added the phosphonium salt from Example Y-3 (56.86 g, 0.11 mol) that had been dried over P₂O₅ under a vacuum in THF (1L). The slurry was cooled to −78° C. in a dry-ice bath. To the cold slurry was added KHMDS (220 mL, 0.22 mol) dropwise so that the temperature did not rise above −72° C. The reaction was stirred at −78° C. for 20 minutes and then −45° C. for 2 hours. The temperature was then dropped back to −78° C. and the aldehyde (15.9 g, 0.047 mol) from Example Y-6 was added in THF (50 mL) dropwise over 45 minutes. The reaction was stirred at −77° C. for 30 minutes then warmed to −50° C. for 1 hour before it was warmed to room temperature over 4 hours. To the reaction was added ethyl acetate (200 mL) and saturated ammonium chloride. The organics were collected, dried over MgSO₄ and concentrated in vacuo. The crude oil was purified on silica chromatography to afford the olefin compound (45.1 g) in 81% yield as a pale yellow viscous oil.

¹H NMR (CDCl₃, δ ppm) 1.4-2.6 (m, 10H), 2.92 (d, 1H), 4.17 (m, 1H), 4.38 (m, 1H), 5.05 (q, 2H), 5.40 (m, 2H), 7.3 (m, 10H).

¹³C NMR (CDCl₃, δ ppm) 29.49, 29.64, 31.32, 39.60, 49.56, 53.98, 61.01, 65.25, 124.14, 127.81, 128.20, 128.55, 128.79, 129.30, 130.96, 135.68, 137.31, 152.59, 157.57, 171.61.

### Example Y)

To a 20 mL vial was added the product from Example Y-7 (19.77 g, 0.039 mol) in Dioxane (50 mL) and 4N aqueous HCl (250 mL). This solution was added a cat. amount of 10% Pd on carbon in a hydrogenation flask. The flask was pressurized with H₂ (50 psi) for five hours. The reaction was monitored by mass spec and the starting material had been consumed. The solution was filtered through a pad of celite and washed with water.

The solvent was removed by lyophollization to afford the title compound (7.52 g) in 81% yield.

MH⁺242.2, MH+NH₄⁺259.2.

¹H NMR (CD₃OD δ ppm) 1.2-2.0 (m, 15H), 2.42 (d, 1H), 2.65 (dd, 1H), 3.49 (m, 1H), 3.98 (t, 1H), 7.26 (s), 8.05 (s), 8.35 (s).

¹³C NMR (CDCl₃, δ ppm) 24.43, 25.58, 26.00, 26.10, 32.75, 33.45, 35.31, 53.76, 54.55, 157.27, 175.13.

### Example Z

### (αS,2S)-α-aminohexahydro-7-imino-1H-azepine-2-hexanoic acid, trihydrate hydrochloride

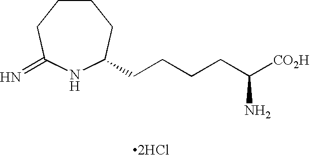

### Example Z-1)

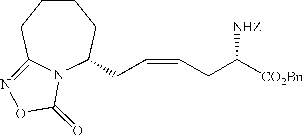

To a 1 L 3-neck flask was added the phosphonium salt from Example Y-3 (21.21 g, 0.041 mol) in THF (200 mL). The slurry was cooled to −78° C. in a dry-ice bath. To the cold slurry was added KHMDS (88 mL, 0.044 mol) dropwise so that the internal temperature did not rise above −72° C. The reaction stirred at −78° C. for 20 minutes then −45° C. for 1 hour. The temperature was then dropped back to −78° C. and the aldehyde (15.9 g, 0.047 mol) (prepared as in Example Y(4-6) using N-benzyloxycarbonyl-L-homoserine lactone) was added in THF (50 mL) dropwise over 45 minutes. The reaction was stirred at −77° C. for 30 minutes then warmed to −50° C. for 30 minutes then warmed to room temperature over 4 hours. To the reaction was added ethyl acetate (100 mL) and saturated ammonium chloride. The organics were collected, dried over MgSO₄ and concentrated in vacuo. The crude oil was purified on silica chromatography to afford the olefin compound (9.0 g) in 45% yield as a pale yellow viscous oil.

¹H NMR (CDCl₃, δppm) 1.4-2.6 (m, 10H), 2.92 (d, 1H), 4.17 (m, 1H), 4.38 (m, 1H), 5.05 (q, 2H), 5.40 (m, 2H), 7.3 (m, 10H).

¹³C NMR (CDCl₃, δ ppm) 29.49, 29.64, 31.32, 39.60, 49.56, 53.98, 61.01, 65.25, 124.14, 127.81, 128.20, 128.55, 128.79, 129.30, 130.96, 135.68, 137.31, 152.59, 157.57, 171.71.

### Example Z)

To a 20 mL vial was added the product from Example Z-1 in dioxane (5 mL) and 4N aqueous HCl (16 mL). This solution was added a cat. amount of 10% Pd on carbon in a hydrogenation flask. The flask was pressurized with H₂ (50 psi) for five hours. The reaction was monitored by mass spec and the starting material had been consumed. The solution was filtered through a pad of ceilite and washed with water. The solvent was removed by lyophilization to afford the title compound (98.7 mg) in 79.4% yield.

MH⁺242.2, MH+NH4⁺259.2.

¹H NMR (CD₃OD, δ ppm) 1.2-2.0 (m, 15H), 2.42 (d, 1H), 2.6 (dd, 1H), 3.49 (m, 1H), 3.98 (t, 1H).

¹³C NMR (CDCl₃, δ ppm) 24.43, 25.58, 26.00, 26.10, 32.75, 33.45, 35.31, 53.76, 54.55, 157.27, 175.13.

### Example AA

### (2S,4Z)-2-amino-6-[(2R)-hexahydro-7-imino-1H-azepin-2-yl]-4-hexenoic acid

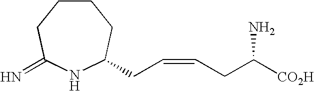

### Example AA-1)

### (2S,4Z)-6-[(2R)-hexahydro-7-imino-1H-azepin-2-yl]-2-[[(phenylmethoxy)carbonyl]amino]-4-hexenoic acid, phenylmethyl ester

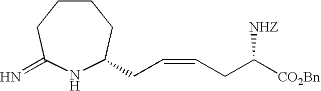

To a 50 mL flask was added a sample of Example Z-1 (1.5 g, 2.97 mmol) in methanol (25 mL). A 60% solution of glacial acetic acid (16 mL) was then added to the reaction mixture. A precipitate was observed. Additional methanol was added to dissolve the solid (1 mL). To the reaction was then added zinc dust (0.200 g). The reaction was sonicated for 4 hours during which the temperature was maintained at 37° C. The reaction was monitored by TLC and MS until the starting material was consumed and a mass corresponding to the product was observed. The solution was decanted from the zinc and a 30% solution of acetonitrile/water (100 mL) was added to the filtrate. The reaction was purified with 52% acetonitrile/water in two runs on the Waters Preparatory HPLC [a gradient of from 20% to 70% acetonitrile over 30 minutes]. Lyophilization of the resulting product afforded the title material of Example AA-1 (1.01 g) in 73% yield as a white solid.

MH⁺464.4, MH+Na⁺486.4.

¹H NMR (CD₃OD, δ ppm): 1.2-2.0 (m, 8H), 2.42 (m, 2H), 2.6 (m, 5H), 3.49 (q, 1H), 4.31 (t, 1H), 5.15 (s, 2H), 5.22 (s, 2H), 5.43 (q, 1H), 5.59 (q, 1H), 7.25 (bs, 10H).

¹³C NMR (CDCl₃, δ ppm): 24.37, 29.61, 30.76, 32.45, 33.73, 34.42, 55.40, 57.09, 68.06, 68.07, 122.3, 124.9, 128.76, 129.09, 129.28, 129.39, 129.51, 129.61, 155.71, 158.35, 173.90.

### Example AA)

To a 250 mL flask was added the product of Example AA-1 (1.0 g, 2.2 mmol) in 4 M HCl (100 mL). The reaction was refluxed overnight, monitored by MS until the starting material had been consumed and the mass for the product was observed. The reaction, without further work up was purified in two runs on the Water's prep reverse phase column using 18% acetonitrile/water [0% to 30% acetonitrile/water over 30 minutes]. Lyophilization of the combined fractions afforded the title product (0.34 g) in 64% yield as a cream colored foam.

MH⁺240.3, MH+Na⁺486.4.

¹H NMR (CD₃OD, δ ppm): 1.2-2.0 (m, 6H), 2.35 (m, 2H), 2.45 (dd, 2H), 2.69 (m, 2H), 3.61 (dt, 1H), 3.98 (t, 1H), 5.59 (m, 1H), 5.65 (m, 1H).

¹³C NMR (CDCl₃, δ ppm): 23.65, 24.66, 32.51, 32.84, 33.1, 33.25, 54.10, 56.1, 126.80, 129.33, 153.33, 172.52.

### Example BB

### (2S,4E)-2-amino-6-[(2R)-hexahydro-7-imino-1H-azepin-2-yl]-4-hexenoic acid

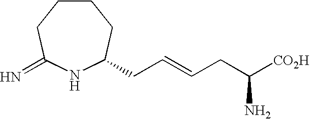

### Example BB-1)

### (2S,4E)-2-[[(phenylmethoxy)carbonyl]amino]-6-[(5R)-6,7,8,9-tetrahydro-3-oxo-3H,5H-[1,2,4]oxadiazolo[4,3-a]azepin-5-yl]-4-hexenoic acid, phenylmethyl ester

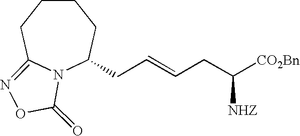

To a 250 mL flask was added Example Z-1 (2.0 g, 3.9 mmol) and phenyl disulfide (0.860 g, 3.9 mmol) in a cyclohexane (70 mL)/benzene(40 mL) solution. Nitrogen was bubbled through the solution to purge the system of oxygen. The reaction was exposed to a short wave UV lamp for the weekend. The reaction was evaluated by normal phase HPLC (ethyl acetate/hexane). 71% of the trans isomer and 29% of the cis isomer was observed. The reaction was subjected to an additional 3 days of UV upon which 84% of the starting material converted to the trans isomer and 16% of the starting cis isomer remained. Purification by chromatography afforded Example BB-1 (0.956g) in 48% yield.

MH⁺506.1, MH+NH4⁺523.2.

¹H NMR (CD₃OD, δ ppm): 1.2-2.0 (m, 8H), 2.42-2.6 (m, 6H), 2.91 (dd, 1H), 4.19 (m, 1H), 4.31 (dt, 1H), 5.09 (s, 2H), 5.11 (s, 2H), 5.18 (dt, 1H), 5.27 (m, 1H), 7.25 (bs, 10H).

### Example BB-2)

(2S,4E)-6-[(2R)-hexahydro-7-imino-1H-azepin-2-yl]-2-[[(phenylmethoxy)carbonyl]amino]-4-hexenoic acid, phenylmethyl ester, monohydrochloride

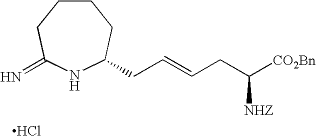

A sample of the product of Example BB-1 (0.956 g, 1.9 mmol) in MeOH (80 mL) was deprotected by method of Example AA-1 with Zn dust (1.5 g) and 60% HOAc/H₂O (40 mL). The resulting product was purified by reverse phase chromatography to afford the title material (0.248 g) in 28% yield.

### Example BB)

The product of Example BB-2 (0.248 g, 0.53 mmol) was transformed into the title product by the method of Example AA using HCl (2 mL), H₂O (2 mL), CH₃CN (4 mL). The crude product was purified by reverse phase chromatography to afford the title product of Example BB (0.073 g) in 57% yield.

MH⁺240.3, MH+Na⁺486.4.

¹H NMR (CD₃OD, δ ppm) 1.2-2.0 (m, 6H), 2.35 (t, 2H), 2.55-2.82 (m, 4H), 3.68 (dt, 1H), 4.05 (t, 1H), 5.65 (m, 2H).

### Example CC

### (E)-2-amino-2-methyl-6-[(1-iminoethyl)amino]-4-hexenoic acid, dihydrochloride

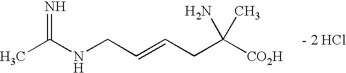

### Example CC-1)

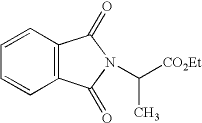

DL-Alanine ethyl ester hydrochloride (5 g, 32.5 mmol) was suspended in toluene (50 mL). Triethyl amine (4.5 mL, 32.5 mmol) was added followed by phthalic anhydride (4.8 g, 32.5 mL). The reaction flask was outfitted with a Dean-Stark trap and reflux condenser and the mixture was heated at reflux overnight. Approximately 10 mL of toluene/water was collected. The reaction mixture was cooled to room temperature and diluted with aqueous NH₄Cl and EtOAc. The layers were separated and the aqueous layer was extracted with EtOAc (3×). The ethyl acetate extract was washed with brine, dried over MgSO₄, filtered and concentrated in vacuo to give the title phthalyl-protected amino ester as a white crystalline solid in near quantitative yield.

¹H NMR (400 MHz, CDCl₃, δ ppm): 1.2 (t, 3H), 1.6 (d, 3H), 4.2 (m, 2H), 4.9 (q, 1H), 7.7 (m, 2H), 7.9 (m, 2H)

### Example CC-2)

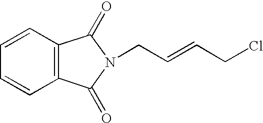

Potassium phthalimide (18.5 g, 0.1 mol) was added to a 250 mL round bottomed flask containing 1,4-butene dichloride (25 g, 0.2 mol). The reaction mixture was heated to 150° C. for 1.5 h. The mixture was cooled to room temperature and was partitioned between brine and Et₂O. The organic layer was dried with MgSO₄, filtered and concentrated in vacuo. The residue was recrystallized from hot ethanol to give the title 1-chloro-4-phthalimidobutene (8.9 g, 39%) as orange crystals.

HRMS calcd. For C₁₂H₁₀ClNO₂: m/z=236.0478 [M+H]. Found: 236.0449

¹H NMR (300 MHz, CDCl₃, δ ppm□ 4.1 (d, 2H), 4.3 (d, 2H), 5.9 (m, 2H), 7.7 (m, 2H), 7.9 (m, 2H)

### Example CC-3)

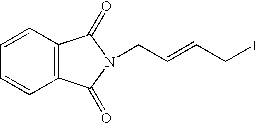

A sample of the product of Example CC-2 (2.3 g, 9.8 mmol) was dissolved in acetone (50 mL). Nal (3.2 g, 21 mmol) was added and the mixture was refluxed overnight. After cooling to room temperature, Et₂O was added and the mixture was washed sequentially with sodium thiosulfate and brine. The organic layer was dried with MgSO₄, filtered and concentrated in vacuo to give the title iodide (2.8 g, 87.5%) as a light yellow solid that was used without further purification.

¹H NMR (400 MHz, CDCl₃, δ ppm): 3.8 (d, 2H), 4.2 (d, 2H), 5.7 (m, 1H), 6.0 (m, 1H), 7.7 (m, 2H), 7.9 (m, 2H)

Mass (M+1)=328

### Example CC-4

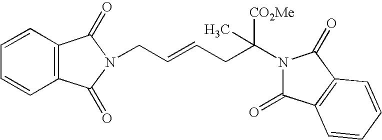

A solution of KHMDS (2.6 g, 13.3 mmol) in THF (50 mL) was cooled to −78° C. A solution of the product of Example CC-1 (2.2 g, 8.87 mmol) in THF (15 mL) was added and 1,3-dimethyl-3,4,5,6-tetrahydro-2(1H)-pyrimidinone (DMPU, 1.0 mL, 8.87 mL) was added immediately thereafter. After the solution was stirred at −78° C. for 40 minutes, a solution of the product of Example CC-3 (2.9 g, 8 87 mmol) in THF (15 mL) was added. The flask was removed from the cold bath and was stirred at room temperature for 3 h. The reaction mixture was partitioned between saturated aqueous NaHCO₃ and EtOAc. The organic extract was washed with brine, dried over MgSO₄, filtered and concentrated in vacuo to give the desired bis-pththalyl protected amino ester as a yellow solid. This residue was chromatographed on silica gel (1:1 hexanes: EtOAc) and gave 1.4 g (35%) of the title material as a white solid.

¹H NMR (300 MHz, CDCl₃, δ ppm□1.2 (t, 3H), 1.6 (d, 3H), 2.8 (dd, 1H), 3.1 (dd, 1H), 4.2 (m, 4H), 5.6 (m, 1H), 5.8 (m, 1H), 7.6 (m, 4H), 7.7 (m, 2H), 7.9 (m, 2H)

Mass (M+H)=447

### Example CC-5

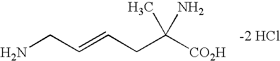

The product of Example CC-4 (0.78 g, 1.76 mmol) was dissolved in a mixture of formic acid (10 mL, 95%) and HCl (20 mL, concentrated HCl) and was refluxed for 3 days. The reaction mixture was cooled to 0° C. and filtered to remove phthalic anhydride. After concentrating in vacuo (T<40° C.), the title unsaturated alpha methyl lysine was obtained as a white solid (0.38 g, 95%), which was used without further purification.

¹H NMR (300 MHz, D₂O, δ ppm): 1.4 (s, 3H), 2.4 (dd, 1H), 2.6 (dd, 1H), 3.5 (d, 2H), 5.7 (m, 2H)

Mass(M+H)=317

### Example CC

The product of Example CC-5 (0.2 g, 0.86 mmol) was dissolved in H₂O (8 mL) and was brought to pH 9 with 2.5 N NaOH. Ethyl acetimidate—HCl (0.42 g, 3.4 mmol) was added in four portions over 1 h. After 1 h, the mixture was acidified to pH 4 with 10% HCl and was concentrated in vacuo. The residue was then passed through a water-washed DOWEX 50WX4-200 column (H form, 0.5 N NH₄OH eluent). The residue was concentrated in vacuo, acidified to pH 4 with 10% HCl, and concentrated to give the title product (17 mg, 6%) as an oil.

HRMS calcd. For C₉H₁₇N₃O₂: m/z=200.1399 [M+H]. Found: 200.1417

¹H NMR (400 MHz, D₂O, δ ppm): 1.4 (s, 3H), 2.1 (s, 3H), 2.5 (dd, 1H), 2.6 (dd,1H), 3.8 (d, 2H), 5.6 (m, 2H)

### Example DD

### (R,E)-2-amino-2-methyl-6-[(1-iminoethyl)amino]-4-hexenoic acid, dihydrochloride

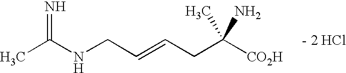

### Example DD-1

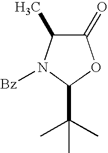

(2S,4S)-3-Benzoyl-2-(tert-butyl)-4-methyl-1,3-oxazolidin-5-one was prepared according to Seebach's procedure. Seebach, D.; Fadel, A. Helvetica Chimica Acta 1985, 68, 1243.

### Example DD-2

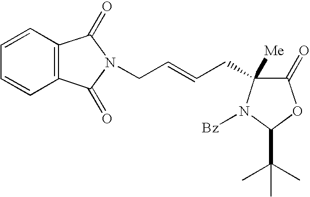

A solution of KHMDS (0.65 g, 3.24 mmol), DMPU (0.33 mL, 2.7 mmol) and THF (40 mL) was cooled to −78° C. A solution of (2S,4S)-3-benzoyl-2-(tert-butyl)-4-methyl-1,3-oxazolidin-5-one (Example DD-1) (0.70 g, 2.7 mmol) in THF (10 mL) was added dropwise. After 45 min, a solution of the product of Example CC-3 (0.88 g, 2.7 mmol) in THF (10 mL) was added. The reaction mixture was stirred at room temperature for 2 h and quenched with saturated aqueous NaHCO₃. The layers were separated and the aqueous layer was extracted with EtOAc. The organic layers were combined and washed with brine, dried over MgSO₄, filtered and concentrated in vacuo. The resulting yellow oil was chromatographed on silica gel (9:1 then 4:1 hexanes/ethyl acetate) to give the title protected unsaturated alpha methyl D-lysine (0.26 g, 20%) as a colorless oil.

HRMS calcd. For C₂₇H₂₈N₂O₅: m/z=461.2076[M+H]. Found: 461.2033

¹H NMR (400 MHz, CDCl₃,δ ppm: 0.9 (s, 9H), 1.5 (s, 3H), 4.3 (m, 2H), 5.5 (m, 2H), 5.6 (m, 2H), 6.1 (m, 1H), 7.5 (m, 5H), 7.7 (m, 2H), 7.9 (m, 2H)

### Example DD-3

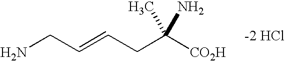

The product of Example DD-2 (0.255 mg, 0.55 mmol) was dissolved in 6N HCl (6 mL) and formic acid (6 mL) and was heated to reflux for 24 h. The reaction mixture was cooled to room temperature and concentrated in vacuo. The residue was suspended in water and washed with CH₂Cl₂. The aqueous layer was concentrated and passed through a water-washed DOWEX 50WX4-200 column (H form, 0.5 N NH₄OH eluent). The residue was concentrated in vacuo, acidified to pH 4 with 10% HCl, and concentrated to give the title unsaturated D-lysine (71 mg, 55%) as an oil which was used without further purification.

¹H NMR (400 MHz, D₂O, δ ppm: 1.4 (s, 3H), 2.5 (dd, 1H), 2.6 (dd, 1H), 3.4 (d, 2H), 5.6 (m, 2H), 5.7 (m, 2H)

### Example DD

The product of Example DD-3 (13 mg, 0.056 mmol) was dissolved in H₂O (5 mL) and was brought to pH 9 with 2.5 N NaOH. Ethyl acetimidate—HCl (27 mg, 0.2 mmol) was added in four portions over 2 h. After 2 h, the mixture was acidified to pH 4 with 10% HCl and was concentrated in vacuo. The residue was passed through a water-washed DOWEX 50WX4-200 column (H form, 0.5 N NH₄OH eluent). The residue was concentrated in vacuo, acidified to pH 4 with 10% HCl, and concentrated to give the title product (45 mg) as an oil.

HRMS calcd. For C₉H₁₇N₃O₂: m/z=200.1399 [M+H]. Found: 200.1386

¹H NMR (400 MHz, D₂O, δ ppm): 1.4 (s, 3H), 2.1 (s, 3H), 2.5 (dd, 1H), 2.6 (dd, 1H), 3.8 (d, 2H), 5.6 (m, 2H)

### Example E

### (S,E)-2-amino-2-methyl-6-[(1-iminoethyl)amino]-4-hexenoic acid, dihydrochloride

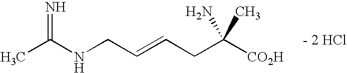

### Example EE-1

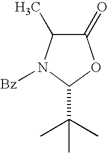

(2R,4R)-3-Benzoyl-2-(tert-butyl)-4-methyl-1,3-oxazolidin-5-one was prepared according to Seebach's procedure. Seebach, D.; Fadel, A. Helvetica Chimica Acta 1985, 68, 1243.

### Example EE-2

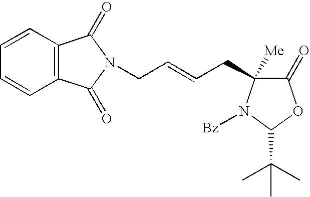

A solution of the (2R,4R)-3-benzoyl-2-(tert-butyl)-4-methyl-1,3-oxazolidin-5-one product of Example EE-1 (2.0 g, 7.6 mmol) in THF (50 mL) was cooled to −78° C. A −78° C. solution of KHMDS (0.65 g, 3.24 mmol) in THF (25 mL) was added dropwise. After 30 min, a solution of the product of Example CC-3 (2.8 g, 8.6 mmol) in THF (25 mL) was added. The reaction mixture was stirred at room temperature for 1 h and quenched with saturated aqueous NaHCO₃. The layers were separated and the aqueous layer was extracted with EtOAc. The organic layers were combined and washed with brine, dried with MgSO₄, filtered and concentrated in vacuo. The resulting orange oil was chromatographed on silica gel (9:1 then 4:1 hexanes/ethyl acetate) to give the protected title unsaturated alpha methyl L-lysine (0.5 g, 15%) as a white solid.

HRMS calcd. For C₂₇H₂₈N₂O₅: m/z=461.2076[M+H]. Found: 461.2043

¹H NMR (400 MHz, CDCl₃, δ ppm): 0.9 (s, 9H), 1.5 (s, 3H), 4.3 (m, 2H), 5.5 (m, 2H), 5.6 (m, 2H), 6.1 (m, 1H), 7.5 (m, 5H), 7.7 (m, 2H), 7.9 (m, 2H)

### Example EE-3

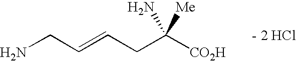

The product of Example EE-2 (0.5 g, 1 mmol) was dissolved in 12N HCl (10 mL) and formic acid (5 mL) and this mixture was heated to reflux for 12 h. The reaction mixture was cooled in the freezer for 3 h and the solids were removed by filtration. The residue was washed with CH₂Cl₂ and EtOAc. The aqueous layer was concentrated in vacuo and gave the title unsaturated alpha methyl L-lysine (0.26 g, 99%) as an oil which was used without further purification.

¹H NMR (300 MHz, D₂O, δ ppm): 1.4 (s, 3H), 2.5 (dd, 1H), 2.6 (dd, 1H), 3.4 (d, 2H), 5.7 (m, 2H)

### Example EE

The product of Example EE-3 (0.13 g, 0.56 mmol) was dissolved in H₂O (1 mL) and was brought to pH 9 with 2.5 N NaOH. Ethyl acetimidate—HCl (0.28 g, 2.2 mmol) was added in four portions over 1 h. After 1 h, the mixture was acidified to pH 4 with 10% HCl and was concentrated in vacuo. The residue was and passed through a water-washed DOWEX 50WX4-200 column (0.5 N NH₄OH eluent). The residue was concentrated in vacuo, acidified to pH 4 with 10% HCl, and concentrated to give the title product as an oil (40 mg).

HRMS calcd. For C₉H₁₇N₃O₂: m/z=222.1218 [M+Na]. Found: 222.1213

¹H NMR (300 MHz, D₂O, δ ppm): 1.4 (s, 3H), 2.1 (s, 3H), 2.4 (dd, 1H), 2.6 (dd, 1H), 3.8 (d, 2H), 5.6 (m, 2H)

### Example FF

### 2-amino-2-methyl-6-[(1-iminoethyl)amino]-4-hexynoic acid, dihydrochloride

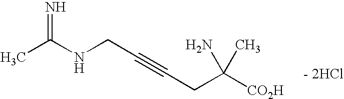

### Example FF-1

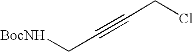

The N-boc-1-amino-4-chlorobut-2-yne was prepared following the procedure described in Tetrahedron Lett. 21, 4263 (1980).

### Example FF-2

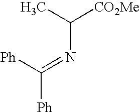

Methyl N-(diphenylmethylene)-L-alaninate was prepared by following the procedure described in J. Org. Chem., 47, 2663 (1982).

### Example FF-3

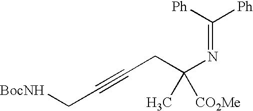

Dry THF (1000 mL) was placed in a flask purged with argon and 60% NaH dispersed in mineral oil (9.04 g, 0.227 mol) was added. To this mixture was added the product of Example FF-2 (30.7 g, 0.114 mol). The reaction mixture was then stirred at 10° C.-15° C. for 30 min. Potassium iodide (4 g) and iodine (2 g) were added and immediately followed by the addition of the product of Example FF-2 (23 g, 0.113 mol in 200 mL THF) in 30 min. The reaction mixture was then stirred at 55° C. until the starting material disappeared (˜2 h). The reaction mixture was then cooled to room temperature and the solvent was evaporated. Ethyl acetate (500 mL) was added and the mixture was carefully washed with 2×200 mL deionized water. The organic layer was dried over anhydrous MgSO₄, filtered and evaporated to give 44 g of crude product. Purification by chromatography using 20% ethyl acetate in hexane afforded the title protected unsaturated alpha-methyl lysine (28 g, 57%).

Anal.Calcd for C₂₆H₃₀N₂O₄ and 0.5 ethylacetate: C,70.42; H, 7.14; N, 5.91.

Found: C, 70.95; H, 7.73; N, 6.09

IR (Neat, λ max, cm⁻¹): 2981, 1714, 1631

¹H NMR (CDCl₃, δ ppm): 1.28 (s, 9H), 1.4 (s, 3H), 2.65-2.76(m, 2H), 3.15 (s, 3H), 3.7 (bs, 2H), 4.6 (bs, 1H), 6.95-7.4 (m, 10H)

¹³C NMR (CDCl₃, δ ppm): 24.29, 28.33, 28.39, 33.24, 51.60, 53.55, 127.79, 127.97, 128.26, 128.36, 128.43, 128.54, 128.66, 130.05, 130.22, 132.39

Mass (M+1)=435

DSC purity: 261.95° C.

### Example FF-4

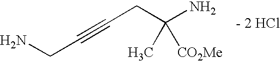

The product of Example FF-3 (16 g, 0.0368 mol) was dissolved in 1N HCl (300 mL) and stirred at 25° C. for 2 h. The reaction mixture was washed with ether (2×150 mL) and the aqueous layer separated and decolorized with charcoal. Concentration afforded ˜9 g (100% yield) of the deprotected unsaturated alpha-methyl lysine ester FF-4 as white foamy solid.

Anal.Calcd for C₈H₁₄N₂O₂ containing 2.26 HCl and 1.19 H₂O: C,35.06; H, 6.86; N, 10.22; Cl, 29.24. Found: C, 35.31; H, 7.38; N, 10.70; Cl, 29.77

¹H NMR (D₂O, δ ppm): 1.56 (s, 3H), 2.8-3.0 (2 dt, 2H), 3.75(s, 2H), 3.79 (s, 3H)

¹³C NMR (D₂O, δ ppm): 23.89, 29.81, 32.05, 57.08, 61.90, 79.57, 82.43, 173.92

Mass (M+1)=171

DSC purity: 114.22° C.

UV=206 nm,abs 0.013

[α]₂₅ in methanol=0 at 365 nm

### Example FF-5

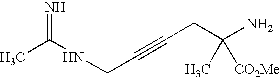

The product of Example FF-4 (2.43 g, 0.01 mol) was dissolved in deionized water (25 mL). A solution of NaOH (400 mg, 0.01 mol) in deionized water (25 mL) was added at 25° C. to bring the pH to ˜7.95 and stirring was continued another 10 min. Ethylacetimidate hydrochloride (988 mg, 0.008 mol) was added to the reaction mixture with simultaneous adjustment of the pH to ˜8.5 by adding 1N NaOH. The reaction mixture was stirred at pH 8 to 8.5 for 3 h following acetimidate addition. 1N HCl was added to the reaction mixture (4.1 pH). The solvent was evaporated at 50° C. to afford a yellow crude hygroscopic residue (4 g, >100% yield). Purification was carried out on the Gilson chromatography system using 0.1% AcOH/CH₃CN/H₂O.

Anal.Calcd for C₁₀H₁₇N₃O₂ containing 2.25 HCl and 1.7 H₂O: C, 37.08; H, 7.05; N, 12.97; Cl, 24.63. Found: C, 37.01; H, 6.79; N, 12.76; Cl, 24.87

IR (Neat, λ max, cm⁻¹): 2953, 2569, 1747, 1681, 1631

¹H NMR (D₂O, δ ppm): 1.52 (s, 3H), 2.12 (s, 3H), 2.74-2.96 (2 dt, 2H), 3.75 (s, 3H), 3.95 (t, 2H)

¹³C NMR (D₂O, δ ppm): 23.89, 29.81, 32.05, 57.08, 61.90, 79.57, 82.43, 173.92

Mass (M+1)=212

### Example FF

The product of Example FF-5 (100 mg, 0.0005 mol) was dissolved in 8N HCl (20 mL) and stirred for 10 h at reflux. The reaction mixture was cooled to room temperature and the aq. HCl was evaporated on rotavap. The residue was dissolved in deionized water (10 mL) and water and reconcentrated under vacuum to afford the title product as a yellow glassy solid in almost quantitative yield (88 mg).

Anal.Calcd for C₉H₁₅N₃O₂ containing 2.4 HCl and 1.8 H₂O: C, 34.08; H, 6.67; N, 13.25; Cl, 26.83. Found: C, 34.32; H, 6.75; N, 13.63; Cl, 26.47

IR (Neat, λ max, cm^{-m}): 1738, 1677, 1628, 1587

¹H NMR (D₂O, δ ppm): 1.6 (s, 3H), 2.24 (s, 3H), 2.8-3.0 (2 dt, 2H), 4.1 (s, 2H)

¹³C NMR (D₂O, δ ppm): 21.22, 24.10, 29.88, 34.58, 80.04, 80.99, 128.39, 168.07, 176.13

Mass (M+1)=198

### Example GG

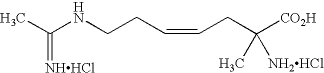

### (2R/S,4Z)-2-amino-2-methyl-7-[(1-iminoethyl)amino]-4-heptenoic acid, dihydrochloride

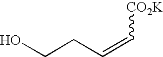

Example GG-1) 5,6 dihydropyran-2-one (49.05 g, 0.5 mol) was dissolved in 200 mL of water. Potassium hydroxide (35 g, 0.625 mol) was added and the reaction mixture stirred at ambient temperature for 5 hours. The solvent was removed in vacuo to yield a colorless glassy solid (65 g, 84%) that was characterized by NMR to be predominantly the cis isomer of the title compound.

¹H NMR (CDCl₃) δ: 2.7 (m, 2H), 3.6 (t, 2H), 5.8-5.85(m, 1H), 5.9-5.97 (m, 1H).

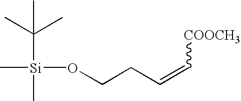

Example GG-2) The product of Example GG-1 was dissolved in 100 mL of dimethyl formamide. Methyl Iodide (52 mL, 0.84 mol) was then added resulting in an exotherm to 40° C. The reaction mixture was stirred at room temperature for 10 hours and partitioned between 150 mL of ethylacetate/diethylether in a 20/80 ratio and ice water. The aqueous layer was separated and re-extracted with 100 mL of diethyl ether. The organic layers were combined, dried (Na₂SO₄), filtered and stripped of all solvent to yield the desired methyl ester product (40 g, 71%). This material was dissolved in 200 mL of methylene chloride and the solution cooled to 0° C. Tertiarybutyl dimethylsilylchloride, triethylamine and dimethylaminopyridine were added. The reaction mixture was slowly warmed to room temperature and stirred for 10 hours under nitrogen. The reaction was extracted with 100 mL of 1N aqueous potassium bisulfate solution. The organic layer was washed with 2×100 mL of brine and then with 3×150 mL of water. The organic layer was dried (Na₂SO₄), filtered and stripped to yield 42 g (56%) of the title material.

¹H NMR (CDCl₃) δ: 0.02 (s, 6H), 0.085 (s, 9H), 2.8-2.85 (m, 2H), 3.65 (s, 3H), 3.66-3.7 (m 2H), 5.8 (m, 1H), 6.3 (m,1H)

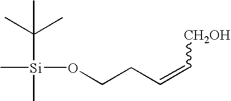

Example GG-3) The material from Example GG-2 was dissolved in 25 mL of toluene and cooled to 0° C. Diisobutylaluminum hydride (1.0 M in toluene, 32 mL, 48 mmol) was added dropwise maintaining the temperature between 5 and −10° C. The reaction mixture was stirred for 1.5 hours between 6 and −8° C. before it was cooled to −25° C. To this mixture was added 100 mL of 0.5N sodium potassium tartarate. The reaction mixture was allowed to warm up to room temperature and stirr for an hour. A gelatinous precipitate was formed which was filtered. The aqueous was extracted with 2×100 mL EtOAc. The combined organic layers were dried (sodium sulfate), filtered and concentrated in vacuo to yield title product (3.45 g, 66%) as a colorless oil.

¹H NMR (CDCl₃) δ: 0.02 (s, 6H), 0.085 (s, 9H), 2.25-2.32 (m, 2H), 2.6 (bs, 1H), 3.6 (t, 2H), 4.08 (d, 2H), 5.45-5.55 (m, 1H), 5.7-5.75 (m, 1H)

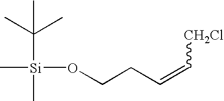

Example GG-4) The product (8 g, 37 mmol) from Example GG-3 was dissolved in 100 mL methylene chloride and this solution was cooled to 0° C. Methanesulfonyl chloride was then added and this mixture was stirred for 5 min. Triethylamine was then added. The temperature maintained between 0 and −10° C. during the addition of the aforementioned reagents. The reaction mixture was subsequently warmed up to room temperature and stirred for 24 hours. It was then extracted with 100 mL of 50% aqueous sodium bicarbonate solution. The organic layer was washed with 100 mL of saturated aqueous brine solution, dried (sodium sulfate), filtered and stripped in vacuo to yield the title material (8.2 g, 94%).

¹H NMR (CDCl₃) δ: 0.02 (s, 6H), 0.085 (s, 9H), 2.25-2.32 (m, 2H), 3.6 (t, 2H), 4.08 (d, 2H), 5.6-5.7 (m, 2H)

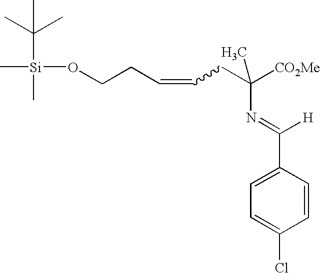

Example GG-5) A solution of N-p-chloro phenylimine alanine methyl ester (8.85 g, 34 mmol) dissolved in 59 mL of tetrahydrofuran was purged with Argon. NaH (1.64 g, 41 mmol) was added whereupon the solution turned bright orange and subsequently a deep red. A solution of the title material from Example GG-4 (8 g, 34 mmol) in 40 mL of tetrahydrofuran was added to the above anionic solution. An exotherm was observed raising the temperature to almost 40° C. The reaction mixture was maintained between 48 and −52° C. for 2 hours. It was then cooled to room temperature and filtered. Filtrate was stripped in vacuo to yield the title material (8.4 g, 50% crude yield) as a yellow oil.

¹H NMR (CDCl₃) δ: 0.02 (s, 6H), 0.085 (s, 9H), 1.45 (s, 3H), 1.6 (s, 1H), 2.2-2.25(m, 2H), 2.65 (d, 2H), 3.55 (m, 2H), 3.7 (s, 3H), 5.45-5.55 (m, 2H), 7.35-7.7 (m, 4H)

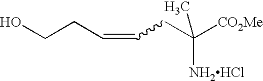

Example GG-6) The title material from Example GG-5 (8.4 g, 18.2 mmol) was treated with 125 mL 1N hydrochloric acid and the reaction was stirred for an hour at room temperature. After the reaction mixture had been extracted 2×75 mL of ethylacetate the aqueous layer was stripped in vacuo at 56° C. to yield 4 g of the title material (100% crude yield).

¹H NMR (CD₃OD) δ: 1.6 (s, 3H), 2.3-2.4 (m, 2H), 2.65-2.8 (m, 2H), 3.6-3.65 (m, 2H), 3.87 (s, 3H), 5.4-5.5 (m, 1H), 5.75-5.85 (m, 1H)

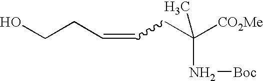

Example GG-7) The title product of Example GG-6 (1.9 g, 8.5 mmol) was dissolved in a mixture of 15 mL dioxane and 8 mL of water. Solid potassium bicarbonate was then carefully added to avoid foaming. The reaction mixture was stirred for 10 min before tertiarybutyloxycarbonyl anhydride was added portion-wise and reaction mixture was stirred at ambient temperature for 24 hours. The reaction mixture was diluted with 100 mL of ethylacetate and 50 mL of water before it was poured into a separatory funnel. The organic layer was separated, dried (Na₂SO₄), filtered and stripped to yield the title material as a colorless oil (1.9 g, 78% crude yield).

¹H NMR (CDCl₃) δ: 1.42 (s, 9H), 1.55 (s, 3H), 2.3-2.36 (m, 2H), 2.58-2.65 (m, 2H), 3.65-3.7 (t, 2H), 3.75 (s, 3H), 5.42-5.5 (m,1H), 5.55-5.62 (m, 1H)

Example GG-8) Another 1.9 g sample of the title material from Example GG-6 was converted by the methods of Example GG-7 to the crude Z/E mixture of the title product of Example GG-7. This material further purified on silica with a solvent system of ethylacetate/hexane in a 20/80 ratio to obtain the minor E-isomer as well as the major Z-isomer.

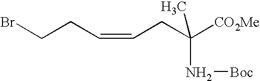

Example GG-9) The title Z-isomer from Example GG-8 (1.8 g, 6.25 mmol) was dissolved in 20 mL of acetonitrile and this solution was cooled to 0° C. Pyridine (0.76 g, 9.4 mmol) was then added followed by the portion-wise addition of solid dibromotriphenylphosphorane (3.46 g, 8.2 mmol) over 10 min. The reaction mixture was stirred under Argon for 24 hours at room temperature. The precipitate that formed was filtered off. The filtrate was concentrated in vacuo to give 2.8 g of an oil that was purified on silica gel using a solvent system of ethylacetate/hexane in a 60/40 ratio. The 1.1 g of title material (50%) was characterized by NMR.

¹H NMR (CDCl₃) δ: 1.44 (s, 9H), 1.55 (s, 3H), 2.6-2.65 (m, 4H), 3.35-3.4 (m, 2H), 3.75 (s, 3H), 5.4-5.45 (m, 1H), 5.55-5.6 (m, 1H)

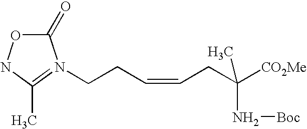

Example GG-10) The title material from Example GG-8 (300 mg, 0.86 mmol) was dissolved in 25 mL of dimethylformamide (DMF). The potassium salt of 3-methyl-1,2,4-oxadiazolin-5-one (130 mg, 0.94 mmol) was added and the reaction mixture was heated to 52° C. and maintained there for 18 hours with stirring. It was then cooled to room temperature before the DMF was stripped in vacuo at 60° C. The residue was purified on silica gel with a gradient of 60/40 to 90/10 ethyl acetate/ hexane to yield 300 mg (95%) of the title material.

¹H NMR (CD₃OD) δ: 1.35 (s, 3H), 1.43 (s, 9H), 2.32 (s, 3H), 2.45-2.55 (m, 4H), 3.65-3.7 (m, 2H), 3.72 (t, 3H), 5.5-5.6 (m, 2H)

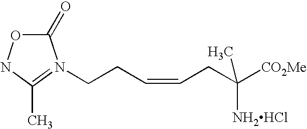

Example GG-11) The product of Example GG-10 (300 mg) was treated with 0.05 N of aqueous HCl and this solution was stirred for 30 min. The solvent was removed in vacuo to afford the desired material in nearly quantitative yield.

¹H NMR (CD₃OD) δ: 1.6 (s, 3H), 2.25 (s, 3H), 2.45-2.55 (m, 2H), 2.7-2.8 (m, 2H), 3.3-3.4(m, 5H), 5.5-5.6 (m, 1H), 5.7-5.8 (m, 1H)

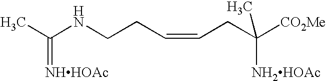

Example GG-12) The title material from Example GG-11 (198 mg, 0.54 mmol) was dissolved in 50 mL of MeOH. Formic acid (40 mg) was then added followed by Palladium on Calcium carbonate (400 mg). The reaction mixture was heated to 65° C. with stirring in a sealed tube for 24 hours. It was then cooled to room temperature and filtered. The filtrate was concentrated in vacuo and the residue purified by reverse phase HPLC to yield 115 mg (75%) of the title material.

¹H NMR (CD₃OD) δ: 1.4 (s, 3H), 1.95 (s, 3H), 2.25 (s, 3H), 2.4-2.52 (m, 4H). 3.25-3.35 (m, 2H), 3.75 (t, 3H), 5.54-5.62 (m, 2H)

Example GG) The title material (75 mg) from Example GG-12 was dissolved in 15 mL of 2N hydrochloric acid. The reaction mixture was heated to a reflux and stirred for 6 hours before ot was cooled to room temperature. The solvent was removed in vacuo. The residue was dissolved in 25 mL of water and stripped on the rotary evaporator to remove excess hydrochloric acid. The residue was dissolved in water and lyophilized to give 76 mg (˜100%) of the title material.

Elemental analyses Calcd for C₁₀H₁₉N₃O₂+2.2HCl+2.2 H₂O: C, 36.06; H, 7.75; N, 12.61. Found for C₁₀H₁₉N₃O₂+2.2HCl+2.2 H₂O: C, 35.91; H, 7.61; N, 12.31

¹H NMR (CD₃OD) δ: 1.47 (s, 3H), 2.32 (s, 3H), 2.45-2.64 (m, 4H), 2.58-2.65 (m, 2H), 3.65-3.7 (t, 2H), 5.55-5.65 (m, 2H)

### Example HH

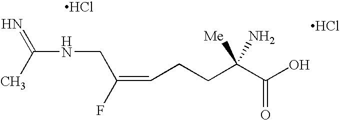

### (2S,5E)-2-amino-2-methyl-6-fluoro-7-[(1-iminoethyl)amino]-5-heptenoic acid, dihydrochloride

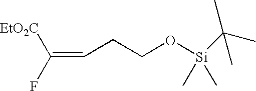

Example-HH-1) To a cold (−78° C.) solution of triethyl 2-fluorophosphonoacetate (25.4 g, 105 mmol) in 100 mL of THF was added n-butyl lithium (63 mL of 1.6 M in hexane, 101 mmol). This mixture was stirred at −78° C. for 20 min producing a bright yellow solution. A solution of crude 3-[(tert-butyldimethylsilyl)oxy]propanal (J. Org. Chem., 1994, 59, 1139-1148) (20.0 g, 105 mmol) in 120 mL of THF was then added dropwise over ten minutes, and the resulting mixture was stirred for 1.5 h at −78° C., at which time analysis by thin layer chromatography (5% ethyl acetate in hexane) showed that no starting material remained. The reaction was quenched at −78° C. with sat. aqueous NH₄Cl (150 mL). The organic layer was collected, and the aqueous layer was extracted with diethyl ether (300 mL). The combined organics were washed with brine (200 mL), dried over MgSO₄, filtered and concentrated. The crude material was filtered through a plug of silica gel (150 g) eluting with hexane (2 L) to give 14.38 g (52%) of the desired (2E)-5-[[(1,1-dimethylethyl)di-methylsilyl]oxy]-2-fluoro-2-pentenoic acid ethyl ester product as a clear oil. ¹H NMR and ¹⁹F NMR indicated that the isolated product had an approximate E:Z ratio of 95:5.

HRMS calcd. for C₁₃H₂₆FO₃Si: m/z=277.1635 [M+H]⁺, found: 277.1645.

¹H NMR (CDCl₃) δ0.06 (s, 6H), 0.94 (s, 9H), 1.38 (t, 3H), 2.74 (m, 2H), 3.70 (m, 2H), 4.31 (q, 2H), 6.0 (dt, vinyl, 1H).

¹⁹F NMR (CDCl₃) δ−129.78 (d, 0.05 F, J=35 Hz, 5% Z-isomer), −121.65 (d, 0.95 F, J=23 Hz, 95% E-isomer).

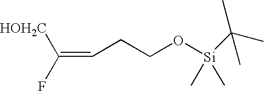

Example-HH-2) To a solution of Example-HH-1 (6.76 g, 24.5 mmol) in 100 mL of methanol at room temperature was added solid NaBH₄ (4.2 g, 220 mmol) in 1.4 g portions over three hours. After 3.5 hours water was added (10 mL). Additional solid NaBH₄ (4.2 g, 220 mmol) was added in 1.4 g portions over three hours. The reaction was quenched with 150 mL of sat. aqueous NH₄Cl and extracted with diethyl ether (2×250 mL). The organic layers were combined, dried over MgSO₄, filtered and concentrated. The crude material, 4.81 g of clear oil, was purified by flash column chromatography on silica gel eluting with 10% ethyl acetate in hexane to give 2.39 g (42%) of the desired (2E)-5-[[(1,1-dimethylethyl)dimethylsilyl]oxy]-2-fluoro-2-penten-1-ol product as a clear oil, that contained an approximate E:Z ratio of 93:7 by ¹⁹F NMR.

HRMS calcd. for C₁₁H₂₄FO₂Si: m/z=235.1530 [M+H]⁺, found: 235.1536.

¹H NMR (CDCl₃) δ0.06 (s, 6H), 0.88 (s, 9H), 2.35 (m, 2H), 3.62 (t, 2H), 4.19 (dd, 2H), 5.2 (dt, vinyl, 1H).

¹⁹F NMR (CDCl₃) δ−120.0 (dt, 0.07F, 7% Z-isomer), −109.82 (q, 0.93 F, J=21 Hz, 93% E-isomer).

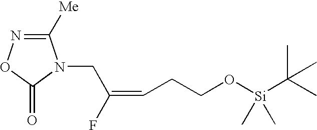

Example-HH-3) To a mixture of Example-HH-2 (2.25 g, 9.58 mmol), polymer-supported triphenylphosphine (3 mmol/g, 1.86 g, 15 mmol) and 3-methyl-1,2,4-oxadiazolin-5-one (1.25 g, 12.5 mmol) in 60 mL of THF was added dropwise diethylazodicarboxylate (2.35 mL, 14.7 mmol). The reaction mixture was stirred for 1 h at room temperature, and additional 3-methyl-1,2,4-oxadiazolin-5-one (0.30 g, 3.0 mmol) was added. After 30 minutes, the mixture was filtered through celite, and the filtrate was concentrated. The resulting yellow oil was triturated with diethyl ether (30 mL) and the solid removed by filtration. The filtrate was concentrated, triturated with hexane (30 mL) and filtered. The filtrates was concentrated to an oil which was purified by flash column chromatography on silica gel eluting with 15% ethyl acetate in hexane to give 1.83 g (60%) of the desired 4-[(2E )-5-[[(1,1-dimethylethyl)dimethylsilyl]oxy]-2-fluoro-2-pentenyl]-3-methyl-1,2,4-oxadi-azol-5(4H)-one product as a clear oil, that contained only the desired E-isomer by ¹⁹F NMR.

HRMS calcd. for C₁₄H₂₆FN₂O₃Si: m/z=317.1697 [M+H]⁺, found: 317.1699.

¹H NMR (CDCl₃) δ0.04 (s, 6H), 0.85 (s, 9H), 2.28 (s, 3H), 2.37 (m, 2H), 3.64 (t, 2H), 4.32 (d, 2H), 5.4 (dt, vinyl, 1H).

¹⁹F NMR (CDCl₃) δ−110.20 (q, 1 F, J=21 Hz).

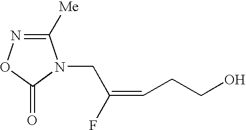

Example-HH-4) A solution of Example-HH-3 (1.83 g, 5.78 mmol) in a mixture of acetic acid (6 mL), THF (2 mL) and water (2 mL) was stirred at room temperature for 2.5 hours. The resulting solution was concentrated in vacuo to an oil which was dissolved in diethyl ether (50 mL). The organic layer was washed with saturated NaHCO₃, and the aqueous layer was extracted with diethyl ether (2×50 mL) and ethyl acetate (2×50 mL). The combined organic layers were dried (MgSO₄), filtered and evaporated to give 1.15 g (98%) of the desired 4-[(2E)-2-fluoro-5-hydroxy-2-pentenyl]-3-methyl-1,2,4-oxadiazol-5(4H)-one product as a clear colorless oil.

HRMS calcd. for C₈H₁₂FN₂O₃: m/z=203.0832 [M+H]⁺, found: 203.0822.

¹H NMR (CDCl₃) δ2.31 (3H), 2.4 (m, 2H), 3.66 (t, 2H), 4.37 (d, 2H), 5.42 (dt, vinyl, 1H). ¹⁹F NMR (CDCl₃) δ−110.20 (q, 1 F, J=21 Hz).

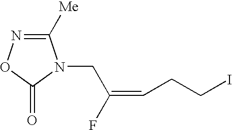

Example-HH-5) To a CH₂Cl₂ (2 mL) solution of triphenylphosphine (238 mg, 0.91 mmol) and imidazole (92 mg) at 0° C. was added solid iodine (230 mg, 0.91 mmol), and the mixture was stirred for 5 minutes. To the resulting yellow slurry was added a CH₂Cl₂ (1.5 mL) solution of Example-HH-4 (0.15 g, 0.74 mmol). The slurry was allowed to warm to room temperature and stirred 30 minutes. The reaction mixture was diluted with CH₂Cl₂ (10 mL), washed with saturated Na₂S₂O₃ (5 mL) and brine (5 mL), dried (MgSO₄), filtered and evaporated to an oil. Addition of diethyl ether (10 mL) to the oil gave a white precipitate that was removed by filtration and the filtrate was concentrated to an oil. The crude material was purified by flash column chromatography on silica gel eluting with 30% ethyl acetate in hexane to give 0.18 g (78%) of the desired 4-[(2E)-2-fluoro-5-iodo-2-pentenyl]-3-methyl-1,2,4-oxadiazol-5(4H)-one product as a clear oil, which solidified upon standing, mp=58.1-58.6° C.

Anal. calcd. for C₈H₁₀FIN₂O₂: C, 30.79; H, 3.23; N, 8.98. Found: C, 30.83; H, 3.11; N, 8.85. HRMS calcd. for C₈H₁₁FIN₂O₂: m/z=330.0115 [M+H]⁺, found: 330.0104.

¹H NMR (CDCl₃) δ2.31 (s, 3H), 2.75 (q, 2H), 3.21 (t, 2H), 4.31 (d, 2H), 5.39 (dt, vinyl, 1H). ¹⁹F NMR (CDCl₃) δ−108.21 (q, 1F, J=21 Hz).

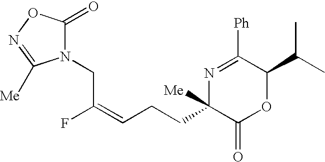

Example-HH-6) To a 1-methyl-2-pyrrolidinone (12 mL) solution of (3S, 6R)-6-isopropyl-3-methyl-5-phenyl-3,6-dihydro-2H-1,4-oxazin-2-one (Synthesis, 1999, 4, 704-717) (1.10 g, 4.76 mmol), LiI (0.63 g, 4.76 mmol) and Example-HH-5 (0.85 g, 2.72 mmol) in an ice bath was added 2-tert-butylimino-2-diethylamino-1,3-dimethylperhydro-1,3,2-diazaphosphorine (1.38 mL, 4.76 mmol). The yellow solution became orange upon addition of the base, and the resulting solution was allowed to stir at room temperature for 1 hour. The reaction mixture was diluted with ethyl acetate (100 mL), washed with water (2×30 mL), dried (MgSO₄), filtered and evaporated to a yellow oil. The crude material was purified by flash column chromatography on silica gel eluting with 30% ethyl acetate in hexane to give 0.64 g (57%) of the desired alkylated product as a clear oil.

¹H NMR (C₆D₆) δ0.57 (d, 3H), 0.89 (d, 3H), 1.30 (s, 3H), 1.65 (s, 3H), 1.8 (m, 2H), 2.0 (m, 2H), 2.1 (m, 1H), 3.22 (m, 2H), 4.88 (dt, vinyl, 1H), 5.49 (d, 1H), 7.1 (m, 3H), 7.6 (m, 2H). ¹⁹F NMR (CDCl₃) δ−110.37 (q, 1 F, J=21 Hz).

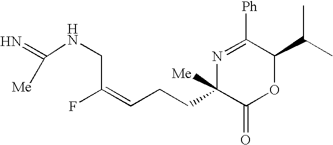

Example-HH-7) To a methanol (20 mL) solution of Example-HH-6 (0.13 g, 0.31 mmol) was added Lindlar catalyst (1.0 g). The stirred slurry was heated to 60° C. for 1 hour, and additional Lindlar catalyst (0.30 g) was added. The slurry was stirred an additional 1 hour at 60° C., then cooled to room temperature. The catalyst was removed by filtration through celite, and the filtrate was stripped to give 0.58 g (100%) of the desired deprotected amidine product as a pale yellow oil.

MS: m/z=374.2 [M+H]⁺

¹H NMR (CD₃OD) δ0.77 (d, 3H), 1.07 (d, 3H), 1.58 (s, 3H), 2.02 (s, 3H), 1.8-2.2 (m, 5H), 3.83 (d, 2H), 5.20 (dt, vinyl, 1H), 5.69 (d, 1H), 7.4 (m, 3H), 7.7 m, 2H)

¹⁹F NMR (CDCl₃) δ−109.4 (m, 1F, J=21 Hz)

Example-HH) A solution of the product from Example-HH-7 (0.58 g, 1.54 mmol) in 1.5 N HCl (25 mL) was washed with diethyl ether (2×20 mL) and refluxed for 1 hour. The solvent was stripped and the crude amino acid ester was dissolved in 6 N HCl (15 mL) and heated to reflux. After six hours, the solvent was removed in vacuo, and the resulting foam was purified by reverse-phase HPLC eluting with a 30 minute gradient of 0-40% CH₃CN/H₂O(0.25% acetic acid). Fractions containing product were combined and concentrated to a foam. The product was dissolved in 1 N HCl and the solvent removed in vacuo (2×) to give 0.15 g (29%) of the desired (2S,5E)-2-amino-2-methyl-6-fluoro-7-[(1-iminoethyl)amino]-5-heptenoic acid, dihydrochloride product.

HRMS calcd. for C₁₀H₁₉FN₃O₂: m/z=232.1461 [M+H]⁺, found: 232.1485.

¹H NMR (D₂O) δ1.43 (s, 3H), 2.10 (s, 3H), 1.8-2.1 (m, 4H), 3.98 (d, 2H) 5.29 (dt, vinyl, 1H). ¹⁹F NMR (CDCl₃) δ−109.97 (q, 1 F, J=21 Hz).

### Example II

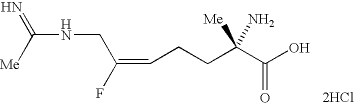

### (2S,5E)-2-amino-2-methyl-6-fluoro-7-[(1-iminoethyl)amino]-5-heptenoic acid, dihydrochloride

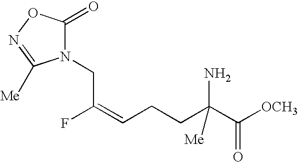

Example-II-1) To a 1-methyl-2-pyrrolidinone (7500 mL) solution of methyl N-[(3,4-dichlorophenyl)-methylene]-alaninate (748.5 g, 2.88 mol) under nitrogen was added LiI (385.5 g, 2.88 mol) and the resulting slurry stirred approximately 20 minutes to give a clear solution. The solid from Example-HH-5 (750 g, 2.40 mol) was then added and the resulting solution cooled in an ice bath to ˜0° C. Neat BTPP (900 g, 2.88 mol) was added dropwise over 25 minutes maintaining the internal temperature below 5° C. After stirring for an additional 1.5 hour at 5° C., the reaction was determined to be complete by HPLC. At this time, 7500 mL of methyl t-butyl ether (MTBE) was added followed by addition of 9750 mL of a water/crushed ice mixture. The temperature rose to 20° C. during this operation. After stirring vigorously for 5-10 minutes, the layers were separated and the aqueous layer washed with twice with 6000 mL of MTBE. The MTBE layers were combined and washed two times with 7500 mL of water. The resulting MTBE solution was then concentrated to ˜5000 mL, treated with 11625 mL of 1.0 N HCl, and stirred vigorously at room temperature for one hour. The layers were separated and the aqueous layer washed with 7500 ml of MTBE. About 1 kg of sodium chloride was added to the aqueous layer and the resulting mixture stirred until all the salt had dissolved. At this point, 7500 mL of ethyl acetate was added, the resulting mixture cooled to 10° C., and 2025 mL of 6.0 N sodium hydroxide added with good agitation. The resulting pH should be about 9. The layers were separated and the aqueous layer was saturated with sodium chloride and extracted again with 7500 mL of ethyl acetate. The combined ethyl acetate extracts were dried (MgSO₄) and concentrated to a light oil. It should be noted that the ethyl acetate was not complete removed. With agitation, 3000 ml of hexane then is added to generate a slurry that was cooled to 10° C. The granular solid was collected by filtration and washed with 1500 mL of hexane. About 564 g (82% yield) of the desired pure aminoester (>95% pure by HPLC) was obtained as a white solid, m.p. 82.9-83.0° C. LCMS: m/z=288.2 [M+H]⁺. Chiral HPLC (Chiralpak-AD normal phase column, 100% acetonitrile, 210 nm, 1 mL/min): Two major peaks at 4.71 and 5.36 min (1:1).

¹H NMR (CDCl₃): δ1.40 (s, 3H), 1.7-1.8 (m, 2H), 2.0 (br s, 2H), 2.2 (m, 2H), 2.29 (s, 3H), 3.73 (s, 3H), 4.34 (dd, 2H), 5.33 (dt, 1H).

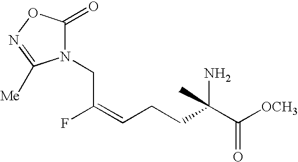

Example-II-2) Separation of the individual enantiomers of the product from Example-II-1 was accomplished on preparative scale using chiral HPLC chromatography (ChiralPak-AD, normal phase column, 100% acetonitrile) to give the desired pure (2S)-2-methyl amino ester product title product. ChiralPak-AD, normal phase column, 100% acetonitrile, 210 nm, 1 mL/min): 5.14 min (99%).

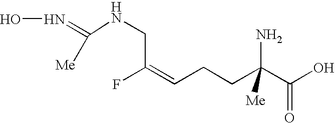

Example-II-3) A slurry of the product of Example-II-2 (2.30 g, 8.01 mmol) in 0.993 M NaOH (30.0 ml, 29.79 mmol) was stirred 2 hours at room temperature. To the resulting clear colorless solution was added 1.023 M HCl (29.10 mL, 29.76 mmol). The-resulting clear solution was concentrated until a precipitate began to form (approx. 30 mL). The slurry was warmed to give a clear solution that was allowed to stand at room temperature overnight. The precipitate was isolated by filtration. The solid was washed with cold water (2×10 mL), cold methanol (2×10 mL) and Et₂O (2×20 mL). The white solid was dried in vacuo at 40° C. 4 hours to give 1.04 g (53%) of the desired N-hydroxy illustrated product. mp=247.2° C.

Anal. calcd. for C₁₀H₁₈FN₃O₃: C, 48.57; H, 7.34; N, 16.99; Cl, 0.0. Found: C, 48.49; H, 7.37; N, 16.91; Cl, 0.0.

HRMS calcd. for C₁₀H₁₉FN₃O₃: m/z=248.1410 [M+H]⁺, found: 248.1390.

¹H NMR (D₂O) δ1.35 (s, 3H), 1.81 (s, 3H), 1.7-2.0 (m, 4H), 3.87 (d, 2H) 5.29 (dt, vinyl, 1H). ¹⁹F NMR (CDCl₃) δ−112.51 (q, 1 F, J=21 Hz).

Example-II-4) To a solution of Example-II-3 in methanol is added Lindlar catalyst. The stirred slurry is refluxed for 2 hours, then cooled to room temperature. The catalyst is removed by filtration through celite, and the filtrate is stripped. The resulting solid is dissolved in water and concentrated repeatedly from 1.0 N HCl to give the desired (2R,5E)-2-amino-2-methyl-6-fluoro-7-[(1-iminoethyl)amino]-5-heptenoic acid, dihydrochloride product.

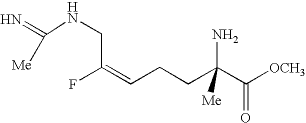

Example-II-5) A solution of 73.5 9 (0.3 mol) of the product from Example-II-2 was dissolved in 300 mL of methanol and added dropwise to a preformed mixture of 13.7 g of Lindlar catalyst and 73.5 g of formic acid (1.53 mol) in 312 mL of methanol while maintaining the reaction temperature between 22° C. and 26° C. After stirring at room temperature for an additional ˜15 hrs, the reaction was determined to be complete by F¹⁹ NMR. The resulting reaction mixture was filtered through celite and the celite washed 3 times with 125 mL of methanol. The methanol filtrates were combined and concentrated to generate 115 g of the desired amidine title product as a viscous oil.

MS: m/z=246 (M+H)⁺.

¹H NMR (CD₃OD) [symbol] , 4H) 2.3 (□, 3H), 3.9 (□, 3H), 4.2 (□, 2H), 5.4 ( [symbol] vinyl), 8.4 (□, 3H).

F¹⁹ NMR (CD₃OD) [symbol] , J=21 Hz) −111.7 (□, J=21 Hz).

In order to remove trace levels of lead, the crude product was dissolved in 750 mL of methanol and 150 g of a thiol-based resin (Deloxan THP 11) was added. After stirring 3 hrs at room temperature, the resin was filtered off and washed 2 times with 500 mL methanol. The filtrates were collected and concentrated to 99 g of the desired amidine title product as a viscous oil.

### Alternatively

A total of 5.0 g of the product from Example-II-2 (0.0174 mole, 1.0 equiv) was mixed with 5.0 g of zinc dust (0.0765 moles, 4.39 equiv) in 40 mL of 1-butanol and 10 mL of acetic acid. After stirring for 5 hrs at 50° C., LC analyses indicated the reaction to be complete. The solids were readily filtered off. The filtrate, after cooling in ice water to 7° C., was treated with 30 mL of 6 N NaOH (0.180 moles) in one portion with vigorous stirring. After cooling the reaction mixture from 33° C. to 20° C., the clear butanol layer was separated off and the aqueous layer extracted again with 40 mL of 1-butanol. The butanol extracts were combined, washed with 30 mL of brine followed by approx 10 mL of 6N HCl. After concentration at 70° C., a clear glass resulted which was identified as the desired amidine title product.

Example-II) A solution of 99 g of the product from Example-II-5 in 6 N HCl was refluxed for 1 hr at which time LC analyses indicated the reaction to be complete. The solvent was removed in vacuo to yield 89.2 g of a glassy oil which was dissolved in a mixture of 1466 mL ethanol and 7.5 ml of deionized water. THF was added to this agitated solution at ambient temperature until the cloud point was reached (5.5 liters). An additional 30 ml of deionized water was added and the solution agitated overnight at room temperature. The resulting slurry was filtered and washed with 200 mL of THF to yield 65 g of a white solid identified as the desired title product.

[□]_{D}²⁵=+7.2 (c=0.9, H₂O)

mp=126-130° C.

MS: m/z=232 (M+H)⁺.

Anal. Calcd for C₁₀H₂₂N₃F₁O₃Cl₂: C, 37.28; H, 6.88; N, 13.04; Cl, 22.01.

Found: C, 37.52, H, 6.84, N, 13.21, Cl, 21.81.

¹H NMR (D₂O) [symbol] 3H), 1.8-2.1 (m, 4H), 1.9 (□,3H), 4.0(□, 2H), 5.3(□t, vinyl, 1H).

F¹⁹NMR (D₂O) [symbol] J=21 Hz) −112.1 (□, J−21 Hz).

### Example JJ

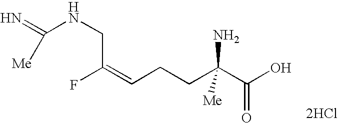

### (2R,5E)-2-amino-2-methyl-6-fluoro-7-[(1-iminoethyl)amino]-5-heptenoic acid, dihydrochloride

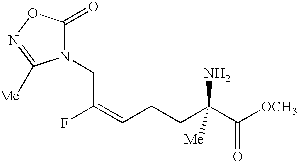

Example-JJ-1) Separation of the individual enantiomers of the product from Example-II-1 was accomplished on preparative scale using chiral HPLC chromatography to give the desired pure (2R)-2-methyl amino ester product.

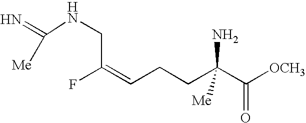

Example-JJ-2) The product from Example-JJ-1 is dissolved in water and acetic acid. Zinc dust is added, and the mixture is heated at 60° C. until HPLC analysis shows that little of the starting material remains. The Zn is filtered through celite from the reaction mixture, and the filtrate is concentrated. The crude material is purified by reverse-phase HPLC column chromatography. Fractions containing product are combined and concentrated affording the desired (2R)-2-methyl acetamidine product.

Example-JJ) A solution of Example-JJ-2 in 2.0 N HCl is refluxed for 2 h. The solvent is removed in vacuo. The resulting solid is dissolved in water and concentrated repeatedly from 1.0 N HCl to give the desired (2R,5E)-2-amino-2-methyl-6-fluoro-7-[(1-iminoethyl)amino]-5-heptenoic acid, dihydrochloride product.

### Example KK

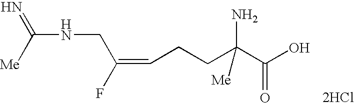

### (2R/S,5E)-2-amino-2-methyl-6-fluoro-7-[(1-iminoethyl)amino]-5-heptenoic acid, dihydrochloride

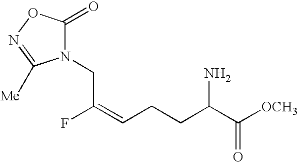

Example-KK-1) To an 1-methyl-2-pyrrolidinone (5 mL) solution of methyl N-[(4-chlorophenyl)methylene]-glycinate (0.33 g, 1.6 mmol), LiI (0.20 g, 1.0 mmol) and a sample of the product of Example-HH-5 (0.30 g, 0.96 mmol) in an ice bath was added 2-tert-butylimino-2-diethylamino-1,3-dimethylperhydro-1,3,2-diazaphosphorine (0.433 mL, 1.5 mmol). The solution was allowed to stir at room temperature for 1.5 hours. The reaction mixture was diluted with ethyl acetate (30 mL), washed with water (2×20 mL), dried (MgSO₄), filtered, and evaporated to give the crude desired racemic alkylated imine as a yellow oil.

The crude material was dissolved in ethyl acetate (10 mL) and 1N HCl (10 mL) was added. The mixture was stirred for 2 hours at room temperature, and the organic layer was separated. The aqueous layer was neutralized with solid NaHCO₃ and extracted with ethyl acetate (2×30 mL). The organic layer was dried (MgSO₄), filtered and evaporated to give 0.13 g of the desired title racemic amino ester product as a yellow oil. This product was used in the next step without further purification. LCMS: m/z=288.2 [M+H]⁺.

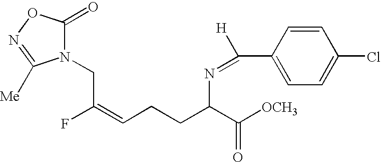

Example-KK-2) To a CH₂Cl₂ (15 mL) solution of Example-KK-1 (1.36 g, 4.98 mmol) was added 4-chlorobenzaldehyde (0.70 g, 5.0 mmol) and MgSO₄ (˜5 g). The slurry was stirred at room temperature for 18 hours. The slurry was filtered, and the filtrate stripped to give 1.98 g (100%) of the desired title imine product as a pale yellow oil. This product was used in the next step without further purification.

¹H NMR (C₆D₆) □ 1.34 (□, 3H), 2.0 (□□ m, 4H), 3.32 □, 3H), 3.42 (m, 2H), 3.83 (t, 1H), 4.98 (□t, vinyl, 1H).

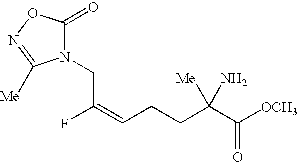

Example-KK-3) To a CH₂Cl₂ (2 mL) solution of the product of Example-KK-2 (0.25 g, 0.63 mmol) was added methyl iodide (0.200 mL, 3.23 mmol) and O(9)-allyl-N-(9-anthracenylmethyl)-cinchonidinium bromide (40 mg, 0.066 mmol). The solution was cooled to −78° C. and neat BTPP (0.289 mL, 0.95 mmol) was added. The resulting orange solution was stirred at −78° C. for 2 hours and allowed to reach −50° C. After 2 hours at −50° C., the solution was diluted with CH₂Cl₂ (10 mL), washed with water (10 mL), dried (MgSO₄), filtered, and evaporated to give the crude desired racemic alkylated imine as a yellow oil.

The crude material was dissolved in ethyl acetate (10 mL) and 1N HCl (10 mL) was added. The mixture was stirred for 1 hour at room temperature, and the organic layer was separated. The aqueous layer was neutralized with solid NaHCO₃ and extracted with ethyl acetate (2×30 mL). The organic layer was dried (MgSO₄), filtered and evaporated to give 0.16 g of the desired racemic 2-methylamino ester product as a yellow oil. The product was used in the next step without further purification. LCMS: m/z=288.2 [M+H]⁺.

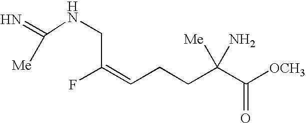

Example-KK-4) The racemic product from Example-KK-3 is dissolved in water and acetic acid. Zinc dust is added, and the mixture is heated at 60° C. until HPLC analysis shows that little of the starting material remains. The Zn dust is filtered through celite from the reaction mixture, and the filtrate is concentrated. The crude material is purified by reverse-phase HPLC column chromatography. Fractions containing product are combined and concentrated affording the desired acetamidine product.

Example-KK) A solution of racemic Example-KK-4 in 2.0 N HCl is refluxed for 1 h. The solvent is removed in vacuo. The resulting solid is dissolved in water and concentrated repeatedly from 1.0 N HCl to give the desired title (2R/S,5E)-2-amino-2-methyl-6-fluoro-7-[(1-iminoethyl)amino]-5-heptenoic acid, dihydrochloride product.

### Example LL

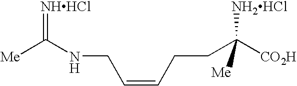

### (2S,5Z)-2-amino-2-methyl-7-[(1-iminoethyl)amino]-5-heptenoic acid, dihydrochloride

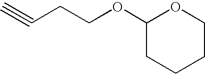

### 4-[(Tetrahydropyranyl)oxy]butyne

Example LL-1) A mixture of 4-dihydro-2H-pyridine (293.2 g 3.5 mol) and concentrated HCl (1.1 mL) was cooled to 5° C. While continuing to cool externally, 3-butyn-1-ol (231.5 g, 3.3 mol) was added over a period of 30 minutes allowing the temperature to reach 50° C. Reaction was held with mixing at room temperature for 2.5 hours before it was diluted with MTBE (1.0 L). The resulting mixture was washed with saturated sodium bicarbonate (2×150 mL). The organic phase was dried over sodium sulfate and concentrated under reduced pressure to afford 500 g (98% crude yield) of product; GC area % of 96%.

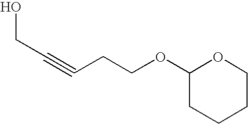

### 5-(Tetrahydro-pyran-2-yloxy)-pent-2-yn-1-ol

Example LL-2) To a solution of the 4-[(tetrahydropyranyl)oxy]butyne product of Example LL-1 (50.0 g, 0.33 mol) in THF (125 mL) was added a solution of 2N EtMgCl in THF (242 mL, 0.48 mol) under a nitrogen atmosphere over a 30 minute period, allowing the temperature to rise to 48° C. Mixture was further heated to 66° C. and was held at this temperature for 2 hours before cooling to ambient temperature. Paraformaldehyde (14.5 g, 0.48 mol) was added (small exotherm was observed) and the resulting mixture was heated to 45° C. After 1 hour of controlling the temperature between 45-55° C., the mixture turned clear. At this point, the mixture was heated up to 66° C. and stirred for 2.5 hours. Mixture was cooled to room temperature and saturated ammonium chloride (125 mL) was added slowly over 30 minutes (strong exotherm was observed) keeping the temperature below 40° C. The liquid phase was separated by decantation; ethyl acetate (250 mL) and brine (50 mL) were added. The organic phase was separated and washed with brine (2×50 mL) and water (1×50 mL). The organic layer was dried over sodium sulfate and concentrated under reduced pressure to afford 51 g of a lightly yellow colored oil (85% crude yield); GC area %=88% title product, 6% starting material.

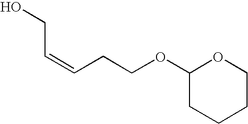

### 5-(Tetrahydro-pyran-2-yloxy)-pent-2-en-1-ol

Example LL-3) To a 500 mL Parr bottle, under a nitrogen atmosphere, was charged the 5-(tetrahydro-pyran-2-yloxy)-pent-2-yn-1-ol product of Example LL-2 (40.2 g, 0.22 mol), Lindlar catalyst (2.0 g), ethanol (120 mL), hexane (120 mL), and 2,6-lutidine (457 mg). Reaction mixture was purged five times each with nitrogen and hydrogen gas. Parr bottle was pressurized with hydrogen to 5 psi and shaken until 98% of the theoretical hydrogen was consumed. Hydrogen was released from the vessel and the reaction was purged with nitrogen five times. Mixture was filtered through a pad of Solka Floc and the catalyst was rinsed with ethanol (2×50 mL). The filtrate and rinses were combined and concentrated under reduced pressure to afford 40.3 g (99% yield) of the title material as a yellow colored oil (GC area %=96%).

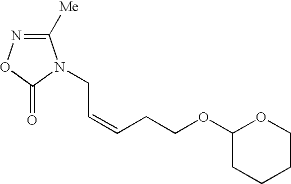

### 3-Methyl-4-[5-(tetrahydro-pyran-2-yloxy)-pent-2-enyl]-4H-[1,2,4]oxadiazol-5-one

Example LL-4) To a solution of the 5-(tetrahydro-pyran-2-yloxy)-pent-2-en-1-ol product of Example LL-3 (11.8 g, 0.063 mol) in toluene (42 mL) was added) triethylamine (6.4 g, 0.063 mol). The mixture was cooled to −5° C. and methanesulfonyl chloride (7.3 g, 0.63 mol) was added via syringe at such rate as to keep the pot temperature below 10° C. The mixture was allowed to warm to room temperature and stirred for two hours. The mixture was filtered by suction and rinsed on the filter with toluene (2×20 mL). The filtrate and washes were added to a mixture of the sodium salt of 3-methyl-1,2,4-oxadiazolin-5-one (8.6 g, 0.063 mol) in DMF (10 mL). The mixture was stirred with a mechanical stirrer and heated at 45° C. for 5 hours. Water (40 mL) was added and the mixture was stirred for 5 minutes and then the layers were separated. The toluene layer was washed with water (3×20 mL), dried over MgSO₄, and concentrated to afford 16.5 g (97.3%) of an orange colored crude product (area % GC consisted of 71% title product, 18% toluene, and 4% of an impurity).

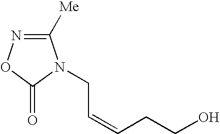

4-(5-Hydroxy-pent-2-enyl)-3-methyl-4H-[1,2,4]oxadiazol-5-one

Example LL-5) To a solution the 3-methyl-4-[5-(tetrahydro-pyran-2-yloxy)-pent-2-enyl]-4H-[1,2,4]oxadi-az-ol-5-one product of Example LL-4 (16 g, 0.06 mol) in methanol (48 mL) was added p-toluenesulfonic acid (0.34 g, 2.0 mmol). The mixture was stirred at room temperature for four hours. Sodium bicarbonate (0.27 g, 3.0 mmol) was added and the mixture was concentrated on a rotary evaporator. The residue was diluted with saturated NaHCO₃ (20 mL) and the resulting mixture was extracted with ethyl acetate (2×60 mL). Extracts were combined and washed with water (2×25 mL), dried over MgSO₄, and concentrated to afford 8.4 g of the crude, orange colored oil title product (area % GC=80%).

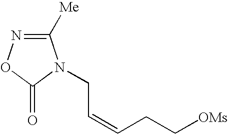

### Methanesulfonic acid 5-(3-methyl-5-oxo-[1,2,4]oxadiazol-4-yl)-pent-3-enyl ester

Example LL-6) To a solution of the 4-(5-Hydroxy-pent-2-enyl)-3-methyl-4H-[1,2,4]oxadiazol-5-one product of Example LL-5 (8.27 g, 0.045 mol) in methylene chloride (33 mL) was added triethylamine (5.0 g, 0.49 mol). The mixture was cooled to −5° C. and methanesulfonyl chloride (5.5 g, 0.048 mol) was added at such rate as to keep the temperature below 8° C. The cooling bath was removed and the mixture was stirred for 3 hours as it warmed up to room temperature. Water (15 mL) was added and the mixture was stirred for 5 minutes and then the layers were separated. The organic phase was washed with water (10 mL), dried over MgSO₄, and concentrated to give a light amber colored residue. The residue was dissolved in ethyl acetate (8 mL) and kept at 5° C. overnight. Precipitated solids were filtered off by suction and rinsed on the filter with minimum volume of ethyl acetate and then air-dried on the filter to afford 6.8 g (58% yield) of the title product.

¹H NMR (CDCl₃) δ5.76 (dtt, J=10.9, 7.5, 1.5 Hz, 1H), δ5.59 (dtt, J=10.9, 7.0, 1.5 Hz, 1H), δ4.31 (t, J=6.3 Hz, 2H), δ4.27 (dd, J=7.0, 1.5 Hz, 2H), δ3.04 (□, 3H), δ2.67 (□, J=6.7 Hz, 2H), δ2.28 (□, 3H)

¹³C (CDCl₃) δ159.0, 156.3, 129.9, 125.1, 68.4, 38.9, 37.2, 27.5, 10.2.

IR (cm⁻¹) 1758, 1605, 1342, 1320, 1170.

Anal. Calcd. for C₉H₁₄N₂O₅S: C, 41.21; H, 5.38; N, 10.68. Found: C, 41.15; H, 5.41; N, 10.51.

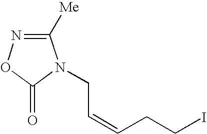

### 4-(5-Iodo-pent-2-enyl)-3-methyl-4H-[1,2,4]oxadiazol-5-one

Example LL-7) To a solution of the methanesulfonic acid 5-(3-methyl-5-oxo-[1,2,4]oxadiazol-4-yl)-pent-3-enyl ester product of Example LL-6 (20.0 g, 0.076 mol) in acetone (160 ml) was added sodium iodide (17.15 g, 0.114 mol). The mixture was heated to reflux and was stirred for 3 hours. External heating was stopped and the mixture was held at room temperature overnight. Solids were removed by filtration and rinsed on the filter. The filtrate and washes were combined and concentrated and the heterogeneous residue was extracted with ethyl acetate (120 mL). The organic layer was washed with water (60 mL), 15% aqueous solution of sodium thiosulfate (60 mL) and water (60 mL); dried over MgSO₄ and concentrated under reduced pressure to afford 22.1 g (98% yield) of the title oil product.

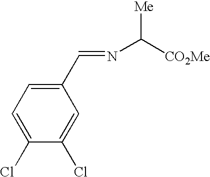

### 2-[(3,4-Dichloro-benzylidene)-amino]-propionic acid methyl ester

Example LL-8) To a mechanically stirred slurry of L-alanine methyl ester hydrochloride (200.0 g, 1.43 mol) in methylene chloride (2.1 L) under a nitrogen atmosphere was added triethylamine (199.7 mL, 1.43 mol) over 12 min (during the addition solids partially dissolved and then reprecipitated). After 10 min, 3,4-dichlorobenzaidehyde (227.5 g, 1.30 mol) and magnesium sulfate (173.0 g, 1.43 mol) were added (temperature increased 6° C. over 30 min). After 2.5 h, the mixture was filtered. The filtrate was washed with water (1×1 L) and brine (1×500 mL), dried over sodium sulfate, filtered and concentrated to give 313.3 g, 92.4% yield of oil product.

¹H NMR (400 MHz, CDCl3) δ8.25 (s, 1H), 7.91 (d, 1H), 7.58 (dd, 1H), 7.49 (d, 1H), 4.17 (t, 1H), 3.76 (s, 3H), 1.53 (d, 3H). Anal. Calcd for C₁₁H₁₁Cl₂NO₂: C, 50.79; H, 4.26; Cl, 27.26; N, 5.38. Found: C, 50.37; H, 4.10; Cl, 26.87; N, 5.38.

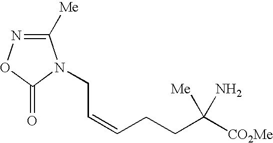

### Rac-2-Amino-2-methyl-7-(3-methyl-5-oxo-[1,2,4]oxadiazol-4-yl)-hept-5-enoic acid methyl ester

Example LL-9) Method 1. A solution of the product of Example LL-7 (114.2 g, 0.39 mol) and the product of Example LL-8 (151.5 g, 0.58 mol) in dimethylformamide (1.4 L) under nitrogen atmosphere was cooled to −8° C. Lithium iodide (78.1 g, 0.58 mol) was then added in 3 equal portions over 19 min. The mixture was stirred for 20 min at −7° C. and then (tert-butylimino)-tris(pyr-rolidino)phosphorane (194.0 mL, 0.62) was added over 36 min (maximum temperature=−2.6° C.). After 10 min, the cooling bath was removed and the solution was stirred at ambient temperature for 1 h. The mixture was then poured into cold water (1.4 L) and extracted with ethyl acetate (2×1.0 L). The combined organic layers were washed with water (2×400 mL) and brine. The ethyl acetate layer was treated with 1 N HCl (780 mL) and stirred for 1 h. The aqueous layer was separated and extracted with ethyl acetate (2×400 mL) and then neutralized with sodium bicarbonate (110 g). The mixture was extracted with ethyl acetate (1×500 mL). The organic layer was dried over sodium sulfate, filtered, concentrated and then treated with methyl t-butyl ether to give a crystalline product: first crop 14.4 g; second crop 6.6 g (GC purity=96.2 and 91.9%, respectively). The aqueous phase was saturated with sodium chloride and extracted with ethyl acetate (4×500 mL). The combined organic layers were dried over sodium sulfate, filtered, concentrated and then treated with methyl t-butyl ether to give a crystalline product: first crop 33.4 g; second crop 10.8 g (GC purity=89.6 and 88.8%, respectively. Total crude yield 65.2 g, 62.4%.

Method 2. To a solution of the product of Example LL-7 (20.7 g, 0.070 mol) and the product of Example LL-8 (22.9 g, 0.088 mol) in dimethylformamide (207 mL) under a nitrogen atmosphere was added cesium carbonate (29.8 g, 0.092). The mixture was stirred at rt for 16 h and then diluted with water (300 mL) and extracted with ethyl acetate (2×200 mL). The combined ethyl acetate layers were washed with water (3×100 mL) and brine and then treated with 1 N HCl (184 mL). After 1 h, the layers were separated and the aqueous layer was extracted with ethyl acetate (3×100 mL) and then neutralized with sodium bicarbonate (15.5 g). The mixture was extracted with ethyl acetate (1×150 mL). The aqueous layer was saturated with sodium chloride and extracted with ethyl acetate (3×100 mL). The combined organic layers were dried over sodium sulfate, filtered and concentrated to give a yellow solid, 11.9 g, 62.9%; GC purity=96.6%. The crude product was recrystallized from warm methyl t-butyl ether or ethyl acetate.

¹H NMR (400 MHz, CDCl₃) δ5.68 (m, 1H), 5.36 (m, 1H), 4.23 (d, 2H), 3.73 (s, 3H), 2.43 (□, 3H), 2.18 (m, 2H), 1.81 (m, 1H), 1.69 (s, br, 2H), 1.66 (m, 1H), (1.36, 3H)

¹³C NMR (400 MHz, CDCl₃) δ177.60, 159.01, 156.10, 135.12, 121.82, 57.48, 52.29, 40.12, 39.00, 26.62, 22.56, 10.41

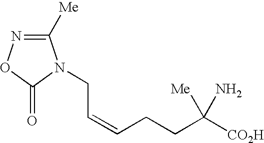

### Rac-2-Amino-2-methyl-7-(3-methyl-5-oxo-[1,2,4]oxadiazol-4-yl)-hept-5-enoic acid

Example LL-10) The product of Example LL-9 (0.269 g, 1 mmol) was dissolved in 5 mL 2 N HCl and heated to reflux under argon. After refluxing for 6 hrs followed by stirring at room temperature for 72 hours, an aliquot was removed and checked by ¹H NMR. Approximately 6% of unreacted starting ester remained along with the desired product (verified by LC-MS). The aqueous portion was removed in vacuo, leaving 0.38 g of a thick, amber oil. After purification via reverse phase chromatography, followed by lyophilization, one obtained 0.23 g, 90.2% of the title compound as white, non-deliquescent solids.

Anal. Calcd. for C₁₁H₁₇N₃O₄.0.77H₂O: C, 49.09; H, 6.94; N, 15.61. Found: C, 48.71; H, 6.94; N, 15.98

Mass spec: M+1=256.

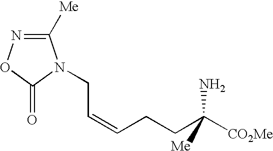

### (2S,5Z)-2-Amino-2-methyl-7-(3-methyl-5-oxo-[1,2,4]oxadiazol-4-yl)-hept-5-enoic acid methyl ester

Example LL-11) The title compound (827.3 g) was separated from its R enantiomer by preparative chiral chromatography using Novaprep 200 instrument with steady state recycling option. The material was dissolved in absolute ethanol at a concentration of 40 mg/ml and loaded on a 50×500 mm prepacked Chiral Technologies stainless steel column. The adsorbent was 20μ ChiralPak AD. The mobile phase was ethanol/triethylamine 100/0.1; the flow rate equaled 125 ml per min. The crude solution (25 mL) was loaded on the column every 12 mins. A steady state recycling technique was used. Solvent was removed using a rotovap. The final product was isolated as gold oil which solidified on standing; 399.0 g (96.4% recovery).

¹H (400 MHz, CD₃OD) δ5.68 (dtt, 1H, J_{olefinic}=10.7 Hz), 5.43 (dtt, 1H, J_{holefinic}=10.7 Hz), 4.82 (s, br, 2H), 4.28 (d, 2H, J=5.5 Hz), 3.73 (s, 3H), 2.27 (s, 3H), 2.26 (m, 1H), 2.14 (m,1 H), 1.82 (ddd, 1H, J=13.6, 11.3, 5.4 Hz), 1.67 (ddd, 1 H, J=13.6, 11.2, 5.5 Hz), 1.34 (s, 3H)

¹³C NMR (400 MHz, CD₃OD) δ178.49, 161.13, 158.70, 135.92, 123.47, 58.55, 52.77, 41.38, 39.96, 26.23, 23.47, 10.23

Anal. Calcd for C₁₂H₁₉N₃O₄: C, 53.52; H, 7.11; N, 15.60. Found: C 52.35; H, 7.20; N, 15.60.

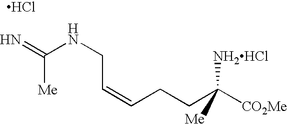

### (2S,5Z)-7-Acetimidoylamino-2-amino-2-methyl-hept-5-enoic acid methyl ester, dihydrochloride hydrate

Example LL-12) To a solution of the product of Example LL-11 (114.5 g, 0.425 mol) in methanol (2.4 L) was added the solid dibenzoyl-L-tartaric acid (152.5 g, 0.425 mol) and 88% formic acid (147 mL, 3.428 mol) at ambient temperature. A slurry of Lindlar catalyst, 5 wt % palladium on calcium carbonate poisoned with lead acetate (37.9 g), in methanol (200 mL) was prepared under nitrogen. The solution of starting material was then added at ambient temperature to the light grey catalyst slurry followed by a methanol rinse (200 mL). The heterogeneous reaction mixture was heated at 45° C. for 1½ hours. Steady gas evolution was observed starting at about 40° C., which indicated the ongoing reaction. The mixture was cooled in an ice/water bath and then filtered through a plug of Supercell HyFlo. The yellow solution was concentrated in vacuo to give a viscous oil, which was dissolved and partitioned between 2 N aqueous HCl (2 L) and ethyl acetate (0.8 L). Layers were separated and the aqueous layer was washed once with ethyl acetate (0.8 L). Solvent and volatiles were removed in vacuo at elevated temperatures (=70° C.). The intermediate product was used in next the step without further purification or characterization. LC-MS [M+H]⁺=228.

Example LL) The crude product of Example LL-12 (170 g) was dissolved in 2 N aqueous HCl (1 L). The resulting orange solution was refluxed overnight before it was allowed to cool back to ambient temperature. The reaction mixture was concentrated to about ⅓ of its volume, and the acidic solution was passed through a solid phase extraction cartridge (25 g of C18 silica) to remove color and other impurities. Solvent was removed in vacuo (=70° C.) to give 208 g of crude product as yellowish gum.

The crude gum (31.3 g) was taken up in water (250 mL) and the material was loaded onto a pretreated ion exchange column packed with the acidic resin Dowex 50WX4-400 (about 600 g). The resin was first washed with water (1 L), then with dilute aqueous HCl (1 L of 10/90 v/v conc. HCl/water). The product was eluted off the resin with higher ion strength aqueous HCl (1.5 L of 20/90 v/v to 25/75 v/v conc. HCl/water). The aqueous solvent was removed in vacuo (=70° C.), and the gummy residue was taken up in 4 vol % aqueous trifluoroacetic acid (100 mL). The aqueous solvent was removed in vacuo (=70° C.), and the procedure was repeated once more. The residue was then dried under high vacuum to give 32.2 g of gum as the trifluoroacetic acid salt.

Crude (2S,5Z)-7-acetimidoylamino-2-amino-2-methyl-hept-5-enoic acid, ditrifluoroace-tic acid salt hydrate (32.2 g) was purified by reverse-phase preparative chromatography. The crude was dissolved in 0.1% aqueous TFA (50 ml) and loaded onto a 2-inch ID×1 meter stainless steel column packed with adsorbent (BHK polar W/S, 50 □, 1.16 kg). The product was eluted at a flow rate of 120 mL/min with a step gradient from 0.1% aqueous TFA to 25/75/0.1 acetonitrile/water/TFA. The loading ratio was 36:1 w/w silica to sample. Solvent was removed in vacuo, and the material was converted into the HCl salt by repeated rinses with dilute aqueous HCl and solvent removals in vacuo. Drying under high vacuum gave 27.4 g of the title dihydrochloride hydrate as yellowish gum.

LC−MS [M+H]⁺=214.16 Da

¹H NMR (D₂O, δ: 1.48 (s, 3H), 1.8-1.9 (AB, 2H), 2.10 (s, 3H), 2.01/2.12 (AB, 2H), 3.78 (d, 2H), rotamere 3.87 (d, 2H), 5.6/5.5 (dt, 2H, 11 Hz)

¹³C NMR (D₂O) δ: 18.7, 21.5, 21.6, 36.4, 39.1, 59.8, 122.6, 134.3, 164.5, 173.7

Elemental Anal. Calcd. for C₁₀H₁₉N₃O₂.2.2HCl.2 H₂O: C, 36.21; H, 8.33; N, 12.67; Cl 23.51. Found: C, 36.03; H, 7.72; N, 12.67; Cl, 23.60.

### Example MM

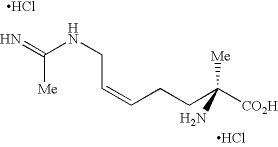

### (2R,5Z)-2-amino-2-methyl-7-[(1-iminoethyl)amino]-5-heptenoic acid, dihydrochloride

The R-enantiomer isolated during the separation described in Example LL-11 (1.13 g, 4.2 mmol) was dissolved in 11 mL 25% aqueous acetic acid and heated to 60° C. Zinc dust (1.10 g) was then added in 4 equal portions at 30-minute intervals. After heating for a total of 3 hours, an aliquot was removed and checked by LC-MS, which indicated only a trace of unreacted starting material remaining, along with desired product. The mixture was cooled to room temperature, filtered and stripped in vacuo, leaving 2.31 g of a slushy white solid. The methyl ester was hydrolysed with dilute hot HCl to the title compound. After purification by reverse phase chromatography followed by lyophilization, 0.31 g of the title compound as a glassy solid was obtained.

Anal. Calcd. for C₁₀H₁₉N₃O₂.1.22 HCl.1.15 H₂O: C, 46.13; H, 8.15; N, 15.09; Cl, 15.53.

Found: C, 46.38; H, 8.51; N, 15.13; Cl, 15.80

Mass spec: M+1=214

### Example NN

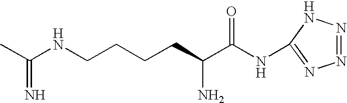

### 2S-amino-6-[(1-iminoethyl)amino]-N-(1H-tetrazol-5-yl)hexanamide, hydrate, dihydrochloride

NN-1 To a stirring solution of Boc-L-Lys(Cbz)-OH (5 g, 13.18 mmol), 5-aminotetrazole monohydrate (1.36 g, 13.18 mmol) and N,N-diisopropylethylamine (DIPEA) (5.1 g, 6.9 mL, 39.54 mmol) in 20 mL of dimethylformamide (DMF) at ambient temperature was added benzotriazol-1-yl-oxy-tris-(dimethylamino)phosphonium hexafluorophosphate (BOP) (6.4 g, 14.49 mmol).

After being stirred for 1 h, the reaction mixture was concentrated under vacuum. The residue was distributed between 60 mL of ethyl acetate (EtOAc) and 50 mL of water. The layers were separated. The organic layer was washed with 50 mL of 1M KHSO₄ solution and 2 times with 50 mL of water. The product started to precipitate and the suspension was concentrated in vacuum giving 9 g of crude compound. After drying, the product was purified by boiling in methylene chloride followed by filtration, giving 3.7 g of 1A (62.7%). The compound was characterized by ¹H NMR.

NN-2 (2 g, 4.5 mmol) was reduced under catalytic hydrogenation conditions using Pd black at 5 psi in 50% EtOH/AcOH solution for 12 h, giving 1.55 g (100%) of NN-2. The compound was characterized by ¹H NMR.

NN-3 To a stirring solution of NN-2 (1.55 g, 4.15 mmol) and methyl acetimidate hydrochloride (0.91 g, 8.31 mmol) in 25 mL of DMF was added triethylamine (TEA) (1.26 g, 1.74 mL, 12.45 mmol). After being stirred 16 h at ambient temperature, the reaction mixture was filtered from triethylamine hydrochloride and the filtrate was concentrated in vacuum. The residue was dissolved in 50% AcOH and lyophilized. The crude product (2 g) was purified using reverse-phase chromatography on a C-18 column giving 0.9 g (52.3%) of 1C. The product was characterized by ¹H NMR.

NN-4 (0.9 g, 2.17 mmol) was dissolved in 30 mL of acetic acid and 3 mL of 4 N HCl/dioxane were added. The reaction was stirred for 20 min. at ambient temperature then 150 mL of ethyl ether were added. After 2 h, the precipitate was filtered, washed with ethyl ether, and dried giving 0.78 g of 1 (96%). Anal. Calcd. for C₉H₁₈N₈O,2HCl, 1.25H₂O: C,30.91; H, 6.48; N, 32.04; Cl, 20.27. Found: C, 31.64; H, 6.43; N, 32.19; Cl, 20.19. DSC mp 144.9° C.

Example NN is a more potent i-NOS inhibitor than 2S-amino-6-[(1-iminoethyl)amino]hexanamide (NIL amide) or NIL dimethylamide Example 1 is also more selective. Example NN is a nicely crystalline product as are all its intermediates. In contrast, NIL is a glass, which makes it difficult to handle.

### c. Biological Data

Some or all of the following assays are used to demonstrate the nitric oxide synthase inhibitory activity of the invention's compounds as well as demonstrate the useful pharmacological properties.

### Citrulline Assay for Nitric Oxide Synthase

Nitric oxide synthase (NOS) activity can be measured by monitoring the conversion of L-[2,3-³H]-arginine to L-[2,3-³H]-citrulline (Bredt and Snyder, Proc. Natl. Acad. Sci. U.S.A., 87, 682-685, 1990 and Moore et al, J. Med. Chem., 39, 669-672, 1996). Human inducible NOS (hiNOS), human endothelial constitutive_NOS (hecNOS) and human neuronal constitutive NOS (hncNOS) are each cloned from RNA extracted from human tissue. The cDNA for human inducible NOS (hiNOS) is isolated from a λcDNA library made from RNA extracted from a colon sample from a patient with ulcerative colitis. The cDNA for human endothelial constitutive NOS (hecNOS) is isolated from a λcDNA library made from RNA extracted from human umbilical vein endothelial cells (HUVEC) and the cDNA for human neuronal constitutive NOS (hncNOS) is isolated from a λcDNA library made from RNA extracted from human cerebellum obtained from a cadaver. The recombinant enzymes are expressed in Sf9 insect cells using a baculovirus vector (Rodi et al, in The Biology of Nitric Oxide, Pt. 4: Enzymology, Biochemistry and Immunology; Moncada, S., Feelisch, M., Busse, R., Higgs, E., Eds.; Portland Press Ltd.: London, 1995; pp 447-450). Enzyme activity is isolated from soluble cell_extracts and partially purified by DEAE-Sepharose chromatography. To measure NOS activity, 10 μL of enzyme is added to 40 μL of 50 mM Tris (pH 7.6) in the presence or absence of test compounds and the reaction initiated by the addition of 50 μL of a reaction mixture containing 50 mM Tris (pH 7.6), 2.0 mg/mL bovine serum albumin, 2.0 mM DTT, 4.0 mM CaCl₂, 20 μM FAD, 100 μM tetrahydrobiopterin, 0.4 mM NADPH and 60 μM L-arginine containing 0.9 μCi of L-[2,3-³H]-arginine. The final concentration of L-arginine in the assay is 30 μM. For hecNOS or hncNOS, calmodulin is included at a final concentration of 40-100 nM. Following incubation at 37° C. for 15 minutes, the reaction is terminated by addition of 400 μL of a suspension_(1 part resin, 3 parts buffer) of Dowex 50W X-8 cation exchange resin in a stop buffer containing 10 mM EGTA, 100 mM HEPES, pH 5.5 and 1 mM L-citrulline. After mixing the resin is allowed to settle and L-[2,3-³H]-Citrulline formation is determined by counting aliquots of the supernatant with a liquid scintillation counter. Results are reported in Table I as the IC₅₀ values of compounds for hiNOS, hecNOS and hncNOS.

### Raw Cell Nitrite Assay

RAW 264.7 cells can be plated to confluency on a 96-well tissue culture plate grown overnight (17 h) in the presence of LPS to induce NOS. A row of 3-6 wells can be left untreated and served as controls for subtraction of nonspecific background. The media can be removed from each well and the cells washed twice with Kreb-Ringers-Hepes (25 mM, pH 7.4) with 2 mg/ml glucose. The cells are then placed on ice and incubated with 50 μL of buffer containing L-arginine (30 μM) ± inhibitors for 1 h. The assay can be initiated by warming the plate to 37° C. in a water bath for 1 h. Production of nitrite by intracellular iNOS will be linear with time. To terminate the cellular assay, the plate of cells can be placed on ice and the nitrite-containing buffer removed and analyzed for nitrite using a previously published fluorescent determination for nitrite. T. P. Misko et al, Analytical Biochemistry, 214, 11-16 (1993).

### Human Cartilage Explant Assay

Bone pieces are rinsed twice with Dulbecco's Phosphate Buffered Saline (GibcoBRL) and once with Dulbecco's Modified Eagles Medium (GibcoBRL) and placed into a petri dish with phenol red free Minimum Essential Medium (MEM) (GibcoBRL). Cartilage was cut into small explants of approximately 15-45 mg in weight and one or two explants per well are placed into either 96 or 48 well culture plates with 200-500 μL of culture media per well. The culture media was either a custom modification of Minimum Essential Medium(Eagle) with Earle's salts (GibcoBRL) prepared without L-Arginine, without L-Glutamine and without phenol red or a custom modification of serumless Neuman and Tytell (GibcoBRL) medium prepared without L-arginine, without insulin, without ascorbic acid, without L-glutamine and without phenol red. Both are supplemented before use with 100 μM L-Arginine (Sigma), 2 mM L-glutamine, 1× HL-1 supplement (BioWhittaker), 50 mg/ml ascorbic acid (Sigma) and 150 pg/ml recombinant human IL-1□ (RD Systems) to induce nitric oxide synthase. Compounds are then added in 10 μL aliquots and the explants incubated at 37° C. with 5% CO₂ for 18-24 hours. The day old supernatant is then discarded and replaced with fresh culture media containing recombinant human IL-1β and compound and incubated for another 20-24 hours. This supernatant is analyzed for nitrite with a fluorometric assay (Misko et al, Anal. Biochem., 214, 11-16, 1993). All samples are done in quadruplicate. Unstimulated controls are cultured in media in the absence of recombinant human IL-1 □. IC₅₀ values (Table II) are determined from plotting the percent inhibition of nitrite production at six different concentrations of inhibitor.

Table II shows examples of biological activity for some of the compounds of the present invention.

TABLE II
Biological Activity: Values represent averages across
all experiments and all lots studied.

| Example | hiNOS | | hncNOS | Human |
|---|---|---|---|---|
| Number of | IC₅₀ | hecNOS IC₅₀ | IC₅₀ | Cartilage IC₅₀ |
| Compound | (μM) | (μM) | (μM) | (μM) |
| Example A | 0.36 | 68 | 3.6 | 0.1 |
| Example B | 2.2 | 195 | 21 | 0.2 |
| Example C | 12 | 303 | 105 | |
| Example D | 8.6 | 112 | 65 | 2.5 |
| Example E | <5 | 279 | 29 | |
| Example I | 3.1 | 77 | 15 | 0.7 |
| Example J | 4.4 | 302 | 58 | 8.2 |
| Example K | 74 | 266 | 86 | |
| Example L | 197 | 1100 | 539 | |
| Example M | 3.4 | 78 | 17 | |
| Example N | 0.9 | 26 | 6.0 | |
| Example O | 7.2 | >100 | 36 | 0.7 |
| Example P | 12 | >100 | 181 | |
| Example Q | 12 | 1080 | 220 | |
| Example S | 172 | 1490 | 523 | |
| Example T | 0.9 | 89 | 8 | 0.1 |
| Example U | 20 | 418 | 150 | |
| Example V | <3 | >30 | >3 | <10 |
| Example W | <5 | >150 | >10 | >30 |
| Example X | <3 | >15 | >3 | <10 |
| Example Y | <3 | >30 | >3 | <10 |
| Example Z | <3 | >15 | >3 | <10 |
| Example AA | <3 | >5 | <3 | <3 |
| Example BB | <10 | >25 | <10 | |
| Example CC | 2.9 | 29 | 9.9 | 0.5 |
| Example DD | 10 | 74 | 31 | 1.8 |
| Example EE | 1.4 | 18 | 5.8 | 0.5 |
| Example FF | 16 | 86 | 45 | |
| Example GG | 34 | 386 | 122 | |
| Example HH | 0.4 | 37 | 7.6 | 0.4 |
| Example JJ | 56 | 352 | 584 | |
| Example KK | 0.57 | 52 | 13 | |
| Example LL | 0.7 | 31 | 12 | 0.8 |
| Example MM | 121 | 1930 | 1480 | |
| Example NN | 21.4 | 2425 | | |

### In vivo Assay

Rats can be treated with an intraperitoneal injection of 1-12.5 mg/kg of endotoxin (LPS) with or without oral administration of the nitric oxide synthase inhibitors. Plasma nitrite/nitrate levels can be determined 5 hours post-treatment. The results can be used to show that the administration of the nitric oxide synthase inhibitors decreases the rise in plasma nitrite/nitrate levels, a reliable indicator of the production of nitric oxide induced by endotoxin. As shown in Table III, Example A ((2S,5E)-2-amino-6-fluoro-7-[(1-iminoethyl)amino]-5-heptenoic acid, dihydrochloride) inhibited the LPS-induced increase in plasma nitrite/nitrate levels with an observed ED₅₀ value of <0.1 mg/kg, demonstrating the ability to inhibit inducible nitric oxide synthase activity in vivo.

TABLE III
ED₅₀'s for Compounds Determined in Endotoxin-Treated Rats
All compounds administered orally unless otherwise noted.

| Compound | ED₅₀ (mg/kg) |
|---|---|
| Example A | <0.1 |
| Example D | >10 |
| Example G | <0.1 |
| Example H | <0.3 |
| Example V | <3 |
| Example W | >10 |
| Example X | <5 |
| Example Y | <3 |
| Example Z | <5 |
| Example AA | <10 |
| Example CC | <3 |
| Example EE | 0.2 |
| Example HH | 0.4 |
| Example KK | 0.3 |
| Example LL | 0.3 |

### Assay for Time Dependent Inhibition

Compounds are evaluated for time dependent inhibition of human NOS isoforms by preincubation of the compound with the enzyme at 37° C. in the presence of the citrulline enzyme assay components, minus L-arginine, for times ranging from 0-60 minutes. Aliquots (10 □L) are removed at 0, 10 ,21 and 60 minutes and immediately added to a citrulline assay enzyme reaction mixture containing L-[2,3-³H]-arginine and a final L-arginine concentration of 30 □M in a final volume of 100 □L. The reaction is allowed to proceed for 15 minutes at 37° C. and terminated by addition of stop buffer and chromatography with Dowex 50W X-8 cation exchange ion exchange resin as described for the citrulline NOS assay. The % inhibition of NOS activity by an inhibitor was taken as the per cent inhibition in activity compared to control enzyme preincubated for the same time in the absence of inhibitor. Data shown in Table IV is the % inhibition after 21 and 60 minutes preincubation of inhibitor with enzyme.

TABLE IV

| Example No. | hiNOS | hecNOS | hncNOS |
|---|---|---|---|
| V | 75%@2.8 μM@21 min | 11%@33 μM@21 min | 0%@5 μM@21 min |
| | 76%@2.8 μM@60 min | 11%@33 μM@60 min | 0%@5 μM@60 min |
| W | 34%@4.2 μM@21 min | 9%@173 μM@21 min | 0%@13 μM@21 min |
| | 38%@4.2 μM@60 min | 0%@173 μM@60 min | 0%@13 μM@60 min |
| X | 86%@2.2 μM@21 min | 18%@15 μM@21 min | 0%@3 μM@21 min |
| | 85%@2.2 μM@60 min | 16%@15 μM@60 min | 0%@3 μM@60 min |
| Y | 75%@2.8 μM@21 min | 11%@33 μM@21 min | 0%@5 μM@21 min |
| | 76%@2.8 μM@60 min | 11%@33 μM@60 min | 0%@5 μM@60 min |
| Z | 86%@2.2 μM@21 min | 18%@15 μM@21 min | 0%@3 μM@21 min |
| | 85%@2.2 μM@60 min | 16%@15 μM@60 min | 0%@3 μM@60 min |
| AA | 96%@2.2 μM@21 min | 58%@5.7 μM@21 min | 34%@0.9 μM@21 min |
| | 97%@2.2 μM@60 min | 55%@2.2 μM@60 min | 0%@0.9 μM@60 min |

### In vitro Studies with Airway Epithelial Cells

Immunocytochemistry on bronchial brushings: Epithelial cells obtained via bronchial brushings from healthy non-asthmatic individuals were prepared using a Shandon Cytospin 3 centrifuge. The slides were air-dried and fixed in ice-cold acetone for 10 min. Endogenous peroxide activity was quenched by immersion of the slides for 1 h in 1% (v/v) H₂O₂ in PBS containing 0.02% (w/v) sodium azide. The slides were subsequently washed three times for 5 min in PBS. The slides were incubated with 5% (v/v) normal swine serum for 20 min in order to prevent non-specific binding. Anti-iNOS antibody (rabbit polyclonal, affinity purified anti human iNOS MoAb 720; Pharmacia Corporation, St. Louis, Mo.) or pre-immune rabbit serum was then applied to the slides at a dilution of 1:50 for 1 h at room temperature. The slides were then washed and incubated with biotinylated swine anti-rabbit IgG (1:200) for 30 min at room temperature and washed. The slides were then incubated in avidin-horse radish peroxidase (1:500) for an additional 30 min. The slides were again washed and the antigen visualized using diaminobenzidine. The slides were counterstained using hematoxylin for 10s, dehydrated in ascending alcohols, cleared using xylene and mounted using DPX. Cells were then observed under light microscopy.

Human primary airway epithelial cell cultures: Human primary airway epithelial cells were cultured as described previously (see Donnelly,L. E. and Barnes,P. J. Am J Respir Cell Mol.Biol 24:295-303 (2000)), and the amount of nitrite, the stable oxidation product of NO, in the cell culture media was measured using a modification of the spectrofluorometric method of Misko et al., Anal. Biochem. 4:11-16 (1993).

Western blotting of iNOS from cultured epithelial cells: Following incubation with the cytokine mixture alone or with various concentrations of L-NIL, epithelial cells were lysed in 50 mM Tris/HCl, pH 7.4, containing 0.25 mM ethylene diaminotetraacetic acid, 0.5 mM phenylmethylsulfonyl fluoride, 5 μg/ml antipain, 5 μg/ml leupeptin and 5 μg/ml benzaminidine. The protein concentration was determined using the BioRad protein assay kit according to the manufacturer's instructions. Cell proteins were solubilized in sodium dodecyl sulfate polyacrylamide gel electrophoresis sample buffer (0.0625 mM Tris/HCl, pH 6.8, containing 10% v/v glycerol, 1% w/v SDS, 1% w/v β-mercaptoethanol, and 0.01% w/v bromophenol blue). The proteins (15 μg per lane) were resolved by electrophoresis in 3-8% (w/v) Tris-acetate SDS-polyacrylamide gels and transferred to Hybond-Enhanced Chemi-luminescence (ECL) nitro-cellulose membranes. Equal protein loading was determined by staining the blot with 0.1% (w/v) Ponceau S in 5% (v/v) acetic acid. The nitro-cellulose was then blocked overnight at 4° C. in 0.5M Tris-HCl,pH 7.4, containing 3% (w/v) normal goat serum, 1% (w/v) bovine serum albumin and 0.05% (v/v) Tween-20. The blots were washed in PBS containing 0.05% (v/v) Tween-20 and incubated for one hour in the presence of the anti-human iNOS primary antibody (1:1000). The blots were washed extensively and then incubated for one hour with anti-rabbit IgG conjugated to horseradish peroxidase (1:4000). The blots were washed extensively again and the bands were visualised using ECL reagent.

Effect of L-NIL on iNOS activity and expression in human primary airway epithelial cells: Brushing cells derived from normal individuals were shown to express iNOS. The protein was evenly distributed throughout the cytoplasm in basal cells but clearly located beneath the cilia in the columnar, ciliated cells. Treatment of airway epithelial cells with 50 ng/ml of IL-1β, TNF-α and IFN-γ (cytomix) for 24 h caused an induction of iNOS protein and an increase in the accumulation of nitrite in cell culture media from 2.20±0.2 μM in the absence of cytomix, to 3.5±0.4 μM in the presence of cytomix.

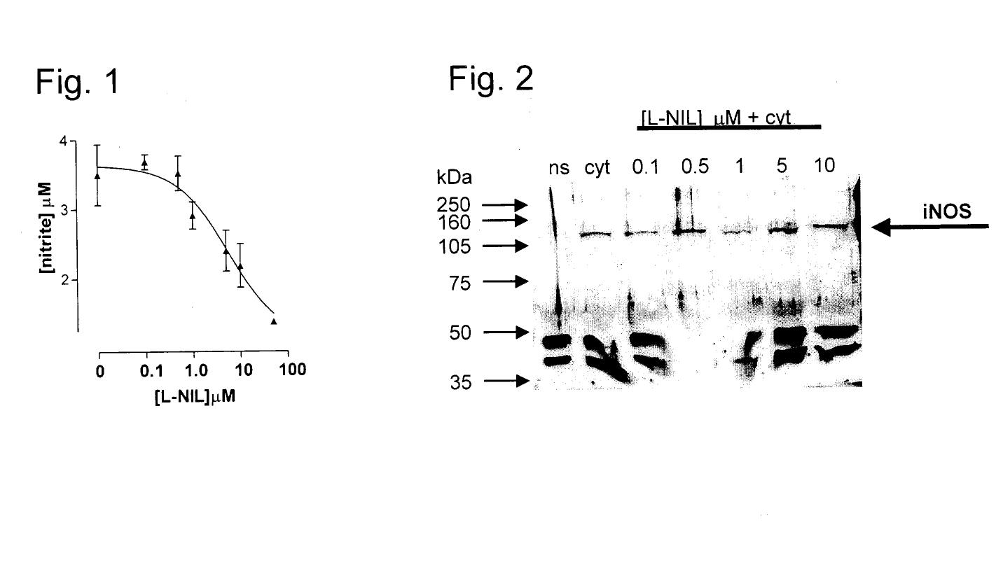

The effect of L-NIL on iNOS activity and expression in human primary airway epithelial cells is shown in FIGS. 1 and 2. Media was harvested and measured for nitrite content (FIG. 1). Cellular proteins were resolved on 3-8% tris-acetate polyacrylamide gels and immunoblotted for iNOS protein (FIG. 2).

FIG. 1 shows that the increase in nitrite in the media was inhibited in a dose-dependent manner by the addition of L-NIL to the cell media (IC_{50˜}5.7 μM). FIG. 2 shows that this decrease in nitrite production is not mediated by an inhibition of iNOS protein expression since the inclusion of L-NIL did not affect iNOS expression as detected by immunoblotting. Rather, as expected, it appears to result from inhibition of enzymatic activity.

### Clinical Studies

A single center study was performed at the National Heart and Lung Institute (NHLI) Clinical Studies Unit in London, UK. The clinical protocol was submitted and accepted by the Ethics Committee of the Royal Brompton Hospital Trust, and the study was carried out according to Good Clinical Practice standards. Each subject provided written informed consent.

Twenty-four, adult, healthy volunteers and 24 patients with mild asthma participated in this study. All subjects were lifelong non-smokers. Asthma patients (n=24) had mild asthma according to American Thoracic Society Criteria, with medication on an as required basis with inhaled albuterol only. Patients had a forced expiratory volume in one second (FEV₁) ≧70%, a documented >15% reversibility with a short-acting β₂-agonist, and a provocative concentration of methacholine or histamine to cause a 20% drop in FEV, (PC₂₀) of ≦8.0 mg/ml. In addition, asthmatics had a documented skin prick test which was positive to at least one common inhaled allergen (Dermatophagoides pteronyssinus, mixed grass pollen or cat dander), and none had an exacerbation of asthma or respiratory tract infection in the 4 weeks preceding the study. All asthmatic patients had baseline exhaled breath NO levels of >15 parts per billion (ppb). Healthy control (n=24) subjects were matched for age and sex, and had no clinically significant disease. Healthy subjects were required to have baseline exhaled NO levels between 4 and 9 ppb.

The study included 2 panels of healthy volunteers and asthmatic patients who received in a double-blind, randomized manner, a single dose of oral compound NN or placebo. The study was carried out in two phases according to the dose of compound NN under investigation. The first panel consisted of patients with mild-to-moderate asthma (n=12) and healthy subjects (n=12) who received single doses of 20 mg of compound NN and placebo, while the second panel of mild-to-moderate asthmatics (n=12) and healthy subjects (n=12) received single doses of 200 mg of compound NN and placebo. No patient or subject participated in both panels of this study. Evaluation of the first panel was completed before moving on to the second panel. The study evaluated the tolerability and pharmacokinetics of orally-administered compound NN and monitored levels of exhaled breath NO.

Measurement of exhaled NO: Exhaled NO was measured using a chemoluminescence analyzer (Logan 2000, Rochester, UK) and methods in accordance with International Guidelines. The analyser was calibrated daily using a certified NO mixture of 50 to 200 ppb (BOC Special gases, Guildford, UK) and ambient air NO recordings made on an hourly basis. Subjects were asked to refrain from strenuous exercise before any measurement. Each subject was seated for at least 5 minutes before any measurement was taken and remained seated throughout the procedure. Subjects began with exhalation to residual volume followed by rapid inhalation to total lung capacity. Inspiration was rapid and was <2.5 secs in healthy subjects and <4 secs in asthmatic patients. After the analyzer had been zeroed for NO, subjects exhaled from total lung capacity over 15-20 secs against low resistance (5-20 cm H₂O) at a constant steady flow rate of 250 ml/sec into a wide bore Teflon tubing attached to the chemiluminescence analyzer. Sampling was performed at a rate of 250 ml/min. The NO plateau response was interpreted as being of at least 10 seconds in duration. Two successive recordings were taken, and the average value was calculated.

Statistical methods for clinical data: Exhaled NO data, including change from baseline and the percentage change, were tested for normality using the Shapiro-Wilks test. Significant deviations were found, therefore non-parametric statistical methods were used. The change from baseline was tested using a Wilcoxon signed-rank test. The area under the curve (AUC) for the change from baseline and the percentage change, and the differences between active and placebo were calculated. The test for normality found significant deviations so non-parametric statistical methods were used. The significance of the overall change was tested by comparing the AUC to zero using a Wilcoxon signed-rank test. Differences between treatments within each panel were tested using non-parametric analysis of cross-over studies. Differences between the 2 active doses in the 2 panels (20 mg and 200 mg) were tested using a Wilcoxon rank sum test.

Forced expiratory volume in 1 sec (FEV₁) and heart rate data had no significant deviations from normality using the Shapiro-Wilks test, therefore parametric statistical methods were used, and the significance of the change from baseline was tested using a paired t-rank test.

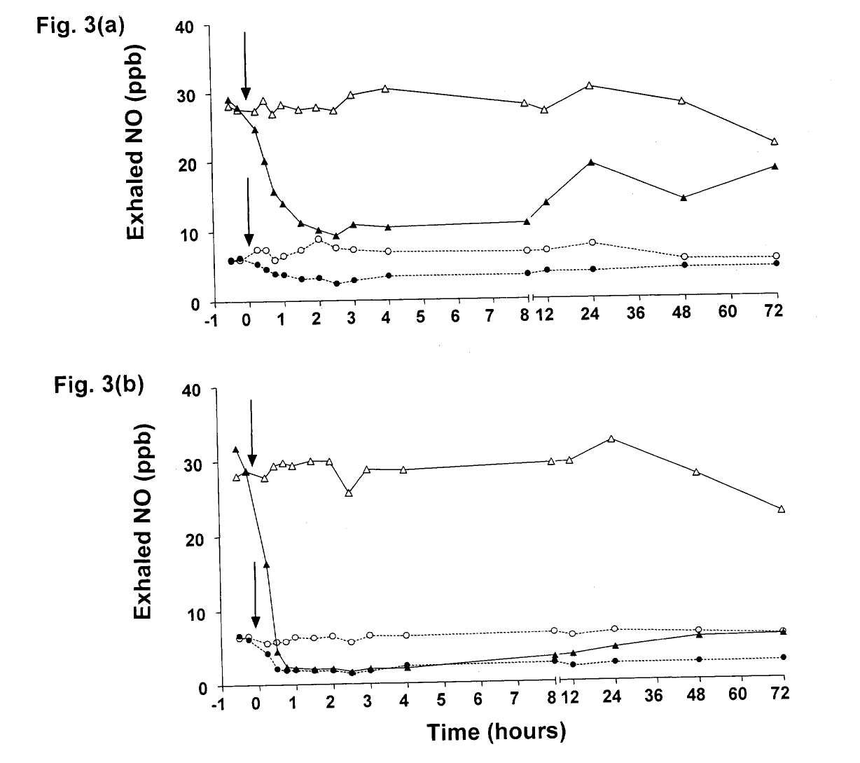

Effects of INOS Inhibition in Normal Subjects and Asthma Patients: The levels of exhaled NO in normal subjects and asthmatic patients following the administration of placebo and doses of either 20 or 200 mg of compound NN are shown in FIG. 3. Change in exhaled breath nitric oxide (NO) levels is shown following oral administration of (A) compound NN (20 mg) and (B) compound NN (200 mg) in patients with mild-to-moderate asthma (closed triangles) compared with placebo (open triangles) and in healthy subjects (closed circles) compared with placebo (open circles). Vertical arrows represent times at which dosing with compound NN or placebo was performed. Mean values are shown (n=12).

FIG. 3 shows that The AUC of change in exhaled NO was not significantly different from zero for placebo in any of the 4 groups of subjects. Following the administration of both doses of compound NN, there was a rapid reduction in exhaled NO which was highly significantly different in both the healthy volunteers and the asthmatics throughout the 72 hour assessment. In addition, the difference in AUC of change in NO for the subjects receiving 200 mg of compound NN was higher in both the healthy volunteers (p<0.001) and the asthmatics (p=<0.01) than the AUC in subjects receiving 20 mg of compound NN (p=0.004).

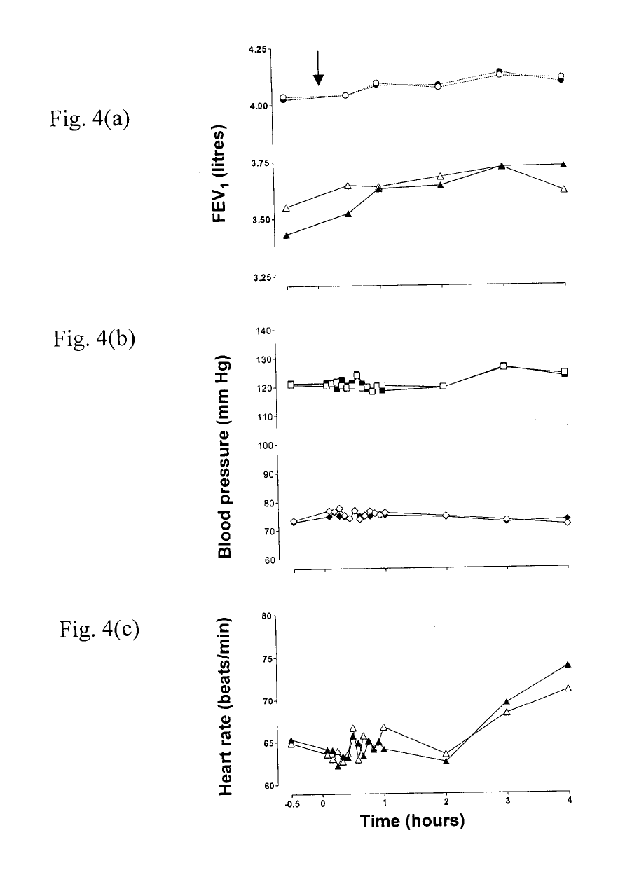

FIG. 4 shows effects of oral administration of compound NN on FEV₁, blood pressure and heart rate. Change in FEV₁ following oral administration of compound NN (200 mg) in patients with (A) mild-to moderate asthma (closed triangles) compared with placebo (open triangles) and in healthy subjects (closed circles) compared with placebo (open circles). (B) Change in systolic blood pressure following oral administration of compound NN (200 mg) (closed squares) compared with placebo (open squares) and change in diastolic blood pressure following oral administration of compound NN (200 mg) (closed diamonds) compared with placebo (open diamonds). (C) Change in heart rate following oral administration of 200 mg compound NN (closed triangles) compared with placebo (open triangles). Vertical arrow represents time at which each dose of compound NN or placebo was administered. Mean values are shown (n=12).

FIG. 4 shows that compound NN was well tolerated with no apparent effects on heart rate, blood pressure and FEV₁. In addition, there was no effect on haematology or blood biochemistry.

The selective iNOS inhibitor compound NN was well tolerated in all subjects and caused a rapid reduction in exhaled breath NO following oral administration to both healthy volunteers and asthmatics. In addition, the decrease in exhaled breath NO lasted for 3 days in both groups. There were no significant changes in lung function, blood pressure and heart rate, or in laboratory hematology and biochemistry parameters. Studies with intravenous administration of L-NMMA, a non-selective inhibitor of NOS, caused hypertension in both animals and man. The description of hypertension in mutant mice lacking the gene for eNOS (Huang,P. L. et al., Nature 377:239-42 (1995)) suggests that L-NMMA is causing hypertension through inhibition of eNOS. Therefore, the excellent tolerability of compound NN suggests that at the doses tested, this compound is not appreciably inhibiting eNOS, and encourages the study of iNOS inhibitors in asthma well as other inflammatory conditions.

The long duration of effects in terms of lowering exhaled NO following a single dose suggests that the dosing regimen for this compound could be adjusted to permit single daily dosing. The effects on NO were demonstrated to be of rapid onset and dose-related. The higher 200 mg dose may be a supra-maximal dose lying on the plateau of the dose response curve. Furthermore, the 200 mg dose caused a 95% inhibition of exhaled NO in asthma, that is greater than the approximately 70% inhibition obtained with high doses of non-specific inhibitors such as L-NMMA (Yates,D. H., et al., Am J Respir Crit Care Med 152:892-896 (1995)) and L-NAME (Gomez,F. P., et al., Eur Respir J 12:865-871 (1998)), as well as with the more selective amino-guanidine (Yates,D. H., et al., Am J Respir Crit Care Med 154:247-250 1996)). Assuming that complete inhibition of iNOS has been selectively obtained, the residual exhaled NO of <1 ppb in healthy volunteers and asthmatics may be produced by constitutive nNOS and eNOS as well as exogenous atmospheric sources.

An expanding body of research supports an important role for NO in human asthma. Selective iNOS inhibitors have been shown to suppress eosinophil infiltration to the lung in rodent models of allergic airway inflammation (Koarai,A., et al., Pulm.Pharmacol Ther. 13:267-275 (2000)), with associated decreased lung chemokine expression (Trifilieff,A., et al, J Immunol 165:1526-33 (2000)), and allergic airway inflammation inhibited in mice deficient in iNOS (Xiong,Y., et al., The Journal of Immunology 162:445-52 (1999)). Gaseous NO is detectable in elevated amounts in exhaled breath from asthmatic patients (see, e.g., Stirling, R. G. et al., Thorax 53:1030-34 (1998)) and peroxynitrite (Sadeghi-Hashjin,G. et al., Clin Exp Allergy 28:1464-73 (1998)) is increased in bronchial biopsies from asthmatics (Saleh,D., et al., FASEB J 12:929-37 (1998)). In addition, nitrotyrosine is increased in exhaled breath condensate (Hanazawa,T. et al., Am J Respir Crit Care Med 162:1273-76 (2000)) and in the lung parenchyma and airways of patients who have died from asthma (Kaminsky,D. A. et al., J. Allergy Clin Immunol 104:747-54 (2000)). Recently, S-nitrosothiols have been shown to signal the ventilatory response to hypoxia, S-nitrosoglutathione being formed when nitric oxide synthase (NOS) is activated in neuronal and other tissue (Lipton, A. J. et al., Nature 413:171-74 (2001)).

A therapeutic rationale also exists for use of selective iNOS therapy in chronic obstructive pulmonary disease (COPD) (see, e.g., Barnes, P. J., N Engl J Med 343:269-80 (2000). Although use of exhaled NO as a practical marker for COPD remains controversial (compare Maziak, W. et al., Am J Respir Crit Care Med 157:998-1002 (1998) with Corradi,M. et al., [In Process Citation]. Thorax 54:572-75 (1999), and with Rutgers,S. R. et al., Thorax 54:576-80 (1999)), although there is elevated nitrotyrosine and iNOS in sputum cells (Ichinose,M. et al., Am J Respir Crit Care Med 162:701-6 (2000)). It remains possible that NO is consumed by reactive oxidant species accounting for the finding that patients with severe stable COPD have reduced levels of exhaled NO (Clini, E. et al., Thorax 53:881-3 (1998). Also, NO inhalation has been employed therapeutically in chronic COPD (Ashutosh, K. et al., Thorax 55:109-13 (2000)).

### c. Dosages, Formulations and Routes of Administration

Many of the INOS selective inhibitor compounds useful in the methods of the present invention can have at least two asymmetric carbon atoms, and therefore include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention. Isomers may include geometric isomers, for example cis-isomers or trans-isomers across a double bond. All such isomers are contemplated among the compounds useful in the methods of the present invention. The methods also contemplate use of tautomers, salts, solvates and prodrugs of iNOS selective inhibitor compounds.

For the methods of the present invention, suitable routes of administration of the selective iNOS inhibitors include any means that produce contact of these compounds with their site of action in the subject's body, for example especially in the airways of the trachea, bronchi and lungs. More specifically, suitable routes of administration include inhalation, including oral inhalation or nasal inhalation, intranasal mucosal administration, oral, intravenous, subcutaneous, rectal, topical, buccal (i.e. sublingual), intramuscular, and intradermal.

For the prophylaxis or treatment of respiratory disease and conditions, conditions, including asthmatic conditions, COPD including chronic bronchitis and emphysema, and cystic fibrosis, as well as other respiratory or lung disorders involving airway or lung inflammation, the methods include use of an iNOS selective inhibitor as the compound per se, or as pharmaceutically acceptable salts thereof. The term “pharmaceutically-acceptable salts” embraces salts commonly used to form alkali metal salts and to form addition salts of free acids or free bases. The nature of the salt is not critical, provided that it is pharmaceutically acceptable. Pharmaceutically acceptable salts are particularly useful as products of the methods of the present invention because of their greater aqueous solubility relative to a corresponding parent or neutral compound. Such salts must have a pharmaceutically acceptable anion or cation. Suitable pharmaceutically acceptable acid addition salts of compounds of the present invention may be prepared from inorganic acid or from an organic acid. Examples of such inorganic acids are hydrochloric, hydrobromic, hydroiodic, nitric, carbonic, sulfuric and phosphoric acid. Appropriate organic acids include from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethylsulfonic, benzenesulfonic, sulfanilic, stearic, cyclohexylaminosulfonic, algenic, galacturonic acid. Suitable pharmaceutically-acceptable base addition salts of compounds of the present invention include metallic salts made from aluminum, calcium, lithium, magnesium, potassium, sodium and zinc or organic salts made from N,N′-dibenzylethyleneldiamine, choline, chloroprocaine, diethanolamine, ethylenediamine, meglumine (N-methylglucamine) and procain. Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic (including carbonate and hydrogen carbonate anions), sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. The chloride salt is particularly preferred for medical purposes. Suitable pharmaceutically acceptable base salts include ammonium salts, alkali metal salts such as sodium and potassium salts, and alkaline earth salts such as magnesium and calcium salts. All of these salts may be prepared by conventional means from the corresponding conjugate base or conjugate acid of the compounds of the present invention by reacting, respectively, the appropriate acid or base with the conjugate base or conjugate acid of the compound.

In one embodiment, the iNOS selective inhibitors useful in the methods of the present invention are presented with an acceptable carrier in the form of a pharmaceutical combination. The carrier must be acceptable in the sense of being compatible with the other ingredients of the pharmaceutical combination and must not be deleterious to the subject. Suitable forms for the carrier include solid or liquid or both, and in an exemplary embodiment the carrier is formulated with the therapeutic compound as a unit-dose combination, for example as a tablet that contains from about 0.05% to about 95% by weight of the active compound. In alternative embodiments, other pharmacologically active substances are also present, including other compounds of the present invention. The pharmaceutical compounds of the present invention are prepared by any of the well-known techniques of pharmacy, consisting essentially of admixing the ingredients.

Preferred unit dosage formulations are those containing an effective dose, as herein below described, or an appropriate fraction thereof, of one or more of the therapeutic compounds of the combinations.

In general, a total daily dose of an iNOS selective inhibitor is in the range of about 0.001 mg/kg body weight/day to about 2500 mg/kg body weight/day. The dose range for adult humans is generally from about 0.005 mg to about 10 g per day. Tablets or other forms of presentation provided in discrete units may conveniently contain an amount of a therapeutic compound that is effective at such dosage, or at a multiple of the same. For instance, selective iNOS inhibitory compounds used in the present invention can be presented in units containing 5 mg to 500 mg, and typically around 10 mg to about 200 mg. PDE inhibitory compounds used in the present invention can be presented in units containing 0.005 mg to 500 mg, and typically around 5 mg to about 200 mg.

In general, a total daily dose of a PDE inhibitor is in the range of about 0.001 mg/kg body weight/day to about 2500 mg/kg body weight/day. The dose range for adult humans is generally from about 0.005 mg to about 10 g per day. In particular, daily dosages for adult humans in the range of about 1 mg to about 500 mg are contemplated. For example, an intravenous preparation of a combination including the PDE-III inhibitor AMRINONE includes 0.5 μg/kg for a bolus injection, followed by a 2-20 μg/kg per minute infusion. An intravenous preparation of a combination including the PDE-III inhibitor MILRINONE includes 50 μg/kg for a bolus injection, followed by a 0.25-1.0 μg/kg per minute infusion.

Daily dosages of PDE IV inhibitors can vary within wide limits and will be adjusted to the individual requirements in each particular case. In general, for administration to adults, an appropriate daily dosage has been described below, although the limits that were identified as being preferred may be exceeded if expedient. The daily dosage can be administered as a single dosage or in divided dosages. Various delivery systems include capsules, tablets, food, and gelatin capsules, for example.

The exact dosage and regimen for administering a PDE IV inhibitor will necessarily depend upon the potency and duration of action of the compounds used, the nature and severity of the illness to be treated, as well as the sex, age, weight, general health and individual responsiveness of the patient to be treated, and other relevant circumstances. While not intended to be limiting, an example of the normally prescribed dosage for the PDE IV inhibitor, roflumilast, has been reported to be about 0.5 mg once daily for human rhinitis treatment. See Schmidt, M. et al., J. Allergy Clin. Immunol. 108(4):530-536 (2001). In humans, roflumilast has been reported as effective when dosed at between about 0.01 and 0.5 mg/kg of body weight for inhalation and between about 0.05 and 2 mg/kg of body weight per day for systemic therapies. See U.S. Pat. No. 5,712,298.

TABLE V

| PDE IV | Dosage | |
|---|---|---|
| Inhibitor | Amount | Reference |
| Ariflo | 20-30 mg | Souness, J., et al., Immunopharmacology, |
| | per day | 47: 127-162 (2000) |
| Rolipram | 0.5-2 mg/kg | Teixeira, M., et al., Memorias do |
| | per | Instituto Oswaldo Cruz, 92(II): |
| | day | 193-196 (1997); Souness, J., et al., |
| | | Immunopharmacology, |
| | | 47: 127-162 (2000) |
| Arofylline | 20 mg per | Souness, J., et al., Immunopharmacology, |
| | day | 47: 127-162 (2000) |
| Ibudilast | 40 mg per | Souness, J., et al., Immunopharmacology, |
| | day | 47: 127-162 (2000) |
| Tibenalast | 150 mg per | Souness, J., et al., Immunopharmacology, |
| | day | 47: 127-162 (2000) |
| Piclamilast | 0.2-0.8 mg | Souness, J., et al., Immunopharmacology, |
| | per day | 47: 127-162 (2000) |
| CDP-840 | 30 mg per | Souness, J., et al., Immunopharmacology, |
| | day | 47: 127-162 (2000) |
| RP 73401 | 2 mg/kg | Teixeira, M., et al., Memorias do Instituto |
| | per day | Oswaldo Cruz, 92(II): 193-196 (1997) |
| NVP- | 0.1-3 mg/kg | Trifilieff, A., et al., J. Pharmacol. |
| ABE171 | per | Exp. Ther., 301(1): 241-248 (2002) |
| | day | |

Tablets or other forms of presentation provided in discrete units may conveniently contain an amount of a therapeutic compound that is effective at such dosage, or at a multiple of the same. For instance, selective iNOS inhibitory compounds used in the present invention can be presented in units containing 5 mg to 500 mg, and typically around 10 mg to about 200 mg.

In the case of pharmaceutically acceptable salts of the therapeutic compounds, the weights indicated above refer to the weight of the acid equivalent or the base equivalent of the therapeutic compound derived from the salt.

For the methods herein described, it should be understood that the amount of a selective iNOS inhibitory compound that is required to achieve the desired biological effect depends on a number of factors, including the specific individual compound or compounds chosen, the specific use, the route of administration, the clinical condition of the subject, and the age, weight, gender, and diet of the subject.

The daily doses described in the preceding paragraphs for the various therapeutic compounds are administered in a single dose, or in proportionate multiple subdoses. Subdoses are administered from two to six times per day. In one embodiment, doses are administered in sustained release form effective to obtain the desired biological effect.

Delivery by inhalation, whether oral or nasal inhalation, according to the methods of the present invention can include formulations as are well known in the art, that are capable of being aerosolized for delivery by inhalation. A metered dose inhaler or a nebulizer provides aerosol delivery. Both devices are capable of providing delivery of a range of particle sizes including particles in the preferred range of about 1 micron to about 5 microns. Particles larger than about 10 microns are deposited primarily in the mouth and oropharynx, while particles smaller than about 0.5 microns are inhaled to the alveolae and then exhaled without significant deposition in the lungs. An alternative device for inhalant therapy is a dry powder inhaler using, for example, lactose or glucose powder to carry the therapeutic compound. For all forms of inhalant therapy, factors other than particle size and type of device also influence the amount of deposition in the lungs, including tidal volume, rate of breathing and breath holding. Therefore, an individual being instructed in inhalation therapy according to the methods of current invention should also be instructed to take slow deep breaths and hold each breath for several seconds, and preferably for about 5-10 seconds. Typically, the total daily dose of the therapeutic compounds according to the methods of the present invention will be administered as 1-4 puffs on a b.i.d-q.i.d. basis (i.e. twice-a-day, three times per day or four times a day), and as needed, or solely on an as-needed basis.

Oral delivery according to the methods of the present invention can include formulations, as are well known in the art, to provide prolonged or sustained delivery of the drug to the respiratory system by any number of mechanisms. These include, but are not limited to, pH sensitive release from the dosage form based on the changing pH of the small intestine, slow erosion of a tablet or capsule, retention in the stomach based on physical properties of the formulation, bioadhesion of the dosage form to the mucosal lining of the intestinal tract, or enzymatic release of the active drug from the dosage form.

Oral delivery according to the methods of the present invention can be achieved using a solid, semi-solid or liquid dosage form. Suitable semi-solid and liquid forms include, for example, a syrup or liquid contained in a gel capsule.

To practice the methods of the present invention, pharmaceutical compositions suitable for oral administration can be presented in discrete units, such as capsules, cachets, lozenges, or tablets, each containing a predetermined amount of at least one of the therapeutic compounds useful in the methods of the present invention; as a powder or in granules; as a solution or a suspension in an aqueous or non-aqueous liquid; or as an oil-in-water or water-in-oil emulsion.

### d. Examples of Embodiments

The following non-limiting examples serve to illustrate various aspects of the present invention.

### Example 1

### Pharmaceutical Compositions

100 mg tablets of the composition set forth in Table VI can be prepared using wet granulation techniques:

TABLE VI

| Ingredient | Weight (mg) |
|---|---|
| Compound NN | 5 |
| Roflumilast | 20 |
| Lactose | 54 |
| Microcrystalline Cellulose | 15 |
| Hydroxypropyl Methylcellulose | 3 |
| Croscarmelose Sodium | 2 |
| Magnesium Stearate | 1 |
| Total Tablet Weight | 100 |

### Example 2

### Pharmaceutical Compositions

100 mg tablets of the composition set forth in Table VII can be prepared using direct compression techniques:

TABLE VII

| Ingredient | Weight (mg) |
|---|---|
| Compound I | 5 |
| Roflumilast | 20 |
| Microcrystalline Cellulose | 69.5 |
| Colloidal Silicon Dioxide | 0.5 |
| Talc | 2.5 |
| Croscarmelose Sodium | 0.5 |
| Magnesium Stearate | 1 |
| Total Tablet Weight | 100 |

### Combinations

Table VIII illustrates, by way of example and not limitation, some of the many combinations of the present invention wherein the combination comprises an amount of an iNOS inhibitor and an amount of a PDE inhibitor, wherein the amount of the iNOS inhibitor and the amount of the PDE inhibitor together constitute a respiratory disease or condition effective amount of the iNOS inhibitor and the PDE inhibitor.

TABLE VIII

| Binary Example Number | Component 1 | Component 2 |
|---|---|---|
| 1 | I | MILRINONE |
| 2 | I | AMRINONE |
| 3 | I | PIMOBENDAN |
| 4 | I | CILOSTAMIDE |
| 5 | I | ENOXIMONE |
| 6 | I | PEROXIMONE |
| 7 | I | VESNARINONE |
| 8 | I | ROLIPRAM |
| 9 | I | RO 20-1724 |
| 10 | I | ROFLULMILAST |

The examples described herein can be performed by substituting the generically or specifically described therapeutic compounds or inert ingredients for those used in the preceding examples.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

What is claimed is:

1. A method for the treatment, prevention or inhibition of a respiratory disease or condition in a subject in need of such treatment, prevention or inhibition, comprising administering to said subject an iNOS blocker or pharmaceutically acceptable salt or prodrug thereof and a phosphodiesterase (PDE) inhibitor or pharmaceutically acceptable salt or prodrug thereof.

2. The method according to claim 1 wherein the iNOS blocker is an iNOS selective inhibitor.

3. The method according to claim 1 wherein the administration of the iNOS blocker or pharmaceutically acceptable salt or prodrug thereof and the PDE inhibitor or pharmaceutically acceptable salt or prodrug thereof together comprise a respiratory disease or condition effective method for the treatment, prevention or inhibition of the respiratory disease or condition.

4. The method according to claim 1 wherein the iNOS inhibitor is represented by the formula: 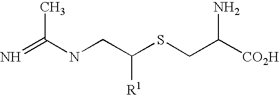
wherein R¹ is selected from C₁₋₄ alkyl, C₃₋₄ cycloalkyl, C₁₋₄ hydroxyalkyl, and C₁₋₄ haloalkyl.
or a pharmaceutically acceptable salt thereof.

5. The method of claim 4 wherein said iNOS inhibitor is selected from the group consisting of:
S—((R)-2-(1-iminoethylamino)propyl)-L-cysteine;
S—((S)-2-(1-iminoethylamino)propyl)-L-cysteine;
S—((R/S)-2-(1-iminoethylamino)propyl)-L-cysteine;
S—((R)-2-(1-iminoethylamino)propyl)-D-cysteine;
S—((S)-2-(1-iminoethylamino)propyl)-D-cysteine;
S—((R/S)-2-(1-iminoethylamino)propyl)-D-cysteine;
S—((R/S)-2-(1-iminoethylamino)butyl)-L-cysteine;
S—((R/S)-2-(1-iminoethylamino,2-cyclopropyl)ethyl)-L-cysteine; and
S—((R/S)-2-(1-iminoethylamino,3-hydroxy)propyl)-L-cysteine,
or a pharmaceutically acceptable salt, solvate, or physiologically functional derivative thereof.

6. The method according to claim 2 wherein the iNOS inhibitor is selected from the group consisting of:
a compound having Formula I 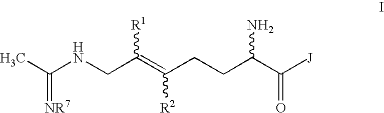
wherein:
R¹ is selected from the group consisting of H, halo and alkyl which may be optionally substituted by one or more halo;
R² is selected from the group consisting of H, halo and alkyl which may be optionally substituted by one or more halo;
with the proviso that at least one of R¹ or R² contains a halo;
R⁷ is selected from the group consisting of H and hydroxy;
J is selected from the group consisting of hydroxy, alkoxy, and NR³R⁴ wherein;
R³ is selected from the group consisting of H, lower alkyl, lower alkylenyl and lower alkynyl;
R⁴ is selected from the group consisting of H, and a heterocyclic ring in which at least one member of the ring is carbon and in which 1 to about 4 heteroatoms are independently selected from oxygen, nitrogen and sulfur and said heterocyclic ring may be optionally substituted with heteroarylamino, N-aryl-N-alkylamino, N-heteroarylamino-N-alkylamino, haloalkylthio, alkanoyloxy, alkoxy, heteroaralkoxy, cycloalkoxy, cycloalkenyloxy, hydroxy, amino, thio, nitro, lower alkylamino, alkylthio, alkylthioalkyl, arylamino, aralkylamino, arylthio, alkylsulfinyl, alkylsulfonyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, alkanoyl, alkenoyl, aroyl, heteroaroyl, aralkanoyl, heteroaralkanoyl, haloalkanoyl, alkyl, alkenyl, alkynyl, alkylenedioxy, haloalkylenedioxy, cycloalkyl, cycloalkenyl, lower cycloalkylalkyl, lower cycloalkenylalkyl, halo, haloalkyl, haloalkoxy, hydroxyhaloalkyl, hydroxyaralkyl, hydroxyalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, cyanoalkyl, dicyanoalkyl, carboxamidoalkyl, dicarboxamidoalkyl, cyanocarboalkoxyalkyl, carboalkoxyalkyl, dicarboalkoxyalkyl, cyanocycloalkyl, dicyanocycloalkyl, carboxamidocycloalkyl, dicarboxamidocycloalkyl, carboalkoxycyanocycloalkyl, carboalkoxycycloalkyl, dicarboalkoxycycloalkyl, formylalkyl, acylalkyl, dialkoxyphosphonoalkyl, diaralkoxyphosphonoalkyl, phosphonoalkyl, dialkoxyphosphonoalkoxy, diaralkoxyphosphonoalkoxy, phosphonoalkoxy, dialkoxyphosphonoalkylamino, diaralkoxyphosphonoalkylamino, phosphonoalkylamino, dialkoxyphosphonoalkyl, diaralkoxyphosphonoalkyl, guanidino, amidino, and acylamino;
a compound having a structure corresponding to Formula II 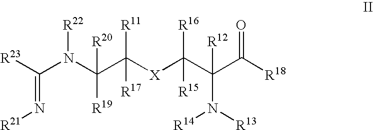
wherein X is selected from the group consisting of —S—, —S(O)—, and —S(O)₂—, R¹² is selected from the group consisting of C₁-C₆ alkyl, C₂-C₆ alkenyl, C₂-C₆ alkynyl, C₁-C₅ alkoxy-C₁ alkyl, and C₁-C₅ alkylthio-C₁ alkyl wherein each of these groups is optionally substituted by one or more substituent selected from the group consisting of —OH, alkoxy, and halogen, R¹⁸ is selected from the group consisting of —OR²⁴ and —N(R²⁵)(R²⁶), and R¹³ is selected from the group consisting of —H, —OH, —C(O)—R²⁷, —C(O)—O—R²⁸, and —C(O)—S—R²⁹; or R¹⁸ is —N(R³⁰)—, and R¹³ is —C(O)—, wherein R¹⁸ and R¹³ together with the atoms to which they are attached form a ring; or R¹⁸ is —O—, and R¹³ is —C(R³¹)(R³²)—, wherein R¹⁸ and R¹³ together with the atoms to which they are attached form a ring, wherein if R¹³ is —C(R3²¹)(R³²)—, then R¹⁴ is —C(O)—O—R³³; otherwise R¹⁴ is —H, R¹¹, R¹⁵, R¹⁶, and R¹⁷ independently are selected from the group consisting of —H, halogen, C₁-C₆ alkyl, C₂-C₆ alkenyl, C₂-C₆ alkynyl, and C₁-C₅ alkoxy-C₁ alkyl, R¹⁹ and R²⁰ independently are selected from the group consisting of —H, C₁-C₆ alkyl, C₂-C₆ alkenyl, C₂-C₆ alkynyl, and C₁-C₅ alkoxy-C₁ alkyl, R²¹ is selected from the group consisting of —H, —OH, —C(O)—O—R³⁴, and —C(O)—S—R³⁵, and R²² is selected from the group consisting of —H, —OH, —C(O)—O—R³⁶, and —C(O)—S—R³⁷; or R²¹ is —O—, and R²² is —C(O)—, wherein R²¹ and R²² together with the atoms to which they are attached form a ring; or R²¹ is —C(O)—, and R²² is —O—, wherein R²¹ and R²² together with the atoms to which they are attached form a ring, R²³ is C₁ alkyl, R²⁴ is selected from the group consisting of —H and C₁-C₆ alkyl, wherein when R²⁴ is C₁-C₆ alkyl, R²⁴ is optionally substituted by one or more moieties selected from the group consisting of cycloalkyl, heterocyclyl, aryl, and heteroaryl, R²⁵ is selected from the group consisting of —H, alkyl, and alkoxy, and R²⁶ is selected from the group consisting of —H, —OH, alkyl, alkoxy, —C(O)—R³⁸, —C(O)—O—R³⁹, and —C(O)—S—R⁴⁰; wherein when R²⁵ and R²⁶ independently are alkyl or alkoxy, R²⁵ and R²⁶ independently are optionally substituted with one or more moieties selected from the group consisting of cycloalkyl, heterocyclyl, aryl, and heteroaryl; or R²⁵ is —H; and R²⁶ is selected from the group consisting of cycloalkyl, heterocyclyl, aryl, and heteroaryl, R²⁷, R²⁸, R²⁹, R³⁰, R³¹, R³², R³³, R³⁴, R³⁵, R³⁶, R³⁷, R³⁸, R³⁹, and R⁴⁰ independently are selected from the group consisting of —H and alkyl, wherein alkyl is optionally substituted by one or more moieties selected from the group consisting of cycloalkyl, heterocyclyl, aryl, and heteroaryl, wherein when any of R¹¹, R¹², R¹³, R¹⁴, R¹⁵, R¹⁶, R¹⁷, R¹⁸, R19⁹, R²⁰, R²¹, R²², R²³, R²⁴, R²⁵, R²⁶, R²⁷, R²⁸, R²⁹, R³⁰, R³¹, R³², R³³, R³⁴, R³⁵ R³⁶, R³⁷, R³⁸, R³⁹, and R⁴⁰ independently is a moiety selected from the group consisting of alkyl, alkenyl, alkynyl, alkoxy, alkylthio, cycloalkyl, heterocyclyl, aryl, and heteroaryl, then the moiety is optionally substituted by one or more substituent selected from the group consisting of —OH, alkoxy, and halogen;
a compound represented by Formula III 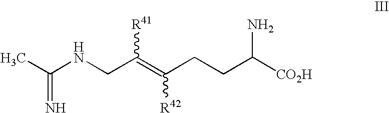
wherein:
R⁴¹ is H or methyl; and
R⁴² is H or methyl;
a compound of formula IV 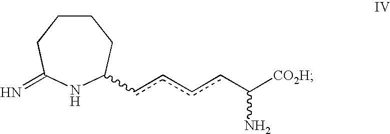
a compound of Formula V: 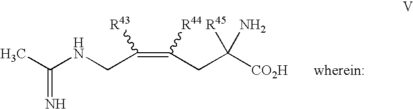
wherein:
R⁴³ is selected from the group consisting of hydrogen, halo, C₁-C₅ alkyl and C₁-C₅ alkyl substituted by alkoxy or one or more halo;
R⁴⁴ is selected from the group consisting of hydrogen, halo, C₁-C₅ alkyl and C₁-C₅ alkyl substituted by alkoxy or one or more halo;
R⁴⁵ is C₁-C₅ alkyl or C₁-C₅ alkyl be substituted by alkoxy or one or more halo;
a compound of Formula VI: 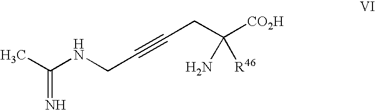
wherein:
R⁴⁶ is C₁-C₅ alkyl, said C₁-C₅ alkyl optionally substituted by halo or alkoxy, said alkoxy optionally substituted by one or more halo;
a compound of Formula VII 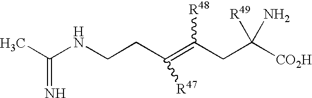
wherein:
R⁴⁷ is selected from the group consisting of hydrogen, halo, C₁-C₅ alkyl and C₁-C₅ alkyl substituted by alkoxy or one or more halo;
R⁴⁸ is selected from the group consisting of hydrogen, halo, C₁-C₅ alkyl and C₁-C₅ alkyl substituted by alkoxy or one or more halo;
R⁴⁹ is C₁-C₅ alkyl or C₁-C₅ alkyl be substituted by alkoxy or one or more halo;
a compound of Formula VIII 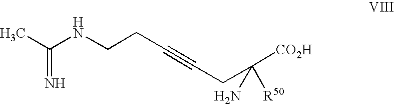
wherein:
R⁵⁰ is C₁-C₅ alkyl, said C₁-C₅ alkyl optionally substituted by halo or alkoxy, said alkoxy optionally substituted by one or more halo;
a compound of formula IX 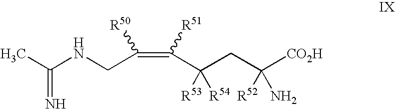
wherein:
R⁵⁰ is selected from the group consisting of hydrogen, halo, and C₁-C₅ alkyl, said C₁-C₅ alkyl optionally substituted by halo or alkoxy, said alkoxy optionally substituted by one or more halo;
R⁵¹ is selected from the group consisting of hydrogen, halo, and C₁-C₅ alkyl, said C₁-C₅ alkyl optionally substituted by halo or alkoxy, said alkoxy optionally substituted by one or more halo;
R⁵² is C₁-C₅ alkyl, said C₁-C₅ alkyl optionally substituted by halo or alkoxy, said alkoxy optionally substituted by one or more halo;
R⁵³ is selected from the group consisting of hydrogen, halo, and C₁-C₅ alkyl, said C₁-C₅ alkyl optionally substituted by halo or alkoxy, said alkoxy optionally substituted by one or more halo; and
R⁵⁴ is selected from the group consisting of halo and C₁-C₅ alkyl, said C₁-C₅ alkyl optionally substituted by halo or alkoxy, said alkoxy optionally substituted by one or more halo;
a compound of formula X 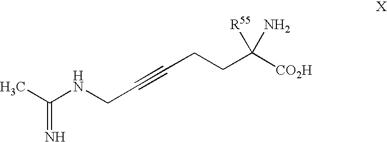
wherein:
R⁵⁵ is C₁-C₅ alkyl, said C₁-C₅ alkyl optionally substituted by halo or alkoxy, said alkoxy optionally substituted by one or more halo.
a compound having the formula XI 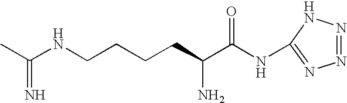
2S-amino-6-[(1-iminoethyl)amino]-N-(1H-tetrazol-5-yl) hexanamide, hydrate, dihydrochloride XI
A compound of formula XII: 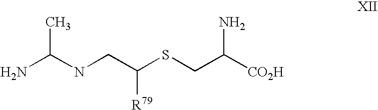
wherein R⁷⁹ is selected from C₁₋₄ alkyl, C₃₋₄ cycloalkyl, C₁₋₄ hydroxyalkyl, and C₁₋₄ haloalkyl;
a compound of Formula XIII, Formula XIV or Formula XV: 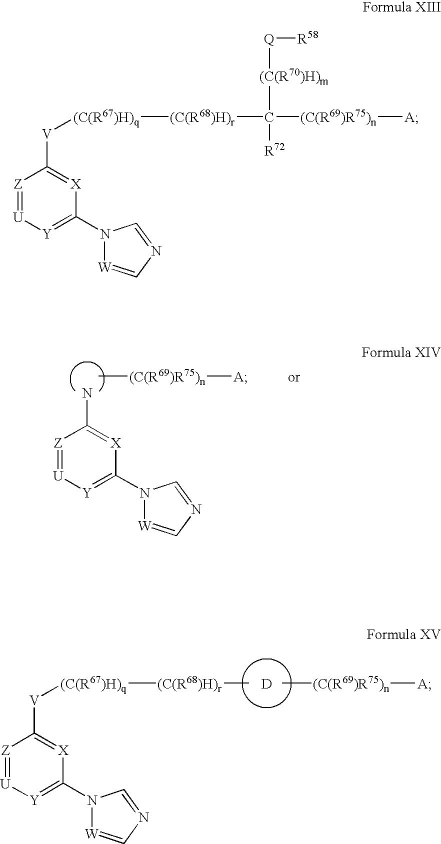
wherein:
A is —R⁵⁶, —OR⁵⁶, C(O)N(R⁵⁶)R⁵⁷, P(O)[N(R⁵⁶)R⁵⁷]₂, —N(R⁵⁶)C(O)R⁵⁷, —N(R⁷⁶)C(O)OR⁵⁶, —N(R⁵⁶)R⁷⁶,
—N(R⁷¹)C(O)N(R⁵⁶)R⁷¹, —S(O)ₜR⁵⁶, —SO₂NHC(O)R⁵⁶, —NHSO₂R⁷⁷, —SO₂NH(R⁵⁶)H, —C(O)NHSO₂R⁷⁷, and —CH═NOR⁵⁶;
each X, Y and Z are independently N or C(R¹⁹);
each U is N or C(R⁶⁰), provided that U is N only when X is N and Z and Y are CR⁷⁴;
V is N(R⁵⁹), S, O or C(R⁵⁹)H;
Each W is N or CH;
Q is chosen from the group consisting of a direct bond, —C(O)—, —O—, —C(═N—R⁵⁶)—, S(O)ₜ, and —N(R⁶¹)—;
m is zero or an integer from 1 to 4;
n is zero or an integer from 1 to 3;
q is zero or one;
r is zero or one, provided that when Q and V are heteroatoms, m, q, and r cannot all be zero;
when A is —OR⁵⁶, N(R⁵⁶)C(O)R⁵⁷, —N(R⁷¹)C(O)OR⁵⁷, —N(R⁵⁶)R⁷⁶, —N(R⁷¹)C(O)N(R⁵⁶)R⁷¹, —S(O)ₜR⁵⁶ (where t is zero), or —NHSO₂R⁷⁷, n, q, and r cannot all be zero; and when Q is a heteroatom and A is —OR⁵⁶, N(R⁵⁶)C(O)R⁵⁷, —N(R⁷¹)C(O)OR⁵⁷, —N(R⁵⁶)R⁷⁶, N(R⁷¹)C(O)N(R⁵⁶)R⁷¹, —S(O)ₜR⁵⁶ (when t is zero), or —NHSO₂R⁷⁷, m and n cannot both be zero;
t is zero, one or two; 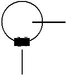
is an optionally substituted N-heterocyclyl; 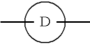
is an optionally substituted carbocyclyl or optionally substituted N-heterocyclyl;
each R⁵⁶ and R⁵⁷ are independently chosen from the group consisting of hydrogen, optionally substituted C₁-C₂₀ alkyl, optionally substituted cycloalkyl,
—[C₀-C₈ alkyl]-R⁶⁴, —[C₂-C₈ alkenyl]-R⁶⁴, —[C₂-C₈ alkynyl]-R⁶⁴, —[C₂-C₈ alkyl]-R⁶⁵ (optionally substituted by hydroxy), —[C₁-C₈]—R⁶⁶ (optionally substituted by hydroxy), optionally substituted heterocyclyl;
or R⁵⁶ and R⁵⁷ together with the nitrogen atom to which they are attached is an optionally substituted N-heterocyclyl;
R⁵⁸ is chosen from the group consisting of hydrogen, alkyl, cycloalkyl, optionally substituted aryl, haloalkyl, —[C₁-C₈ alkyl]—C(O)N(R⁵⁶)R⁵⁷, —[C₁-C₈ alkyl]- N(R⁵⁶)R⁵⁷, —[C₁-C₈ alkyl]-R⁶³, —[C₂-C₈ alk2yl]-R⁶⁵, —[C₁-C₈ alkyl]-R⁶⁶, and heterocyclyl (optionally substituted by one or more substitutents selected from the group consisting of halo, alkyl, alkoxy and imidazolyl);
or when Q is —N(R⁵⁸)— or a direct bond to R⁵⁸, R⁵⁸ may additionally be aminocarbonyl,
alkoxycarbonyl, alkylsulfonyl, monoalkylaminocarbonyl, dialkylaminocarbonyl and —C(═NR⁷³)—NH₂;
or -Q-R⁵⁸ taken together represents —C(O)OH, —C(O)N(R⁵⁶)R⁵⁷ or 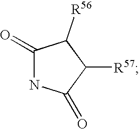
R⁵⁹ is chosen from the group consisting of hydrogen, alkyl, aryl, aralkyl and cycloalkyl;
Provided that when A is —R⁵⁶ or —OR⁵⁶, R⁵⁹ cannot be hydrogen, and when V is CH, R⁵⁹ may additionally be hydroxy;
R⁶⁰ is chosen from the group consisting of hydrogen, alkyl, aryl, aralkyl, haloalkyl,
optionally substituted aralkyl, optionally substituted aryl, —OR⁷¹, —S(O)ₜ—R⁷¹, N(R⁷¹)R⁷⁶, N(R⁷¹)C(O)N(R⁵⁶)R⁷¹, N(R⁷¹)C(O)OR⁷¹, N(R⁷¹)C(O) R⁷¹, —[C₀-C₈ alkyl]—C(H)[C(O)R⁷¹]₂ and —[C₀-C₈ alkyl]- C(O)N(R⁵⁶)R⁷¹;
R⁶¹ is chosen from the group consisting of hydrogen, alkyl, cycloalkyl, —[C₁-C₈ alkyl]-R⁶³, —[C₂-C₈]alkyl]-R⁶⁵, —[C₁-C₈ alkyl]-R⁶⁶, acyl, —C(O)R⁶³,
—C(O)— —[C₁-C₈ alkyl]-R⁶³, alkoxycarbonyl, optionally substituted aryloxycarbonyl, optionally substituted aralkoxycarbonyl, alkylsulfonyl, optionally substituted aryl, optionally substituted heterocyclyl, alkoxycarbonylalkyl, carboxyalkyl, optionally substituted arylsulfonyl, aminocarbonyl, monoalkylaminocarbonyl, dialkylaminocarbonyl, optionally substituted arylaminocarbonyl, aminosulfonyl, monoalkylaminosulfonyl dialkylaminosulfonyl, arylaminosulfonyl, arylsulfonylaminocarbonyl, optionally substituted N-heterocyclyl, —C(═NH)—N(CN)R⁵⁶, —C(O)R⁷⁸—N(R⁵⁶)R⁵⁷, —C(O)—N(R⁵⁶)R⁷⁸—C(OOR⁵⁶;
each R⁶³ and R⁶⁴ are independently chosen from the group consisting of haloalkyl,
cycloalkyl, (optionally substituted with halo, cyano, alkyl or alkoxy), carbocyclyl (optionally substituted with one or more substituents selected from the group consisting of halo, alkyl and alkoxy) and heterocyclyl (optionally substituted with alkyl, aralkyl or alkoxy);
each R⁶⁵ is independently chosen from the group consisting of halo, alkoxy, optionally
substituted aryloxy, optionally substituted aralkoxy, optionally substituted —S(O)ₜ—R⁷⁷, acylamino, amino, monoalkylamino, dialkylamino, (triphenylmethyl)amino, hydroxy, mercapto, alkylsulfonamido;
each R⁶⁶ is independently chosen from the group consisting of cyano, di(alkoxy)alkyl, carboxy, alkoxycarbonyl, aminocarbonyl, monoalkylaminocarbonyl and dialkylaminocarbonyl;
each R⁶⁷, R⁶⁸, R⁶⁹, R⁷⁰, R⁷², and R⁷⁵ are independently hydrogen or alkyl;
each R⁷¹ is independently hydrogen, alkyl, optionally substituted aryl, optionally substituted aralkyl or cycloalkyl;
R⁷³ is hydrogen, NO₂, or toluenesulfonyl;
each R⁷⁴ is independently hydrogen, alkyl (optionally substituted with hydroxy), cyclopropyl, halo or haloalkyl;
each R⁷⁶ is independently hydrogen, alkyl, cycloalkyl, optionally substituted aryl, optionally substituted aralkyl, —C(O)R⁷⁷ or —SO₂R⁷⁷;
or R⁷⁶ taken together with R⁵⁶ and the nitrogen to which they are attached is an optionally substituted N-heterocyclyl;
or R⁷⁶ taken together with R⁷¹ and the nitrogen to which they are attached is an optionally substituted N-heterocyclyl;
each R⁷⁷ is independently alkyl, cycloalkyl, optionally substituted aryl or optionally substituted aralkyl; and
R⁷⁸ is an amino acid residue; and 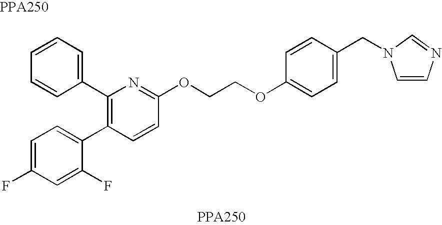
or a pharmaceutically acceptable salt or prodrug of any of said inducible nitric oxide synthase inhibitors.

7. The method according to claim 1 wherein the PDE inhibitor or pharmaceutically acceptable salt or prodrug thereof is selected from the group consisting of PDE-III inhibitors and PDE-IV inhibitors.

8. The method according to claim 1 wherein the PDE inhibitor or pharmaceutically acceptable salt or prodrug thereof is selected from the group consisting of PDE-III inhibitors.

9. The method according to claim 1 wherein the PDE inhibitor or pharmaceutically acceptable salt or prodrug thereof is selected from the group consisting of PDE-IV inhibitors.

10. The method according to claim 1 wherein the PDE inhibitor or pharmaceutically acceptable salt or prodrug thereof comprises Roflumilast having the following structure: 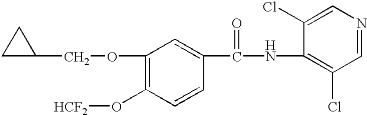
or a pharmaceutically acceptable salt or prodrug thereof.

11. The method according to claim 1 wherein the respiratory disease or condition is selected from the group consisting of allergen-induced asthma, exercise-induced asthma, pollution-induced asthma, cold-induced asthma, viral-induced-asthma, chronic bronchitis with normal airflow, chronic obstructive bronchitis, emphysema, asthmatic bronchitis, bullous disease, cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, post-cardiac surgery, acute pulmonary hypertension, persistent pulmonary hypertension of the newborn, perinatal aspiration syndrome, hyaline membrane disease, acute pulmonary thromboembolism, heparin-protamine reactions, sepsis, status asthmaticus and hypoxia.

12. The method according to claim 1 wherein the respiratory disease or condition is selected from the group consisting of an asthmatic condition and COPD.

13. The method of claim 1 wherein the respiratory condition is an asthmatic condition.

14. The method of claim 13 wherein the asthmatic condition is allergen-induced asthma.

15. The method of claim 13 wherein the asthmatic condition is pollution-induced asthma.

16. The method of claim 13 wherein the asthmatic condition is exercise-induced asthma.

17. The method of claim 13 wherein the asthmatic condition is viral-induced asthma.

18. The method of claim 13 wherein the asthmatic condition is cold-induced asthma.

19. The method of claim 1 wherein the respiratory condition is chronic obstructive pulmonary disease (COPD).

20. The method of claim 1 wherein the respiratory condition is emphysema.

21. The method of claim 1 wherein the respiratory condition is chronic bronchitis.

22. The method of claim 21 wherein the respiratory condition is chronic bronchitis with normal airflow.

23. The method of claim 21 wherein the respiratory condition is chronic obstructive bronchitis.

24. The method of claim 1 wherein the respiratory condition is asthmatic bronchitis.

25. The method of claim 1 wherein the respiratory condition is bullous disease.

26. The method of claim 1 wherein the respiratory condition is cystic fibrosis.

27. The method of claim 1 wherein the respiratory condition is bronchiectasis.

28. The method according to claim 1 wherein administering an iNOS selective inhibitor or pharmaceutically acceptable salt or prodrug thereof and a PDE inhibitor or pharmaceutically acceptable salt or prodrug thereof comprises administering to the subject orally, by inhalation, enterally or parenterally in at least one dose per day.

29. The method according to claim 1 wherein the iNOS selective inhibitor or pharmaceutically acceptable salt or prodrug thereof and the PDE inhibitor or pharmaceutically acceptable salt or prodrug thereof are administered to the subject substantially simultaneously.

30. The method according to claim 1 wherein the iNOS selective inhibitor or pharmaceutically acceptable salt or prodrug thereof and the PDE inhibitor or pharmaceutically acceptable salt or prodrug thereof are administered to the subject sequentially.

31. A method for the treatment, prevention or inhibition of a respiratory disease or condition having an inflammatory component in a subject in need of such treatment, prevention or inhibition, said method comprising administering to the subject a dose of an iNOS selective inhibitor or pharmaceutically acceptable salt or prodrug thereof and a dose of a PDE inhibitor or pharmaceutically acceptable salt or prodrug thereof, wherein together the dose of the iNOS selective inhibitor or pharmaceutically acceptable salt or prodrug thereof and the dose of the PDE inhibitor or pharmaceutically acceptable salt or prodrug thereof constitute a therapeutically effective dose for the treatment, prevention or inhibition of the respiratory disease or condition.

32. A composition for the treatment, prevention or inhibition of a respiratory disease or condition in a subject in need of such treatment, prevention or inhibition comprising an amount of an iNOS selective inhibitor or pharmaceutically acceptable salt or prodrug thereof and an amount of a PDE inhibitor or pharmaceutically acceptable salt or prodrug thereof.

33. A composition according to claim 32 wherein the amount of the iNOS selective inhibitor or pharmaceutically acceptable salt or prodrug thereof and the amount of the PDE inhibitor or pharmaceutically acceptable salt or prodrug thereof together constitute a respiratory diease or condition suppression, prevention or inhibition effective amount.

34. A composition according to claim 32 further comprising a pharmaceutically acceptable aerosolizing agent for aerosolizing the composition for delivery to the subject by inhalation.

35. A composition according to claim 32 wherein the iNOS selective inhibitor is represented by the formula: 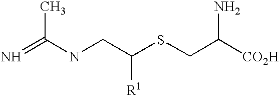
wherein R¹ is selected from C₁₋₄ alkyl, C₃₋₄ cycloalkyl, C₁₋₄ hydroxyalkyl, and C₁₋₄ haloalkyl.
or a pharmaceutically acceptable salt thereof.

36. The method of claim 4 wherein said iNOS inhibitor is selected from the group consisting of:
S—((R)-2-(1-iminoethylarmino)propyl)-L-cysteine;
S—((S)-2-(1-iminoethylamino)propyl)-L-cysteine;
S—((R/S)-2-(1-iminoethylamino)propyl)-L-cysteine;
S—((R)-2-(1-iminoethylamino)propyl)-D-cysteine;
S—((S)-2-(1-iminoethylamino)propyl)-D-cysteine;
S—((R/S)-2-(1-iminoethylamino)propyl)-D-cysteine;
S—((R/S)-2-(1-iminoethylamino)butyl)-L-cysteine;
S—((R/S)-2-(1-iminoethylamino,2-cyclopropyl)ethyl)-L-cysteine; and
S—((R/S)-2-(1-iminoethylamino,3-hydroxy)propyl)-L-cysteine,
or a pharmaceutically acceptable salt, solvate, or physiologically functional derivative thereof.

37. A composition according to claim 32 wherein the iNOS selective inhibitor is selected from the group consisting of:
a compound having Formula I 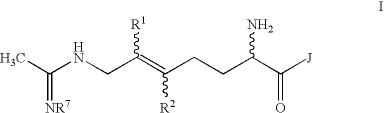
wherein:
R¹ is selected from the group consisting of H, halo and alkyl which may be optionally substituted by one or more halo;
R² is selected from the group consisting of H, halo and alkyl which may be optionally substituted by one or more halo; with the proviso that at least one of R¹ or R² contains a halo;
R⁷ is selected from the group consisting of H and hydroxy;
J is selected from the group consisting of hydroxy, alkoxy, and NR³R⁴ wherein;
R³ is selected from the group consisting of H, lower alkyl, lower alkylenyl and lower alkynyl;
R⁴ is selected from the group consisting of H, and a heterocyclic ring in which at least one member of the ring is carbon and in which 1 to about 4 heteroatoms are independently selected from oxygen, nitrogen and sulfur and said heterocyclic ring may be optionally substituted with heteroarylamino, N-aryl-N-alkylamino, N-heteroarylamino-N-alkylamino, haloalkylthio, alkanoyloxy, alkoxy, heteroaralkoxy, cycloalkoxy, cycloalkenyloxy, hydroxy, amino, thio, nitro, lower alkylamino, alkylthio, alkylthioalkyl, arylamino, aralkylamino, arylthio, alkylsulfinyl, alkylsulfonyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, alkanoyl, alkenoyl, aroyl, heteroaroyl, aralkanoyl, heteroaralkanoyl, haloalkanoyl, alkyl, alkenyl, alkynyl, alkylenedioxy, haloalkylenedioxy, cycloalkyl, cycloalkenyl, lower cycloalkylalkyl, lower cycloalkenylalkyl, halo, haloalkyl, haloalkoxy, hydroxyhaloalkyl, hydroxyaralkyl, hydroxyalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, cyanoalkyl, dicyanoalkyl, carboxamidoalkyl, dicarboxamidoalkyl, cyanocarboalkoxyalkyl, carboalkoxyalkyl, dicarboalkoxyalkyl, cyanocycloalkyl, dicyanocycloalkyl, carboxamidocycloalkyl, dicarboxamidocycloalkyl, carboalkoxycya nocycloalkyl, carboalkoxycycloalkyl, dicarboalkoxycycloalkyl, formylalkyl, acylalkyl, dialkoxyphosphonoalkyl, diaralkoxyphosphonoalkyl, phosphonoalkyl, dialkoxyphosphonoalkoxy, diaralkoxyphosphonoalkoxy, phosphonoalkoxy, dialkoxyphosphonoalkylamino, diaralkoxyphosphonoalkylamino, phosphonoalkylamino, dialkoxyphosphonoalkyl, diaralkoxyphosphonoalkyl, guanidino, amidino, and acylamino;
a compound having a structure corresponding to Formula II 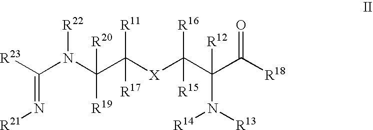
wherein X is selected from the group consisting of —S—, —S(O)—, and —S(O)₂—, R¹² is selected from the group consisting of C₁-C₆ alkyl, C₂-C₆ alkenyl, C₂-C₆ alkynyl, C₁-C₅ alkoxy-C₁ alkyl, and C₁-C₅ alkylthio-C₁ alkyl wherein each of these groups is optionally substituted by one or more substituent selected from the group consisting of —OH, alkoxy, and halogen, R¹⁸ is selected from the group consisting of —OR²⁴ and —N(R²⁵)(R²⁶), and R¹³ is selected from the group consisting of —H, —OH, —C(O)—R²⁷, —C(O)—O—R²⁸, and —C(O)—S—R²⁹; or R¹⁸ is —N(R³⁰)—, and R¹³ is —C(O)—, wherein R¹⁸ and R¹³ together with the atoms to which they are attached form a ring; or R¹⁸ is —O—, and R¹³ is —C(R³¹)(R³²)—, wherein R¹⁸ and R¹³ together with the atoms to which they are attached form a ring, wherein if R¹³ is —C(R³²¹)(R³²)—, then R¹⁴ is —C(O)—O—R³³; otherwise R¹⁴ is —H, R¹¹, R¹⁵, R¹⁶, and R¹⁷ independently are selected from the group consisting of —H, halogen, C₁-C₆ alkyl, C₂-C₆ alkenyl, C₂-C₆ alkynyl, and C₁-C₅ alkoxy-C₁ alkyl, R¹⁹ and R²⁰ independently are selected from the group consisting of —H, C₁-C₆ alkyl, C₂-C₆ alkenyl, C₂-C₆ alkynyl, and C₁-C₅ alkoxy-C₁ alkyl, R²¹ is selected from the group consisting of —H, —OH, —C(O)—O—R³⁴, and —C(O)—S—R³⁵, and R²² is selected from the group consisting of —H, —OH, —C(O)—O—R³⁶, and —C(O)—S—R³⁷; or R²¹ is —O—, and R²² is —C(O)—, wherein R²¹ and R²² together with the atoms to which they are attached form a ring; or R²¹ is —C(O)—, and R²² is —O—, wherein R²¹ and R²² together with the atoms to which they are attached form a ring, R²³ is C₁ alkyl, R²⁴ is selected from the group consisting of —H and C₁-C₆ alkyl, wherein when R²⁴ is C₁-C₆ alkyl, R²⁴ is optionally substituted by one or more moieties selected from the group consisting of cycloalkyl, heterocyclyl, aryl, and heteroaryl, R²⁵ is selected from the group consisting of —H, alkyl, and alkoxy, and R²⁶ is selected from the group consisting of —H, —OH, alkyl, alkoxy, —C(O)—R³⁸, —C(O)—O—R³⁹, and —C(O)—S—R⁴⁰; wherein when R²⁵ and R²⁶ independently are alkyl or alkoxy, R²⁵ and R²⁶ independently are optionally substituted with one or more moieties selected from the group consisting of cycloalkyl, heterocyclyl, aryl, and heteroaryl; or R²⁵ is —H; and R²⁶ is selected from the group consisting of cycloalkyl, heterocyclyl, aryl, and heteroaryl, R²⁷, R²⁸, R²⁹, R³⁰, R³¹, R³², R³³, R³⁴, R³⁵, R³⁶, R³⁷, R³⁸, R³⁹, and R⁴⁰ independently are selected from the group consisting of —H and alkyl, wherein alkyl is optionally substituted by one or more moieties selected from the group consisting of cycloalkyl, heterocyclyl, aryl, and heteroaryl, wherein when any of R¹¹, R¹², R¹³, R¹⁴, R¹⁵, R¹⁶, R¹⁷, R¹⁸, R19⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, and R⁴⁰ independently is a moiety selected from the group consisting of alkyl, alkenyl, alkynyl, alkoxy, alkylthio, cycloalkyl, heterocyclyl, aryl, and heteroaryl, then the moiety is optionally substituted by one or more substituent selected from the group consisting of —OH, alkoxy, and halogen;
a compound represented by Formula III 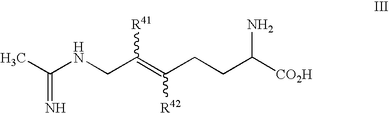
wherein:
R⁴¹ is H or methyl; and
R⁴² is H or methyl;
a compound of formula IV 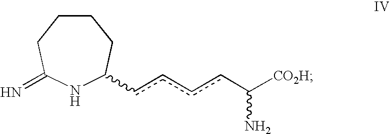
a compound of Formula V: 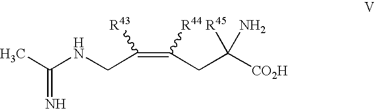
wherein:
R⁴³ is selected from the group consisting of hydrogen, halo, C₁-C₅ alkyl and C₁-C₅ alkyl substituted by alkoxy or one or more halo;
R⁴⁴ is selected from the group consisting of hydrogen, halo, C₁-C₅ alkyl and C₁-C₅ alkyl substituted by alkoxy or one or more halo;
R⁴⁵ is C₁-C₅ alkyl or C₁-C₅ alkyl be substituted by alkoxy or one or more halo;
a compound of Formula VI: 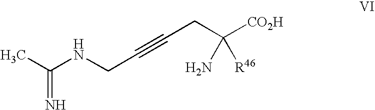
wherein:
R⁴⁶ is C₁-C₅ alkyl, said C₁-C₅ alkyl optionally substituted by halo or alkoxy, said alkoxy optionally substituted by one or more halo;
a compound of Formula VII 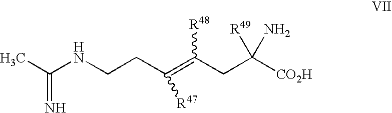
wherein:
R⁴⁷ is selected from the group consisting of hydrogen, halo, C₁-C₅ alkyl and C₁-C₅ alkyl substituted by alkoxy or one or more halo;
R⁴⁸ is selected from the group consisting of hydrogen, halo, C₁-C₅ alkyl and C₁-C₅ alkyl substituted by alkoxy or one or more halo;
R⁴⁹ is C₁-C₅ alkyl or C₁-C₅ alkyl be substituted by alkoxy or one or more halo;
a compound of Formula VIII 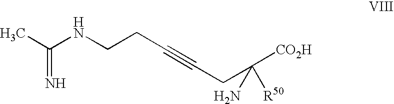
wherein:
R⁵⁰ is C₁-C₅ alkyl, said C₁-C₅ alkyl optionally substituted by halo or alkoxy, said alkoxy optionally substituted by one or more halo;
a compound of formula IX 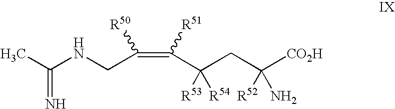
wherein:
R⁵⁰ is selected from the group consisting of hydrogen, halo, and C₁-C₅ alkyl, said C₁-C₅ alkyl optionally substituted by halo or alkoxy, said alkoxy optionally substituted by one or more halo;
R⁵¹ is selected from the group consisting of hydrogen, halo, and C₁-C₅ alkyl, said C₁-C₅ alkyl optionally substituted by halo or alkoxy, said alkoxy optionally substituted by one or more halo;
R⁵² is C₁-C₅ alkyl, said C₁-C₅ alkyl optionally substituted by halo or alkoxy, said alkoxy optionally substituted by one or more halo;
R⁵³ is selected from the group consisting of hydrogen, halo, and C₁-C₅ alkyl, said C₁-C₅ alkyl optionally substituted by halo or alkoxy, said alkoxy optionally substituted by one or more halo; and
R⁵⁴ is selected from the group consisting of halo and C₁-C₅ alkyl, said C₁-C₅ alkyl optionally substituted by halo or alkoxy, said alkoxy optionally substituted by one or more halo;
a compound of formula X 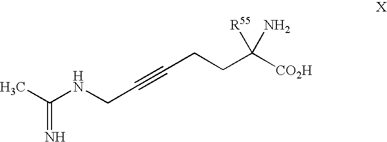
wherein:
R⁵⁵ is C₁-C₅ alkyl, said C₁-C₅ alkyl optionally substituted by halo or alkoxy, said alkoxy optionally substituted by one or more halo.
a compound having the formula XI 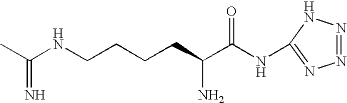
2S-amino-6-[(1-iminoethyl)amino]-N-(1H-tetrazol-5-yl) hexanamide, hydrate, dihydrochloride XI
A compound of formula XII: 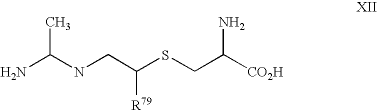
wherein R⁷⁹ is selected from C₁₋₄ alkyl, C₃₋₄ cycloalkyl, C₁₋₄ hydroxyalkyl, and C₁₋₄ haloalkyl;
a compound of Formula XIII, Formula XIV or Formula XV: 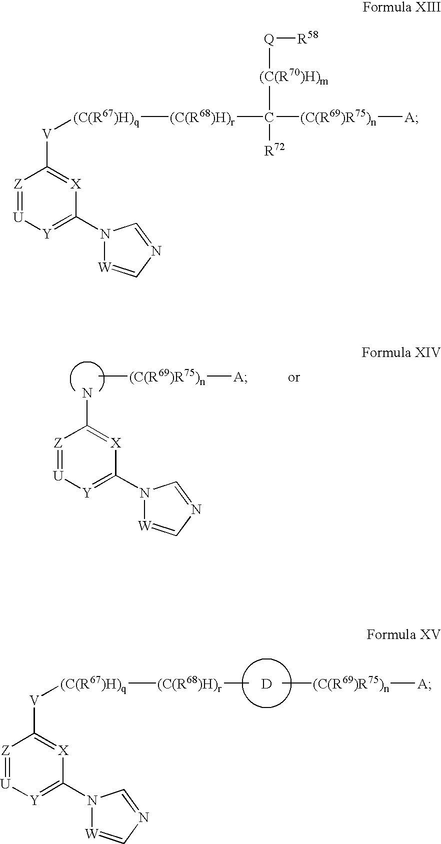
wherein:
A is —R⁵⁶, —OR⁵⁶, C(O)N(R⁵⁶)R⁵⁷, P(O)[N(R⁵⁶)R⁵⁷]₂, —N(R⁵⁶)C(O)R⁵⁷, —N(R⁷⁶)C(O)OR⁵⁶, —N(R⁵⁶)R⁷⁶,
—N(R⁷¹)C(O)N(R⁵⁶)R⁷¹, —S(O)ₜR⁵⁶, —SO₂NHC(O)R⁵⁶, —NHSO₂R⁷⁷, —SO₂NH(R⁵⁶)H, —C(O)NHSO₂R⁷⁷, and —CH═NOR⁵⁶;
each X, Y and Z are independently N or C(R¹⁹);
each U is N or C(R⁶⁰), provided that U is N only when X is N and Z and Y are CR⁷⁴;
V is N(R⁵⁹), S, O or C(R⁵⁹)H;
Each W is N or CH;
Q is chosen from the group consisting of a direct bond, —C(O)—, —O—, —C(═N—R⁵⁶)—, S(O)ₜ, and —N(R⁶¹)—;
m is zero or an integer from 1 to 4;
n is zero or an integer from 1 to 3;
q is zero or one;
r is zero or one, provided that when Q and V are heteroatoms, m, q, and r cannot all be zero;
when A is —OR⁵⁶, N(R⁵⁶)C(O)R⁵⁷, —N(R⁷¹)C(O)OR⁵⁷, —N(R⁵⁶)R⁷⁶, —N(R⁷¹)C(O)N(R⁵⁶)R⁷¹, —S(O)ₜR⁵⁶ (where t is zero), or —NHSO₂R⁷⁷, n, q, and r cannot all be zero; and when Q is a heteroatom and A is —OR⁵⁶, N(R⁵⁶)C(O)R⁵⁷, —N(R⁷¹)C(O)OR⁵⁷, —N(R⁵⁶)R⁷⁶, N(R⁷¹)C(O)N(R⁵⁶)R⁷¹, —S(O)ₜR⁵⁶ (when t is zero), or —NHSO₂R⁷⁷, m and n cannot both be zero;
t is zero, one or two; 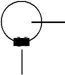
is an optionally substituted N-heterocyclyl; 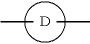
is an optionally substituted carbocyclyl or optionally substituted N-heterocyclyl;
each R⁵⁶ and R⁵⁷ are independently chosen from the group consisting of hydrogen, optionally substituted C₁-C₂₀ alkyl, optionally substituted cycloalkyl,
—[C₀-C₈ alkyl]-R⁶⁴, —[C₂-C₈ alkenyl]-R⁶⁴, —[C₂-C₈ alkynyl]-R⁶⁴, —[C₂-C₈ alkyl]-R⁶⁵ (optionally substituted by hydroxy), —[C₁-C₈]-R⁶⁶ (optionally substituted by hydroxy), optionally substituted heterocyclyl;
or R⁵⁶ and R⁵⁷ together with the nitrogen atom to which they are attached is an optionally substituted N-heterocyclyl;
R⁵⁸ is chosen from the group consisting of hydrogen, alkyl, cycloalkyl, optionally substituted aryl, haloalkyl, —[C₁-C₈ alkyl]—C(O)N(R⁵⁶)R⁵⁷, —[C₁-C₈ alkyl]- N(R⁵⁶)R⁵⁷, —[C₁-C₈ alkyl]-R⁶³, —[C₂-C₈ alk2yl]-R⁶⁵, —[C₁-C₈ alkyl]-R⁶⁶, and heterocyclyl (optionally substituted by one or more substitutents selected from the group consisting of halo, alkyl, alkoxy and imidazolyl);
or when Q is —N(R⁵⁸)— or a direct bond to R⁵⁸, R⁵⁸ may additionally be aminocarbonyl, alkoxycarbonyl, alkylsulfonyl, monoalkylaminocarbonyl, dialkylaminocarbonyl and —C(═NR⁷³)—NH₂;
or -Q-R⁵⁸ taken together represents —C(O)OH, —C(O)N(R⁵⁶)R⁵⁷ or 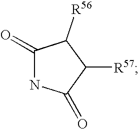
R⁵⁹ is chosen from the group consisting of hydrogen, alkyl, aryl, aralkyl and cycloalkyl;
Provided that when A is —R⁵⁶ or —OR⁵⁶, R⁵⁹ cannot be hydrogen, and when V is CH, R⁵⁹ may additionally be hydroxy;
R⁶⁰ is chosen from the group consisting of hydrogen, alkyl, aryl, aralkyl, haloalkyl,
optionally substituted aralkyl, optionally substituted aryl, —OR⁷¹, —S(O)ₜ—R⁷¹, N(R⁷¹)R⁷⁶, N(R⁷¹)C(O)N(R⁵⁶)R⁷¹, N(R⁷¹)C(O)OR⁷¹, N(R⁷¹)C(O) R⁷¹, —[C₀-C₈ alkyl]-C(H)[C(O)R⁷¹]₂ and —[C₀-C₈ alkyl]- C(O)N(R⁵⁶)R⁷¹;
R⁶¹ is chosen from the group consisting of hydrogen, alkyl, cycloalkyl, —[C₁-C₈ alkyl]-R , —[C₂-C₈]alkyl]-R⁶⁵, —[C₁-C₈ alkyl]-R⁶⁶, acyl, —C(O)R⁶³, —C(O)— —[C₁-C₈ alkyl]-R⁶³, alkoxycarbonyl, optionally substituted aryloxycarbonyl, optionally substituted aralkoxycarbonyl, alkylsulfonyl, optionally substituted aryl, optionally substituted heterocyclyl, alkoxycarbonylalkyl, carboxyalkyl, optionally substituted arylsulfonyl, aminocarbonyl, monoalkylaminocarbonyl, dialkylaminocarbonyl, optionally substituted arylaminocarbonyl, aminosulfonyl, monoalkylaminosulfonyl dialkylaminosulfonyl, arylaminosulfonyl, arylsulfonylaminocarbonyl, optionally substituted N-heterocyclyl, —C(═NH)—N(CN)R⁵⁶, —C(O)R⁷⁸—N(R⁵⁶)R⁵⁷, —C(O)—N(R⁵⁶)R⁷⁸—C(O)OR⁵⁶;
each R⁶³ and R⁶⁴ are independently chosen from the group consisting of haloalkyl, cycloalkyl, (optionally substituted with halo, cyano, alkyl or alkoxy), carbocyclyl (optionally substituted with one or more substituents selected from the group consisting of halo, alkyl and alkoxy) and heterocyclyl (optionally substituted with alkyl, aralkyl or alkoxy);
each R⁶⁵ is independently chosen from the group consisting of halo, alkoxy, optionally substituted aryloxy, optionally substituted aralkoxy, optionally substituted —S(O)ₜ—R⁷⁷, acylamino, amino, monoalkylamino, dialkylamino, (triphenylmethyl)amino, hydroxy, mercapto, alkylsulfonamido;
each R⁶⁶ is independently chosen from the group consisting of cyano, di(alkoxy)alkyl, carboxy, alkoxycarbonyl, aminocarbonyl, monoalkylaminocarbonyl and dialkylaminocarbonyl;
each R⁶⁷, R⁶⁸, R⁶⁹, R⁷⁰, R⁷², and R⁷⁵ are independently hydrogen or alkyl;
each R⁷¹ is independently hydrogen, alkyl, optionally substituted aryl, optionally substituted aralkyl or cycloalkyl;
R⁷³ is hydrogen, NO₂, or toluenesulfonyl;
each R⁷⁴ is independently hydrogen, alkyl (optionally substituted with hydroxy), cyclopropyl, halo or haloalkyl;
each R⁷⁶ is independently hydrogen, alkyl, cycloalkyl, optionally substituted aryl, optionally substituted aralkyl, —C(O)R⁷⁷ or —SO₂R⁷⁷;
or R⁷⁶ taken together with R⁵⁶ and the nitrogen to which they are attached is an optionally substituted N-heterocyclyl;
or R⁷⁶ taken together with R⁷¹ and the nitrogen to which they are attached is an optionally substituted N-heterocyclyl;
each R⁷⁷ is independently alkyl, cycloalkyl, optionally substituted aryl or optionally substituted aralkyl; and
R⁷⁸ is an amino acid residue; and 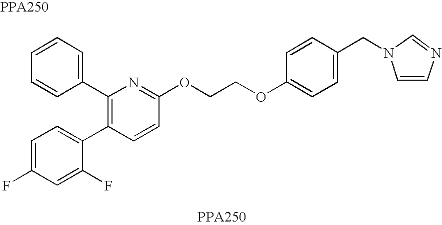
or a pharmaceutically acceptable salt or prodrug of any of said inducible nitric oxide synthase inhibitors.

38. The composition according to claim 32 wherein the PDE inhibitor or pharmaceutically acceptable salt or prodrug thereof is selected from the group consisting of PDE-III inhibitors.

39. The composition according to claim 32 wherein the PDE inhibitor or pharmaceutically acceptable salt or prodrug thereof is selected from the group consisting of PDE-IV inhibitors.

40. The composition according to claim 32 wherein the PDE inhibitor or pharmaceutically acceptable salt or prodrug thereof is selected from the group consisting of PDE-III/IV dual inhibitors.

41. The composition according to claim 32 wherein the PDE inhibitor or pharmaceutically acceptable salt or prodrug thereof comprises Roflumilast having the following structure: 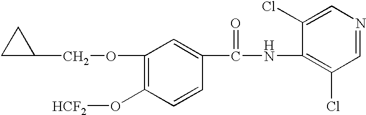
or a pharmaceutically acceptable salt or prodrug thereof.

42. A kit for treating, preventing or inhibiting a respiratory disease or condition in a subject in need of such treatment, prevention or inhibition comprising a first dosage form comprising an iNOS selective inhibitor or pharmaceutically acceptable salt or prodrug thereof and a second dosage form comprising a PDE inhibitor or pharmaceutically acceptable salt or prodrug thereof, wherein together the dosages comprise a therapeutically effective amount of the iNOS selective inhibitor or pharmaceutically acceptable salt or prodrug thereof and the PDE inhibitor or pharmaceutically acceptable salt or prodrug thereof for the treatment, prevention or inhibition of the respiratory disease or condition.

43. The kit of claim 42, wherein the iNOS selective inhibitor or pharmaceutically acceptable salt or prodrug thereof and the PDE inhibitor or pharmaceutically acceptable salt or prodrug thereof are in separate dosage forms.

44. The kit of claim 42, wherein iNOS selective inhibitor or pharmaceutically acceptable salt or prodrug thereof and the PDE inhibitor or pharmaceutically acceptable salt or prodrug thereof are in a single dosage form.

45. The kit of claim 42, further comprising an inhaler device.

46. The kit of claim 42, further comprising a nebulizer.
